(19) United States
(12) Patent Application Publication
(10) Pub. No.: US 2004/0048311 A1
(43) Pub. Date: Mar. 11, 2004

(54) Use of collections of binding sites for sample profiling and other applications

(76) Inventors: Dana Ault-Riche, Los Gatos, CA (US); Paul D. Kassner, San Mateo, CA (US)

Correspondence Address: HELLER EHRMAN WHITE & MCAULIFFE LLP, 4350 LA JOLLA VILLAGE DRIVE, 7TH FLOOR, SAN DIEGO, CA, 92122-1246, US

(21) Appl. No.: 10/351,891

(22) Filed: Jan. 24, 2003

Related U.S. Application Data
(60) Provisional application No. 60/352,011, filed on Jan. 24, 2002.

(51) Int. Cl. G01N 33/53; G01N 33/543

(52) U.S. Cl. 435/7.1; 436/518

(57) ABSTRACT

Provided are the use of collections of binding proteins, called capture agents, and their cognate binding partners, called tagged protein libraries, herein for profiling samples. Methods for generating the capture agents, tagged protein libraries and samples are also provided.

### RELATED APPLICATIONS

Benefit of priority under 35 U.S.C. §119(e) to U.S. provisional application Serial No. 60/352,011, filed Jan. 24, 2002, to Ault-Riche, et al., entitled “USE OF COLLECTIONS OF BINDING PROTEINS AND TAGS FOR SAMPLE PROFILING” is claimed.

This application is related to U.S. application Ser. No. 09/910,120, filed Jul. 18, 2001, to Dana Ault-Riche and Paul D. Kassner, entitled “COLLECTIONS OF BINDING PROTEINS AND TAGS AND USES THEREOF FOR NESTED SORTING AND HIGH THROUGHPUT SCREENING”, to U.S. provisional application Serial No. 60/219,183, filed Jul. 19, 2000, to Dana Ault-Riche entitled “COLLECTIONS OF ANTIBODIES FOR NESTED SORTING AND HIGH THROUGHPUT SCREENING”, and to International PCT application No. WO 02/06834. This application is also related to U.S. provisional application Serial No. 60/422,923, filed Oct. 30, 2002, to Dana Ault-Riche and Bruce Atkinson, entitled “METHODS FOR PRODUCING POLYPEPTIDE-TAGGED COLLECTIONS AND CAPTURE SYSTEMS CONTAINING THE TAGGED POLYPEPTIDES”, and to provisional U.S. application Serial No. 60/423,018, filed Oct. 30, 2002 to Dana Ault-Riche, Bruce Atkinson, Krishnanand Kumble, Lynne Jersaitis and Gizette Sperinde entitled “SYSTEMS FOR CAPTURE AND ANALYSIS OF BIOLOGICAL PARTICLES AND METHODS USING THE SYSTEMS”. This application is also related to PCT International Application Attorney docket no. 25885-1753PC, filed this same day to Ault-Riche et al., entitled “USE OF COLLECTIONS OF BINDING PROTEINS AND TAGS FOR SAMPLE PROFILING”. The subject matter of each of the above-noted applications and provisional applications is incorporated in its entirety by reference thereto.

### FIELD OF INVENTION

The present invention relates to collections of binding proteins, called capture agents herein, and methods of use thereof for profiling samples. The methods and collection technology integrate robotic high throughput screening and array and related techniques.

### BACKGROUND

There are a multitude of technologies designed to gather biological information on a faster and faster scale. Robotics and miniaturization technologies lead to advances in the rate at which information on complex samples is generated. High throughput screening technologies permit routine analysis of tens of thousands of samples; microfluidics and DNA microarray technologies permit information from a single sample to be gathered in a massively parallel manner. DNA arrays, such as microarray chips, simultaneously can measure the quantity of more than 10,000 different RNA molecules in a sample in a single experiment.

The sequencing of the human genome has led to the identification of approximately 30,000 genes. These 30,000 genes can generate many-fold greater diversity in message RNA transcripts through alternate splicing reactions. Even more diversity is created through processing of the message RNA into proteins and further post-translational modifications. The combination of these chemical processes (alternative RNA splicing, protein processing and post-translational modifications) increase the diversity of chemical entities into the millions. New tools are therefore needed to begin to understand this molecular complexity.

The chemical environment of a cell is largely controlled by the proteins in the cell. Therefore, information about the abundance, modification state, and activity of the proteins in a cellular sample is extremely valuable in understanding cellular biology. This information is needed to develop new pharmaceuticals and better diagnostic tests for the treatment of disease. DNA microarray technologies provide tools for measuring the abundance of messenger RNA in a sample. There is little correlation between the abundance of messenger RNA for a given protein and the amount of actual protein in the sample. DNA microarrays provide no information about the abundance, modification state or activities of the proteins in a sample.

A core practice of biochemistry is the separation of complex solutions and the detection of the separated materials. In chromatography, complex solutions are bound to a solid support and then separated by differential elution. The eluted material is then detected by spectroscopic techniques such as UV and visible light absorption or mass spectrometry. In immuno-chromotography, a complex solution is exposed to a solid support containing a single antibody. The specificity of the molecular interactions between the antibodies and the chemical entities in the sample solution that bind to the antibody (antigen) can permit a single chemical entity to be separated from a very complex sample.

Proteomics, the large-scale parallel study of proteins, is built upon technologies that simultaneously separate and detect multiple proteins in a solution. The need for technologies that allow highly parallel quantitation of specific proteins in a rapid, low-cost and low-sample-volume format has become increasingly apparent with the growing recognition of the importance of global approaches to molecular characterization of physiology, development, and disease (Abbott Nature 402: 715-720 (1999); and Humphrey-Smith et al. J. Protein Chem. 16: 537-544 (1997)). The ability to quantitate multiple proteins simultaneously has applications in basic biological research, molecular classification and diagnosis of disease, identification of therapeutic markers and targets, and profiling of response to toxins and pharmaceuticals. Many standard assays are amenable to parallel analysis in microtiter plates, but sample and reagent consumption can be prohibitive in large-scale studies. Two-dimensional gels are now widely used for large-scale protein analysis in cancer research (Emmert-Buck et al. Mol. Carcinog. 27: 158-165 (2000) and other areas of biology (Pandey et al. Nature 405: 837-846 (2000)). Two-dimensional gels have been used to separate and visualize 2,000-10,000 proteins in a single experiment (Rabilloud Anal. Chem. 72: 48A-55A (2000)), and subsequent excision of protein bands and detection by mass spectrometry can enable identification of the proteins (Patterson et al. Electrophoresis 16: 1791-1814 (1995)).

Ordered arrays of peptides and proteins provide the basis of another strategy for parallel protein analysis. DNA arrays have demonstrated the effectiveness of this approach in many areas of biological research (see, e.g., Khan et al. Biochim. Biophys. Acta 1423: M17-M28 (1999); DeRisi et al. Nat. Genet. 14: 457-460 (1996); and Debouck et al. Nat. Genet. 21: 48-50 (1999)). Protein assays using ordered arrays have been explored since the development of multipin synthesis (Geysen et al. Proc. Natl. Acad. Sci. USA 81: 3998-4002 (1984)) and spot synthesis (Frank Tetrahedron 48: 9217-9232 (1992) of peptides on cellulose supports. Protein arrays on membranes have been used to screen binding specificities of a protein expression library (Buessow et al. Nuc. Acid Res. 26: 5007-5008 (1998); Lueking et al. Anal. Biochem. 270: 103-111 (1999); and Buessow et al. Genomics 65: 1-6 (2000)) and to detect DNA-, RNA-, and protein-binding targets (Ge Nuc. Acids Res. 28: e3 (2000)). Arrays of clones from phage-display libraries can be probed with an antigen-coated filter for high-throughput antibody screening (de Wildt et al. Nature Biotechnology 18: 989-994 (2000)). Antibodies bound to glass can be used as a flow-cell array immunosensor (Rowe et al. Anal. Chem. 71: 433-439 (1999)), and antibodies spotted into glass-bottom microwells have been used for miniaturized, high-throughput ELISA (Mendoza et al. Biotechniques 27: 778-788 (1999)). Multiple antigens and antibodies have been patterned onto polystyrene using a desktop jet printer (Silzel et al. Clinical Chemistry 44: 2036-2043 (1998)) and onto glass by covalent attachment to polyacrylamide gel pads (Arenkov et al. Anal. Biochem. 278: 123-131 (2000)) for parallel immunoassays. Proteins covalently attached to glass slides through aldehyde-containing silane reagents have been used to detect protein-protein interactions, enzymatic targets, and protein-small molecule interactions (MacBeath et al. Science 289: 1760-1763 (2000)).

Other approaches employ microarrays of antibodies. In these antibodies of known specificity are arrayed at discrete physical locations on a solid surface and reacted with antigen-containing mixtures. Unbound material is washed off and the amount of bound antigens is detected. Detection can be effected by indirect detection methods such as reaction with a secondary antibody labeled to produce a fluorescent or chemiluminescent signal, or direct detection such as by detecting changes in the surface plasmon resonance or optical properties of the surface.

Improved methods for the separation and detection of components of complex mixtures can provide improved diagnostic tests. For example, in cancer research, technology using DNA arrays provides a systematic method to identify key markers for prognosis and treatment response by profiling thousands of genes expressed in a single cancer. Hence, there remains a need for new methods to separate and detect chemical entities in complex mixtures. Therefore, it is the object herein to provide methods and products for identifying characteristic molecular profiles for complex samples.

### SUMMARY OF THE INVENTION

Provided herein are combinations, collections, kits and methods for identifying molecular profiles characteristic for a specific sample. Provided are addressable arrays that display diverse collections of binding sites. The binding sites can be used to capture components of samples. The resulting binding profiles provide a detectable pattern. Such patterns have diagnostic and prognostic uses as well as in drug discovery. These addressable arrays contain collections of capture agents with tagged reagents, such as scFv libraries, bound thereto.

The collections of capture agents (i.e., receptors, such as antibodies or other receptors) specifically bind to identifiable binding partners, such as polypeptides, designated tags herein. Each capture agent is selected or designed to bind with high affinity, selectivity, and specificity to a pre-selected tag, such as a polypeptide, epitope, ligand or portion thereof, which binds to the capture agent. The tags, such as polypeptide tags, are then used to tag diverse populations of molecules, such as cDNA libraries, or biological particles for the purpose of displaying a diverse collection of binding sites. The collections and resulting arrays of binding sites, produced upon binding of the tagged molecules or biological particles, contain identifiable capture agents, such as antibodies, provided in any suitable format. Suitable formats include, but are not limited to, liquid phase and solid phase formats, as long as the capture agents, such as antibodies, are identifiable (addressable).

Provided herein are methods for profiling a sample using the combinations, collections and kits described herein, which include some or all of the steps of (1) providing an addressable collection comprising a plurality binding sites, wherein the collection comprises a plurality of capture agents, such as antibodies, which are pre-selected to specifically bind to a pre-selected tag and a plurality of tagged reagents, each of which includes a molecule or biological particle and one of the tags, pre-selected to specifically bind to a capture agent; (2) contacting the collection of binding sites with a sample under conditions whereby components of the sample specifically bind to binding sites of the collection; and (3) detecting binding of the components, wherein loci to which the components bind provides a profile of the sample. Each locus in the collection includes the same capture agent and a plurality of different tagged reagents containing the same pre-selected tag. Each different locus includes a different capture agent. Each tag is bound to a capture agent thereby forming a complex of the tagged reagent with a capture agent. Samples for profiling with the methods provided herein include, but are not limited to, cell lystates, cells, body fluids, such as blood, plasma, serum, cerebrospinal fluid, synovial fluid, urine and sweat, tissue and organ samples from animals and plants, environmental samples, such as soil and water, viruses, bacteria, fungi algae, protozoa and components thereof. In one embodiment, the tagged reagents include scFvs or T cell receptors. In another embodiment, the capture agents include antibodies or fragments thereof. In another embodiment, a perturbation, such as a candidate compound, a condition or both, is added to the collection of binding sites prior to, simultaneously with or after contacting the sample to the collection of binding sites.

In one embodiment, the collection of addressable binding sites is produced by mixing capture agents and tagged reagents, where the each tagged reagent is specific for only one capture agent. In another embodiment, the collection of addressable binding sites is produced by mixing capture agents and tagged reagents, and the sample is added simultaneously to addition of the tagged reagents so that sample is added with the tagged reagents to a collection of capture agents, such as antibodies, and the collection of addressable binding sites with bound sample components is produced. The tags used in the methods provided herein can have one or more domains or regions, such as a divider region (D) or a common region (C) as described below. In another embodiment, the method of profiling a sample, such as cell lystates, cells, body fluids, such as blood, plasma, serum, cerebrospinal fluid, synovial fluid, urine and sweat, tissue and organ samples from animals and plants, environmental samples, such as soil and water, viruses, bacteria, fungi algae, protozoa and components thereof, further includes the step of detecting or identifying the pattern of loci to which components of the sample bind. The pattern can, optionally, be produced by comparing the results from the test sample to a control sample. In another embodiment, the profile and/or pattern produced can be stored in a database. Also, provided herein are computer systems or computer readable medium containing the database including binding profiles and/or patterns produced by the methods of sample profiling provided herein.

Combinations provided herein include addressable collections of binding sites containing: a plurality of capture agents, such as antibodies, wherein each capture agent is preselected to specifically bind to a pre-selected tag; a plurality of tagged reagents, each comprising one of the pre-selected tags, such as polypeptide tags; and one or more of software comprising instructions for pattern recognition and an imager for detecting patterns. Each locus in the collection of capture agents, such as antibodies, contains the same capture agent, which binds specifically to a pre-selected tag, such as a polypeptide tag, that is conjugated to a molecule or biological particle to form a tagged reagent. Each locus further includes a plurality of tagged reagents, such as tagged scFv and T cell receptor libraries, where each of the different molecules or biological particles at each locus includes the same pre-selected tag and the tagged reagents are bound to a capture agent, such as an antibody, forming a complex of the tagged reagent with the capture agent. Also provided herein are kits containing these combinations suitably packaged for use in a laboratory and optionally containing instructions for use are also provided.

Also provided herein are positionally addressable collections of binding sites, which includes a plurality of capture agents bound to a solid support, on which each capture agent is pre-selected to specifically bind to a pre-selected tag and each locus that contains the capture agents is within 1 mm or less from a neighboring locus; and a plurality of tagged reagents, which include one of the pre-selected tags and a molecule or biological particle. Each locus in the collection comprises the same capture agent and the capture agents at each different locus are different. Each tag is pre-selected to specifically bind to a capture agent and each tag is bound to a capture agent thereby forming a complex of the tagged reagent with the capture agent. Each locus comprises a plurality of tagged reagents and each of the different molecules at each locus comprises the same pre-selected tag. In one embodiment, molecules or biological particles in the tagged reagents are selected from the group consisting of a polypeptide, a nucleic acid, a carbohydrate, a lipid, a polysaccharide, a metal, an antibody, a cell membrane receptor, antiserum reactive with specific antigenic determinants, a lectin, a sugar, a polysaccharides, a cell, a cellular membranes and an organelle. In another embodiment, the capture agents and/or tagged reagents are antibodies or fragments thereof, scFvs or T cell receptors. In another embodiment, the diversity of the molecule or biological particles in the tagged reagents is 10¹², 10¹³, 10¹⁴, 10¹⁵ or higher.

Also provided herein are methods for screening, which include steps of (1) providing a collection of binding sites prepared by the methods provided herein; (2) contacting the collections of binding sites provided herein to a sample under conditions whereby components of the sample specifically bind to binding sites of the collection; (3) removing components of the sample which are not bound to the collection of binding sites; and (4) identifying components that are bound to the collection of binding sites. In one embodiment, a perturbation, such as a candidate compound, a condition or both, is added to the collection of binding sites prior to, simultaneously with or after contacting the sample to the collection of binding sites. In another embodiment, the diversity of the binding sites in the collection of binding sites includes at least 10², 10³, 10⁴, 10⁵, 10⁶, 10⁷, 10⁸, 10⁹, 10¹⁰, 10¹¹ and 10¹² or more. In another embodiment, steps (1) through (4) are repeated with a sub-set of binding sites which were shown to bind to components from the sample as identified by the first screening. In another embodiment, the sample includes, but is not limited to, cell lystates, cells, bodily fluids, such as blood, plasma, serum, cerebrospinal fluid, synovial fluid, urine, sweat, tissues and organs from animals and plants, environmental samples, such as soil and water, viruses, bacteria, fungi algae, protozoa and components thereof. In another embodiment, the capture agents are antibodies or fragments thereof, and the tagged reagents include scFvs or T cell receptors.

The capture agents and tagged reagents included in the combinations, collections, kits and methods provided herein can include, but are not limited to, a polypeptide, a nucleic acid, a carbohydrate, a lipid, a polysaccharide, a metal, an antibody, a cell membrane receptor, antiserum reactive with specific antigenic determinants, a lectin, a sugar, a polysaccharides, a cell, a cellular membranes and an organelle. The tagged reagents and capture agents can optionally be covalently linked upon or following complex formation. In one embodiment, the capture agents are antibodies or fragments thereof, the tags are polypeptide tags and the molecules are libraries of scFvs. In another embodiment, the capture agent and/or tag is a nucleic acid and the tagged reagent is a nucleic acid binding protein. In another embodiment, the capture agent or tagged reagent is an aptamer or a nucleic acid that specifically binds to a zinc finger domain, a leucine zipper or a modified restriction site. In another embodiment, the tagged reagent, such as a polypeptide, requires enzymatic modification for specific binding to a capture agent, such as an antibody.

The collections of capture agents, such as antibodies, provided in the combinations, collections, kits and methods herein, are tools that can be used in a variety of processes, including, but not limited to, rapid identification of antibodies or fragments thereof, such as scFvs, for therapeutics, diagnostics, research reagents, proteomics affinity matrices; enzyme engineering to identify improved catalysts, for antibody affinity maturation, for small molecule capture proteins and sequence-specific DNA binding proteins; for protein interaction mapping; and for development and identification of high affinity T cell receptors (see, e.g., Shusta et al. (2000) Directed evolution of a stable scaffold for T-cell receptor engineering, Nature Biotechnology 18:754-759). In particular herein, the capture agents are employed to capture tagged reagents to create diverse collections of binding sites.

The pre-selected tags, such as polypeptide tags, in the combinations, collections, kits and methods provided herein are linked to the molecules, such as proteins, or biological particles to be sorted and displayed by the capture agents, such as antibodies. Such linkage can be effected by any method, such as chemical conjugation or preparation of protein fusions, and can be conveniently effected using an amplification scheme or ligation with amplification that incorporates nucleic acids encoding the tags into nucleic acids that encode the proteins to be screened.

The tags, such as the polypeptide tags, also called epitope tags, also can be linked to longer polypeptides that specifically bind to the capture agents and that are linked to the molecules to be sorted and displayed. The tags are correlated, such as in a database, with the polypeptides to which they are linked. In such instances the tags can be selected to be encoded by conveniently amplifiable sequences of nucleic acids. Thus, the displayed molecules can be identified by virtue of locus to which the linked tag binds.

The tags, such as polypeptide tags, can be introduced into or onto molecules or biological particles by any suitable method, including chemical linkage and protein fusions. These methods include, for example, introduction of the tag into nucleic acid encoding the proteins by amplification with primers that encode the tags or by ligation of the oligonucleotides, optionally followed by an amplification, or by cloning into sets of plasmids encoding the tags. For example, the tags, such as polypeptide tags, are introduced into proteins by amplification, typically PCR, from cDNA libraries using primers that are designed to introduce the tags into the resulting amplified nucleic acid. A plurality of such tags are ultimately introduced into the nucleic acid, to permit sorting upon translation of the nucleic acids and to provide sequences for selective amplification of nucleic acids encoding desired proteins.

The tags, such as polypeptide tags, include a sequence of amino acids (designated “E” herein and for purposes herein generically called epitopes, but including sequence of amino acids to which any capture agent binds), to which the capture agents, such as antibodies, are designed or selected to bind. The tag, such as a polypeptide tag, is encoded by nucleic acid that includes at least one domain, which is a sequence of amino acids that specifically binds to a capture agent. In other embodiments, the tag can include at least two domains: one domain that encodes a sequence of amino acids that specifically binds to a capture agent (E portion); and a second domain that serves a primer site for specific amplification of the binding amino acids and any other amino acids fused thereto. The second domain may or may not be translated into a protein, a portion of can be translated, it can include other functional signals, such as stop codons, or ribosome binding sites, translation initiation sites and other such sites. The two domains can be adjacent to each other or separated or overlapping. In some embodiments, the second domain, is referred to herein as an R-tag.

The E portion (as noted generally referred to herein as an epitope, but not limited to sequences of amino acids that bind to antibodies or that are antigenic) of the tag includes a sufficient number of amino acids to selectively bind to a capture agent. It also, optionally, includes in certain embodiments, a sequence referred to herein as a divider (D) sequence, which can be 5′ or 3′ of the E portion and includes one or more amino acids, typically, at least three amino acids, and generally includes at least 4, 6, 8, 10, 14, 15, 16, 20 or more amino acids. The polypeptides that include the sequece of amino acids to which a capture agent binds (also referred to herein as an epitope) (E) and divider (D) sequences can include more amino acids and additional regions, as needed, for amplification of DNA encoding such tags or for other purposes. The tag, such as a polypeptide tag, can also include a common region designated “C”, which can be 5′ or 3′ of the E portion and/or D portion and includes one or more amino acids, typically, at least three amino acids, and generally includes at least 4, 6, 8, 10, 14, 15, 16, 20 or more amino acids.

For example, in one embodiment, the tags, such as polypeptide tags, are encoded by oligonucleotides that include the formula:

5′-Eₘ-3′

wherein each E encodes a sequence of amino acids to which a capture agent, such as an antibodies, binds, each such sequence of amino acids is unique in the set, and m is, independently, an integer of 2 or higher. In another embodiment, each oligonucleotide encoding the tag, such as a polypeptide tag, further includes a common region C of the formula:

5′C-Eₘ3′

wherein the common region is shared by each of the oligonucleotides in a set, and is of a sufficient length to serve as a unique priming site for amplifying nucleic acid molecules that include the sequence of nucleotides that includes the common region. In another embodiment, the tags, such as polypeptide tags, are encoded by oligonucleotides that include formula:

5′-Dₙ-Eₘ-3′

wherein each D is a unique sequence among the set of oligonucleotides and contains at least about 10 nucleotides, each E encodes a sequence of amino acids to which a capture agent binds with each such sequence of amino acids being unique in the set and each of n and m is, independently, an integer of 2 or higher. In another embodiment, m is the number of capture agents, such as antibodies, with different polypeptide specificity, and n is from about 2 up to and including 10⁶. In another embodiment m is the number of capture agents, such as antibodies, with different polypeptide specificity, and n is from about 2 up to and including 10⁶, from about 2 up to and including 10⁴, from about 2 up to and including 10² or from about 2 up to and including 10³.

In one embodiment, the tags, such as polypeptide tags, used in the combinations, collections, kits and methods provided herein are produced by a method of incorporating each one of a set of oligonucleotides into a nucleic acid molecule in a library of nucleic acid molecules, such as a cDNA, scFv or T cell receptor library, to create a tagged library where the set of oligonucleotides has the formula:

5′-Dₙ-Eₘ-3′

and each D is a unique sequence, which contains at least about 10 nucleotides, among the set of oligonucleotides, each E encodes an a sequence of amino acids that includes a sequence of amino acids that specifically binds to a capture agent (herein referred as an epitope) in the collection, each epitope is unique in the set and includes a sequence to which a capture agent binds, n is 0 or is an integer of 2 or higher, m is an integer of 2 or higher and the oligonucleotides are single-stranded, double-stranded, and/or partially double-stranded. In one embodiment, m×n is between about 10 to about 10¹², about 10 to about 10⁹ or about 10 to about 10⁶. In another embodiment, the library contains scFvs or T cell receptors.

In another embodiment, oligonucleotide further includes a common region C, and includes formula:

5′C-Dₙ-Eₘ3′

and the common region, which is of a sufficient length to serve as a unique priming site for amplifying nucleic acid molecules that include the sequence of nucleotides that includes the common region, is shared by each of the oligonucleotides in the set. In another embodiment, the library contains scFvs or T cell receptors.

The collections of capture agents, such as antibodies, used in the combinations, collections, kits and methods provided herein, can be arranged in an array, which can optionally be addressable, such as positionally or addressably tagged by linking the capture agents, such as antibodies, to electronic, chemical, optical or color-coded labels. In another embodiment, the collections of capture agents are provided in a solid phase format, linked directly or indirectly to a solid support, and can be organized as an addressable array in which each locus can be identified. Bar codes or other symbologies or indicia of identity can also be included on the solid phase arrays to aid in orientation or positioning of the antibodies. A plurality of such arrays can be included on a single matrix support. In one embodiment, the arrays are arranged and are of a size that matches, for example a 96-well, 384-well, 1536-well or higher density format. In another embodiment, for example, 24 such arrays, with 30 to 1000 antibody loci, such as 30, 100, 200, 250, 500, 750, 1000 or other convenient number, each are in such arrangement. In one embodiment, for example, 96 or more arrays, with 30 to 1000 antibody loci, such as 30, 100, 200, 250, 500, 750, 1000 or other convenient number, each are in such arrangement.

In another embodiment, the solid supports constitute coded particles (beads), such as microspheres that can be handled in liquid phase and then layered into a two dimensional array. The particles, such as microspheres, are encoded optically, such as by color or bar coded, chemically coded, electronically coded or coded using any suitable code that permits identification of the bead and capture agent bound thereto. The capture agent, such as an antibody, is coated on or otherwise linked to the support.

The collections of capture agents, such as antibodies, used in the combinations, collections, kits and methods provided herein with bound tags, such as polypeptide tags (or binding partners), linked to molecules are tools for the display of a large collection of proteins containing the tag sequences to which the capture agents bind, herein referred to as the tagged reagents. By exposing the collection of capture agents to different sets of tagged reagents, either simultaneously or separately, a large diversity of different tagged reagents can be reproducibly displayed on addressable loci. Contacting the resulting addressed tagged reagents (collection of binding sites) with a complex solution of chemical entities, such as a biological sample, and letting the chemical entities in the solution bind to the binding sites formed by the tag-containing proteins and then washing away unbound material and detecting the bound material, results in a complex yet reproducible profile, such as a pattern, of binding that is characteristic of the solution contacted with the tag-containing proteins. Comparing the profiles of characteristic binding, herein referred to as binding profiles, between and among different samples leads to the identification of tag-containing proteins, or collections of tag-containing proteins, that can be used to uniquely identify the samples.

The methods herein exemplified with respect to arrays can be practiced with any other format, including capture agents, such as antibodies, linked to RF tags, detectable beads, bar-coded beads and other such formats. The collections can serve as devices to profile samples, including, but not limited to cell lysate, cells, blood, plasma, serum, cerebrospinal fluid, synovial fluid, urine, sweat and tissue and organ samples from animals and plants, for identification of sample components that vary from sample to sample due to variations, such as disease or exposure to a pharmaceutical compound, among the samples. The collections of binding sites can also serve as devices to sort and identify molecules, such as proteins and genes, from within diverse collections, such as a scFv or a T cell receptor library (see, e.g., copending U.S. application Ser. No. 09/910,120 and corresponding published International PCT application No. WO 02/06834; U.S. provisional application Serial No. 60/422,923; and U.S. provisional application Serial No. 60/423,018). For purposes herein, the devices are employed for their ability to specifically bind to polypeptide (or otherwise)-tagged molecules, such as scFvs, to produce a diverse collection of binding sites.

In one embodiment, the addressable capture agents, such as antibodies, are provided as an array as described above, which contains a plurality of capture agents, that are provided on discrete addressable loci on a solid phase. Each address on the array contains capture agents, such as antibodies, that bind to a specific pre-selected tag. Generally all capture agents, such as antibodies, at each locus are identical or substantially identical, but it is only necessary for each agent to have specific high binding affinity (kₐ is generally at least about 10⁻⁷ to 10⁻⁹), to selectively bind to a molecule, generally a protein, that bears the predesigned or preselected tag, such as a polypeptide tag. In another embodiment, the addressable capture agents are addressably tagged by linking the capture agents, such as antibodies, to electronic, chemical, optical or color-coded labels.

Also provided herein are methods of sorting using the tag, such as polypeptide, labeled collections. Hence, provided herein are methods for identification of proteins with desired properties from large, diverse collections of proteins by sorting. Critical to the methods and the addressable collections of binding proteins (capture agents) provided herein is the selection of capture agents, such as antibodies or other binding proteins, that bind to a set of pre-selected tags, such as polypeptides, of known sequence. The polypeptide tags include a sufficient number of amino acids to specifically bind to the capture agent, such as an antibody. The collections of capture agents, such as antibodies, contain at least about 10, more least about 30, 50, 100, 200, 250, and more, such as at least about 500, 1000, or more, different capture agents, such as antibodies, which bind to different members of the set of polypeptide tags. Methods for producing collections of the capture agents, such as antibodies, are provided herein.

In one embodiment the addressable capture agents, such as antibodies, are provided as an array, which contains a plurality of capture agents, that are provided on discrete addressable loci on a solid phase. Each address on the array contains capture agents, such as antibodies, that bind to a specific pre-selected tag. Generally all capture agents, such as antibodies, at each locus are identical or substantially identical, but it is only necessary for each agent to have specific high binding affinity (kₐ is generally at least about 10⁻⁷ to 10⁻⁹), to selectively bind to a molecule, generally a protein, that bears the predesigned or preselected poly-peptide tag.

In practice, to produce the collection of binding sites, tagged reagents, such as proteins, with the pre-selected tags, such as polypeptide tags, are bathed over an array of capture agents or reacted with the collection of capture agents linked to identifiable supports, such as beads, under suitable binding conditions. By virtue of the binding specificity of the pre-selected tags for particular capture agents, the tagged reagents are sorted according their pre-selected tag and displayed at each locus. The identity of the tag is then known, since it reacts with a particular capture agent whose identity is known by virtue of its position in the array or its identifier, such as its linkage to an optically coded, including as color coded or bar coded, or an electronically-tagged, such as a microwave or radio frequency (RF)-tagged, particle. In one embodiment, the diversity of the binding sites prepared using the methods provided herein includes at least 10², 10³, 10⁴, 10⁵, 10⁶, 10⁷, 10⁸, 10⁹, 10¹⁰, 10¹¹ and 10¹² or more.

Methods for selecting and preparing the capture agent, such as antibody, members of the collections are also provided. Methods for designing polypeptide tags and for preparing antibodies that specifically bind to the tags are provided. Methods for preparing primers and sets of primers are also provided.

Oligonucleotides and sets thereof for introducing the tags for performing the sorting and recovery processes are also provided. Sets of oligonucleotides, which are single-stranded for embodiments in which they are used as primers or double-stranded (or partially double-stranded) for embodiments in which they are introduced by ligation, for preparation of tagged proteins are also provided. Methods for designing the primers are also provided.

Combinations of an array or set of beads (i.e., particulate supports) linked or coated with collections capture agents, such as antibodies (i.e., antibodies that specifically bind to polypeptide tags), and the polypeptide tags to which the capture agents specifically bind or a set of expression vectors encoding the polypeptide tags are provided. The vectors optionally contain a multiple cloning site for insertion of a cDNA library of interest. The combinations can further include enzymes and buffers that are necessary for the subcloning, and competent cells for transformation of the library and oligonucleotide primers to use for recovery of the sublibrary of interest. Also provided are combinations containing two or more of the array or set of beads coated with or linked to the capture agents, such as anti-tag antibodies, a set of oligonucleotides encoding the polypeptide tags, any common regions necessary for appending to a cDNA library of interest, and optionally any enzymes and buffers that are used in the ligation, ligase chain reaction (LCR), polymerase chain reaction (PCR), and/or recombination necessary for appending the panel of tags to the cDNA in a library. The combinations can further include a system for in vitro transcription and translation of the protein products of the tagged cDNA, and optionally oligonucleotide primers to use for recovery of the sub-library of interest.

Kits containing these combinations suitably packaged for use in a laboratory and optionally containing instructions for use are also provided.

In one embodiment, combinations of the collections of capture agents, such as antibodies, and oligonucleotides that encode tags, such as polypeptide tags, to which the capture agents selectively bind are provided. Kits containing the oligonucleotides and capture agents, such as antibodies, and optionally containing instructions and/or additional reagents are provided. The combinations include a collection of capture agents, such as antibodies, that specifically bind to a set of pre-selected tags, such as polypeptides, and a set of oligonucleotides that encode each of the tags. The oligonucleotides are single-stranded, double-stranded or include double-stranded and single-stranded portions, such as single-stranded overhangs created by restriction endonuclease cleavage.

### DESCRIPTION OF THE DRAWINGS

FIG. 1 illustrates the concept of nested sorting.

FIG. 2 also illustrates nested sorting; this sort is identical to the sort illustrated in FIG. 1 except that the F2 and F3 sub-libraries have been arranged into arrays.

FIG. 3 illustrates the use antibody arrays as a tool for nested sorts of high diversity gene libraries.

FIG. 4 illustrates application of the methods provided herein for searching libraries of mutated genes.

FIG. 5 illustrates a method for constructing recombinant antibody libraries.

FIG. 6 depicts one method for incorporating polypeptide (epitope) tags into recombinant antibodies using primer addition.

FIG. 7 depicts an alternative scheme using linker addition.

FIG. 8 depicts application of the methods herein for searching recombinant antibody libraries.

FIG. 9 schematically depicts elements of the primers provided herein and the sets of primers required.

FIGS. 10 and 11 depict alternative methods for constructing the ED and EDC primers; in FIG. 10 oligonucleotides are chemically synthesized 3′ to 5′ on a solid support; in the method in FIG. 11, the oligonucleotides self-assemble based upon overlapping hybridization.

FIG. 12 depicts a high throughput screen for discovering immunoglobulin (Ig) produced from hybridoma cells for use in the arrays.

FIGS. 13A and 13B depict exemplary primers (see SEQ ID Nos. 12-73) for amplification of antibody chains for preparation of recombinant human antibodies (see Table 33, pages 87-88 in McCafferty et al. (1996) Antibody engineering: A practical Approach, Oxford University Press, Oxford, see also, Marks et al. (1992) Bio/Technology 10:779-783; and Kay et al. (1996) Phage Display of Peptides and Proteins: A Laboratory Manual, Academic Press, San Diego).

FIGS. 14A-14D depict use of the methods herein for antibody engineering.

FIG. 15 depicts use of the methods herein for identification of antibodies with modified specificity (or any protein with modified specificity).

FIG. 16 depicts use of the methods herein for simultaneous antibody searches.

FIG. 17 depicts use of the methods herein in enzyme engineering protocols

FIG. 18 depicts use of the methods herein in protein interaction mapping protocols.

FIG. 19 depicts the rate of and increase in the number of tags when multiple polypeptide tags are used for sorting.

FIGS. 20A-20H depict exemplary embodiments in which the tag includes the epitope (i.e., region that specifically binds to a capture agent) and a recover tag for identification of the linked protein.

FIG. 21 depicts an collection of capture agents with bound tagged-agents, showing the diversity of tagged reagent on the surface. Each tag is bound to a plurality of different agents resulting in a surface with a large diversity of binding sites.

FIG. 22 depicts an exemplary procedure for preparing a collection, such as that of FIG. 21, and then the use thereof for profiling a sample.

FIG. 23 depicts the use of the tags in a collection, such as that of FIG. 21, for identifying the tagged reagent using the polypeptide tag, such as the myc peptide (SEQ ID No. 91), to create primers for amplification of nucleic acid encoding the agents. Further purification, if desired, can identify the particular agents that bind to components of the sample.

FIG. 24 depicts an exemplary use of the collections for profiling in which the sample is tissue from a diseased or drug-treated subject, is compared to a healthy control. The two profiles are compared and differences are representative of disease or health. The samples are reacted with either a collection of capture agents, but generally a collection of capture agents with bound tagged-agents, since the latter presents a more diverse set of binding sites for a sample. The profiles can be identified by eye, but generally using an imager and computer programmed for profile, such as pattern, recognition.

FIG. 25 depicts exemplary applications of the profiling embodiments.

FIGS. 26A and 26B depict steps for evenly distributing tags throughout a collection of polypeptides.

FIG. 27 depicts Idiotype receptors from cell lystates that have been specifically captured by anti-Idiotype antibodies on arrays.

FIGS. 28A and 28B depicts exemplary methods for isolating capture agent/tag pairs; FIG. 28A shows a panning method and FIG. 28B shows an immunization method.

For clarity of disclosure, and not by way of limitation, the detailed description is divided into the subsections that follow.

### DETAILED DESCRIPTION

A. Definitions

B. Collections of Binding Sites (Capture Systems)

1. Capture Agents

2. Tags and Formats for Tags

3. Covalent Interactions Between Capture Agents and Tags

4. Methods for Tag (Polypeptide Tag) Incorporation

a. Ligation to Create Circular Plasmid Vectors for Introduction of Tags

b. Ligation of Sequences Resulting in Linear Tagged cDNA Molecules

c. Primer Extension and PCR for Tag Incorporation

d. Insertion by Gene Shuffling

e. Recombination Strategies

f. Incorporation by Transposases

g. Incorporation by Splicing

h. Alternative Method for Distribution of Tags

(1) Determination of the Required Diversity of the Master Library

(2) Creation of the Master Library and Division into Sub-Libraries

(3) Adjustment of the Diversity of a Master Library so that the Diversity is about Equal to the Number of Members of the Library

(4) Division of the Master Library into Sub-Libraries

(5) Creation of Tagged Libraries

(6) Mixing Some or All of the Tagged Sub-Libraries to Produce a Mixed Library, where the Number of Tagged Nucleic Acid Molecules Added from Each Tagged Sub-Library is the Same

(7) Splitting the Mixed Library into “q” Array Libraries, wherein q is from 1 to a Predetermined Number of Arrays

(8) Expression of the Array Libraries and Purification of Tagged Molecules to Produce Collections of Tagged Molecules with Even Distribution of Tags

5. Preparation of Capture Agents

a. Antibodies and Collections of Addressable Anti-tag Antibodies

b. Preparation of the Capture Agents

c. Preparation of the Capture Agent Arrays

d. Preparation of Other Collections

6. Supports for Immobilization of Capture Agents

a. Natural Support Materials

b. Synthetic Supports

c. Immobilization and Activation

7. Detection of Bound Antigen(s)

a. Methods of Staining

b. Molecules for Staining

c. Immunostaining

(1) Enzymes and Chromagens for Immunostaining

(i) Luminescent Labels

(ii) Horseradish Peroxidase (HRP)

(iii) Alkaline Phosphatase (AP)

(2) Biotin-Avidin Staining Methods

(3) Chain Polymer-Conjugation Methods

C. Use of the Collections of Capture Agents for Profiling

1. Exemplary Profiling Methods

2. Prognosis and Diagnosis

3. Drug Discovery

D. Identification and Recovery of Tagged Molecules or Biological Particles Using Nested Sorting

1. Overview

2. Recovery of Identified Tagged Molecules

a. Design and Preparation of Oligonucleotides/Primers

(1) Primers

(2) Preparation of the Oligonucleotides/Primers

b. Use of Multiple Tags in a Single Fusion Protein

3. Sorting Methods Dividing the Master Library

4. Creating the Master Library for Sorting

5. The First Sorting Step

6. The Second Sorting Step

E. Use of the Methods for Identification of Proteins of Desired Properties from a Library

1. Arraying Capture Agents

2. Exemplary Use of Identification of Genes from a Library of Mutated Genes

F. Identification of Recombinant Antibodies

G. Examples

A. DEFINITIONS

Unless defined otherwise, all technical and scientific terms used herein have the same meaning as is commonly understood by one of skill in the art to which this invention belongs. All patents, patent applications, publications and published nucleotide and amino acid sequences (e.g., sequences available in GenBank or other databases) referred to herein are incorporated by reference. Where reference is made to a URL or other such identifier or address, it is understood that such identifier can change and particular information on the internet can come and go, but equivalent information can be found by searching the internet. Reference thereto evidences the availability and public dissemination of such information.

As used herein, profiling refers to detection and/or identification of a plurality of components, generally 3 or more, such as 4, 5, 6, 7, 8, 10, 50, 100, 500, 1000, 10⁴, 10⁵, 10⁶, 10⁷ or more, in a sample. A profile refers to the identified loci to which components of a sample detectably bind. The profile can be detected as a pattern on a solid surface, such as in embodiments when the addressable collection includes an array of capture agents on a solid support, in which case the profile can be presented as an visual image. In embodiments, such as those in which the capture agents and bound tagged molecules are on color-coded beads or are otherwise detectably labeled, a profile or binding profile refers to the identified tags and/or capture agents to which component(s) is (are) detectably bound, which can be in the form of a list or database or other such compendium.

As used herein, an image refers to a collection of datapoints representative of the profile. An image can be a visual, graphical, tabular, matrix or other depiction of such data. It can be stored in a database.

As used herein, nested sorting refers to the process of decreasing diversity using the addressable collections of antibodies provided herein.

As used herein, a database refers to a collection of data items.

As used herein, a relational database is a collection of data items organized as a set of formally-described tables from which data can be accessed or reassembled in many different ways without having to reorganize the database tables. Such databases are readily available commercially, for example, from Oracle, IBM, Microsoft, Sybase, Computer Associates, SAP, or multiple other vendors. Databases can be stored on computer-readable media, such floppy disks, compact disks, digital video disks, computer hard drives and other such media.

As used herein, an addressable collection of capture agents (also referred to herein as an addressable collection of anti-tag capture agents or anti-tag receptors) protein agents (i.e., receptors), such as antibodies, that specifically bind to pre-selected polypeptide tags that contain epitopes (sequences of amino acids, such as epitopes in antigens) in which each member of the collection is labeled and/or is positionally located to permit identification of the capture agent, such as the antibody, and tag. The addressable collection is typically an array or other encoded collection in which each locus contains receptors, such as antibodies, of a single specificity and is identifiable. The collection can be in the liquid phase if other discrete identifiers, such as chemical, electronic, colored, fluorescent or other tags are included. Capture agents, include antibodies and other anti-tag receptors. Any moiety, such as a protein, nucleic acid or other such moiety, that specifically binds to a pre-determined sequence of amino acids, such as an epitope, is contemplated for use as a capture agent.

As used herein, an address refers to a unique identifier whereby an addressed entity can be identified. An addressed moiety is one that can be identified by virtue of its address. Addressing can be effected by position on a surface or by other identifier, such as a tag encoded with a bar code or other symbology, a chemical tag, an electronic, such RF tag, a color-coded tag or other such identifier.

As used herein, a molecule, such as capture agent, that specifically binds to a polypeptide, such as a polypeptide tagged molecule provided herein, typically has a binding affinity (Kₐ) of at least about 10⁶ l/mol, 10⁷ l/mol, 10⁸ l/mol, 10⁹ l/mol, 10¹⁰ l/mol or greater (generally 10⁸ or greater) and binds generally with greater affinity (typically at least 10-fold, generally 100-fold or) than to the molecules and biological particles that are to be detected or assessed in the methods that employ the employ the capture systems. Thus, affinity refers to the strength of interaction between a capture agent and a polypeptide tag.

As used herein, specificity (or selective binding or selectively binding) with respect to binding of tags to capture agents refers to the greater affinity the tag and capture agent exhibit for each other compared to the molecules and biological particles that are to be detected by the capture systems.

As used herein, used to “bind” to a capture system means to interact with sufficient affinity to immobilize the bound moiety (such as a biogical particle or molecule) temporarily under the conditions of a particular experiment. For purposes herein, it is an interaction that permits biological particles, such as cells, or biological molecules to be retained at a locus when biological particles or molecules are contacted with the capture systems so that they no longer move by Brownian motion or other microcurrents in a composition.

As used herein, a canvas is a collection of arrays, such as those provided herein. The size of each array and number in a canvas can vary and is at least two.

As used herein, a landscape is the information produced or presented on a canvas or array.

As used herein, a binding partner or a tag is any moiety that specifically binds to a capture agent. The binding partner constitute or include tags that are the portion that specifically binds to a capture agent. The tags can be any molecule, compound, substance that will specifically bind to a capture agent and also can be provided or produced in a form that permits its linkage to molecules (or other entities, including biological particles, such as cells and virions) that are intended for display in the collections of binding sites. Typically, although not necessarily, the tags are included as portions or as polypeptides. Polypeptides advantageously can be selected and/or designed to specifically bind to a capture agent and also are readily linked other molecules, as fusions or as conjugates in which they linked via covalent, ionic and other chemical interactions.

As used herein, polypeptide tags generically refer to the binding partners that include a sequence of amino acids that specifically bind to a capture agent. The polypeptide tags are also referred to herein as epitope tags or tags. It emphasized that epitope as used herein is not necessarily an antigenic sequence of amino acids, but one that specifically binds to a capture agent.

As used herein, an epitope tag generally refers to a sequence of amino acids that includes the sequence of amino acids, herein referred to as an epitope, to which an anti-tag capture agent, such as an antibody specifically binds. The epitope can be other than a polypeptide; as long as at least a portion of it specifically binds to a capture agent. Furthermore, as described in more detail below, epitope tags can include two domains: a tag-specific amplification sequence (herein referred to as an R-tag) and a ligand-binding domain.

For polypeptide (epitope) tags, the specific sequence of amino acids to which each binds is referred to herein generically as an epitope. Any sequence of amino acids that binds to a receptor therefor is contemplated. For purposes herein the sequence of amino acids of the tag, such as epitope portion of the epitope tag, that specifically binds to the capture agent is designated “E”, and each unique epitope is an Eₘ. Depending upon the context “Eₘ” can also refer to the sequences of nucleic acids encoding the amino acids constituting the epitope. The polypeptide tag, i.e., the epitope tag, can also include amino acids that are encoded by the divider region. In particular, the epitope tag is encoded by the oligonucleotides provided herein, which are used to introduce the tag. When reference is made to an epitope tag (i.e., binding pair for a particular receptor or portion thereof) with respect to a nucleic acid, it is nucleic acid encoding the tag to which reference is made. For simplicity each polypeptide tag is referred to as Eₘ; when nucleic acids are being described the Eₘ is nucleic acid and refers to the sequence of nucleic acids that encode the epitope; when the translated proteins are described Eₘ refers to amino acids (the actual epitope). The number of E's corresponds to the number of antibodies in an addressable collection. “m” is typically at least 10, 30 or more, 50 or 100 or more, and can be as high as desired and as is practical. Generally “m” is about a 1000 or more. As discussed below, other moieties that function as binding partners for capture agents also are contemplated.

The epitope tag is encoded by nucleic acid that includes at least two domains: one domain that encodes a sequence of amino acids that specifically binds to a capture agent; and a second domain that serves a primer site for specific amplification of the binding amino acids and any other amino acids fused thereto. The second domain can or can not be translated into a protein, a portion of can be translated, it can include other functional signals, such as stop codons, or ribosome binding sites, translation initiation sites and other such sites. The two domains can be adjacent to each other or separated or overlapping. In some embodiments, the second domain, is referred to herein as an R-tag.

As used herein, tagged reagent refers to a conjugated molecule or biological particle and a tag, such as a polypeptide tag, which bind specifically to a capture agent. The molecule or biological particle can be linked to a particular tag, such as a polypeptide tag, directly through a chemical conjugation, such as hydrophobic, ionic, covalent and van der Waals interactions, or can be linked by producing fusion proteins from nucleic acid encoding the tag linked directly or indirectly to nucleic acid encoding the molecule. The tag is conjugated to the molecule or biological particle with a sufficient K_{d} so that interaction is stable upon binding of the tag to the capture agents. Further, the conjugates are such that the tag are conjugated to the molecules or biological particles such that the tags retain their specificity for their capture agent.

As used herein, Dₙ refers to a divider sequence that is optionally present in an oligonucleotide that encodes a polypeptide tag. As described herein in certain embodiments in which division is effected by other methods Dₙ is optional. As with each Eₘ the Dₙ is either nucleic acid or amino acids depending upon the context. Each Dₙ is a divider sequence that is encoded by a nucleic acid that serves as a priming site to amplify a subset of nucleic acids. The resulting amplified subset of nucleic acids contains all of the collection of Eₘ sequences and the Dₙ sequences used as a priming site for the amplification. As described herein, the nucleic acids include a portion, generally at the end, that encodes each EₘDₙ. Generally the encoding nucleic acid is 5′-Eₘ-Dₙ-3′ on the nucleic acid molecules in the library). D is an optional unique sequence of nucleotides for specific amplification to create the sub-libraries. For large libraries, the original library can be divided into sub-libraries and then the tag-encoding sequences added, rather than adding the tag-encoding sequences to the master library, The size of D is a function of the library to be sorted, since the larger the library the longer the sequence needed to specify a unique sequence in the library. Generally D, depending upon the application, should be at least 14 to 16 nucleic acid bases long and it can or can not encode a sequence of amino acids, since its function in the method is to serve as a priming site for PCR amplification, D is 2 to n, where n is 0 or is any desired number and is generally 10 to 10,000, 10 to 1000, 50 to 500, and about 100 to 250. The number of D can be as high as 10⁶ or higher. The divider sequences D are used to amplify each of the “n” samples from the tagged master library, and generally is equal to the number of antibody collections, such as arrays, used in the initial sort. The more collections (divisions) in the initial screen, the lower diversity per addressable locus. The initial division number is selected based upon the diversity of the library and the number of capture agents. The more E's, the fewer D's are needed, and vice versa, for a library having a particular diversity (Div).

As used herein, diversity (Div) refers to the number of different molecules in a library, such as a nucleic acid library. Diversity is distinct from the total number of molecules in any library, which is greater. The greater the diversity, the lower the number of actual duplicates there are. Ideally the (number of different molecules)/(total molecules) is approximately 1. If the number of molecules that are randomly tagged to create the master library, is less than the initial diversity, then statistically each of the molecules in the master library should be different.

As used herein, an addressable collection of binding sites refers to the resulting sites produced upon binding of the capture agents provided herein to tagged reagents, such as molecules and biological particles. Each capture agent sorts reagents by virtue of their tags, such as polypeptide tags, each unique tagged reagent is linked to a plurality of different molecules, generally polypeptides. As a result, upon sorting the capture agent and tagged-reagent form a complex and the resulting complex can bind further molecules. Since the reagents specific for each capture agent can contain a plurality of different molecules that share the same tag, when bound to a plurality of different capture agents the resulting collection can presents (or display) a collection of binding sites. The collection is addressable because the identity of the tags, such as polypeptide tags, is known or can be ascertained. The molecules and biological particles or any other moieties that are displayed in the collections provided herein are displayed in order to present binding sites for capturing components of a sample. Hence, such molecules and biological particles are selected for the ability to bind to components of samples.

As used herein, a capture system refers to an addressable collection of capture agents and tagged molecules (or biological particles), such as polypeptide tagged molecules, bound thereto, where each different polypeptide tag specifically binds to a different capture agent. Hence, when a capture system displays tagged molecules (or biological particles) it is a collection of binding sites.

As used herein, highly diverse can refer to the diversity of the collections of binding sites provided herein. Because each tag is specific for a single capture agent, the collections include a plurality of addressable capture agents, such as 10, 50, 100, 250, 500, 1000 or more, and each tag is linked to collections of molecules that can have high diversity, such as 10⁶, 10⁷, 10⁸, 10⁹, 10¹⁰, 10¹¹, 10¹² and more, the resulting collections of binding sites display diversities of (10⁶, 10⁷, 10⁸, 10⁹, 10¹⁰, 10¹¹, 10¹² and more) times the number of different capture agents. Thus, the collections and methods herein provide for highly diverse collections.

As used herein, highly diverse refers to diversities that can be greater than the highest diversity found in particular collection. The diversity will be increased by a factor equal to the number of different tags (and/or capture agents).

As used herein, an array refers to a collection of elements, such as antibodies, containing three or more members. An addressable array is one in which the members of the array are identifiable, typically by position on a solid phase support or by virtue of an identifiable or detectable label, such as by color, fluorescence, electronic signal (i.e., RF, microwave or other frequency that does not substantially alter the interaction of the molecules of interest), bar code or other symbology, chemical or other such label. Hence, in general the members of the array are immobilized to discrete identifiable loci on the surface of a solid phase or directly or indirectly linked to or otherwise associated with the identifiable label, such as affixed to a microsphere or other particulate support (herein referred to as beads) and suspended in solution or spread out on a surface. A microarray, which is used by those of skill in the art, generally is a positionally addressable array, such as an array on a solid support, in which the loci of the array are at high density. For example, an array can be formed on a surface the size of a standard 96 well microtiter plate with 96 loci, 384, or 1536. Such arrays are not considered microarrays by those of skill in the art. Arrays at higher densities, however, generally greater than 5,000 or typically 10,000 and more loci per plate are considered microarrays. Typically for an positionally addressable array to be a microarray, the elements (spots) in a microarray are about 1 mm or less apart.

As used herein, a support (also referred to as a matrix support, a matrix, an insoluble support or solid support) refers to any solid or semisolid or insoluble support to which a molecule of interest, typically a biological molecule, organic molecule or biospecific ligand is linked or contacted. Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacrylamide, silicon, rubber, and other materials used as supports for solid phase syntheses, affinity separations and purifications, hybridization reactions, immunoassays and other such applications. The matrix herein can be particulate or can be a be in the form of a continuous surface, such as a microtiter dish or well, a glass slide, a silicon chip, a nitrocellulose sheet, nylon mesh, or other such materials. When particulate, typically the particles have at least one dimension in the 5-10 mm range or smaller. Such particles, referred collectively herein as “beads”, are often, but not necessarily, spherical. Such reference, however, does not constrain the geometry of the matrix, which can be any shape, including random shapes, needles, fibers, and elongated. Roughly spherical “beads”, particularly microspheres that can be used in the liquid phase, are also contemplated. The “beads” can include additional components, such as magnetic or paramagnetic particles (see, e.g., Dyna beads (Dynal, Oslo, Norway)) for separation using magnets, as long as the additional components do not interfere with the methods and analyses herein.

As used herein, matrix or support particles refers to matrix materials that are in the form of discrete particles. The particles have any shape and dimensions, but typically have at least one dimension that is 100 mm or less, 50 mm or less, 10 mm or less, 1 mm or less, 100 μm or less, 50 μm or less and typically have a size that is 100 mm³ or less, 50 mm³ or less, 10 mm³ or less, and 1 mm³ or less, 100 μm³ or less and may be order of cubic microns. Such particles are collectively called “beads.”

As used herein, a capture agent, which is used interchangeably with a receptor, refers to a molecule that has an affinity for a given ligand or a with a defined sequence of amino acids. Capture agents can be naturally-occurring or synthetic molecules, and include any molecule, including nucleic acids, small organics, proteins and complexes that specifically bind to specific sequences of amino acids. Capture agents are receptors and are also referred to in the art as anti-ligands. As used herein, the terms, capture agent, receptor and anti-ligand are interchangeable. Capture agents can be used in their unaltered state or as aggregates with other species. They can be attached or in physical contact with, covalently or noncovalently, a binding member, either directly or indirectly via a specific binding substance or linker. Examples of capture agents, include, but are not limited to: antibodies, cell membrane receptors surface receptors and internalizing receptors, monoclonal antibodies and antisera reactive or isolated components thereof with specific antigenic determinants (such as on viruses, cells, or other materials), drugs, polynucleotides, nucleic acids, peptides, cofactors, lectins, sugars, polysaccharides, cells, cellular membranes, and organelles. For example, the capture agents can specifically bind to DNA binding proteins, such as zinc fingers, leucine zippers and modified restriction enzymes.

Examples of capture agents, include but are not restricted to:

a) enzymes and other catalytic polypeptides, including, but are not limited to, portions thereof to which substrates specifically bind, enzymes modified to retain binding activity lack catalytic activity;

b) antibodies and portions thereof that specifically bind to antigens or sequences of amino acids;

c) nucleic acids;

d) cell surface receptors, opiate receptors and hormone receptors and other receptors that specifically bind to ligands, such as hormones. For the collections herein, the other binding partner, referred to herein as a polypeptide tag for each refers the substrate, antigenic sequence, nucleic acid binding protein, receptor ligand, or binding portion thereof.

As noted, contemplated herein, are pairs of molecules, generally proteins that specifically bind to each other. One member of the pair is a polypeptide that is used as a tag and encoded by nucleic acids linked to the library; the other member is anything that specifically binds thereto. The collections of capture agents, include receptors, such as antibodies or enzymes or portions thereof and mixtures thereof that specifically bind to a known or knowable defined sequence of amino acids that is typically at least about 3 to 10 amino acids in length. Other examples of capture agents are set forth throughout the disclosure.

As used herein, printing refers to immobilization of capture agents onto a solid support, such as, but not limited to, a microarray.

As used herein, master library refers to a collection of molecules, such as a cDNA library encoding proteins, to be analyzed or displayed or assessed. These molecules do not contain polypeptide tags nor nucleic acid molecules encoding the tags. In the methods provided herein, for evenly distributing tags in libraries the master libraries are libraries of nucleic acid molecules, such as cDNA libraries.

As used herein, a biological particle refers to a virus, such as a viral vector or viral capsid with or without packaged nucleic acid, phage, including a phage vector or phage capsid, with or without encapsulated nucleic acid, a single cell, including eukaryotic and prokaryotic cells or fragments thereof, a liposome or micellar agent or other packaging particle, and other such biological materials.

As used herein, a conjugate or cross-linked complex refers to a complex between a binding partner and a molecule or biological particle. The binding partner is conjugated to the molecule or biological particle with a sufficient K_{d} so that interaction is stable upon binding of the binding partner to the capture agents in the array. Further, the conjugates are such that the binding partners are conjugated to the molecules or biological particles such that the binding partners retain their specificity for their capture agent.

As used herein, sub-library refers to the initial collection of different libraries produced by subdividing a master library. The sub-libraries are created by physical separation of a master library into “n” number of discrete collections.

As used herein, tagged library refers to the resulting collections of molecules after the sub-libraries have been separately tagged with tags, such as polypeptide tags.

As used herein, normalized tagged libraries refers to resulting collections of molecules after the number of molecules in each tagged library has been estimated and then adjusted such that each normalized tagged library contains approximately the same diversity and number of molecules.

As used herein, mixed library refers to the resulting collection of molecules after normalized tag libraries have been combined.

As used herein, array library refers to the collections of molecules created by physical separation of the mixed library into q number of discrete collections. The array libraries serve as the genetic source for the tagged molecules to be expressed and purified and contacted with arrays of capture agents. Nucleic acid molecules from these libraries also serve as the source of template DNA used in the amplification protocols to recover the desired tagged molecules once identified using the arrays.

As used herein, transformation efficiency refers to the number of bacterial colonies produced per mass of plasmid DNA transformed (colony forming units (cfu) per mass of transformed plasmid DNA).

As used herein, titer with reference to phage refers to the number of colony forming units (cfu) per ml of transformed cells.

As used herein, normalization refers to the equilibration of the titer or concentration of all members of a tag library so that the number of tagged members in two samples or portions are about the same.

As used herein, the total display refers to the total diversity of molecules being displayed on the arrays.

As used herein, a B cell refers to a lymphocyte that develops from hemopoietic stem cells in the bone marrow of adults and the liver of fetuses and is responsible for the production of circulating antibodies.

As used herein, a T cell refers to a lymphocyte that develops in thymus from precursor cells that migrate there from the hemopoietic tissues via the blood. T cells fall into two main classes, cytotoxic T cells and helper T cells. Cytotoxic T cells kill infected cells, whereas helper T cells help to activate macrophages, B cells and cytotoxic T cells.

As used herein, a T cell receptor (TCR) refers to an antigen receptor found on the surface of both cytotoxic and helper T cells. T cell receptors (TCRs) are similar to antibodies and are composed of two disulfide-linked polypeptide chains, each of which contains two immunoglobulin-like domains, one variable domain and one constant domain.

As used herein, antibody refers to an immunoglobulin, whether natural or partially or wholly synthetically produced, including any derivative thereof that retains the specific binding ability of the antibody. Hence antibody includes any protein having a binding domain that is homologous or substantially homologous to an immunoglobulin binding domain. For purposes herein, antibody includes antibody fragments, such as Fab fragments, which are composed of a light chain and the variable region of a heavy chain Antibodies include members of any immuno-globulin class, including IgG, IgM, IgA, IgD and IgE. Also contemplated herein are receptors that specifically binding to a sequence of amino acids.

Hence for purposes herein, any set of pairs of binding members, referred to generically herein as a capture agent/polypeptide tag, can be used instead of antibodies and epitopes per se. The methods herein rely on the capture agent/tag, such as and antibody/polypeptide tag, for their specific interactions, any such combination of receptors/ligands (tag) can be used. Furthermore, for purposes herein, the capture agents, such as antibodies employed, can be binding portions thereof.

As used herein, a monoclonal antibody refers to an antibody secreted by a hybridoma clone. Because each such clone is derived from a single B cell, all of the antibody molecules are identical. Monoclonal antibodies can be prepared using standard methods known to those with skill in the art (see, e.g., Kohler et al. Nature 256:495 (1975) and Kohler et al. Eur. J. Immunol. 6:511 (1976)). For example, an animal is immunized by standard methods to produce antibody-secreting somatic cells. These cells are then removed from the immunized animal for fusion to myeloma cells.

Somatic cells with the potential to produce antibodies, particularly B cells, are suitable for fusion with a myeloma cell line. These somatic cells can be derived from the lymph nodes, spleens and peripheral blood of primed animals. Specialized myeloma cell lines have been developed from lymphocytic tumors for use in hybridoma-producing fusion procedures (Kohler and Milstein, Eur. J. Immunol. 6:511 (1976); Shulman et al. Nature 276: 269 (1978); Volk et al. J. Virol. 42: 220 (1982)). These cell lines have been developed for at least three reasons. The first is to facilitate the selection of fused hybridomas from unfused and similarly indefinitely self-propagating myeloma cells. Usually, this is accomplished by using myelomas with enzyme deficiencies that render them incapable of growing in certain selective media that support the growth of hybridomas. The second reason arises from the inherent ability of lymphocytic tumor cells to produce their own antibodies. The purpose of using monoclonal techniques is to obtain fused hybrid cell lines with unlimited life spans that produce the desired single antibody under the genetic control of the somatic cell component of the hybridoma. To eliminate the production of tumor cell antibodies by the hybridomas, myeloma cell lines incapable of producing endogenous light or heavy immunoglobulin chains are used. A third reason for selection of these cell lines is for their suitability and efficiency for fusion. Other methods for producing hybridomas and monoclonal antibodies are well known to those of skill in the art.

As used herein, antibody fragment refers to any derivative of an antibody that is less than full length, retaining at least a portion of the full-length antibody's specific binding ability. Examples of antibody fragments include, but are not limited to, Fab, Fab′, F(ab)₂, single-chain Fvs (scFv), Fv, dsFv diabody and Fd fragments. The fragment can include multiple chains linked together, such as by disulfide bridges. An antibody fragment generally contains at least about 50 amino acids and typically at least 200 amino acids.

As used herein, a Fv antibody fragment is composed of one variable heavy domain (V_{H}) and one variable light (V_{L}) domain linked by noncovalent interactions.

As used herein, a dsFv refers to a Fv with an engineered intermolecular disulfide bond, which stabilizes the V_{H}-V_{L} pair.

As used herein, an F(ab)₂ fragment is an antibody fragment that results from digestion of an immunoglobulin with pepsin at pH 4.0-4.5; it can be recombinantly produced.

As used herein, a Fab fragment is an antibody fragment that results from digestion of an immunoglobulin with papain; it can be recombinantly produced.

As used herein, scFvs refer to antibody fragments that contain a variable light chain (V_{L}) and variable heavy chain (V_{H}) covalently connected by a polypeptide linker in any order. The linker is of a length such that the two variable domains are bridged without substantial interference. Exemplary linkers are (Gly-Ser)ₙ residues with some Glu or Lys residues dispersed throughout to increase solubility.

As used herein, hsFv refers to antibody fragments in which the constant domains normally present in an Fab fragment have been substituted with a heterodimeric coiled-coil domain (see, e.g., Arndt et al. (2001) J Mol Biol. 7:312:221-228).

As used herein, diabodies are dimeric scFv; diabodies typically have shorter peptide linkers than scFvs, and they preferentially dimerize.

As used herein, humanized antibodies refer to antibodies that are modified to include “human” sequences of amino acids so that administration to a human does not provoke an immune response. Methods for preparation of such antibodies are known. For example, the hybridoma that expresses the monoclonal antibody is altered by recombinant DNA techniques to express an antibody in which the amino acid composition of the non-variable regions is based on human antibodies. Computer programs have been designed to identify such regions.

As used herein, idiotype refers to a set of one or more antigenic determinants specific to the variable region of an immunoglobulin molecule.

As used herein, anti-idiotype antibody refers to an antibody directed against the antigen-specific part of the sequence of an antibody or T cell receptor. In principle an anti-idiotype antibody inhibits a specific immune response.

As used herein, phage display refers to the expression of proteins or peptides on the surface of filamentous bacteriophage.

As used herein, panning refers to an affinity-based selection procedure for the isolation of phage displaying a molecule with a specificity for a desired capture molecule or epitope.

As used herein, screening refers to the process analyzing molecules, such as sets of molecules and library compounds, by methods that include, but are not limited to, ultraviolet-visible (UV-VIS) spectroscopy, infra-Red (1R) spectroscopy, fluorescence spectroscopy, fluorescence resonance energy transfer (FRET), NMR spectroscopy, circular dichroism (CD), mass spectrometry, other analytical methods, high throughput screening, combinatorial screening, enzymatic assays, antibody assays and other biological and/or chemical screening methods or any combination thereof.

As used herein, staining refers to the visualization of molecules bound to the capture system. Staining can be non-specific, semi-specific or specific depending on what is labelled in a sample and when it is detected. Non-specific staining refers to the labelling of non-fractionated or all components in a particular sample generally, although not necessarily, prior to exposure to the capture system. Semi-specific staining as used herein refers to labelling of a portion of a sample, such as, but not limited to, the proteins located on the cell surface or on cellular membranes, either before, during or after e exposure to the capture system. Specific staining as used herein refers to the labelling of a specific component of a sample, typically after the exposure of the sample to the capture system. The stain can be any molecule that associates with that permits visualization or detection of bound molecules. As used herein, self-sorting refers to separation of a library of epitope-tagged molecules based on the affinity of the epitope for a specific capture agent.

As used herein, biological sample refers to any sample obtained from a living or viral source and includes any cell type or tissue of a subject from which nucleic acid or protein or other macromolecule can be obtained. Biological samples include, but are not limited to, cell lystates, cells, body fluids, such as blood, plasma, serum, cerebrospinal fluid, synovial fluid, urine and sweat, tissue and organ samples from animals and plants. Also included are soil and water samples and other environmental samples, viruses, bacteria, fungi algae, protozoa and components thereof. Hence bacterial and viral and other contamination of food products and environments can be assessed. The methods herein are practiced using biological samples and in some embodiments, such as for profiling, can also be used for testing any sample.

As used herein, macromolecule refers to any molecule having a molecular weight from the hundreds up to the millions. Macromolecules include peptides, proteins, nucleotides, nucleic acids, and other such molecules that are generally synthesized by biological organisms, but can be prepared synthetically or using recombinant molecular biology methods.

As used herein, the term “biopolymer” is used to mean a biological molecule, including macromolecules, composed of two or more monomeric subunits, or derivatives thereof, which are linked by a bond or a macromolecule. A biopolymer can be, for example, a polynucleotide, a polypeptide, a carbohydrate, or a lipid, or derivatives or combinations thereof, for example, a nucleic acid molecule containing a peptide nucleic acid portion or a glycoprotein, respectively. Biopolymer include, but are not limited to, nucleic acid, proteins, polysaccharides, lipids and other macromolecules. Nucleic acids include DNA, RNA, and fragments thereof. Nucleic acids can be derived from genomic DNA, RNA, mitochondrial nucleic acid, chloroplast nucleic acid and other organelles with separate genetic material.

As used herein, a biomolecule is any compound found in nature, or derivatives thereof. Biomolecules include but are not limited to: oligonucleotides, oligonucleosides, proteins, peptides, amino acids, peptide nucleic acids (PNAs), oligosaccharides and monosaccharides.

As used herein, the term “nucleic acid” refers to single-stranded and/or double-stranded polynucleotides such as deoxyribonucleic acid (DNA), and ribonucleic acid (RNA) as well as analogs or derivatives of either RNA or DNA. Also included in the term “nucleic acid” are analogs of nucleic acids such as peptide nucleic acid (PNA), phosphorothioate DNA, and other such analogs and derivatives or combinations thereof. Thus, the term also should be understood to include, as equivalents, derivatives, variants and analogs of either RNA or DNA made from nucleotide analogs, single (sense or antisense) and double-stranded polynucleotides, including double-stranded RNA. Deoxyribonucleotides include deoxyadenosine, deoxycytidine, deoxyguanosine and deoxythymidine. For RNA, the uracil base is uridine.

As used herein, the term “polynucleotide” refers to an oligomer or polymer containing at least two linked nucleotides or nucleotide derivatives, including a deoxyribonucleic acid (DNA), a ribonucleic acid (RNA), and a DNA or RNA derivative containing, for example, a nucleotide analog or a “backbone” bond other than a phosphodiester bond, for example, a phosphotriester bond, a phosphoramidate bond, a phophorothioate bond, a thioester bond, or a peptide bond (peptide nucleic acid). The term “oligonucleotide” also is used herein essentially synonymously with “polynucleotide,” although those in the art recognize that oligonucleotides, for example, PCR primers, generally are less than about fifty to one hundred nucleotides in length.

Nucleotide analogs contained in a polynucleotide can be, for example, mass modified nucleotides, which allows for mass differentiation of polynucleotides; nucleotides containing a detectable label such as a fluorescent, radioactive, luminescent or chemiluminescent label, which allows for detection of a polynucleotide; or nucleotides containing a reactive group such as biotin or a thiol group, which facilitates immobilization of a polynucleotide to a solid support. A polynucleotide also can contain one or more backbone bonds that are selectively cleavable, for example, chemically, enzymatically or photolytically. For example, a polynucleotide can include one or more deoxyribonucleotides, followed by one or more ribonucleotides, which can be followed by one or more deoxyribonucleotides, such a sequence being cleavable at the ribonucleotide sequence by base hydrolysis. A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well known methods (see, for example, Weiler et al. Nucleic acids Res. 25: 2792-2799 (1997)).

As used herein, oligonucleotides refer to polymers that include DNA, RNA, nucleic acid analogues, such as PNA, and combinations thereof. For purposes herein, primers and probes are single-stranded oligonucleotides or are partially single-stranded oligonucleotides.

As used herein, production by recombinant means by using recombinant DNA methods means the use of the well known methods of molecular biology for expressing proteins encoded by cloned DNA.

As used herein, substantially identical to a product means sufficiently similar so that the property of interest is sufficiently unchanged so that the substantially identical product can be used in place of the product.

As used herein, equivalent, when referring to two sequences of nucleic acids, means that the two sequences in question encode the same sequence of amino acids or equivalent proteins. When “equivalent” is used in referring to two proteins or peptides, it means that the two proteins or peptides have substantially the same amino acid sequence with only conservative amino acid substitutions (see, e.g., Table 1, below) that do not substantially alter the activity or function of the protein or peptide. When “equivalent” refers to a property, the property does not need to be present to the same extent but the activities are generally substantially the same. “Complementary,” when referring to two nucleotide sequences, means that the two sequences of nucleotides are capable of hybridizing, generally with less than 25%, with less than 15%, and even with less than 5% or with no mismatches between opposed nucleotides. Generally to be considered complementary herein the two molecules hybridize under conditions of high stringency.

As used herein, to hybridize under conditions of a specified stringency is used to describe the stability of hybrids formed between two single-stranded DNA fragments and refers to the conditions of ionic strength and temperature at which such hybrids are washed, following annealing under conditions of stringency less than or equal to that of the washing step. Typically high, medium and low stringency encompass the following conditions or equivalent conditions thereto:

1) high stringency: 0.1×SSPE or SSC, 0.1% SDS, 65° C.

2) medium stringency: 0.2×SSPE or SSC, 0.1% SDS, 50° C.

3) low stringency: 1.0×SSPE or SSC, 0.1% SDS, 50° C.

Equivalent conditions refer to conditions that select for substantially the same percentage of mismatch in the resulting hybrids. Additions of ingredients, such as formamide, Ficoll, and Denhardt's solution affect parameters such as the temperature under which the hybridization should be conducted and the rate of the reaction. Thus, hybridization in 5×SSC, in 20% formamide at 42° C. is substantially the same as the conditions recited above hybridization under conditions of low stringency. The recipes for SSPE, SSC and Denhardt's and the preparation of deionized formamide are described, for example, in Sambrook et al. (1989) Molecular Cloning, A Laboratory Manual, Cold Spring Harbor Laboratory Press, Chapter 8; see, Sambrook et al., vol. 3, p. B.13, see, also, numerous catalogs that describe commonly used laboratory solutions). It is understood that equivalent stringencies can be achieved using alternative buffers, salts and temperatures.

The term “substantially” identical or homologous or similar varies with the context as understood by those skilled in the relevant art and generally means at least 70%, preferably means at least 80%, more preferably at least 90%, and most preferably at least 95% identity.

As used herein, a reporter gene construct is a nucleic acid molecule that includes a nucleic acid encoding a reporter operatively linked to a transcriptional control sequences. Transcription of the reporter gene is controlled by these sequences. The activity of at least one or more of these control sequences is directly or indirectly regulated by a cell surface protein or other protein that interacts with tagged molecules or other molecules in the capture system. The transcriptional control sequences include the promoter and other regulatory regions, such as enhancer sequences, that modulate the activity of the promoter, or control sequences that modulate the activity or efficiency of the RNA polymerase that recognizes the promoter, or control sequences are recognized by effector molecules, including those that are specifically induced by interaction of an extracellular signal with a cell surface protein. For example, modulation of the activity of the promoter can be effected by altering the RNA polymerase binding to the promoter region, or, alternatively, by interfering with initiation of transcription or elongation of the mRNA. Such sequences are herein collectively referred to as transcriptional control elements or sequences. In addition, the construct can include sequences of nucleotides that alter translation of the resulting mRNA, thereby altering the amount of reporter gene product.

As used herein, “reporter” or “reporter moiety” refers to any moiety that allows for the detection of a molecule of interest, such as a protein expressed by a cell, or a biological particle. Typical reporter moieties include, include, for example, fluorescent proteins, such as red, blue and green fluorescent proteins (see, e.g., U.S. Pat. No. 6,232,107, which provides GFPs from Renilla species and other species), the lacZ gene from E. coli, alkaline phosphatase, chloramphenicol acetyl transferase (CAT) and other such well-known genes. For expression in cells, nucleic acid encoding the reporter moiety can be expressed as a fusion protein with a protein of interest or under to the control of a promoter of interest.

As used herein, the phrase “operatively linked” generally means the sequences or segments have been covalently joined into one piece of DNA, whether in single or double stranded form, whereby control or regulatory sequences on one segment control or permit expression or replication or other such control of other segments. The two segments are not necessarily contiguous. It means a juxtaposition between two or more components so that the components are in a relationship permitting them to function in their intended manner. Thus, in the case of a regulatory region operatively linked to a reporter or any other polynucleotide, or a reporter or any polynucleotide operatively linked to a regulatory region, expression of the polynucleotide/reporter is influenced or controlled (e.g., modulated or altered, such as increased or decreased) by the regulatory region. For gene expression a sequence of nucleotides and a regulatory sequence(s) are connected in such a way to control or permit gene expression when the appropriate molecular signal, such as transcriptional activator proteins, are bound to the regulatory sequence(s). Operative linkage of heterologous nucleic acid, such as DNA, to regulatory and effector sequences of nucleotides, such as promoters, enhancers, transcriptional and translational stop sites, and other signal sequences refers to the relationship between such DNA and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA in reading frame.

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences can be cis acting or can be responsive to trans acting factors. Promoters, depending upon the nature of the regulation, can be constitutive or regulated.

As used herein, the term “regulatory region” means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present, or at increased concentration, gene expression increases. Regulatory regions also include sequences that confer repression of gene expression (i.e., a substance or stimulus decreases transcription). When a repressor is present or at increased concentration, gene expression decreases. Regulatory regions are known to influence, modulate or control many in vivo biological activities including cell proliferation, cell growth and death, cell differentiation and immune-modulation. Regulatory regions typically bind one or more trans-acting proteins which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to an including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding sites (IRES) elements for the creation of multigene, or polycistronic, messages, polyadenylation signals to provide proper polyadenylation of the transcript of a gene of interest and stop codons and can be optionally included in an expression vector.

As used herein, regulatory molecule refers to a polymer of deoxyribonucleic acid (DNA) or ribonucleic acid (RNA), or an oligonucleotide mimetic, or a polypeptide or other molecule that is capable of enhancing or inhibiting expression of a gene.

As used herein, a composition refers to any mixture. It can be a solution, a suspension, liquid, powder, a paste, aqueous, non-aqueous or any combination thereof.

As used herein, a combination refers to any association between among two or more items. The combination can be two or more separate items, such as two compositions or two collections, can be a mixture thereof, such as a single mixture of the two or more items, or any variation thereof.

As used herein, a kit refers to a packaged combination, optionally including instructions and/or reagents for their use.

As used herein, a fluid refers to any composition that can flow. Fluids thus encompass compositions that are in the form of semi-solids, pastes, solutions, aqueous mixtures, gels, lotions, creams and other such compositions.

As used herein, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition., 1987, The Benjamin/Cummings Pub. Co., p.224).

Such substitutions can be made in accordance with those set forth in TABLE 1 as follows:

TABLE 1

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions are also permissible and can be determined empirically or in accord with known conservative substitutions.

As used herein, the amino acids, which occur in the various amino acid sequences appearing herein, are identified according to their well-known, three-letter or one-letter abbreviations. The nucleotides, which occur in the various DNA fragments, are designated with the standard single-letter designations used routinely in the art.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

The methods and collections herein are described and exemplified with particular reference to antibody capture agents, and polypeptide tags that include epitopes to which the antibodies bind, but is it to be understood that the methods herein can be practiced with any capture agent and any polypeptide tag therefor. It also to be understood that combinations of collections of any capture agents and polypeptide tag therefor are contemplated for use in any of the embodiments described herein. It is also to be understood that reference to array is intended to encompass any addressable collection, whether it is in the form of a physical array or labeled collection, such as capture agents bound to colored beads.

B. Collections of Binding Sites

Provided are collections binding sites (also referred to herein as capture systems) and methods using the collections. These collections contain addressable collections of capture agents that are bound to preselected tags, such as polypeptide tags. The tags are linked to molecules, biological particles or other moieties that are then displayed upon binding of the tags to the collections of capture agents. Because each tag can be linked to diverse collections of molecules, such as a molecular library with, for example, a diversity of 10⁴10¹², that bind to other molecules and biological particles, when the each tag is then captured by the addressable collection of capture agents, containing, for example, 10, 100, 200, 300, 400, 500, 1000 or more members, a highly diverse collection of binding sites can be displayed. Each locus in the collection is adderssable because each capture agent is addressable and each tag, such as a polypeptide tag, is specific for one capture agent. These addressable arrays contain collections of capture agents with tagged reagents bound thereto.

Practice of the methods provided herein involve some or all of the following steps: (1) identifying and obtaining capture agent—epitope pairs, such as antibodies and antigens; (2) identifying and obtaining a collection of molecules, such as a cDNA library, to display in the collection of binding sites; (3) conjugating the collection of molecules to different tags, such as polypeptide tags; and (4) contacting the tagged collections of molecules with the addressable collections of capture agents thereby sorting the tagged molecules due to the interaction between the collections of addressable capture agents, wherein each type of capture agent interacts specifically with a particular tag, such as a polypeptide tag, and producing a diverse collection of binding sites. The resulting diverse collections of binding sites can then be used in the methods provided herein to profile a sample by (5) contacting the addressable binding sites with a biological or chemical sample, including, but not limited to, cell lystates, cells, blood, plasma, serum, cerebrospinal fluid, synovial fluid, urine, sweat and tissue and organ samples from animals and plants, containing a complex mixture of components; (6) removing the unbound sample components; and (7) detecting the bound sample components, thereby producing a binding profile of the sample. Optionally, the some or all of following additional steps can be performed: (8) identifying a perturbation, such as a candidate compound, a condition, or both, that alters the binding profile of the sample; (9) exposing the collections of binding sites to a perturbation; and (10) detecting and/or monitoring the alterations in the binding profile of the sample in the presence of the perturbation. These optional steps can be performed before, after or during any of steps (4)-(7) or any other steps in such method. Other optional additional steps include labelling of the candidate compound (Step (8)). Further, the steps of the methods of profiling a sample provided herein can be used iteratively. A variation in the binding profile or a perturbation identified by the methods herein can be again subjected to some or all of the above noted steps to further identify the variations or perturbations.

In practice, to begin the method, a collection of molecules, such as a cDNA library, is identified and selected. The collection of molecules can include molecules with similar characteristics, such as three dimensional structure, chemical activity and physical location within a cellular environment, or can be vastly varied from one another. The molecule within the collection, such as a scFv library, can be identified by a variety of methods, including from the sources described herein, other methods described herein and by methods apparent to those skilled in the art based upon the description herein. For example, databases of literature, molecules and biological particles can be mined randomly for target interactions of interest. Empirical methods can also be employed for the identification of collections of molecule. A collection of molecule can be selected based on a variety of criteria, including, but not limited to, availability, cost, improving the understanding of the problem to be solved and applicability to a larger system. Other criteria for the selection of collections of molecule, such as a scFv library, is described herein, and apparent to those skilled in the art based description herein.

Following identification of a collection of molecules, the members of the collection of molecule are identified, selected and obtained. The number of molecule within the collection can vary depending on factors, such as the diversity of binding sites to be displayed, the physical size of the array to be printed and the number of capture agent/binding tag pairs available. Members of a collection of molecules obtained by a variety of methods, including, but not limited to, isolation from complex mixtures, commercial sources, other methods described herein and by methods apparent to those with skill in the art based upon the description herein. For example, databases of biomolecules can be mined for molecules of interest, such as, but not limited to a specific protein, nucleic acid, antibody, virus, cell, and enzyme.

Once the members of the collection of molecules is obtained, the members are conjugated to a specific tag, such as a polypeptide tag, including, but not limited to, a peptide, a protein or an antibody. The members are conjugated such that the aspect that makes them of interest, such as their 3-D structure or biological activity, is not impaired. Further, the members are conjugated with a tag, such as a polypeptide tag, that is specific for a capture agent that has been or will be addressably arrayed. Optionally, the members can be labelled with a detectable label, such as a luminescent label and a secondary antibody, to enable detection of the molecule or biological particle on the microarray. Conjugation of the members with the tag, such as an epitope tag, can, optionally, introduce additional domains into the conjugated complex, such as domains for the amplification of the complex and domains for the recovery of the complex from the collection. The conjugated members are then contacted with the addressable collections of capture agents that interact with the tag, such as a polypeptide tag, to produce the diverse collection of binding sites. Contact of the conjugated members, such as a scFv library, with the collections of capture agents can be performed individually or as a batch sample.

These collections of binding sites have a variety of applications, and are particularly useful for profiling complex samples. For example, the binding sites can be used to capture components of biological or chemical samples. Once captured by the diverse binding sites, the unbound molecules or biological particles from the sample can be removed and the components of the sample remaining can be detected. The components that remain bound to the binding sites are detected by any method known to those of skill in the art, such as luminescently, radioactivity and spectroscopically. The resulting pattern that is detected is the binding profile of the sample. Optionally, a perturbation, such as a candidate compound or a condition, can be added to the collection of binding sites prior to, simultaneously with or following the contact of the conjugated members with the capture agents or the sample with the collection of binding sites, to identify compounds and/or conditions that alter the binding profile of the sample. Such binding profiles and variations in the binding profiles as a result of a change in the sample or the addition of a perturbation have diagnostic and prognostic uses as well as in drug discovery.

1. Capture Agents

Capture agent refers to a molecule that has an affinity for a given ligand or with a defined sequence of amino acids. Capture agent, receptor and anti-ligand are interchangeable. In addition to antibodies and binding fragments thereof, any agent that specifically binds with reasonable affinity to tags, such as polypeptide tags, to subdivide a tagged library is a capture agent. Capture agents can be naturally-occurring or synthetic molecules, and include any molecule, including nucleic acids, small organics, proteins and complexes that specifically bind to specific sequences of amino acids. Capture agents are receptors and are also referred to in the art as anti-ligands. Capture agents can be used in their unaltered state or as aggregates with other species. They can be attached or in physical contact with, covalently or noncovalently, a binding member, either directly or indirectly via a specific binding substance or linker. Examples of capture agents, include, but are not limited to: antibodies, cell membrane receptors surface receptors and internalizing receptors, monoclonal antibodies and antisera reactive or isolated components thereof with specific antigenic determinants (such as on viruses, cells, or other materials), drugs, polynucleotides, nucleic acids, peptides, cofactors, lectins, sugars, polysaccharides, cells, cellular membranes, and organelles.

Examples of capture agents, also include but are not restricted to:

a) enzymes and other catalytic polypeptides, including, but are not limited to, portions thereof to which substrates specifically bind, enzymes modified to retain binding activity lack catalytic activity;

b) antibodies and portions thereof that specifically bind to antigens or sequences of amino acids;

c) nucleic acids;

d) cell surface receptors, opiate receptors and hormone receptors and other receptors that specifically bind to ligands, such as hormones. For the collections herein, the other binding partner, referred to herein as a polypeptide tag for each refers the substrate, antigenic sequence, nucleic acid binding protein, receptor ligand, or binding portion thereof.

As noted, contemplated herein, are pairs of molecules, generally proteins that specifically bind to each other. One member of the pair is a polypeptide that is used as a tag and encoded by nucleic acids linked to the library; the other member is anything that specifically binds thereto. The collections of capture agents, include receptors, such as antibodies or enzymes or portions thereof and mixtures thereof that specifically bind to a known or knowable defined sequence of amino acids that is typically at least about 3 to 10 amino acids in length. These agents include immunoglobulins of any subtype (IgG, IgM, IgA, IgE, IgE) or those of any species (such as IgY of avian species (Romito et al. (2001) Biotechniques 31:670, 672, 674-670, 672, 675.; Lemamy et al. (1999) Int. J. Cancer 80:896-902; Gassmann et al. (1990) FASEB J. 4:2528-2532), or the camelid antibodies lacking a light chain (Sheriff et al. (1996) Nat. Struct. Biol. 3:733-736; Hamers-Casterman et al. (1993) Nature 363:446-448) can be raised against virtually limitless entities. Polyclonal and monoclonal immunoglobulins can be used as capture agents. Additionally fragments of immunoglobulins derived by enzymatic digestion (Fv, Fab) or produced by recombinant means (scFv, diabody, Fab, dsFv, single domain Ig) (Arbabi et al. (1997) FEBS Lett. 414:521-526; Martin et al. (1997) Protein Eng 10:607-614; Holt et al. (2000) Curr. Opin. Biotechnol. 11:445-449) are suitable capture agents. Additionally, entirely new synthetic proteins and peptide mimetics and analogs can be designed for use as capture agents (Pessi et al. (1993) Nature 362:367-369).

Many different protein domains have been engineered to introduce variable regions to mimic the diversity seen in antibody molecules. Lipocalin (Skerra (2000) Biochim. Biophys. Acta 1482:337-350), fibronectin type III domains (Koide et al. (1998) J. Mol. Biol. 284:1141-1151), protein A domains (Nord et al. (2001) Eur. J. Biochem. 268:4269-4277; Braisted et al. (1996) Proc. Natl. Acad. Sci. U.S.A. 93:5688-5692), protease inhibitors (Kunitz domains, cysteine knots (Skerra (2000) J. Mol. Recognit. 13:167-187; Christmann et al. (1999) Protein Eng 12:797-806), thioredoxin (Xu et al. (2001) Biochemistry 40:4512-4520; Westerlund-Wikstrom, B (2000) Int. J. Med. Microbiol. 290:223-230), and GFP (Peelle et al. (2001) Chem. Biol. 8:521-534; Abedi et al. (1998) Nucleic Acids Res. 26:623-630) have been modified to function as binding agents. Many domains in proteins have been implicated in direct protein-protein interactions. With modifications, these interactions can be manipulated and controlled. For example, it is known that src homology-2 (SH2) domains are known to bind proteins containing a phosphorylated tyrosine (Ward et al. (1996) J. Biol. Chem. 271:5603-5609). The phosphotyrosine alone does not determine specificity, but amino acids surrounding it contribute to the binding affinity and specificity (Songyang et al. (1993) Cell 72:767-778). The SH2 domain can function as a capture agent. For example, altering amino acids in the binding pocket were new specificities result. Similarly, src homology 3 domains, SH3 domains bind a ten-residue consensus sequence, XPXXPPPFXP (where X is any amino acid residue, F is phenylalanine and P is proline; SEQ ID No. 102) (Sparks et al. (1998) Methods Mol. Biol. 84:87-103) can function as capture agents. Mutant SH3 domains can be selected to bind to tags with the above consensus sequence. The epidermal growth factor (EGF) domain has a two-stranded beta-sheet followed by a loop to a C-terminal short two-stranded sheet. This domain has been implicated in many protein-protein interactions, it can form the basis for a family of capture agents following manipulation of the loop between the two beta sheets. Long alpha-helical coils are known to interact with other alpha-helical segments to cause proteins to dimerize and trimerize. These coiled-coil interactions can be of very high affinity and specificity (Arndt et al. (2000) J. Mol. Biol. 295:627-639), and therefore can be used as capture agents when paired with complementary tags, such as epitope tags. Nearly any protein domain can be modified such that the variability introduced into one or more exposed regions of the molecule can constitute a potential binding site. Mutant enzymes, designated substrate trapping enzymes, that do not exhibit catalytic activity but retain substrate binding activity can be used (see, e.g., International PCT application No. WO 01/02600).

While most of the reagents used for affinity interactions with proteins are themselves proteins, there are many other potential protein-binding agents. Nucleic acids constitute a family of molecules that have inherent diversity of structure. Although there are only five naturally occurring subunits (ATP, CTP, TTP, GTP and UTP) compared to the twenty naturally occurring amino acids that make up proteins, they have the potential to fold into an immense variety of different structures capable of binding to a huge number of protein elements. Selection strategies for single-stranded RNA (Sun (2000) Curr. Opin. Mol. Ther. 2:100-105; Hermann et al. (2000) Science 287:820-825; Cox et al. (2001) Bioorg. Med. Chem. 9:2525-2531) and single-stranded DNA (or RNA) aptamers (Ellington et al. (1992) Nature 355:850-852) have been developed. These methods have proven successful for discovery of high affinity binders to small molecules as well as proteins. Using these methods, aptamers that bind with high specificity and affinity to tags, such as polypeptide tags, can be selected and then used as capture agents.

Single-stranded DNA or RNA can fold into diverse structures. Double-stranded nucleic acids, while more restricted in overall structure, can be used as capture agents with the correct tags, such as polypeptide tags. DNA binding proteins such as proteins containing zinc finger domains (Kim et al. (1998) Proc. Natl. Acad. Sci. U.S.A. 95:2812-2817) and leucine zippers (Alber (1992) Curr. Opin. Genet. Dev. 2:205-210) domains bind with high specificity to double stranded DNA molecules of defined sequence. Zinc finger domains bind to dsDNA in an arrayed format (see, e.g., Bulyk et al. (2001) Proc. Natl. Acad. Sci. U.S.A. 98:7158-7163). Additionally, DNA modifying enzymes can be modified for use as tags to bind to DNA used as an affinity capture agent. For example, the DNA restriction endonuclease BamHI has specific target sequence of GGATCC, but with mutation of the active site, a new enzyme is created that recognizes the sequence GCATGC. It also has been demonstrated that basepairs outside the specific target sequence play an important roll in the binding affinity, and that the catalytic event can be eliminated in the absence of the cofactor Mg²⁺ (Engler et al. (2001) J. Mol. Biol. 307:619-636). Mutations in some restriction enzymes abolish the cleavage event and leave the DNA binding domain bound to the dsDNA target (Topal et al. (1993) Nucleic Acids Res. 21:2599-2603; Mucke et al. (2000) J. Biol. Chem. 275:30631-30637). Thus panels of double-stranded nucleic acids can serve as capture agents.

Small chemical entities also can be designed to be capture agents. The highest affinity non-covalent interaction involving a protein is between proteins such as egg-white avidin or the bacterial streptavidin and the small, naturally-occurring chemical entity biotin. Biotin-like molecules can be used as capture agents if the tags are avidin-like proteins. Panels of chemically synthesized biotin analogs, and a corresponding panel of avidin mutants each capable of specific, high affinity binding to those biotin analogs can be employed. Other chemical entities have specific affinity for protein sequences. For example, immobilized metal affinity chromatography has been widely used for purification of proteins containing a hexa-histidine tag. Iminodiacetic acid, NTA or other metal chelators are used. The metal used determines the strength of interaction and possibly the specificity. Similarly, proteins that bind to other metals (Patwardhan et al. (1997) J. Chromatogr. A 787:91-100) can be selected.

Similarly, digoxin and a panel of digoxin analogs can be used as capture agents if the tags, such as polypeptide tags, are designed to bind to those analogs. Antibodies and scFvs have been created that bind with high specificity to these analogs (Krykbaev et al. (2001) J. Biol. Chem. 276:8149-8158) and the recombinant scFvs can themselves be used as tags. Carbohydrates, lipids, gangliosides can be used as capture agents for tags in the form of lectins (Yamamoto et al. (2000) J. Biochem. (Tokyo) 127:137-142; Swimmer et al. (1992) Proc. Natl. Acad. Sci. U.S.A. 89:3756-3760), fatty acid binding proteins (Serrero et al. (2000) Biochim. Biophys. Acta 1488, 245-254.) and peptides (Matsubara et al. (1999) FEBS Lett. 456:253-256).

2. Tags (Binding Partners) and Formats for Tags

As described above, any moiety, generally a protein, that specifically binds to a capture agent is contemplated as a tag, such as an epitope tag, also termed a binding partner. The term “epitope” is not to be construed as limited to an antibody-binding polypeptide, but as any specifically binding moiety. A polypeptide tag refers to a sequence of amino acids that includes the sequence of amino acids, herein referred to as an epitope, to which an anti-tag capture agent, such as an antibody and any agent described above, specifically binds. For tags and polypeptide tags, the specific sequence of amino acids to which each binds is referred to herein generically as an epitope. Any sequence of amino acids that binds to a receptor therefor is contemplated. For purposes herein the sequence of amino acids of the tag, such as epitope portion of the tag, that specifically binds to the capture agent is designated “E”, and each unique epitope is an Eₘ. Depending upon the context “Eₘ” can also refer to the sequences of nucleic acids encoding the amino acids constituting the epitope. The tag, such as a polypeptide tag, can also include amino acids that are encoded by the divider region.

In particular, the tag, such as a polypeptide tag, is encoded by the oligonucleotides provided herein, which are used to introduce the tag. When reference is made to a tag (i.e., binding pair for a particular receptor or portion thereof) with respect to a nucleic acid, it is the nucleic acid encoding the tag to which reference is made. Each tag, such as a polypeptide tag, is referred to as Eₘ (again E is not intended to limit the tags to “epitopes”, but include any sequence of amino acids that specifically binds to a capture agent); when nucleic acids are being described the Eₘ is the nucleic acid and refers to the sequence of nucleic acids that encodes the epitope; when the translated proteins are described Eₘ refers to amino acids (the actual epitope). The number of E's corresponds to the number of capture agents, such as antibodies, in an addressable collection. “m” is typically at least 10, 30 or more, 50 or 100 or more, and can be as high as desired and as is practical. Generally “m” is about a 1000 or more.

Any of the proteins described as possible capture agents can be used as tags, and vice versa, as long as the capture agents are addressable, such as by arraying, labeling with nanobarcodes or other such codes, encoded with colored beads and other such addressing products. The tags, such as polypeptide tags, are not necessarily small peptide sequences.

In some cases, it may be necessary or desirable to have the DNA sequences used for sub-division of a library or recovery of a sub-library be distinct from the protein encoding tags, such as epitope tags. Furthermore, particularly for certain applications, such as profiling (described in detail below), the tag, such as a polypeptide tag, is not required to be genetically fused to the library of interest such that a single protein is synthesized. It is possible to prepare tags, such as polypeptide tags, that are encoded as a separate protein that remains physically or otherwise associated with the library member. For example, dimerizing domains can be used to couple two separate proteins expressed in the same cell (Chao et al. (1998) J. Chromatogr. B Biomed. Sci. Appl. 715:307-329; Hodges (1996) Biochem. Cell Biol. 74, 133-154; Alber (1992) Curr. Opin. Genet Dev. 2:205-210). One of the dimerizing-domains is fused to the library protein, and its partner dimerizing-domain is fused to the tag protein. The dimerizing domains causes association of the library protein and tag, such as a polypeptide tag. These tags serve the same purpose of subdivision of the library on the addressable array. Also, the DNA for this tag is still associated with one specific subset of the total DNA library (since it is in the same plasmid or linear expression construct), and therefore indicates which subset to recover.

Another example, of a two-domain tag, such as a polypeptide tag, one in which DNA sequences used for subdivision of a library or recovery of a sub-library are distinct from the protein-encoding portion, tags, is larger proteins. For example, a larger protein such as a series of zinc finger (ZF) domains can be used as a polypeptide tag capable of binding to double stranded DNA (dsDNA, used as a capture agent). Specific fingers can be selected that bind to dsDNA sequences (Wu et al. (1995) Proc. Natl. Acad. Sci. U.S.A. 92:344-348; Jamieson et al./(1994) Biochemistry 33:5689-5695; and Rebar (199) Science 263:671-673). These zinc fingers are modular and can be combined to give increased specificity and affinity for the dsDNA target (Isalan et al. (2001) Nat. Biotechnol. 19:656-660; Kim (1998) Proc. Natl. Acad. Sci. U.S.A. 95:2812-2817).

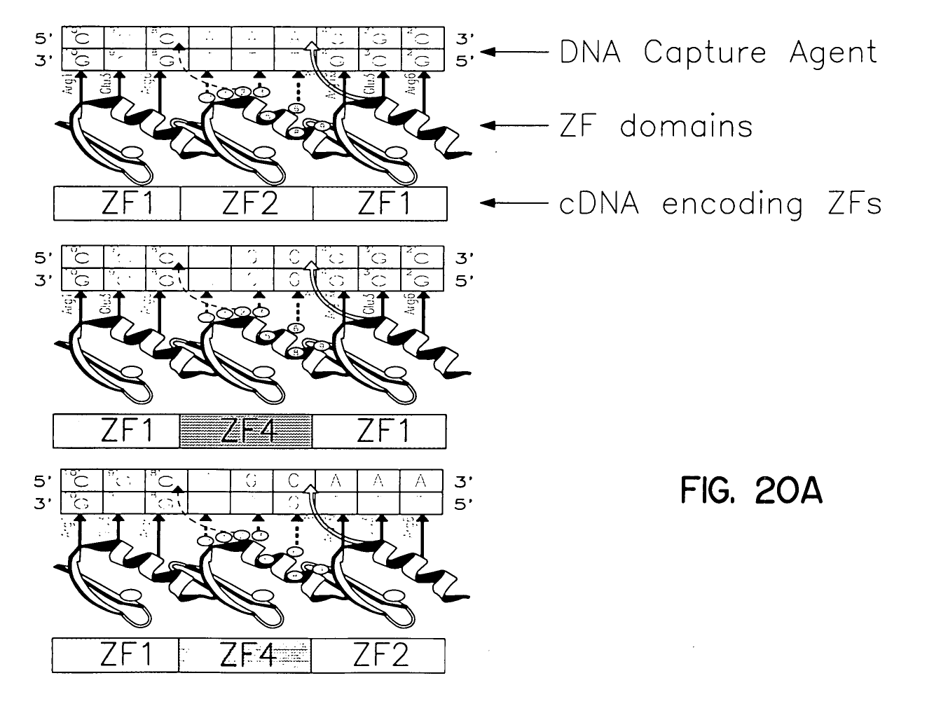

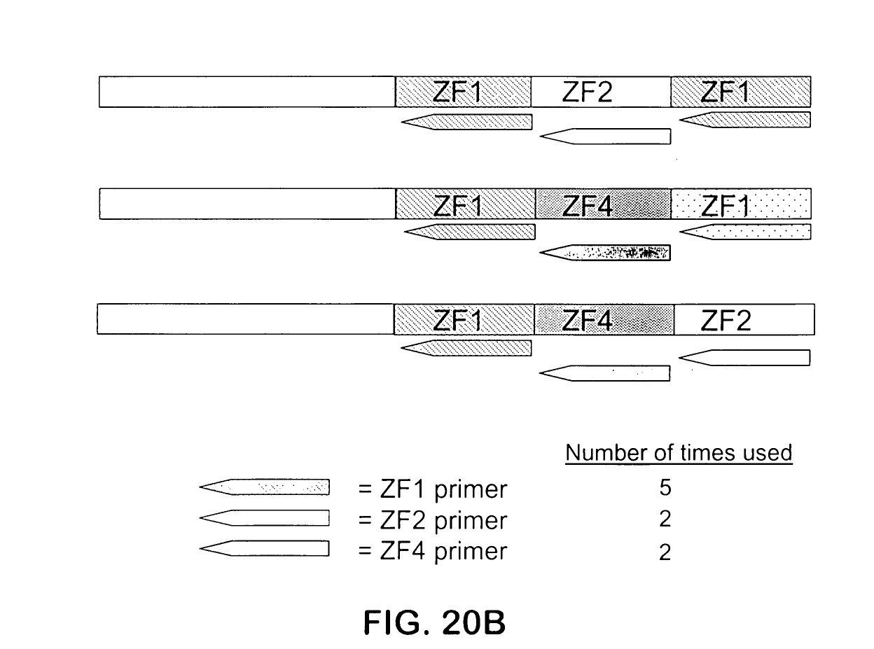

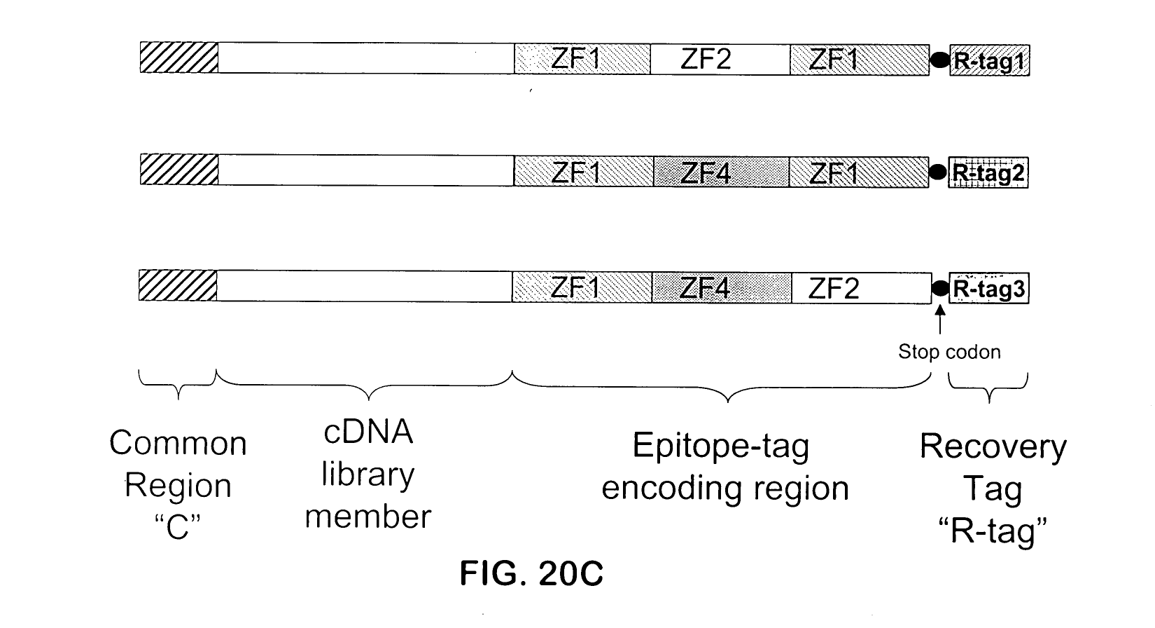

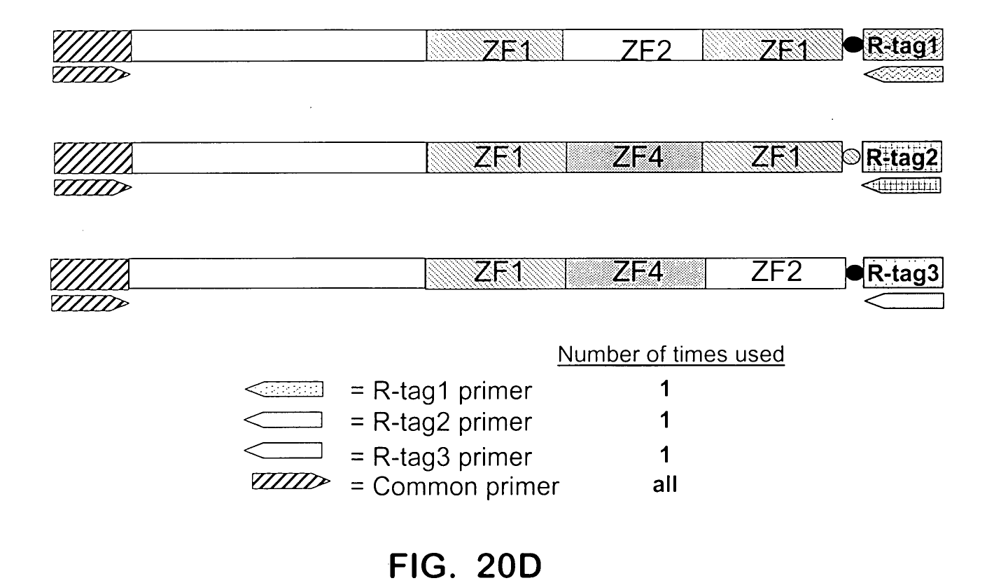

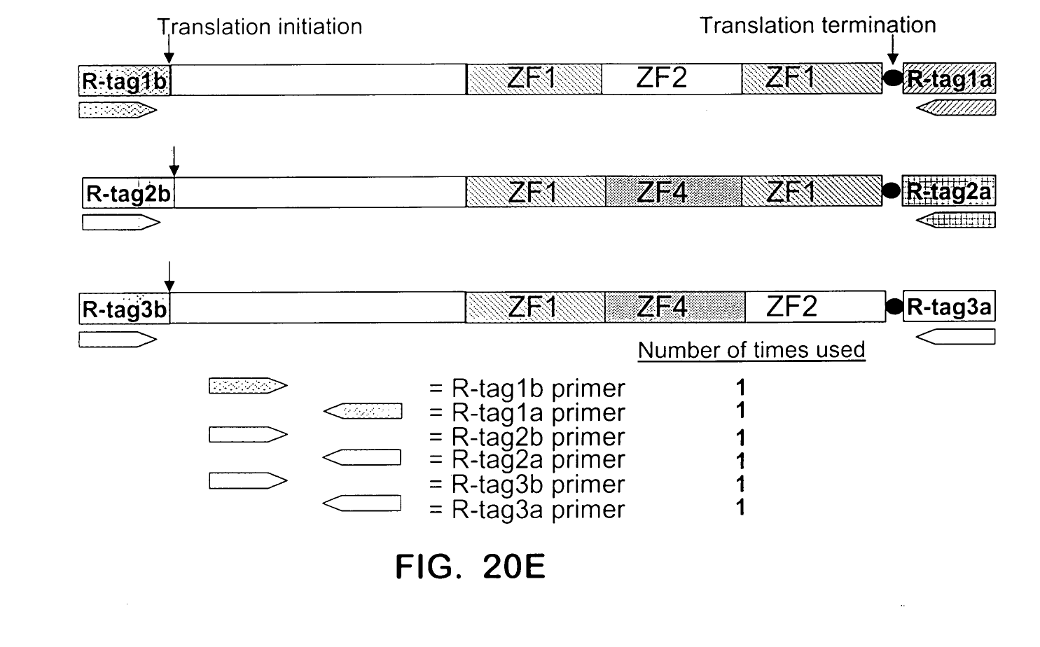

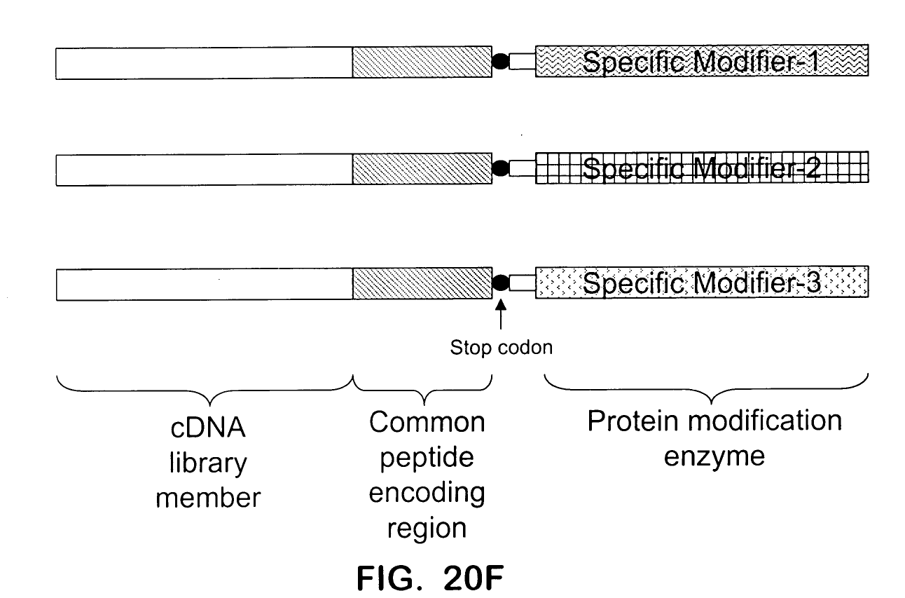

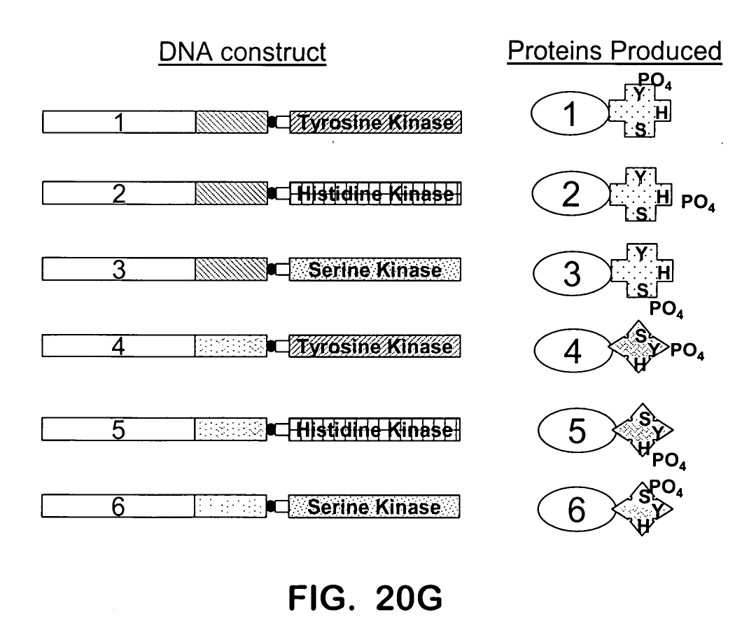

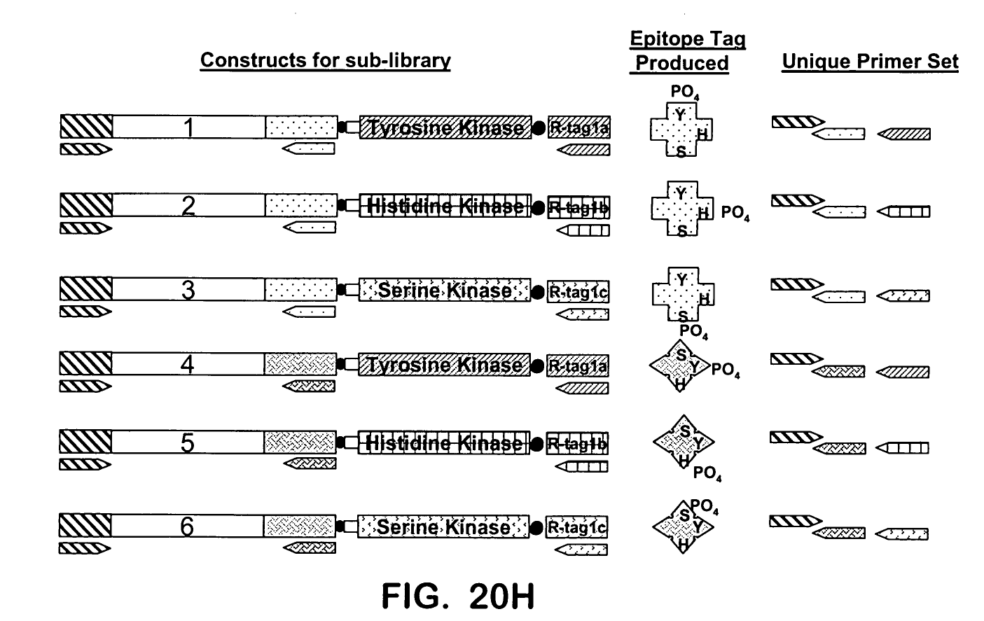

Due to the modular nature of these domains (see FIG. 20A, reproduced from Bulyk et al. (2001) Proc. Natl. Acad. Sci. U.S.A. 98:7158-7163 and modified), the conserved sequences in each module, and the overall size, it could be difficult to design oligonucleotide primers that correspond to the protein-encoding region and specifically amplify only a single class of tags. Shown schematically in FIG. 20A are three specific tags and their cognate capture agents (dsDNA sequences). Each tag is a DNA binding protein composed of three zinc finger domains that are arranged in a different order. The order as well as the composition of each domain will determine the specificity for the dsDNA capture agent. As indicated in FIG. 20B, oligonucleotide primers specific for a single domain could still amplify multiple different tags, such as polypeptide tags. Therefore, attempts to recover a specific sub-library could be inefficient.

Effective recovery of a single sub-library involves exclusive hybridization of an oligonucleotide with the target of interest. As shown, the repetitive use of single domains in multiple different tags, such as polypeptide tags, renders this exclusive hybridization doubtful. As noted, the nucleic acid encoding a tag, such as a polypeptide tag, includes a tag-specific amplification sequence (R-tag) that can be associated with a specific tag in a predetermined manner. This R-tag can encode protein, but does not need to be part of the binding portion of the encoded polypeptide tag. An R-tag does not necessarily encode protein, and can be located prior to the translational start site, or following the translational termination site or elsewhere. For example, as shown in FIG. 20C, a different recovery tag is associated with each tag. By separating the amplification portion from the epitope-encoding portion, it is possible to optimize each for the desired function, i.e., the R-tag portion can be an optimal amplification sequence, and the capture-agent-binding portion can be optimized for binding to a selected capture agent.

| Tag | Recovery tag |
|---|---|
| ZF1-ZF2-ZF1 | R-tag1 |
| ZF1-ZF4-ZF1 | R-tag2 |
| ZF1-ZF4-ZF2 | R-tag3 |

Therefore, while no oligonucleotide corresponding to a single domain in the tag, such as a polypeptide tag, could be used to specifically amplify a given sub-library (see FIG. 20B), each of the R-tags could be used to specifically amplify its corresponding sub-library (see FIG. 20D). Because the R-tags do not need to encode protein, there is considerable flexibility in designing sequences that will allow the specific hybridization (and through PCR, thus amplification) of only the correct corresponding sequences. Many available DNA sequence analysis software packages (Lasergene's DNAStar, Informax's Vector NTi, etc.) allow the analysis of oligonucleotides for melting temperature, primer-dimer formation, hairpin formation as well as cross-reactivity and mis-priming.

To increase specificity further, two specific R-tags can be associated with each particular tag such that one is prior to the translation initiation site, and the other is following the translation termination signal (FIG. 20E). Therefore, neither R-tag is encoded into the protein, but the inclusion of a second R-tag will increase the stringency to ensure recovery of only the correct corresponding sub-libraries. Instead of flanking the cDNA library and tag, such as a polypeptide tag, encoding regions, the two recovery tags associated with each tag sub-library could be in the format of nested primers on only one side of the protein-encoding region. These nested primers are used in succession in two sequential reactions.

Furthermore, tags, such as epitope tags, are not necessarily polypeptides. It is possible that the ligand for the capture agent is a protein modification such as a phosphorylated amino acid. Capture agents can distinguish combinations of phosphorylated and non-phosphorylated residues contained in a peptide. For example, mutated SH2 domains are arrayed as capture agents such that one can bind the sequence His-PO₄Tyr-Ser-Thr-Leu-Met, a second can bind His-Tyr-PO₄Ser-Thr-Leu-Met and a third can bind His-Tyr-Ser-PO₄Thr-Leu-Met and a fourth PO₄His-Tyr-Ser-Thr-Leu-Met. Each of these peptide sequences is the same yet the position of the phosphate group will determine the specificity. In each of these cases, the peptide is fused to the library member, but an additional encoded protein (Serine, Histidine, Threonine, or Tyrosine kinases) directs the phosphorylation event separately (FIGS. 20F and 20G).

In this case the tag, such as an epitope tag, has two separate determinates, the peptide sequence and the kinase responsible for the phosphorylation event thus recovery entails two sequential PCR steps (See FIG. 20H). As for the above example, these tags serve the same purpose of subdivision of the library in the addressable collection. Also, the DNA for this tag (the peptide and the kinase) are associated with one specific subset of the total DNA library (by nature of being in the same plasmid or linear expression construct), and therefore indicate which subset to recover. Other protein modifying enzymes include, but are not limited to, those that are involved fatty acid acylation, glycosylation, and methylation.

While the above descriptions and figures exemplify systems in which design of primers may be difficult, it may also be desirable to use a non-encoding associated R-tag even with simple linear capture-agent binding sequences. R-tags in some instances could be design for the PCR amplification steps since they are not constrained by the amino acids used in the tag, such as a polypeptide tag. The R-tag is associated with its corresponding capture agent-binding portion during the library creation process. For example, in embodiments in which cDNA is subcloned into a panel of vectors each containing a tag, the R-tag is also included in the vector.

In addition, modifications of the use of an enzyme modification of the tags before binding the capture agent can alter binding specificity. In such embodiments, the enzyme is not required to be physically linked to the tag, such as a polypeptide tag, as depicted in FIG. 20H. The enzyme-catalyzed modification is used to alter specificity of the tag for the capture agent or of a capture agent for a tag.

3. Covalent Interactions between Capture Agents and Tags

Generally the interaction between the capture agent and the tag, such as a polypeptide tag, involves reversible binding, such as the interaction between an antibody and an epitope, with an association constant sufficient for detection of the binding event.

Capture agents, however, can be modified such that following the specific affinity interaction, a crosslinking between the tagged reagent and the capture agent occurs. A covalent cross-linking reagent (through chemical, electrical, or photoactivatable methods) is often used to stabilize interactions between proteins (Besemer et al. (1993) Cytokine 5:512-519; Meh et al. (1996) J. Biol. Chem. 271:23121-23125; Behar et al. (2000) J. Biol. Chem. 275:9-17; Huber et al. (1993) Eur. J. Biochem. 218, 1031-1039). A cross-link ensures that the interaction between the capture agent and tag, such as a polypeptide tag, is long lasting and stable. The initial interaction between the capture agent and the tag, such as a polypeptide tag, determine the specificity while the cross-linking agent provides infinite affinity (Chmura et al. (2001) Proc. Natl. Acad. Sci. U.S.A. 98:8480-8484). This can be an added synthetic bi-functional cross-linking agent (Besemer et al. (1993) Cytokine 5:512-519; Meh et al. (1996) J. Biol. Chem. 271:23121-23125; Behar et al. (2000) J. Biol. Chem. 275:9-17; Huber et al. (1993) Eur. J. Biochem. 218, 1031-1039), or through a reactive group incorporated into the capture agent and the corresponding tag (Chmura et al. (2002) J. Control Release 78:249-258; Kiick et al. (2002) Proc. Natl. Acad. Sci. U.S.A. 99:19-24; Saxon et al. (2000) Org. Lett. 2:2141-2143; Lemieux et al. (1998) Trends Biotechnol. 16:506-513).

The covalent cross-link can be due to the enzymatic function of the tag, such as a polypeptide tag, or capture agent. For example, self-splicing proteins known as inteins have been used for the ligation of peptides to a larger protein (Ayers et al. (2000) J. Biol. Chem. 275:9-17), and for the ligation of two subunits of a split-intein protein (Wu et al. (1998) Biochim. Biophys. Acta 1387:422-432; Southworth et al. (1998) EMBO J. 17:918-926). Alternately, several DNA modifying enzymes use a mechanism that involves an intermediate in which the enzyme is covalently bound to its DNA substrate (Chen et al. (1995) Nucleic Acids Res. 23:1177-1183; Topal et al. (1993) Nucleic Acids Res. 21:2599-2603; Thomas et al. (1990) J. Biol. Chem. 265:5519-5530). It is likely that mutation of these enzymes can result in the stabilization of that intermediate, and thus the covalent linkage is retained. These modifying enzymes are highly sequence specific, and presumably can be mutated to create enzymes with distinct specificities. Thus, dsDNA can be used as an effective capture agent with a restriction enzyme or topoisomerase (or binding domain thereof as a tag, such as an epitope tag.

4. Methods for Tag (Binding Partner) Incorporation

Any method known to one of skill in the art to link a nucleic acid molecule encoding a polypeptide to another nucleic acid or to link polypeptide to another molecule is contemplated. For exemplification, a variety of such methods are described. As noted, they are described with particular reference to antibody capture agents, and polypeptide tags that include epitopes to which the antibodies bind, but is it to be understood that the methods herein can be practiced with any capture agent and polypeptide tag therefor.

a. Ligation to Create Circular Plasmid Vector for Introduction of Tags

As noted above, in addition to use of amplification protocols for introducing the primers into the library members, the primers can be introduced by direct ligation, such as by introduction into plasmid vectors that contain the nucleic acid that encode the tags and other desired sequences. Subcloning of a cDNA into double stranded plasmid vectors is well known to those skilled in the art. One method involves digesting purified double stranded plasmid with a site-specific restriction endonuclease to create 5′ or 3′ overhangs also known as sticky ends. The double stranded cDNA is digested with the same restriction endonuclease to generate complementary sticky ends. Alternately, blunt ends in both vector DNA and cDNA are created and used for ligation. The digested cDNA and plasmid DNA is mixed with a DNA ligase in an appropriate buffer (commonly, T4 DNA ligase and buffer obtained from New England Biolabs are used) and incubated at 16° C. to allow ligation to proceed. A portion of the ligation reaction is transformed into E. coli that has been rendered competent for uptake of DNA by a variety of methods (electroporation, or heat shock of chemically competent cells are two common methods). Aliquots of the transformation mix are plated onto semi-solid media containing the antibiotic appropriate for the plasmid used. Only those bacteria receiving a circular plasmid gives rise to a colony on this selective media. Creation of a library of unique members is performed in a similar manner, however the cDNA being inserted into the vector is a mixture of different cDNA clones. These different cDNA clones are created via a wide variety of methods known to those skilled in the art.

For directional cloning of cDNA clones, which is desirable for the creation of a library used for expression of proteins from the cDNA library, two different restriction endonucleases which generate different sticky ends are used for digestion of the plasmid. The cDNA library members are created such that they contain these two restriction endonuclease recognition sites at opposite ends of the cDNA. Alternately, different restriction endonucleases that generate complementary overhangs are used (for example digestion of the plasmid with NgoMIV and the cDNA with BspEI both leave a 5′CCGG overhang and are thus compatible for ligation). Furthermore, directional insertion of the cDNA into the plasmid vector brings the cDNA under the control of regulatory sequences contained in the vector. Regulatory sequences can include promoter, transcriptional initiation and termination sites, translational initiation and termination sequences, or RNA stabilization sequences. If desired, insertion of the cDNA also places the cDNA in the same translational reading frame with sequences coding for additional protein elements including those used for the purification of the expressed protein, those used for detection of the protein with affinity reagents, those used to direct the protein to subcellular compartments, those that signal the post-translational processing of the protein.

For example, the pBAD/gIII vector (Invitrogen, Carlsbad Calif.) contains an arabinose inducible promoter (araBAD), a ribosome binding sequence, an ATG initiation codon, the signal sequence from the M13 filamentous phage gene III protein, a myc polypeptide tag, a polyhistidine region, the rrnB transcriptional terminator, as well as the araC and beta-lactamase open reading frames, and the ColE1 origin of replication. Cloning sites useful for insertion of cDNA clones are designed and/or chosen such that the inserted cDNA clones are not internally digested with the enzymes used and such that the cDNA is in the same reading frame as the desired coding regions contained in the vector. It is common to use SfiI and NotI sites for insertion of single chain antibodies (scFv) into expression vectors. Therefore, to modify the pBAD/gIII vector for expression of scFvs, oligonucleotides SfiINotIFor (SEQ ID No. 6) and SfiINotIRev (SEQ ID no. 7) are hybridized and inserted into NcoI and HindIII digested pBAD/gIII DNA. The resultant vector permits insertion of scFvs (created with standard methods such as the “Mouse scFv Module” from Amersham-Pharmacia) in the same reading frame as the gene III leader sequence and the tag.

For use herein, a library of expressed proteins is subdivided using a plurality of tags, such as polypeptide tags, and the antibodies that recognize them. To create the library for expressing proteins with a plurality of tags, slight modifications of the subcloning techniques described above are used. A plurality of cDNA clones are inserted into a mixture of different plasmid vectors (instead of a single type of plasmid vector) such that the resulting library contains cDNA clones tagged with the different tags, such as polypeptide tags, and each tag is represented equally. Multiple plasmid vectors are created such that they differ in the tag that is translated in fusion with the inserted cDNA member. For example, if there are 1000 tag sequences, 1000 different vectors are constructed; if there are 250 tag sequences, 250 different vectors are constructed. Those skilled in the art understand that there are a variety of methods for construction of these vectors. For illustration, the myc epitope encoding region of the pBAD/gIII plasmid is removed by digestion with XbaI and SalI restriction enzymes, and the large 4.1 kb fragment is isolated. The hybridization of oligonucleotides HAFor (SEQ ID No. 8) and HARev2 (SEQ ID No. 74) creates overhangs compatible with XbaI and Sail, such that the product is inserted directionally, and encodes the epitope for the HA11 antibody (see table below). Insertion of the hybridization product of M2For (SEQ ID No. 10) and M2Rev2 (SEQ ID No. 11) results in a vector with the FLAG M2 epitope (see tables 2 and 3 below) in frame with the inserted cDNA. Insertion of the hybridization product of V5For (SEQ ID No. 75) and V5Rev (SEQ ID No. 76) results in a vector with the V5 epitope (see table below) in frame with the inserted cDNA. Hybridization and insertion of pairs of oligos listed in Table 2 below result in the creation of the epitopes (Table 3) in frame with the cDNA.

TABLE 2
Oligonucleotides

| | | SEQ |
|---|---|---|
| Oligo Name | Sequence 5′ to 3′ | ID No. |
| SfiINotIFor | catggcggcccagccggcctaatgagcggccgca | 6 |
| SfiINotIRev | agcttgcggccgctcattaggccggctgggccgc | 7 |
| HAFor | ctagaatatccgtatgatgtgccggattatgcgaatagcgccg | 8 |
| HARev | tcgacggcgctattcgcataatccggcacatcatacggataaa | 9 |
| HARev2 | tcgacggcgctattcgcataatccggcacatcatacggatatt | 74 |
| M2For | ctagaagattataaagatgacgacgataaaaatagcgccg | 10 |
| M2Rev2 | tcgacggcgctatttttatcgtcgtcatctttataatctt | 11 |
| V5for | CTAGAAggtaagcctatccctaaccctctcctcggtctcgattctacgAATAGCGCCG | 75 |
| V5rev | TCGACGGCGCTATTcgtagaatcgagaccgaggagagggttagggataggcttaccTT | 76 |
| StagFor | CTAGAAaaagaaaccgctgctgctaaattcgaacgccagcacatggacagcAGCGCCG | 77 |
| StagRev | TCGACGGCGCTgctgtccatgtgctggcgttcgaatttagcagcagcggtttctttTT | 78 |
| HSVtagFor | CTAGAAcagccggaactggcgccggaagatccggaagatAATAGCGCCG | 79 |
| HSVtagRev | TCGACGGCGCTATTatcttccggatcttccggcgccagttccggctgTT | 80 |
| T7tagFor | CTAGAAatggctagcatgactggtggacagcaaatgggtAATAGCGCCG | 81 |
| T7tagRev | TCGACGGCGCTATTacccatttgctgtccaccagtcatgctagccatTT | 82 |
| GluGluFor | CTAGAAgaagaggaggaatatatgccgatggaaAATAGCGCCG | 83 |
| GluGluRev | TCGACGGCGCTATTttccatcggcatatattcctcctcttcTT | 84 |
| KT3For | CTAGAAaaaccgccgaccccgccgccggaaccggaaaccAATAGCGCCG | 85 |
| KT3Rev | TCGACGGCGCTATTggtttccggttccggcggcggggtcggcggtttTT | 86 |
| EtagFor | CTAGAAggtgcgccggtgccgtatccggatccgctggaaccgcgtAATAGCGCCG | 87 |
| EtagRev | TCGACGGCGCTATTacgcggttccagcggatccggatacggcaccggcgcaccTT | 88 |
| VSVGfor | CTAGAAtacaccgacatcgaaatgaaccgtctgggtaaaAATAGCGCCG | 89 |
| VSVGrev | TCGACGGCGCTATTtttacccagacggttcatttcgatgtcggtgtaTT | 90 |
| Ab2For | ctagaaTTGACTCCTCCTATGGGTCCTGTTATTGATCAGCGGc | 129 |
| Ab2Rev | tcgagCCGCTGATCAATAACAGGACCCATAGGAGGAGTCAAtt | 130 |
| Ab4For | ctagaaTATAATATGGAATCGTATCTGTGGTATTTGGCGCCGc | 131 |
| Ab4Rev | tcgagCGGCGCCAAATACCACAGATACGATTCCATATTATAtt | 132 |
| B34For | ctagaaGATCTTCATGATGAGCGTACTCTTCAGTTTAAGCTTc | 133 |
| B34Rev | tcgagAAGCTTAAACTGAAGAGTACGCTCATCATGAAGATCtt | 134 |
| P5D4aFor | ctagaaCATCCGAATTTGCCTGAGACTCGTCGTTATGCGCTGc | 135 |
| P5F4aRev | tcgagCAGCGCATAACGACGAGTCTCAGGCAAATTCGGATGtt | 136 |
| P5D4bFor | ctagaaTCTTATACTGGGATTGAGTTTGATCGTTTGTCGAATc | 137 |
| P5D4bRev | tcgagATTCGACAAACGATCAAACTCAATCCCAGTATAAGAtt | 138 |
| 4C10For | ctagaaATGGTGGATCCTGAGGCGCAGGATGTGCCGAAGTGGc | 139 |
| 4C10Rev | tcgagCCACTTCGGCACATCCTGCGCCTCAGGATCCACCATtt | 140 |

TABLE 3
Antibody Epitopes

| Antibody | Epitope name | Sequence | SEQ ID |
|---|---|---|---|
| anti-9E10 | myc | EQKLISEEDL | 91 |
| anti-HA.11, HA.7, or 12CA5 | HA | YPYDVPDYA | 92 |
| anti-M1, M2, M5 | FLAG | DYKDDDDK | 93 |
| anti-GluGlu | GluGlu | EEEEYMPME | 94 |
| anti-V5-tag | V5 | GKPIPNPLLGLDST | 95 |
| anti-T7-tag | T7 | MASMTGGQQMG | 96 |
| anti-HSV-tag | HSV | QPELAPEDPED | 97 |
| S protein (not an antibody) | S-tag | KETAAAKFERQHMDS | 98 |
| anti-KT3 | KT3 | KPPTPPPEPET | 99 |
| anti-E-tag | E-tag | GAPVPYPDPLEPR | 100 |
| anti-P5D4 | VSV-g | YTDIEMNRLGK | 101 |
| anti-B34 | B34 | DLHDERTLQFKL | 106 |
| anti-P5D4-A | VSV-1 | HPNLPETRRYAL | 107 |
| anti-P5D4-B | VSV-2 | SYTGIEFDRLSN | 108 |
| anti-4C10 | 4C10 | MVDPEAQDVPKW | 109 |
| anti-AB2 | AB2 | LTPPMGPVIDQR | 110 |
| anti-AB4 | AB4 | QPQSKGFEPPPP | 111 |
| anti-AB3 | AB3 | YEYAKGSEPPAL | 112 |
| anti-AB6 | AB6 | AGTQWCLTRPPC | 113 |
| anti-KT3-A | KT3-A | KLMPNEFFGLLP | 114 |
| anti-KT3-B | KT3-B | KLIPTQLYLLHP | 115 |
| anti-KT3-C | KT3-C | SFMPIEFYARKL | 116 |
| anti-7.23 | 7.23 | TNMEWMTSHRSA | 117 |
| anti-S1 | S1 | NANNPDWDF | 118 |
| anti-E2 | E2 | SSTSSDFRDR | 119 |
| anti-His tag | His tag | HHHHHHGS | 120 |
| anti-AU1 | AU1 | DTYRYI | 121 |
| anti-AU5 | AU5 | TDFYLK | 122 |
| anti-IRS | IRS | RYIRS | 123 |
| anti-NusA | NusA | NusA Protein | 124 |
| anti-MBP | MBP | Maltose Binding Protein | 125 |
| anti-TBP | TBP | TATA-box Binding Protein | 126 |
| anti-TRX | TRX | Thioredoxin | 127 |
| anti-HOPC1 | HOPC1 | MPQQGDPDWVVP | 128 |

Each of these vectors still shares the SfiI and NotI restriction endonuclease sites to allow subcloning of cDNA clones into the vectors. Similarly, additional oligonucleotides can be designed to encode a wide variety of tags, such as epitope tags, that can be inserted in the same position to create a collection of different vectors.

Plasmid DNA corresponding to the vectors containing different tags, such as epitope tags, is prepared using methods known to those in the art (Qiagen columns, CsCl density gradient purification, etc). Purified double stranded DNA from each of the plasmids is quantified by OD260 and ethidium bromide staining on an agarose gel confirms quantification. Other methods can be used for quantification of plasmid DNA. Purified plasmid DNA corresponding to each of the tag-containing vectors is combined in equivalent amounts (1 μg for each plasmid) prior to digestion with the two restriction enzymes. For example, if 10 tag containing plasmid vectors are used, 10 μg of total DNA is incubated for 2 hours at 50° C. in a volume of 100 μl with 100 Units of SfiI (New England Biolabs) in 50 mM NaCl, 10 mM Tris-HCl, 1 mM MgCl₂₁ 1 mM dithiothreitol (DTT) pH 7.9 supplemented with 100 μg/ml bovine serum albumin (BSA). Following digestion with SfiI, the reaction is supplemented with additional H₂O, MgCl₂, Tris-HCl, NaCl, DTT, BSA, and NotI (New England Biolabs) such that the reaction volume is 150 μl containing 100 Units of NotI in 100 mM NaCl, 50 mM Tris-HCl, 1 mM MgCl₂, 1 mM DTT pH 7.9 and 100 μg/ml BSA. This reaction is incubated at 37° C. for 2 hours. Calf intestinal phosphatase (25 Units CIP, New England Biolabs) is added to the reaction and incubated at 37° C. for an additional 1 hour. The cDNA clones of interest are also digested with the same restriction enzymes under similar conditions. Digested plasmid DNA and cDNA clones are separated on agarose gels to remove unwanted sticky ends and purified from agarose slices using standard methods (Qiagen gel purification kit, GeneClean kit, etc). The cDNA clones and the mixture of plasmids are reacted in 1× ligase buffer at a 3:1 molar ratio (insert to vector) with T4 DNA ligase (New England Biolabs). Typically, a ligation reaction contains about 10 ng/μl plasmid DNA and 0.5 units/μl of T4 DNA ligase in a suitable buffer, and is incubated at 16° C. for 12 to 16 hours. The reaction is diluted 8-10 fold with sterile water, and aliquots are transformed by electroporation into TOP10F′ (electrocompetant E. coli cells from Invitrogen, or other similar cells). Liquid medium such as SOC (see, Sambrook et al. (1989) Molecular Cloning: A Laboratory Manual, 2nd Edition, Cold Spring Harbor Laboratory Press; SOC is 2% (w/v) tryptone, 0.5% (w/v) yeast extract, 8.5 mM NaCl, 2.5 mM KCl, 10 mM MgCl₂ and 20 mM glucose at pH 7) is added, and cells are allowed to recover for 1 hour at 37° C. An aliquot of the transformation mixture is plated on LB-agar plates containing 100 μg/ml ampicillin. Plates are incubated at 37° C. for 12 to 16 hours, and then individual clones are analyzed. This analysis indicates that each of the tags present in the initial mixture is represented equally in the final library.

For example, a series of plasmid vectors containing the EDC sequences is created such that each vector in the series contains a single combination of EDC sequences. For example, if there are 1000 E sequences in combination with 1000 D sequences and a single C sequence, there are 10⁶ (1000×1000×1) possible combinations and therefore 10⁶ vectors are created. Each of these vectors shares restriction endonuclease sites to allow subcloning (generally directional) of cDNA clones into the vectors. Purified plasmid DNA from all 10⁶ vectors is mixed and then digested with the restriction endonucleases. Alternatively, DNA representing each vector is digested and then mixed to create the pool of recipient vectors. Double stranded cDNA representing the library of interest is also digested with restriction endonucleases to create ends that are compatible for ligation to the ends created by vector digestion. This is accomplished by using the same enzymes for vector and cDNA digestion or by using those that generate complementary overhangs (for example NgoMIV and BspEI both leave a 5′CCGG overhang and are thus compatible for ligation). Alternately, blunt ends in both vector DNA and cDNA are created and used for ligation. Digested cDNA clones and digested vector DNAs are ligated using a DNA ligase such as T4 DNA ligase, E. coli DNA ligase, Taq DNA ligase or other comparable enzyme in an appropriate reaction buffer. The resultant DNA is transformed into bacteria, yeast, or used directly as template for in vitro transcription of RNA. The design of the vectors is such that insertion of the cDNA at the restriction endonuclease sites places the cDNA under control of promoter sequences to allow expression of the cDNA. Additionally the cDNA are in the same reading frame as the E sequence such that upon protein expression from this vector, a fusion protein containing the cDNA-encoded polypeptide fused to the tag is produced. The E sequence is positioned in the vector such that the encoded tag is fused to either the N or the C terminus of the resultant protein. (for restriction enzyme digestion, DNA ligation, and transformation, see, e.g., see, Sambrook et al. (1989) Molecular Cloning: A Laboratory Manual, 2nd Edition, Cold Spring Harbor Laboratory Press, Chapter 1).

b. Ligation of Sequences Resulting in Linear Tagged cDNA

Following creation of the cDNA library, sequences are appended to cDNA clones via ligation. Linear, double stranded DNA containing each of the EDC sequence combinations is created via various methods (synthesis, digestion out of plasmid containing the sequences, assembly of shorter oligonucleotides, etc.). These linear dsDNAs containing the different EDC sequences, are mixed such that each individual is equally represented in the mixture. This mixture is combined with the double stranded cDNA library and ligated using a nucleic acid ligase in an appropriate buffer. This is generally a DNA ligase, but an RNA ligase is used if the EDC tags are composed of RNA or are RNA/DNA hybrid molecules and the library is also in the form of an RNA or RNA/DNA hybrid. In one embodiment, the EDC sequence is blunt-ended on both ends yet only one end is phosphorylated such that ligation occurs in a directional manner (with respect to the EDC sequence) and the E sequence are brought into the same reading frame as the cDNA (at either the N or C terminus of the resulting protein). In another embodiment, the EDC sequence is blunt-ended at one end and has an overhang on the other end such that ligation occurs in a directional manner (see, Sambrook et al. (1989) Molecular Cloning: A Laboratory Manual, 2nd Edition, Cold Spring Harbor Laboratory Press Chapter 8). The EDC sequences can be continuously double stranded, or partially double stranded with a single stranded central portion.

In another embodiment, the cDNA library is created to contain a restriction endonuclease site and the same restriction site is included in the EDC sequences such that upon digestion of each with the appropriate enzyme, compatible ends are created. The digested library is ligated to a mixture of digested EDC sequences using a DNA ligase in an appropriate buffer. In another embodiment, the cDNA library is created to contain a restriction endonuclease site and the EDC sequences are designed to contain a restriction site that leaves an overhang compatible to the overhang generated on the cDNA. Upon ligation of these two compatible sites, a sequence is generated that is not susceptible to cleavage with either of the enzymes used to generate the overhangs. In this case, the products of the ligation reaction are digested with the enzymes used to generate the overhangs. Alternately, the ligation reaction occurs in the presence of the enzymes used to generate the overhangs (Biotechniques (1999) August 27(2): 328-30 and 332-4; and Biotechniques (1992) January 12(1): 28 and 30).

This method reduces and/or eliminates the ligation of cDNA to cDNA or EDC sequence to EDC sequence, and thus enrich for the cDNA-EDC product. Pairs of enzymes capable of generating such compatible overhangs include AgeI/XmaI, AscI/MluI, BspEI/NgoMIV, NcoI/PciI and others (New England Biolabs 2000-2001 catalog p184 and 218 for partial list). The EDC sequences and the cDNA are designed such that they are in the same reading frame following ligation. Therefore, upon protein expression from this construct, a fusion protein containing the cDNA-encoded polypeptide fused to the tag is produced. The E sequence is positioned in the final construct such that the encoded tag, such as an epitope tag, is fused to either the N or the C terminus of the resultant protein.

In another embodiment, the cDNA, the EDC sequence or both are created such that they contain a region with RNA hybridized to DNA. The RNA can be removed by digestion with the appropriate RNAse (including type 2 RNAse H) such that a single stranded DNA overhang results. This overhang can be ligated to compatible overhangs generated either by the above method or by restriction endonuclease digestion. Additionally, overhangs and flanking sequence are designed in such a way that if an EDC sequence is ligated to another EDC sequence, the resulting sequence is susceptible to digestion with a particular restriction enzyme. Likewise, if a cDNA is ligated to another cDNA, the resulting sequence is susceptible to cleavage by another restriction enzyme. Ligation reactions occur in the presence of those restriction enzymes, or are subsequently treated with those enzymes to reduce the incidence of cDNA-cDNA or EDC-EDC ligation events (see enzymes pairs and references above). The EDC sequences and the cDNA are designed such that they are in the same reading frame following ligation. Therefore, upon protein expression from this construct, a fusion protein containing the cDNA-encoded polypeptide fused to the tag is produced. The E sequence is positioned in the final construct such that the encoded tag is fused to either the N or the C terminus of the resultant protein. In another embodiment, PCR is used to generate the cDNA and the various EDC sequences using PCR primers that contain regions of RNA sequence that cannot be copied by certain thermostable DNA polymerases. Therefore RNA overhangs remain that can be ligated to complementary overhangs generated by the same method or by restriction enzyme digestion. RNA or DNA overhang cloning is described by Coijee et al. (Nat Biotechnol (2000) July 18(7): 789-91).

In another embodiment, an EDC sequence is brought into close apposition to a cDNA sequence by hybridization to a splint oligonucleotide that is complementary to the 3′ region of the cDNA and also the 5′ region of the EDC sequence (Landegen et al., Science 241:487, 1988). Joining of the cDNA and EDC is accomplished by a nucleic acid ligase under appropriate reaction conditions. In another embodiment, the splint oligonucleotide is complementary to the 5′ region of the cDNA and the 3′ region of the EDC sequence. In both cases, the different members of the cDNA library share a common sequence (at the 3′ or 5′ end), and the different EDC sequences also share a common sequence (at the 5′ or 3′ end), such that a single splint oligonucleotide sequence can hybridize to any member of the cDNA library and also to any individual of the series of EDC sequences. In each of these embodiments, the splint oligonucleotide, the cDNA and the EDC sequences can be single or double stranded DNA, or combinations of DNA and RNA. Mixtures of cDNA, EDC sequences and splint oligonucleotides are denatured at elevated temperatures to eliminate secondary structure and existing hybridization. The reaction is then cooled to allow hybridization to occur. In cases where the splint oligonucleotide is present in molar excess, a hybridization product containing the three desired components (cDNA, EDC and splint oligonucleotide) is obtained. A nucleic acid ligase is added and the reaction is incubated under appropriate conditions.

In another embodiment, the splint oligonucleotide, cDNA library and EDC sequences are designed as in the above example. The ligase chain reaction (see, e.g., LCR, F. Barany (1991) The Ligase Chain Reaction in a PCR World, PCR Methods and Applications, vol. 1 pp. 5-16; see, also, U.S. Pat. No. 5,494,810) is then performed using multiple cycles of denaturation, hybridization, and ligation with a thermostable ligase. For geometric amplification of cDNA-EDC product, double stranded cDNA and double stranded EDC sequences are needed.

c. Primer Extension and PCR for Tag Incorporation

In another embodiment, the EDC sequences are appended to the cDNA clones during the creation of the cDNA library. In this case, the EDC sequence is designed such that it can hybridize to a desired population of mRNA. This EDC serves as a primer and the RNA serves as a template for synthesis of DNA using reverse transcriptase (AMV-RT, M-MuLV-RT or other enzyme that synthesizes DNA complementary to RNA as template). The newly synthesized cDNA is complementary to the RNA and has an EDC sequence at the 5′end. Second strand synthesis using a DNA polymerase results in double stranded DNA with the EDC at the end corresponding to the 3′ end of the RNA. In this embodiment, all members in the series of EDC sequences share a common 3′ end for hybridization to the RNA (e.g., in the case of a library of similar members of a gene family). Alternately, EDC sequences have a sequence of random nucleotides at the 3′ end for random priming of RNA (Molecular cloning: a laboratory manual 2^{nd} edition, Sambrook et al, Chapter 8).

In another embodiment, the polymerase chain reaction (PCR) is used to append EDC sequences to cDNA clones. A cDNA library is created in such a way that all members share a common sequence at the 3′ end (e.g., prime first strand cDNA synthesis with an oligonucleotide containing this common sequence, or ligation of linker sequences to double stranded cDNA clones). Additionally, each member of the cDNA library share a different common sequence (“C”) at the 5′ end. Each unique member in the series of EDC sequences have a common 3′ end that is complementary to one of the common regions in the cDNA. This mixture of EDC sequences serve as one of the amplification primers in a polymerase chain reaction. An oligonucleotide complementary to the common region at the opposite end of the cDNA serve as the second amplification primer. The cDNA library is mixed with the series of EDC amplification primers, the second primer and a thermostable polymerase (Taq, Vent, Pfu, etc) in the appropriate buffer conditions and multiple cycles of denaturation, hybridization, and DNA polymerization are executed. Alternatively, the cDNA library is subdivided after the addition of the common sequences, and aliquots are combined with individual EDC sequences, the second primer and a thermostable polymerase (Taq, Vent, Pfu, etc) in the appropriate buffer conditions and multiple cycles of denaturation, hybridization, and DNA polymerization are executed.

d. Insertion by Gene Shuffling

In another embodiment, EDC sequences are appended to cDNA clones via “DNA shuffling” or molecular breeding (see, e.g., Gene (1995) October 16 164(1): 49-53; Proc. Natl. Acad. Sci. USA (1994) October 25 91(22): 10747-51; U.S. Pat. No. 6,117,679). Each member in the series of EDC sequences have a common 3′ end that is complementary to one of the common regions in the cDNA library members. During creation, or mutagenesis of the cDNA library, EDC sequences are included in the PCR reaction to allow the EDC sequences to be assembled along with the fragments of the cDNA clones.

e. Recombination Strategies

Recombination strategies can also be used for introduction of tags into cDNA clones. For example, triple-helix induced recombination is used to append EDC sequences to cDNA clones. A cDNA library is created in such a way that all members share a common sequence at one end. The series of EDC sequences is designed to include a region with considerable homology to the common sequence in the cDNA library. The EDC sequences and the cDNA library are combined in a cell free recombination system (J Biol Chem (2001) May 25 276(21): 18018-23) with a third homologous oligonucleotide and recombination is allowed to occur.

In another embodiment, site-specific recombination is used to append EDC sequences to cDNA clones. Site specific recombination systems include loxP/cre (U.S. Pat. No. 6,171,861; and U.S. Pat. No. 6,143,557), FLP/FRT (Broach et al. Cell 29: 227-234 (1982)), the Lambda integrase with attB and attP sites (U.S. Pat. No. 5,888,732), and a multitude of others. The series of EDC sequences as well as the members of the cDNA library are designed to include a common sequence recognized by the recombinase protein (e.g., loxP sites). The EDC sequences and the cDNA library are combined in a cell free recombination system (Protein Expr Purif (2001) June 22(1): 135-40) including the site specific recombinase (e.g., cre recombinase) under appropriate conditions to allow recombination to take place. Alternately, the recombination events take place inside cells such as bacteria, fungus, or higher eukaryotic cells expressing the desired recombinase (see, for example, U.S. Pat. Nos. 5,916,804, 6,174,708 and 6,140,129).

In another embodiment, homologous recombination in cells is used to append EDC sequences to cDNA clones. E. coli (Nat. Genet. (1998) October 20(2):123-8), yeast (Biotechniques (2001) March 30(3): 520-3), and mammalian cells (Cold Spring Harb Symp Quant Biol. (1984) 49:191-7) are used for recombination of DNA segments. The EDC sequences are designed to contain both 5′ and 3′ regions with homology to two separate regions in a plasmid vector containing the cDNA. The lengths of homologous regions are dependent on the cell type being used. The cDNA and the EDC sequences are co-transformed into the cells and homologous recombination is carried out by recombination/repair enzymes expressed in the cell (see, e.g., U.S. Pat. No. 6,238,923).

f. Incorporation by Transposases

In another embodiment, transposases are used to transfer EDC sequences to cDNA clones. Integration of transposons can be random or highly specific. Transposons such as Tn7 is highly site-specific and is used to move segments of DNA (Lucklow et al. J. Virol. 67: 4566-4579 (1993)). The EDC sequences are contained between inverted repeat sequences (specific to the transposase used). The members of the cDNA library (or the plasmid vectors they are in) contain the target sequence recognized by the transposase (e.g., attTn7). In vitro or in vivo transposition reactions insert the EDC sequences into this site.

g. Incorporation by Splicing

In another embodiment, EDC sequences flanked by RNA splice acceptor and donor sequences are inserted into the genome of various cell lines in such a way as to incorporate them into the mRNA being transcribed and translated (See U.S. Pat. No. 6,096,717 and U.S. Pat. No. 5,948,677). Proteins isolated from these organisms, or cell lines therefore contain the tags and are amenable to separation by our collection of antibodies.

In another embodiment, EDC sequences are appended to library members via trans-splicing of RNA. The RNA form of EDC sequences, and preceded by RNA splice acceptor sequences, or followed by splice donor sequences are expressed in cells that then receive the library of cDNA clones. Trans-splicing of RNA (Nat. Biotechnol. (1999) March 17(3): 246-52, and U.S. Pat. No. 6,013,487) append the EDC sequence to the library member.

h. An Alternative Method for Distribution of Tags

Alternative methods for effecting even distribution have been described (see, e.g., published International PCT application No. WO 02/06834; published U.S. application Ser. No. US20020137053; U.S. provisional application Serial No. 60/422,923; and U.S. provisional application Serial No. 60/423,018). In these methods, the tags were linked to molecules in the master library, prior to sub-division. This method, which can be practiced to distribute any type of tag on any collection of molecules, is particularly adaptable for instances in which the master library is a nucleic acid library and the tags that bind to the capture agents are polypeptide tags. In this method, described with reference nucleic acid, such as DNA libraries, the nucleic acid library is subdivided, tags are added to produce tagged sub-libraries, in which the nucleic acid encodes the same tag for all members of the sub-library, the tagged sub-libraries are pooled to form a mixed tag library such that the same number of tagged molecules is added from each sub-library. This can be achieved by adjusting the concentration of each tagged sub-library or an aliquot thereof or determining the concentration of tagged molecules each sub-library and pooling equivalent numbers of tagged molecules. The mixed tag library is contacted with addressed collection of capture agents in which the capture agents at or of each loci bind to the same tag, which generally differs from the tag to which the agents at other loci bind. Alternatively, the mixed library is divided or aliquots are removed and contacted with a predetermined number “q”, where q is from 2 or more, generally, 2 to 10, 20, 30, 50, 100, 200, 250, 300, 500, 1000, 2000, 3000, 4000, 5000, 10,000 and more, of addressable arrays, generally, although not necessarily, replicate arrays, of capture agents. As noted, generally, in the addressed collection of capture agents, the capture agents at or of each loci bind to the same tag, which generally differs from the tag to which the agents at other loci bind.

The method for even distributing tags on tagged-molecules that is provided herein includes some or all of the following steps:

a) determining the diversity of molecules required;

b) producing or obtaining a master library;

c) optionally, adjusting the diversity of a master library so that the diversity is substantially equal to, typically within an order of magnitude (i.e., within one order of magnitude, typically within 0.5 orders of magnitude or 0.1 orders of magnitude), the number of members of the library;

d) dividing the master library into “n” sublibraries designated 1−n, where n is equal to or less than the number of different tags, i.e., nucleic acid molecules having different sequences encoding different polypeptide tags in the exemplified embodiment;

e) attaching a nucleic acid molecule encoding a polypeptide tag (or attaching a tag) to members of each sublibrary to produce “n” tagged sublibraries containing encoded tagged members, whereby the polypeptide tag encoding portion is in reading frame with a polypeptide encoded by the nucleic acid molecule, and such that the encoded polypeptide tag is unique to each sublibrary;

f) mixing some or all of the tagged sublibraries to produce a mixed library, where the number of tagged molecules added from each sublibrary is the about the same (i.e., within one order of magnitude, typically within 0.5 orders of magnitude or 0.1 orders of magnitude);

g) splitting the mixed library into “q” array libraries, where q is from 1 to a predetermined number of arrays; and

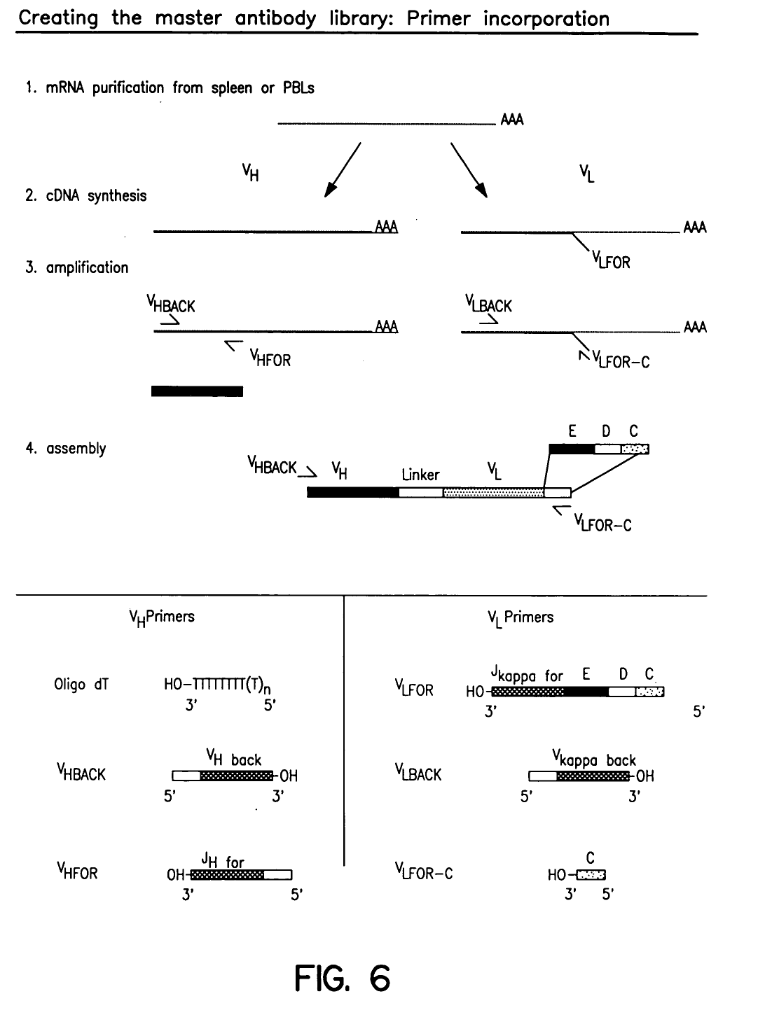

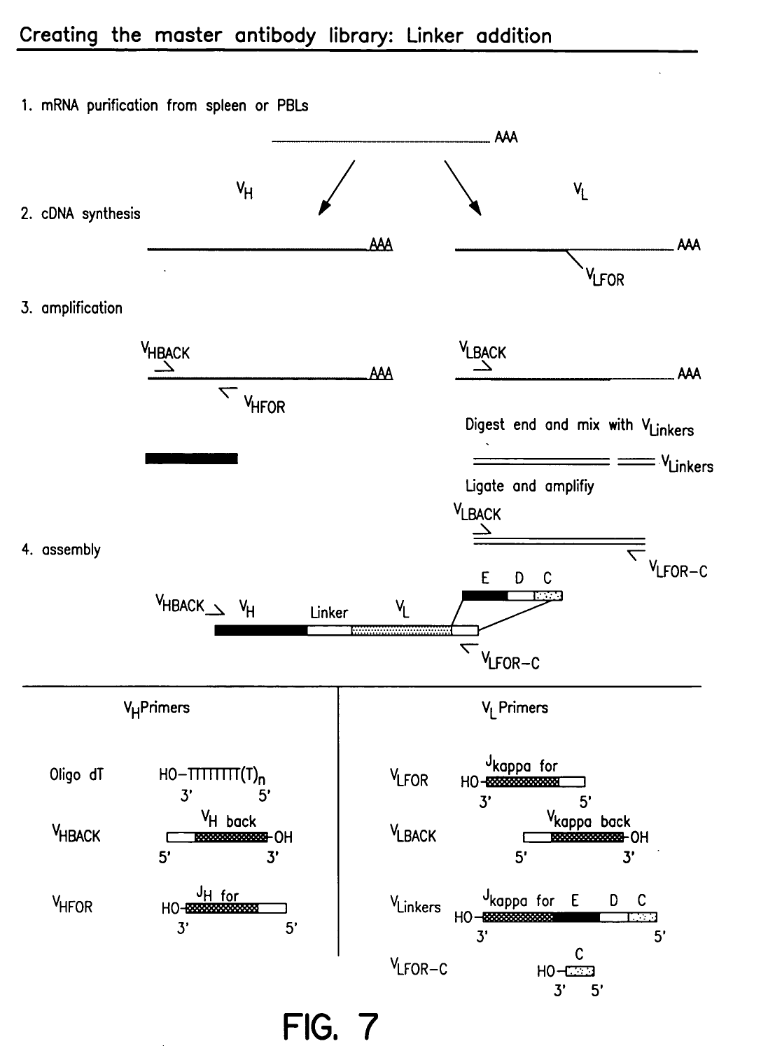

h) if the libraries are nucleic acid libraries, producing the tagged polypeptides in each array library. An exemplary embodiment of the process is outlined in FIGS. 6 and 7. Application of the method for evenly distributing polypeptide tags on proteins encoded by a master library is described. It is noted that practice of this method is not limited to polypeptide tagged proteins, but can be adapted for distribution of any tags on any collection of molecules. In all instances, the methods include steps in which molecules in library are separated into a predetermined number of sublibraries less than or equal to the number of different tags, and then, after attaching a tag members of each sublibrary, equal numbers of tagged molecules are mixed to produce a mixed tagged collection of molecules.

As noted the following sections describe the process with reference for exemplification purposes to evenly distributing polypeptide tags on collections of polypeptides that are encoded by a master library.

(1) Determining the Required Diversity of the Master Library

Prior to preparing or obtaining the Master library for tag incorporation, the diversity of molecules required for a particular intended application can be determined. This value either is predetermined or calculated based on one or more parameters, which include, for example, the total display desired for the arrayed capture system, the number of arrays to be screened, the number of loci per array and the diversity of molecules to be displayed on each locus. These factors are interrelated and can be defined before preparing the capture system using the equations set forth below.

The total display of the arrayed capture system is dependent on the number of arrays of capture systems, the number of loci per array and the diversity per locus:

Total Display=(Arrays)(Loci)(Diversity per Locus) EQ 1

The number of arrays and the number of loci can be decided and the array meeting the specifications prepared or can be a function of materials available for production of the arrays. For example, if an experimental setup includes 500 arrays with 10 loci per array and a diversity of 1000 per spot, then the total diversity displayed is equal to (500)(10)(1000) or 5×10⁶. As stated above, the diversity per locus is a function of the information required from the arrayed capture systems. If the system is being used to immobilize a specific molecule followed for purposes of monitoring a secondary reaction at the surface, then the diversity per locus required can be reduced. If the system is being used for high throughput screening of a particular pharmacological compound, then a higher diversity of potential reactants and, thus, the molecules displayed on the arrays may be desired. When determining the diversity to be displayed per spot, dilution of the signal or falsely positive signals are can be considered.

Number of Loci=Number of Tags EQ 2

The number of loci per array is constrained by the number of unique capture agent-tag pairs available and the mechanical ability to localize loci within an array. For example, if there are 1000 known capture agent-tag pairs, then each array can have a maximum of 1000 loci. The array can have less than 1000 loci. More than 1000 loci will reduce the sorting capabilities of the tagged molecules as some loci within the array will share common immobilized capture agents, resulting in two addresses for the complementary tagged molecules.

An array library is formed from a splitting of the mixed library into q subsets of tagged molecules wherein q is the number of arrays. The diversity of an array library is therefore dependent only on the parameters present within an individual array, the number of loci and the diversity of displayed molecules on each spot.

Diversity of Array libraries=(Loci)(Diversity per Spot) EQ 3

For example, if an array has 10 loci and each locus has a diversity of 1000 then the array library has a diversity of 10⁴.

The mixed library results from the pooling of an equal number of molecules from each tagged library, which is, in turn, formed from the insertion of a nucleic acid molecules encoding an polypeptide tag into individual sub-libraries of the master library. Thus, the diversity of the mixed library is equal to the diversity of the total display (EQ 4), which is equal to the sum of the diversities of each array library (EQ 5):

Diversity of Mixed library=Total Display EQ 4

Total Display=(Arrays)(Loci)(Diversity per spot) EQ 5

For example, if an experimental setup has 500 arrays with 10 loci per array and each locus has a diversity of 1000 then the total diversity displayed and the diversity of the mixed libraries equals (500)(10)(1000) or 5×10⁶. The tagged libraries are formed directly from the incorporation of unique tags into the individual sub-libraries.

Div of Tagged libraries=(Arrays)(Div per Spot) EQ 6

Div of Tagged Libraries=(Total Display)/(Loci) EQ7

Div of Tagged Libraries=((Div of Array libraries)(Arrays))/Loci EQ 8

Incorporation of the polypeptide tags into the members of the sub-libraries is governed by a Gaussian distribution. In addition, cloning efficiency and the efficiency other steps in the methods are 100%. Correction factors, which if necessary can be empirically determined, and included in the calculation of the diversity of the molecules within the sub-libraries. For the exemplified embodiment, it is recognized by those of skill in the art that cloning efficiency is about 10%. For different systems, efficiency can be empirically determined if needed. It is understood, since in general very large numbers of molecules are involved and the method do not require a precise determination of diversity, precise determination of such numbers and correction factors is not necessary to achieve the desired result. Thus, the diversity of the sub-libraries is determined by the diversity of the tagged libraries with a correction for inefficiencies, such as inefficiencies in ligation or transfection or other processes, which for purposes herein in the exemplified embodiment and other embodiments where it has not been empirically determined, can be assumed to be about 10%.

Div of Sub-libraries=(Div of Tagged libraries)(1.0/Cloning efficiency) EQ 9

For example, if the diversity of the tagged libraries is 5×10⁵ and the cloning efficiency is assumed to be about 0.1, then the diversity of the sub-libraries is 5×10⁶. This decrease in diversity from the sub-libraries to the tagged libraries results from known and recognized inefficiencies in the ligation and transformation process. The diversity of the sub-libraries also can be determined from the diversity of the source of the sub-libraries, the master library, divided by the number of loci in the array.

Div of the Sub-libraries=(Div of Master library/Loci) EQ 10

The master library is subdivided into sub-libraries. The number of sub-libraries is dependent on the number of unique tags and ultimately the number of capture agent/tag pairs. The number of loci in an array is determined by the number of different capture agents, which depends on the number of different tags. Therefore, as stated above, the number of loci is equal to the number of tags and the diversity of the sub-libraries is indirectly proportionally to the number of loci. If the number of loci per array increases, the number of sub-libraries also increases resulting in a decrease in the diversity of each sub-library. For example, if the diversity of the master library is 5×10⁷ and there are 10 loci per array then the diversity of the sub-libraries is (5×10⁷)/(10) or 5×10⁶. If the diversity of the master library is 5×10⁷ and the number of loci per array is increased to 250, then there are 250 sub-libraries each with a diversity of 2×10⁵.

Using the inverse of the equation above, the diversity of the master library can be calculated from the number of loci (or the number of sub-libraries) and the diversity of each sub-library.

Div of Master Library=(Div of Sub-libraries)(Loci) EQ 11

For example, if there are 50 sub-libraries or loci and each sub-library has a diversity of 1×10⁵, then the master library has to have a diversity of (50)(1×10⁵) or 5×10⁶.

If the diversity is known, then the number of arrays required, the number of loci per array, the diversity per locus or the total display of the arrayed capture systems can be calculated. Alternatively, any of the other parameters mentioned 4000 arrays with 100 loci and each locus is required to have a diversity of 500, then a master library has to be prepared or commercially obtained that has a diversity of 2×10⁸. If a master library is obtained that has a diversity of 2×10⁸, a diversity of 1000 per locus is required and the slide has space for 1000 arrays, then 250 loci need to be placed in each array. Table 4 below shows other examples of the relationships among the parameters defining the arrayed capture system. One of skill in the art can recognize that diversity of the master library, the number of arrays and loci per array and the diversity per locus can all be defined adjusted to suit any experimental situation.

TABLE 4

| Total Display | 5 × 10⁶ | 10⁷ | 2.5 × 10⁸ | 10⁹ | 2 × 10⁸ | 10⁹ | 10⁹ |
|---|---|---|---|---|---|---|---|
| Arrays | 500 | 1000 | 1000 | 4000 | 4000 | 2000 | 4000 |
| Loci | 10 | 10 | 250 | 250 | 100 | 500 | 500 |
| Div per Locus | 1000 | 1000 | 1000 | 1000 | 500 | 1000 | 500 |
| Master Library | 5 × 10⁷ | 10⁸ | 2.5 × 10⁹ | 10¹⁰ | 2 × 10⁹ | 10¹⁰ | 10¹⁰ |
| Sub-libraries | 5 × 10⁶ | 10⁷ | 10⁷ | 4 × 10⁷ | 2 × 10⁷ | 2 × 10⁷7 | 2 × 10⁷7 |
| Tag libraries | 5 × 10⁵ | 10⁶ | 10⁶ | 4 × 10⁶ | 2 × 10⁶ | 2 × 10⁶ | 2 × 10⁶7 |
| Mixed Libraries | 5 × 10⁶ | 10⁷ | 2.5 × 10⁸ | 10⁹ | 2 × 10⁸ | 10⁹ | 10⁹ |
| Array Libraries | 10⁴ | 10⁴ | 2.5 × 10⁵ | 2.5 × 10⁵ | 5 × 10⁴ | 5 × 10⁵ | 2.5 × 10⁵7 |

(2) Creation of the Master Library and Division into Sub-Libraries

A master library is a collection of molecules such as, but not limited to, organic compounds, inorganic compounds, polypeptides and nucleic acids. Examples of master libraries for use with the methods provided herein include, but are not limited to, cDNA libraries, combinatorial small molecule and peptide libraries and BAC and PAC libraries. These master libraries can be produced synthetically using any method known to those skilled in the art (see, e.g., EXAMPLE 6), or can be purchased commercially from companies such as Invitrogen (www.resgen.com/intro/libraries.php3) and Jerini Peptide Technology (www.jerini.de/base.htm). For exemplification of the methods herein, the master library is a collection of nucleic acid molecules that encode polypeptides. The diversity of the master library is equal to the number of unique members within the collection. The diversity of the master library can be determined by empirical methods or is known when the library is constructed or obtained. The master library is then diluted such that the diversity of the library is equal to or nearly equal to the number of molecules within the library so that each molecule is represented once.

The diluted master library is then divided into sublibraries numbered 1 to n, wherein n is equal to the total number of sublibraries. Each of the sublibraries can then be contacted with a tag such that each sublibrary is covalently attached to a unique tag, yielding a set of tagged libraries.

A master library can contain typically from 10⁴ to 10¹², generally 10⁶ to 10¹² different (i.e., unique) members. The particular manner in which the libraries are prepared for the methods described herein is a function of the library. For example, for cloning into a selected vector, such as a plasmid for bacterial expression, suitable restriction sites can be included as needed. Other modifications are routine and known to those of skill in the art.

In some embodiments, the libraries have fewer than the selected diversity. In such instances, different libraries can be obtained or generated and then combined, or, as described herein, separately used to produce the sublibraries. This permits generation of tagged libraries, and ultimately arrays and canvases, of high diversity.

Nucleic acid libraries are contacted with nucleic acid molecules encoding the polypeptide tag sequences such that, when translated, encoded members of each sub-library are attached to the same polypeptide tag. Due to inefficiencies in ligation and transformation during cloning in the methods for evenly distributing tags, the diversity of tagged libraries is lower, estimated for purposes herein to about 10%, of the diversity of each sub-library. Although 10% generally serves as a good estimate, if needed the precise numbers can be empirically determined for a particular sublibrary and tagged library.

(3) Adjusting the Diversity of a Master Library so that the Diversity is about Equal the Number of Members of the Library

If necessary, the diversity of a master library is adjusted so that its diversity is approximately equal to the number members of the library. Typically, approximately equal is within one order of magnitude or less, such as 0.5 orders of magnitude and generally, 0.1 orders of magnitude. This adjustment can be accomplished, for example, by estimating the diversity of the library and estimating the total number of molecules in the library. It is understood that determination of diversity and numbers of members in a library are estimates, not exact determination. A composition is prepared such that the number of estimated molecules and the estimated diversity is the about same (i.e., within about an order of magnitude, 0.5 order of magnitude or generally 0.1 order of magnitude). For example, if the diversity of the library is estimated to be 2.5×10¹⁰, then a sample containing 2.5×10¹⁰ molecules is prepared.

Diversity can be estimated by any method known to those of skill in the art and is a function of the type of library. For example, for single chain antibody encoding library, the diversity is estimated to be the number of transformants produced upon introduction of the library into a bacterial host. It is assumed by those of skill in the art that each transformant is unique.

(4) Dividing the Master Library into Sub-Libraries

The master library is divided into up to “n” sub-libraries designated 1 . . . n, where n is equal to or less than the number of different nucleic acid molecules that encode different tags. Where the diversity of the master library is equal to the number of molecules within the collection, the sub-libraries are all of equal volume, number of molecules and diversity. If the diversity does not equal the number of molecules in the collection, then appropriate adjustment of the volume of the sublibraries may be required.

Separation of a master library can be accomplished, for example, by initially estimating the diversity of molecules in a master library and then preparing a solution in which the number of molecules is equal to, or nearly equal to, the diversity of molecules the Master library. For example, if the diversity of molecules in the Master library is estimated to be 2.5×10¹⁰, then a composition of 2.5×10¹⁰ molecules is prepared. The resulting composition is then physically divided into n number of aliquots of each of equal volume such that each aliquot contains approximately the same number of molecules. The molecules contained in these aliquoted solutions are the sub-libraries.

As stated above, the number of different tag-encoding nucleic acid molecules can be predetermined, and constrains the number of sub-libraries prepared from the master library. The number of sub-libraries is typically equal to, but can be less than, the number of unique tag-encoding nucleic acid molecules.

(5) Creation of Tagged Libraries

Tagged libraries are produced by attaching, directly or indirectly, a a nucleic acid molecule encoding a tag to members of each sublibrary to produce “n” tagged sublibraries containing tagged members, whereby the polypeptide (epitope) tag encoding portion of the tag is in frame with a polypeptide encoded by the nucleic acid molecule. The encoded polypeptide tag is unique to each sublibrary

As noted, division of the master library into sub-libraries is based on the number of unique tags encoding nucleic acid molecules available. Preparation of the tagged library results from the incorporation of a sequence of nucleotides that encodes a unique tag into the molecules of each sub-library. Any methods known to those of skill in the art to add and incorporate a double stranded DNA fragment into nucleic acid can be used. In the method provided herein, the tag-containing fragments are ligated directly or via linkers to the molecular members of the sub-libraries (exemplified herein). The amplified or ligated product, if needed, can be further amplified or manipulated such as by the ligation of additional tags or insertion of other properties using methods that can be readily devised by those of skill in the art in light of the description herein.

In the initial tagging step, when adding the tag encoding set of oligonucleotides on the constituent members of the nucleic acid sublibrary, a goal is to get an even distribution of all nucleic acid molecules encoding the tags, so that on the average each different molecule has a unique nucleic acid tag. To effect this, the master library is divided into sublibraries, identified as S₁-Sₙ, wherein n is equal to or less than number of unique encoded tags. Each sub-library is then contacted labeled with a unique polypeptide tag, yielding a collection of sub-libraries each tagged with a unique tag.

Any method known to one of skill in the art to link a tag, such as a nucleic acid molecule encoding a tag, such as a polypeptide tag, to another molecule, such as a nucleic acid or a polypeptide is contemplated. For exemplification, a variety of such methods are described above, such as ligation to create circular plasmid vectors; ligation of sequences resulting in linear tagged cDNA molecules; primer extension and PCR for tag incorporation; insertion by gene shuffling; recombination strategies; incorporation by transposases; and incorporation by splicing. As noted, they are described with particular reference to antibody capture agents, and polypeptide tags that include epitopes to which the antibodies bind, but it is to be understood that the methods herein can be practiced with any capture agent and polypeptide tag therefor.

For example, in addition to use of amplification protocols for introducing the primers into the library members, the primers can be introduced by direct ligation, such as by introduction into plasmid vectors that contain the nucleic acid that encode the tags and other desired sequences. Subcloning of a nucleic acid molecule, such as a cDNA molecule, into double stranded plasmid vectors is well known to those skilled in the art, and is exemplified herein in Examples 5-7 below. Any suitable vector for such subcloning can be used, and includes any that infect bacteria or that can be propagated in eukaryotic cells. Plasmids (designed 1−n, wherein is the number of unique polypeptide tags to be distributed among members of the library) with nucleic acid encoding the each of the tags are prepared kept separate. Nucleic acid from the master library is introduced into the 1−n plasmids such that encoded polypeptides are in reading frame, although not necessarily adjacent, with the polypeptide tag, such that upon expression of the nucleic acid molecule a polypeptide with the tag, typically at one end is produced.

As exemplified, digesting purified double stranded plasmid with a site-specific restriction endonuclease creates 5′ or 3′ overhangs also known as sticky ends. Double-stranded members of a DNA library are digested with the same restriction endonuclease to generate complementary sticky ends. Alternately, blunt ends in the vector DNA and DNA in the library are created and used for ligation. The digested DNA and plasmid DNA are mixed with a DNA ligase in an appropriate buffer (commonly, T4 DNA ligase and buffer obtained from New England Biolabs are used) and incubated (typically at 16° C.) to allow ligation to proceed. A portion of the ligation reaction is transformed into a suitable host, such as E. coli, that has been rendered competent for uptake of DNA by any of a variety of methods, such as, but are not limited to, electroporation, calcium phosphate update, lipid-mediated transfection and heat shock of chemically competent cells are two common methods.

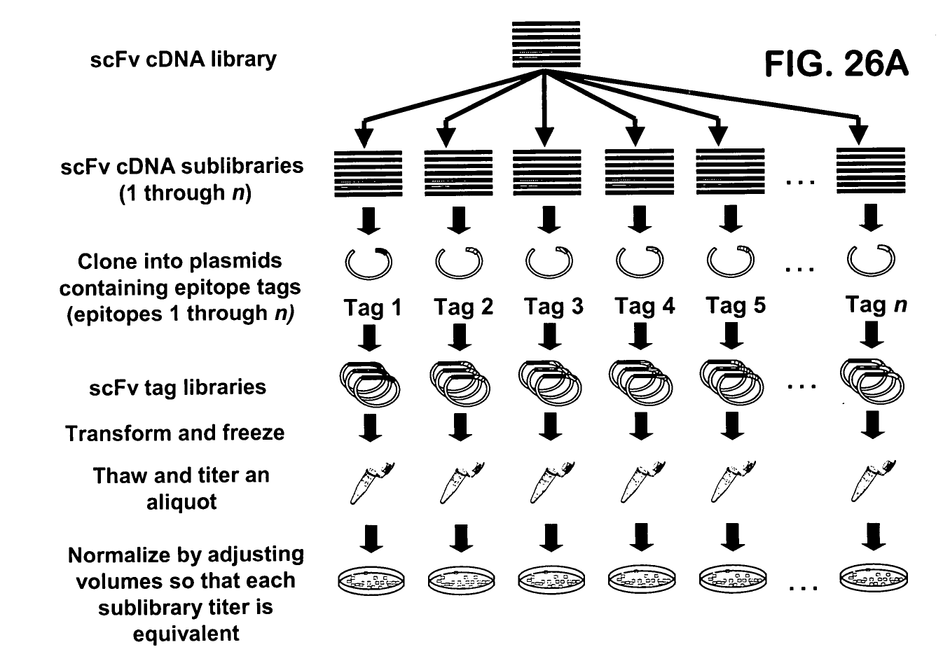

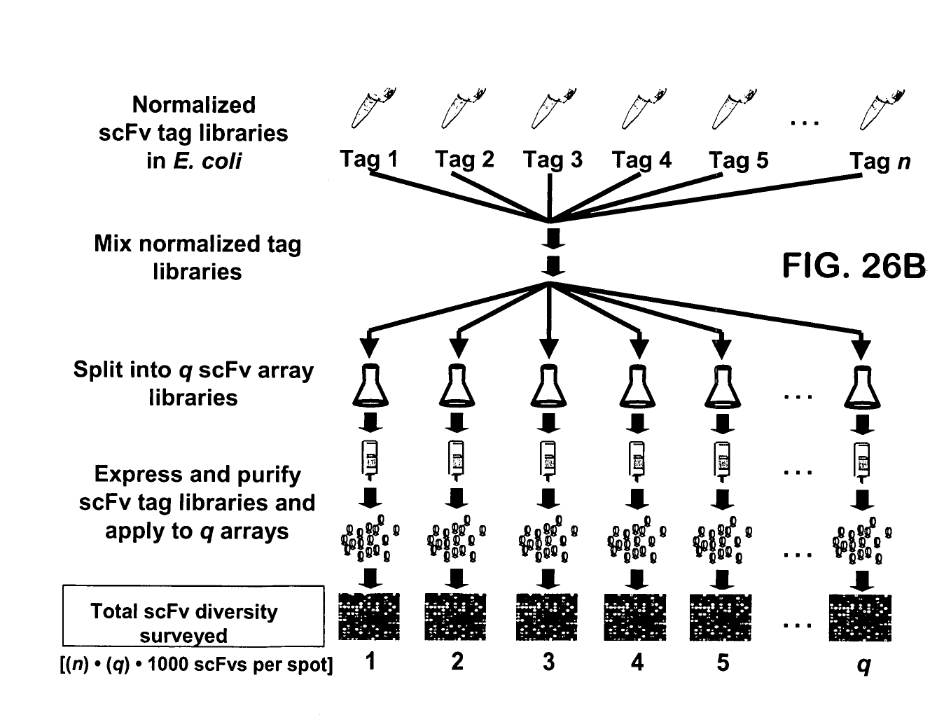

Aliquots of the transformation mixture can be plated onto semi-solid selective medium, such as medium containing the antibiotic appropriate for the plasmid used. Only those bacteria receiving a circular plasmid gives rise to a colony on this selective medium. For each set of plasmids that encode a tag, samples of the DNA library are inserted (see, e.g., FIGS. 26A and 26B).

For directional cloning of cDNA clones, which is desirable for the creation of a library used for expression of proteins from the cDNA library in reading frame with a tag, two different restriction endonuclease, which generate different sticky ends can be used for digestion of the plasmid. The cDNA library members are created such that they contain these two restriction endonuclease recognition sites at opposite ends of the cDNA. Alternately, for example, different restriction endonuclease that generate complementary overhangs are used (for example digestion of the plasmid with NgoMIV and the cDNA with BspEI leave a 5′CCGG overhang and are thus compatible for ligation). Furthermore, directional insertion of the cDNA into the plasmid vector brings the cDNA under the control of regulatory sequences contained in the vector. Regulatory sequences can include promoter, transcriptional initiation and termination sites, translational initiation and termination sequences and RNA stabilization sequences. If desired, insertion of the cDNA also places the cDNA in the same translational reading frame with sequences coding for additional protein elements including those used for the purification of the expressed protein, those used for detection of the protein with affinity reagents, those used to direct the protein to subcellular compartments, those that signal the post-translational processing of the protein.

For example, as described in Examples 6 and 7, the pBAD/gIII vector (Invitrogen, Carlsbad Calif.) was used as an expression vector for the scFv cDNA library obtained from mouse spleens (see Examples). This vector contains cloning sites that are useful for insertion of cDNA clones. When ligating a nucleic acid library into an expression vector, the cloning sites can be designed and/or chosen such that the inserted cDNA clones are not internally digested with the enzymes used and such that the cDNA is in the same reading frame as the desired coding regions contained in the vector. For example, it is common to use SfiI and NotI sites for insertion of single chain antibodies (scFv) into expression vectors. Therefore, to modify the pBAD/gIII vector for expression of scFvs, oligonucleotides containing these restriction sites were hybridized and inserted into restriction site already present in the vector. The resultant vector permits insertion of scFvs (created with standard methods such as the “Mouse scFv Module” from Amersham-Pharmacia) in the same reading frame as the gene III leader sequence and the polypeptide tag.

As exemplified herein, a library of expressed proteins is subdivided using a plurality of polypeptide tags and the antibodies that recognize them. To create the library for expressing proteins with a plurality of polypeptide tags, slight modifications of the subcloning techniques described above are used. A plurality of cDNA clones are divided into sublibraries and each sublibrary is inserted into a distinct plasmid vector containing a unique polypeptide tag encoding nucleic acid sequence (instead of a single type of plasmid vector) such that the resulting library contains cDNA clones tagged with the different polypeptide tags, and each polypeptide tag is represented equally. Multiple plasmid vectors are created such that they differ in the polypeptide tag that is translated in frame with the inserted cDNA member. For example, if there are 1000 polypeptide tag sequences, 1000 different vectors are constructed; if there are 250 polypeptide tag sequences, 250 different vectors are constructed.

There are a variety of methods for construction of these vectors known to those of skill in the art. For illustration the myc epitope encoding region of the pBAD/gIII plasmid is removed by digestion with XbaI and SalI restriction enzymes, and the large 4.1 kb fragment is isolated. The hybridization of oligonucleotides HAFor (SEQ ID No. 8) and HARev2 (SEQ ID No. 74) creates overhangs compatible with XbaI and SalI, such that the product is inserted directionally, and encodes the epitope for the HA11 antibody (see Tables 2 and 3 above). Insertion of the hybridization product of M2For (SEQ ID No. 10) and M2Rev2 (SEQ ID No. 11) results in a vector with the FLAG M2 epitope (see Tables 2 and 3 above) in frame with the inserted cDNA. Insertion of the hybridization product of V5For (SEQ ID No. 75) and V5Rev (SEQ ID No. 76) results in a vector with the V5 epitope (see table below) in frame with the inserted cDNA. Hybridization and insertion of pairs of oligos listed below result in the creation of the epitopes in frame with the cDNA.

Each of these vectors still shares the SfiI and NotI restriction endonuclease sites to allow subcloning of cDNA clones into the vectors. Similarly, additional oligonucleotides can be designed to encode a wide variety of polypeptide tags that can be inserted in the same position to create a collection of different vectors.

Plasmid DNA corresponding to the vectors containing different polypeptide tags is prepared using methods known to those in the art (Qiagen columns, CsCl density gradient purification, etc). Purified double stranded DNA from each of the plasmids is quantified by OD260 and ethidium bromide staining on an agarose gel confirms quantification. Other methods know to those skilled in the art can be used for quantification of plasmid DNA.

In order to evenly distribute the polypeptide tags among the cDNA clones, a series of plasmid vectors encoding the polypeptide tag sequences is created such that each vector in the series contains a unique polypeptide tag encoding sequence. Each of these vectors shares restriction endonuclease sites to allow subcloning (generally directional) of cDNA clones into the vectors. Double stranded cDNA representing the library of interest is also digested with restriction endonuclease to create ends that are compatible for ligation to the ends created by vector digestion. This is accomplished by using the same enzymes for vector and cDNA digestion or by using those that generate complementary overhangs (for example NgoMIV and BspEI both leave a 5′CCGG overhang and are thus compatible for ligation). Alternately, blunt ends in both vector DNA and cDNA are created and used for ligation. Digested cDNA clones and digested vector DNAs are ligated using a DNA ligase such as T4 DNA ligase, E. coli DNA ligase, Taq DNA ligase or other comparable enzyme in an appropriate reaction buffer. The resultant DNA is transformed into bacteria, yeast, or used directly as template for in vitro transcription of RNA. The design of the vectors is such that insertion of the cDNA at the restriction endonuclease sites places the cDNA under control of promoter sequences to allow expression of the cDNA. Additionally the cDNA are in the same reading frame as the nucleic acid sequence encoding the polypeptide tag such that upon protein expression from this vector, a fusion protein containing the cDNA-encoded polypeptide fused to the polypeptide tag is produced. The E sequence is positioned in the vector such that the encoded polypeptide tag is fused to either the N or the C terminus of the resultant protein. (for restriction enzyme digestion, DNA ligation, and transformation, see, e.g., see, Sambrook et al. (1989) Molecular Cloning: A Laboratory Manual, 2nd Edition, Cold Spring Harbor Laboratory Press, Chapter 1).

(6) Mixing Some or All of the Tagged Sub-Libraries to Produce a Mixed Library, Where the Number of Tagged Nucleic Acid Molecules Added from Each Tagged Sub-Library is the Same

Tagged libraries are combined to produce a mixed library such that the each tagged molecule is approximately equally represented. As a result, tags are evenly distributed among the member tagged molecules of the mixed library. The determination of the number of tagged members within each tagged library and the mixing of the tagged libraries to give a mixed library can be accomplished by any suitable method. For example, the concentration of tagged molecules in sublibraries to be mixed is determined and equal numbers are mixed. Concentration is determined by any suitable method such as by titering the number of transformants or colony forming units produced upon introduction of the tagged molecule into an appropriate host. Other methods of concentration determination include spectrometric and physical assay, such as the Bradford assay. Spectrometric methods monitor the increase or decrease in absorbance of light at a particular wavelength. According to Beer's Law, the absorbance of a molecule at a particular wavelength is proportional to its extinction coefficient, the pathlength of the light and the concentration of the absorbing species. Therefore, determination of ultra violet or visible light at a predetermined wavelength can be used to calculate the concentration of the absorbing species within a known volume. Fluorescent molecules, such as GFP, emit light at a particular wavelength.

Prior to determining the concentration of the tagged libraries, separation of the fused molecule-tag product from the non-combined molecule and tag reactants may be required. Any method of separation known to those skilled in the art can be used. For example, electorphoretic methods can be used to identify and separate the fused nucleic acid molecules that encode the molecule and tag from the individual components. Other methods, such as, but not limited to, transformation of the complex into a suitable host followed by antibiotic or other selection method, affinity chromatography, and co-expression of a detectable molecule such as GFP, are also contemplated. As stated above, the polypeptide tag itself can contain secondary tags that can be used for selection of fused molecule—polypeptide tag molecules.

Once the concentration of tagged molecules in each tagged library is known, an aliquot from each tagged sublibrary which contains the same number of tagged members can be pooled to give the mixed library. Optionally, the tagged libraries can be normalized prior to mixing such that the tagged libraries all contain an equivalent number of tagged members. An aliquot of equal volume from each of the normalized tagged sublibraries can then be combined to give a mixed library.

(7) Splitting the Mixed Library into “q” Array Libraries, Wherein q is from 1 to a Predetermined Number of Arrays

The mixed library is split into q array libraries wherein q is equal to the number of arrays to be developed. As stated above, the number of arrays present is predetermined based on the number of loci per array, the desired diversity per locus and the diversity of the master library. Once this value has been determined, the pooled mixed library is split into aliquots of equal volume wherein the number of aliquots is equal to or less than the number of arrays.

(8) Expression of Array Libraries and Purification of Tagged Molecules to Produce Collections of Tagged Molecules with Even Distributions of Tags

The tagged members of the array libraries are translated and the resulting polypeptides are purified yielding a collection of tagged molecules wherein the distribution of polypeptide tags is even throughout the collection of molecules. The purification of the molecules can performed by any method known to those skilled in the art, such as, for example affinity purification.

5. Preparation of Capture Agents

As described above, a capture agent refers to any molecule that has an affinity for a given ligand or with a defined sequence of amino acids. In particular, any molecules that specifically binds with reasonable affinity to tags, such as epitope tags, to subdivide a tagged library is a capture agent. For exemplary purposes herein, reference is made to antibodies and tags that encode epitopes to which the antibody specifically binds.

a. Antibodies and Collections of Addressable Anti-Tag Antibodies

The methods herein, rely upon the ability of the capture agents, such as antibodies, to specifically bind to the polypeptide tags, which are linked to libraries (or collections) of molecules, particularly proteins. The specificity of each antibody (or other receptor in the collection) for a particular tag is known or can be readily ascertained, such as by arraying the antibodies so that all of the antibodies at a locus in the array are specific for a particular tag, such as an epitope tag.

Alternatively, each antibody can be identified, such as by linkage to optically encoded tags, including colored beads or bar coded beads or supports, or linked to electronic tags, such as by providing microreactors with electronic tags or bar coded supports (see, e.g., U.S. Pat. No. 6,025,129; U.S. Pat. No. 6,017,496; U.S. Pat. No. 5,972,639; U.S. Pat. No. 5,961,923; U.S. Pat. No. 5,925,562; U.S. Pat. No. 5,874,214; U.S. Pat. No. 5,751,629; U.S. Pat. No. 5,741,462), or chemical tags (see, U.S. Pat. No. 5,432,018; U.S. Pat. No. 5,547,839) or colored tags or other such addressing methods that can be used in place of physically addressable arrays. For example, each antibody type can be bound to a support matrix associated with a color-coded tag (i.e., a colored sortable bead) or with an electronic tag, such as an radio-frequency tag (RF), such as IROR1 MICROKANS® and MICROTUBES® microreactors (see, U.S. Pat. No. 6,025,129; U.S. Pat. No. 6,017,496; U.S. Pat. No. 5,972,639; U.S. Pat. No. 5,961,923; U.S. Pat. No. 5,925,562; U.S. Pat. No. 5,874,214; U.S. Pat. No. 5,751,629; U.S. Pat. No. 5,741,462; International PCT application No. WO98/31732; International PCT application No. WO98/15825; and, see, also U.S. Pat. No. 6,087,186). For the methods and collections provided herein, the antibodies of each type can be bound to the MICROKAN or MICROTUBE microreactor support matrix and the associate RF tag, bar code, color, colored bead or other identifier to serves to identify the receptors, such as antibodies, and hence the tag to which the receptor, such as an antibody, binds.

For exemplary purposes herein, reference is made to antibodies and tags that encode epitopes to which the antibody specifically binds. It is understood that any pair of molecules that specifically bind are contemplated; for purposes herein the molecules, such as antibodies, are designated receptors, and the molecules, such as ligands, that bind thereto are epitopes. The epitopes are typically short sequences of amino acids that specifically bind to the receptor, such as an antibody or specific binding fragment thereof.

Also, for exemplary purposes herein, reference is made to positional arrays. It is understood, however, that such other identifying methods can be readily adapted for use with the methods herein. It is only necessary that the identity (i.e., epitope-tag specificity) of the receptor, such as an antibody, is known. The resulting collections of addressable receptors (i.e., antibodies), whether in a two-dimensional or three-dimensional array, or linked to optically encoded beads or colored supports or RF tags or other format, can be employed in the methods herein.

By reacting a collection of antibodies with libraries of polypeptide tag-labeled molecules, and then performing screening assays to identify the members of the collection of the antibodies to which epitope-labeled molecules of a desired property have bound, a reduction in the diversity of the library of molecules is achieved. Each collection of antibodies serves as a sorting device for effecting this reduction in diversity. Repeating the process a plurality of times can effect a rapid and substantial reduction in diversity.

b. Preparation of the Capture Agents

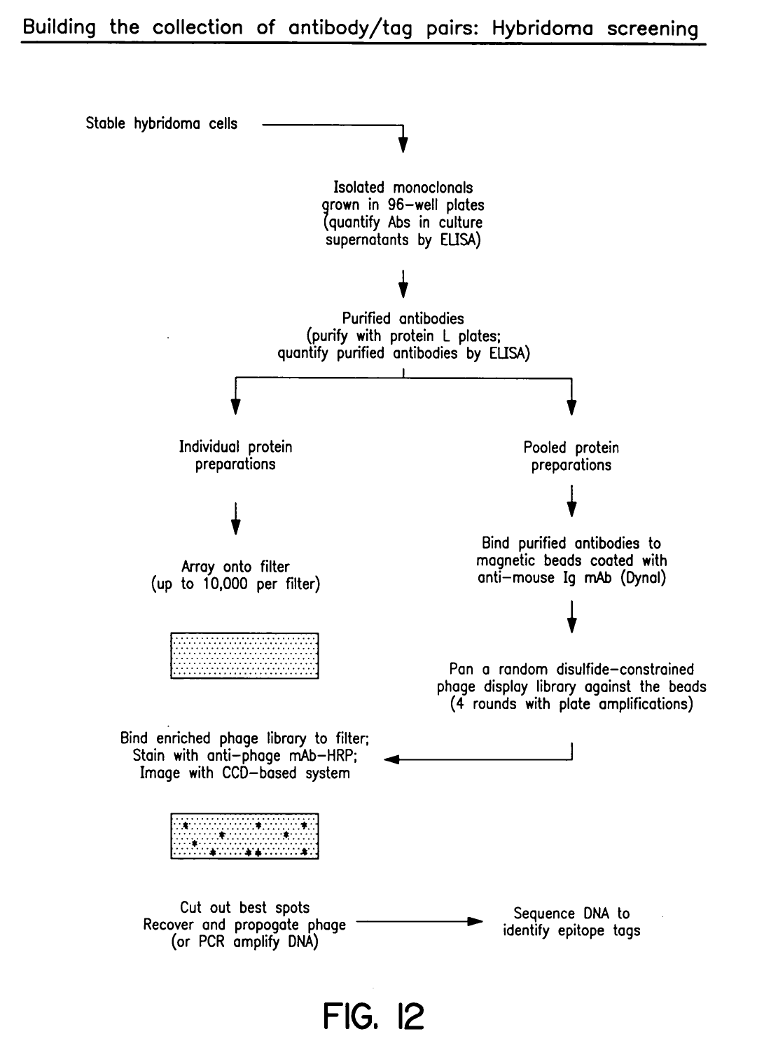

The quality of the sorts is dependent on the quality of the collection of capture agents, such as antibodies, that make up the sorting array. In addition to requirements on binding affinity and specificity, the epitopes bound by the capture agents (antibodies) in the array determine the E, FA and FB sequences used as priming sites for the amplification reactions (PCRs). FIG. 12 outlines a high throughput screen for discovering immunoglobulin (Ig) produced from hybridoma cells for use in generating antibodies for use in the collections.

Hybridoma cells are created either from non-immunized mice or mice immunized with a protein expressing a library of random disulfide-constrained heptmeric epitopes or other random peptide libraries. Stable hybridoma cells are initially screened for high Ig production and epitope binding. Immunoglobulin (Ig) production is measured in culture supernatants by ELISA assay using a goat anti-mouse IgG antibody. Epitope binding is also measured by ELISA assay in which the mixture of haptens (epitope tagged proteins) used for immunization are immobilized to the ELISA plate and bound IgG from the culture supernatants is measured using a goat anti-mouse IgG antibody. Both assays are done in 96-well formats or other suitable formats. For example, approximately 10,000 hybridomas are selected from these screens.

Next, the Ig are separately purified using 96-well or higher density purification plates containing filters with immobilized Ig-binding proteins (proteins A, G or L). The quantity of purified Ig is measured using a standard protein assay formatted for 96-well or higher density plates. Low microgram quantities of Ig from each culture are expected using this purification method.

The purified Ig are spotted separately onto a nitrocellulose filter using a standard pin-style arraying system. The purified Ig are also combined to produce a mixture with equal quantities of each Ig. The mixed Ig are bound to paramagnetic beads which are used as a solid-phase support to pan a library of bacteriophage expressing the random disulfide-constrained heptmeric epitopes. The batch panning enriches the phage display library for phage expressing epitopes to the purified Ig. This enrichment dramatically reduces the diversity in the phage library.

The enriched phage display library is then bound to the array of purified Ig and stringently washed. Ig-binding phage are detected by staining with an anti-phage antibody-HRP conjugate to produce a chemiluminescent signal detectable with a charge coupled device (CCD)-based imaging system. Spots in the array producing the strongest signals are cut out and the phage eluted and propagated. Epitopes expressed by the recovered phage are identified by DNA sequencing and further evaluated for affinity and specificity. This method generates a collection of high-affinity, high-specificity antibodies that recognize the cognate epitopes. Continued screening produces larger collections of antibodies of improved quality.

c. Preparation of Capture Agent Arrays

Each spot contains a multiplicity of capture agents, such as antibodies, with a single specificity. Each spot is of a size suitable for detection. Spots on the order of 1 to 300 microns, typically 1 to 100, 1 to 50, and 1 to 10 microns, depending upon the size of the array, target molecules and other parameters. Generally the spots are 50 to 300 microns. In preparing the arrays, a sufficient amount is delivered to the surface to functionally cover it for detection of proteins having the desired properties. Generally the volume of antibody-containing mixture delivered for preparation of the arrays is a nanoliter volume (1 up to about 99 nanoliters) and is generally about a nanoliter or less, typically between about 50 and about 200 picoliters. This is very roughly about 10 million to 100,000 molecules per spot, where each spot has capture agents, such as antibodies, that recognize a single epitope. For example, if there are 10 million molecules and 1000 different ones in the protein mixture reacting with the locus, there are 10⁴ of each type of molecule per spot. The size of the array and each spot should be such that positive reactions in the screening step can be imaged, generally by imaging the entire array or a plurality thereof, such as 24, 96, or more arrays, at the same time.

A support (see below for exemplary supports), such as KODAK paper plus gelatin or other suitable matrix can be used, and then ink jet and stamping technology or other suitable dispensing methods and apparatus, are used to reproducibly print the arrays. The arrays are printed with, for example, a piezo or inkjet printer or other such nanoliter or smaller volume dispensing device. For example, arrays with 1000 spots can be printed. A plurality of replicate arrays, such as 24 or 48, 96 or more can be placed on a sheet the size of a conventional 96 well plate.

Among the embodiments contemplated herein, are sheets of arrays each with replicates of the capture agent, such as antibody, array. These are prepared using, for example, a piezo or inkjet dispensing system. A large number, for example, 1000, can be printed at a time using, for example a print head with 1000 different holes (like a stamp with 500 μM holes). It can be fabricated from, for example, molded plastic with many holes, such as 1000 holes, each filled with 1000 different capture agents, such as antibodies. Each hole can be linked to reservoirs that are linked to conduits of decreasing size, which ultimately dispense the capture agents, such as antibodies into the print head. Each array on the sheet can be spatially separated, and/or separated by a physical barrier, such as a plastic ridge, or a chemical barrier, such a hydrophobic barrier (i.e., hydrogels separated by hydrophobic barriers). The sheets with the arrays can be conveniently the size of a 96 well plate or higher density. Each array contains a plurality of addressable anti-tag antibodies specific for the pre-selected set of tags, such as polypeptide tags. For example, 33×33 arrays contain roughly 1000 antibodies, each spot on each array containing antibodies that specifically bind to a single pre-selected epitope. A plurality of arrays separated by barriers can be employed.

For dispensing the antibodies onto the surface, the goal is functional surface coverage, such that a screened desired protein is detectable. To achieve this, for example, about 1 to 2 mgs/ml from the starting collection are used and about 500 picoliters per antibody are deposited per spot on the array. The exact amount(s) can be empirically determined and depend upon several variables, such as the surface and the sensitivity of the detection methods. The antibodies are generally covalently linked, such as by free sulfhydryl linkages to maleimides or free amine linkage to NHS-esters on the surface.

Other exemplary dispensing and immobilizing systems include, but are not limited to, for example, systems available from Genometrix, which has a system for printing on glass; from Illumina, which employs the tips of fiber optic cables as supports; from Texas Instruments, which has chip surface plasmon resonance (i.e., protein derivatized gold); inkjet systems, such as those from Microfab Technologies, Plano Tex.; Incyte, Palo Alto, Calif., Protogene, Mountain View, Calif., Packard BioSciences, Meriden Conn., and other such systems for dispensing and immobilizing proteins to suitable support surfaces. Other systems such as blunt and quill pins, solenoid and piezo nanoliter dispensers and others are also contemplated.

d. Preparation of Other Collections

The capture agents are linked to beads or other particulate supports that are identifiable. For example, the capture agents are linked to optically encoded microspheres, such as those available from Luminex, Austin Tx, the contain fluorescent dyes encapsulated therein. The microsphere, which encapsulate dyes, are prepared from any suitable material (see, e.g., International PCT application Nos. WO 01/13119 and WO 99/19515; see description below), including stryene-ethylene-butylene-styrene block copolymers, homopolymers, gelatin, polystyrene, polycarbonate, polyethylene, polypropylene, resins, glass, and any other suitable support (matrix material), and are of a size of a about a nanometer to about 10 millimeters in diameter. By virtue of the combination of, for example two different dyes at ten different concentrations, a plurality microspheres (100 in this instance), each identifiable by a unique fluorescence, are produced.

Alternatively, combinations of chromophores or colored dyes or other colored substances are encapsulated to produce a variety of different colors encapsulated in microspheres or other particles, which are then used as supports for the capture agents, such as antibodies. Each capture agent, such as an antibody, is linked to a particular colored bead, and, is thereby identifiable. After producing the beads with linked capture agents, such as antibodies, reaction with the tagged molecules can be performed in liquid phase. The beads that react with the epitopes are identified, and as a result of the color of the bead the particular epitope and is then known. The sublibrary from which the linked molecule is derived is then identified.

6. Supports for Immobilization of Capture Agents

Supports for immobilizing the capture agents, such as antibodies, are any of the insoluble materials known for immobilization of ligands and other molecules, used in many chemical syntheses and separations, such as in affinity chromatography, in the immobilization of biologically active materials, and during chemical syntheses of biomolecules, including proteins, amino acids and other organic molecules and polymers. Suitable supports include any material, including biocompatible polymers, that can act as a support matrix for attachment of the antibody material. The support material is selected so that it does not interfere with the chemistry or biological screening reaction.

Supports that are also contemplated for use herein include fluorophore-containing or fluorophore-impregnated supports, such as microplates and beads (commercially available, for example, from Amersham, Arlington Heights, Ill.; plastic scintillation beads from Nuclear Technology, Inc., San Carlos, Calif. and Packard, Meriden, Conn., and colored bead-based supports (fluorescent particles encapsulated in microspheres) from Luminex Corporation, Austin, Tex. (see, International PCT application No. WO/0114589, which is based on U.S. application Ser. No. 09/147,710; see International PCT application No. WO/Ol 13119, which is U.S. application Ser. No. 09/022,537). The microspheres from Luminex, for example, are internally color-coded by virtue of the encapsulation of fluorescent particles and can be provided as a liquid array. The capture agents, such as antibodies, are linked directly or indirectly by any suitable method and linkage or interaction to the surface of the bead and bound proteins can be identified by virtue of the color of the bead to which they are linked. Detection can be effected by any method, and can be combined with chromogenic or fluorescent detectors or reporters that result in a detectable change in the color of the microsphere (bead) by virtue of the colored reaction and color of the bead. For the bead-based arrays, the capture agents are attached to the color-coded beads in separate reactions. The code of the bead identifies the capture agent, such as an antibody, attached to it. The beads then can be mixed and subsequent binding steps performed in solution. They then can be arrayed, for example, by packing them into a microfabricated flow chamber, with a transparent lid, that permits only a single layer of beads to form resulting in a two-dimensional array. The beads to which a protein is bound are identified, thereby identifying the capture agent and the tag, such as an epitope tag. The beads are imaged, for example, with a CCD camera to identify beads that have reacted. The codes of such beads are identified, thereby identifying the capture agent, which in turn identifies the polypeptide tag and, ultimately, the protein of interest.

The support can also be a relatively inert polymer, which can be grafted by ionizing radiation to permit attachment of a coating of polystyrene or other such polymer that can be derivatized and used as a support. Radiation grafting of monomers allows a diversity of surface characteristics to be generated on supports (see, e.g., Maeji et al. (1994) Reactive Polymers 22:203-212; and Berg et al. (1989) J. Am. Chem. Soc. 111:8024-8026). For example, radiolytic grafting of monomers, such as vinyl momomers, or mixtures of monomers, to polymers, such as polyethylene and polypropylene, produce composites that have a wide variety of surface characteristics. These methods have been used to graft polymers to insoluble supports for synthesis of peptides and other molecules

The supports are typically insoluble substrates that are solid, porous, deformable, or hard, and have any required structure and geometry, including, but not limited to: beads, pellets, disks, capillaries, hollow fibers, needles, solid fibers, random shapes, thin films and membranes, and most generally, form solid surfaces with addressable loci. The supports can also include an inert strip, such as a teflon strip or other material to which the capture agents antibodies and other molecules do not adhere, to aid in handling the supports, and can include an identifying symbology.

The preparation of and use of such supports are well known to those of skill in this art; there are many such materials and preparations thereof known. For example, naturally-occurring materials, such as agarose and cellulose, can be isolated from their respective sources, and processed according to known protocols, and synthetic materials can be prepared in accord with known protocols. These materials include, but are not limited to, inorganics, natural polymers, and synthetic polymers, including, but are not limited to: cellulose, cellulose derivatives, acrylic resins, glass, silica gels, polystyrene, gelatin, polyvinyl pyrrolidone, co-polymers of vinyl and acrylamide, polystyrene cross-linked with divinylbenzene or the like (see, Merrifield (1964) Biochemistry 3:1385-1390), polyacrylamides, latex gels, polystyrene, dextran, polyacrylamides, rubber, silicon, plastics, nitrocellulose, celluloses, natural sponges, and many others. Selection of the supports is governed, at least in part, by their physical and chemical properties, such as solubility, functional groups, mechanical stability, surface area swelling propensity, hydrophobic or hydrophilic properties and intended use.

a. Natural Support Materials

Naturally-occurring supports include, but are not limited to agarose, other polysaccharides, collagen, celluloses and derivatives thereof, glass, silica, and alumina. Methods for isolation, modification and treatment to render them suitable for use as supports is well known to those of skill in this art (see, e.g., Hermanson et al. (1992) Immobilized Affinity Ligand Techniques, Academic Press, Inc., San Diego). Gels, such as agarose, can be readily adapted for use herein. Natural polymers such as polypeptides, proteins and carbohydrates; metalloids, such as silicon and germanium, that have semiconductive properties, can also be adapted for use herein. Also, metals such as platinum, gold, nickel, copper, zinc, tin, palladium, silver can be adapted for use herein. Other supports of interest include oxides of the metal and metalloids such as Pt—PtO, Si—SiO, Au—AuO, TiO₂, Cu—CuO, and the like. Also compound semiconductors, such as lithium niobate, gallium arsenide and indium-phosphide, and nickel-coated mica surfaces, as used in preparation of molecules for observation in an atomic force microscope (see, e.g., III et al. (1993) Biophys J. 64:919) can be used as supports. Methods for preparation of such matrix materials are well known.

For example, U.S. Pat. No. 4,175,183 describes a water insoluble hydroxyalkylated cross-linked regenerated cellulose and a method for its preparation. A method of preparing the product using near stoichiometric proportions of reagents is described. Use of the product directly in gel chromatography and as an intermediate in the preparation of ion exchangers is also described.

b. Synthetic Supports

There are innumerable synthetic supports and methods for their preparation known to those of skill in this art. Synthetic supports typically produced by polymerization of functional matrices, or copolymerization from two or more monomers from a synthetic monomer and naturally occurring matrix monomer or polymer, such as agarose.

Synthetic matrices include, but are not limited to: acrylamides, dextran-derivatives and dextran co-polymers, agarose-polyacrylamide blends, other polymers and co-polymers with various functional groups, methacrylate derivatives and co-polymers, polystyrene and polystyrene copolymers (see, e.g., Merrifield (1964) Biochemistry 3:1385-1390; Berg et al. (1990) in Innovation Perspect. Solid Phase Synth. Collect. Pap., Int. Symp., 1st, Epton, Roger (Ed), pp. 453-459; Berg et al. (1989) in Pept., Proc. Eur. Pept. Symp., 20th, Jung, G. et al. (Eds), pp. 196-198; Berg et al. (1989) J. Am. Chem. Soc. 111:8024-8026; Kent et al. (1979) Isr. J. Chem. 17:243-247; Kent et al. (1978) J. Org. Chem. 43:2845-2852; Mitchell et al. (1976) Tetrahedron Lett. 42:3795-3798; U.S. Pat. No. 4,507,230; U.S. Pat. No. 4,006,117; and U.S. Pat. No. 5,389,449). Methods for preparation of such support matrices are well-known to those of skill in this art.

Synthetic support matrices include those made from polymers and co-polymers such as polyvinylalcohols, acrylates and acrylic acids such as polyethylene-co-acrylic acid, polyethylene-co-methacrylic acid, polyethylene-co-ethylacrylate, polyethylene-co-methyl acrylate, polypropylene-co-acrylic acid, polypropylene-co-methyl-acrylic acid, polypropylene-co-ethyl-acrylate, polypropylene-co-methyl acrylate, polyethylene-co-vinyl acetate, polypropylene-co-vinyl acetate, and those containing acid anhydride groups such as polyethylene-co-maleic anhydride, polypropylene-co-maleic anhydride and the like. Liposomes have also been used as solid supports for affinity purifications (Powell et al. (1989) Biotechnol. Bioeng. 33:173).

For example, U.S. Pat. No. 5,403,750, describes the preparation of polyurethane-based polymers. U.S. Pat. No. 4,241,537 describes a plant growth medium containing a hydrophilic polyurethane gel composition prepared from chain-extended polyols; random copolymerization can be performed with up to 50% propylene oxide units so that the prepolymer is a liquid at room temperature. U.S. Pat. No. 3,939,123 describes lightly crosslinked polyurethane polymers of isocyanate terminated prepolymers containing poly(ethyleneoxy) glycols with up to 35% of a poly(propyleneoxy) glycol or a poly(butyleneoxy) glycol. In producing these polymers, an organic polyamine is used as a crosslinking agent. Other supports and preparation thereof are described in U.S. Pat. Nos. 4,177,038, 4,175,183, 4,439,585, 4,485,227, 4,569,981, 5,092,992, 5,334,640, 5,328,603.

U.S. Pat. No. 4,162,355 describes a polymer suitable for use in affinity chromatography, which is a polymer of an aminimide and a vinyl compound having at least one pendant halo-methyl group. An amine ligand, which affords sites for binding in affinity chromatography is coupled to the polymer by reaction with a portion of the pendant halo-methyl groups and the remainder of the pendant halo-methyl groups are reacted with an amine containing a pendant hydrophilic group. A method of coating a substrate with this polymer is also described. An exemplary aminimide is 1,1-dimethyl-1-(2-hydroxyoctyl)amine methacrylimide and vinyl compound is a chloromethyl styrene.

U.S. Pat. No. 4,171,412 describes specific supports based on hydrophilic polymeric gels, generally of a macroporous character, which carry covalently bonded D-amino acids or peptides that contain D-amino acid units. The basic support is prepared by copolymerization of hydroxyalkyl esters or hydroxyalkylamides of acrylic and methacrylic acid with crosslinking acrylate or methacrylate comonomers are modified by the reaction with diamines, amino acids or dicarboxylic acids and the resulting carboxyterminal or aminoterminal groups are condensed with D-analogs of amino acids or peptides. The peptide containing D-amino-acids also can be synthesized stepwise on the surface of the carrier.

U.S. Pat. No. 4,178,439 describes a cationic ion exchanger and a method for preparation thereof. U.S. Pat. No. 4,180,524 describes chemical syntheses on a silica support.

Immobilized Artificial Membranes (IAMs; see, e.g., U.S. Pat. Nos. 4,931,498 and 4,927,879) can also be used. IAMs mimic cell membrane environments and can be used to bind molecules that preferentially associate with cell membranes (see, e.g., Pidgeon et al. (1990) Enzyme Microb. Technol. 12:149).

Among the supports contemplated herein are those described in International PCT application Nos WO 00/04389, WO 00/04382 and WO 00/04390; KODAK film supports coated with a matrix material; see also, U.S. Pat. Nos. 5,744,305 and 5,556,752 for other supports of interest. Also of interest are colored “beads”, such as those from Luminex (Austin, Tex.).

c. Immobilization and Activation

Numerous methods have been developed for the immobilization of proteins and other biomolecules onto solid or liquid supports (see, e.g., Mosbach (1976) Methods in Enzymology 44; Weetall (1975) Immobilized Enzymes, Antigens, Antibodies, and Peptides; and Kennedy et al. (1983) Solid Phase Biochemistry, Analytical and Synthetic Aspects, Scouten, ed., pp. 253-391; see, generally, Affinity Techniques. Enzyme Purification: Part B. Methods in Enzymology, Vol. 34, ed. W. B. Jakoby, M. Wilchek, Acad. Press, N.Y. (1974); Immobilized Biochemicals and Affinity Chromatography, Advances in Experimental Medicine and Biology, vol. 42, ed. R. Dunlap, Plenum Press, N.Y. (1974)).

Among the most commonly used methods are absorption and adsorption or covalent binding to the support, either directly or via a linker, such as the numerous disulfide linkages, thioether bonds, hindered disulfide bonds, and covalent bonds between free reactive groups, such as amine and thiol groups, known to those of skill in art (see, e.g., the PIERCE CATALOG, ImmunoTechnology Catalog & Handbook, 1992-1993, which describes the preparation of and use of such reagents and provides a commercial source for such reagents; and Wong (1993) Chemistry of Protein Conjugation and Cross Linking, CRC Press; see, also DeWitt et al. (1993) Proc. Natl. Acad. Sci. U.S.A. 90:6909; Zuckermann et al. (1992) J. Am. Chem. Soc. 114:10646; Kurth et al. (1994) J. Am. Chem. Soc. 116:2661; Ellman et al. (1994) Proc. Natl. Acad. Sci. U.S.A. 91:4708; Sucholeiki (1994) Tetrahedron Lttrs. 35:7307; and Su-Sun Wang (1976) J. Org. Chem. 41:3258; Padwa et al. (1971) J. Org. Chem. 41:3550 and Vedejs et al. (1984) J. Org. Chem. 49:575, which describe photo-sensitive linkers).

To effect immobilization, a solution of the protein or other biomolecule is contacted with a support material such as alumina, carbon, an ion-exchange resin, cellulose, glass or a ceramic. Fluorocarbon polymers have been used as supports to which biomolecules have been attached by adsorption (see, U.S. Pat. No. 3,843,443; Published International PCT Application WO/86 03840)

A large variety of methods are known for attaching biological molecules, including proteins and nucleic acids, molecules to solid supports (see, e.g., U.S. Pat. No. 5,451,683). For example, U.S. Pat. No. 4,681,870 describes a method for introducing free amino or carboxyl groups onto a silica support. These groups can subsequently be covalently linked to other groups, such as a protein or other anti-ligand, in the presence of a carbodiimide. Alternatively, a silica matrix can be activated by treatment with a cyanogen halide under alkaline conditions. The anti-ligand is covalently attached to the surface upon addition to the activated surface. Another method involves modification of a polymer surface through the successive application of multiple layers of biotin, avidin and extenders (see, e.g., U.S. Pat. No. 4,282,287); other methods involve photoactivation in which a polypeptide chain is attached to a solid substrate by incorporating a light-sensitive unnatural amino acid group into the polypeptide chain and exposing the product to low-energy ultraviolet light (see, e.g., U.S. Pat. No. 4,762,881). Oligonucleotides have also been attached using photochemically active reagents, such as a psoralen compound, and a coupling agent, which attaches the photoreagent to the substrate (see, e.g., U.S. Pat. No. 4,542,102 and U.S. Pat. No. 4,562,157). Photoactivation of the photoreagent binds a nucleic acid molecule to the substrate to give a surface-bound probe.

Covalent binding of the protein or other biomolecule or organic molecule or biological particle to chemically activated solid matrix supports such as glass, synthetic polymers, and cross-linked polysaccharides is a more frequently used immobilization technique. The molecule or biological particle can be directly linked to the matrix support or linked via a linker, such as a metal (see, e.g., U.S. Pat. No. 4,179,402; and Smith et al. (1992) Methods: A Companion to Methods in Enz. 4:73-78). An example of this method is the cyanogen bromide activation of polysaccharide supports, such as agarose. The use of perfluorocarbon polymer-based supports for enzyme immobilization and affinity chromatography is described in U.S. Pat. No. 4,885,250). In this method the biomolecule is first modified by reaction with a perfluoroalkylating agent such as perfluorooctylpropylisocyanate described in U.S. Pat. No. 4,954,444. Then, the modified protein is adsorbed onto the fluorocarbon support to effect immobilization.

The activation and use of supports are well known and can be effected by any such known methods (see, e.g., Hermanson et al. (1992) Immobilized Affinity Ligand Techniques, Academic Press, Inc., San Diego). For example, the coupling of the amino acids can be accomplished by techniques familiar to those in the art and provided, for example, in Stewart and Young, 1984, Solid Phase Synthesis, Second Edition, Pierce Chemical Co., Rockford.

Molecules can also be attached to supports through kinetically inert metal ion linkages, such as Co(II), using, for example, native metal binding sites on the molecules, such as IgG binding sequences, or genetically modified proteins that bind metal ions (see, e.g., Smith et al. (1992) Methods: A Companion to Methods in Enzymology 4, 73 (1992); III et al. (1993) Biophys J. 64:919; Loetscher et al. (1992) J. Chromatography 595:113-199; U.S. Pat. No. 5,443,816; Hale (1995) Analytical Biochem. 231:46-49).

Other suitable methods for linking molecules and biological particles to solid supports are well known to those of skill in this art (see, e.g., U.S. Pat. No. 5,416,193). These linkers include linkers that are suitable for chemically linking molecules, such as proteins and nucleic acid, to supports include, but are not limited to, disulfide bonds, thioether bonds, hindered disulfide bonds, and covalent bonds between free reactive groups, such as amine and thiol groups. These bonds can be produced using heterobifunctional reagents to produce reactive thiol groups on one or both of the moieties and then reacting the thiol groups on one moiety with reactive thiol groups or amine groups to which reactive maleimido groups or thiol groups can be attached on the other. Other linkers include, acid cleavable linkers, such as bismaleimideothoxy propane, acid labile-transferrin conjugates and adipic acid diihydrazide, that are cleaved in more acidic intracellular compartments; cross linkers that are cleaved upon exposure to UV or visible light and linkers, such as the various domains, such as C_{H}1, C_{H}2, and C_{H}3, from the constant region of human IgG₁ (see, Batra et al. (1993) Molecular Immunol. 30:379-386).

Exemplary linkages include direct linkages effected by adsorbing the molecule or biological particle to the surface of the support. Other exemplary linkages are photocleavable linkages that can be activated by exposure to light (see, e.g., Baldwin et al. (1995) J. Am. Chem. Soc. 117:5588; Goldmacher et al. (1992) Bioconj. Chem. 3:104-107, which linkers are herein incorporated by reference). The photocleavable linker is selected such that the cleaving wavelength that does not damage linked moieties. Photocleavable linkers are linkers that are cleaved upon exposure to light (see, e.g., Hazum et al. (1981) in Pept., Proc. Eur. Pept. Symp., 16th, Brunfeldt, K (Ed), pp. 105-110, which describes the use of a nitrobenzyl group as a photocleavable protective group for cysteine; Yen et al. (1989) Makromol. Chem 190:69-82, which describes water soluble photocleavable copolymers, including hydroxypropylmethacrylamide copolymer, glycine copolymer, fluorescein copolymer and methylrhodamine copolymer; Goldmacher et al. (1992) Bioconj. Chem. 3:104-107, which describes a cross-linker and reagent that undergoes photolytic degradation upon exposure to near UV light (350 nm); and Senter et al. (1985) Photochem. Photobiol 42:231-237, which describes nitrobenzyloxycarbonyl chloride cross linking reagents that produce photocleavable linkages). Other linkers include fluoride labile linkers (see, e.g., Rodolph et al. (1995) J. Am. Chem. Soc. 117:5712), and acid labile linkers (see, e.g., Kick et al. (1995) J. Med. Chem. 38:1427)). The selected linker depends upon the particular application and, if needed, can be empirically selected.

7. Detection of Bound Antigen(s)

Bound tagged reagents, such as tagged polypeptides, can be detected by any suitable method known to those of skill in the art and is a function of the target molecules. Exemplary detection methods include the use of chemiluminescence and bioluminescence generating reagents, such as horse radish peroxidase (HRP) systems and luciferin/luciferase systems, alkaline phosphatase (AP), labeled antibodies, fluorophores and isotopes. These can be detected using film, photon collection, scanning lasers, waveguides, ellipsometry, CCDs and other imaging techniques.

As noted, uses of the addressable capture agent collections include, but are not limited to: searching a recombinant antibody scFv library to identify scFv includes, but is not limited to, finding single antigen or multiple antigens; searching mutation libraries, including tagging mutant libraries; mutation by error prone PCR; mutation by gene shuffling for searching for small molecule binders, searching for increased antibody affinity, searching for enhanced enzymatic properties (alkaline phosphatase (AP), horse radish peroxidase (HRP), luciferase and photoproteins, fluorescent proteins, such as green, blue or red fluorescent proteins (GFP, BFP, RFP); searching for sequence-specific DNA binding proteins; searching a cDNA library for protein-protein interactions; and any other such application.

a. Methods of Staining

The staining of the sample can be non-specific, semi-specific or specific depending on when the sample is stained and what is stained. The staining of the sample, such as molecules or biological particles, can occur prior to, subsequent or during contacting the capture agents with the tagged-molecules. Samples can be non-differentially or differentially stained. In each instance, the level of specificity of the molecules assessed varies.

For example, a cellular culture can be disrupted and the resulting lysate can be non-selectively stained, such as by biotinylation. The stained solution or lysate can then be contacted with the arrayed capture agents or tagged molecules, and the stained components are visualized by exposure to a horseradish peroxidase (HRP) conjugated anti-biotin antibody. Alternatively, the biological particles themselves are stained, such as by biotinylation, and then cells are lysed and, optionally, receptors are liberated from the membrane. In this instance, not all the sample components applied to the arrayed capture agents or tagged molecules are stained, so only stained particles that resided on the surface of the biological particle are detected. Therefore, subfractions can be semi-specifically stained and analyzed. For example, proteins and other molecules present on the cell surface can be identified. In other applications, organelles can be prepared and molecules on the surfaces of the organelle can be identified.

In other embodiments, the sample is contacted with the arrayed capture agents or tagged molecules and then stained, such as by visualization with a specific stain. Specific staining results in the visualization of a specific molecule or class of molecules to which a stain can bind specifically. The stain for a specific molecule can be any molecule or compound which interacts exclusively with the molecule or class of molecules of interest. To stain for a class of molecules, such as the immunoglobulins, the class of molecules contains a constant domain to which the stain can bind specifically and a variable domain which can interact with the capture system. Once the sample is overlayed on the array, the arrays are stained with a label, such as, but not limited to, an antibody, specific for a particular molecule or class of molecules. Thus, only the specific molecule or class of molecules stained is visualized on the array.

Specific staining can be used to assess and monitor changes in the levels of a specific molecule or class of molecules within a sample as the result of, for example, time, exposure to a condition or perturbation and the propagation of a diseased state. For example, when B cells initially develop, an IgM immunoglobulin is displayed on the surface of the cell. IgM is a member of the immunoglobulin superfamily, where all members possess similar structure by virtue of a contain a constant domain and a variable domain. Different classes of immunoglobins (IgG, IgA, IgE, IgD and IgM) vary in the amino acid sequence of their respective constant domains. Also, each immunoglobulin generally has different isotypic constant domains. For example, IgG has multiple isoforms including IgG1, IgG4 and IgGA. T cells and MHC molecules, which also belong to the immunoglobulin superfamily, have variable regions attached to a constant region but these regions do not have homology with each other or the members of other classes of the immunoglobulin superfamily. These differences in the constant regions of the various members of the members of this diverse family allow for the specific staining of a particular class of immunoglobulins of interest.

For example, to monitor alterations in the idiotype of a subject, the B cells of a subject can be harvested, combined and lysed to obtain a lystate containing all of the IgM molecules present on the surface of the B cells. The lysate can then be overlayed on arrays displaying a library of scFv molecules such that the variable regions of the various IgM molecules interact with their complementary scFvs on the arrays. The immobilized IgM molecules can then be specifically stained with an anti-Ig-Fc antibody which recognizes the constant region (Fc) of the all the IgM molecules attached to the arrays. The stain is specific for the IgM molecules because the constant region of the various immunoglobulins such as IgG, IgA, IgE and IgD are different from one another. The resulting pattern visualized on the arrays presents an image of the variable regions present in the IgM molecules within the sample due to their interaction with the scFvs displayed on the arrays. This pattern can then be used as a baseline for monitoring changes in the idiotypic landscape of the subject, for example, over time, following the administration of a drug molecule or during the course of a disease. Further, this pattern can be compared to similar samples from other subjects to assess the effect of varied environments on the display of IgM molecules by the B cells. Once IgM molecules are identified as being of interest, the arrays can be tailored to allow for the monitoring of the levels of IgM produced as a result of a change in the environment of the subject.

In a similar manner, the interaction between T cell receptors (TCR) and the scFv library can be monitored by specific staining. T cell receptors contain a constant domain and a variable domain which can be exploited for specific staining using an anti-TCR constant domain antibody. TCR are responsible for the recognition of fragments of protein antigens on the surfaces of antigen presenting cells, which results in the activation of the T cell. The patterns discerned from arrays overlayed with a sample containing T cells can be used to assess and monitor the immune state and response of a subject at a particular time or over an extended time period. Variations in the pattern also can be used to monitor the effect of various drug molecules on a disease state or the progression or regression of a disease on the immune system response. Identification and monitoring of a particular TCR or group of TRCs of interest also can be performed utilizing the arrayed capture agents or tagged molecules and specific staining.

Presentation of peptide fragments of antigens by an antigen-presenting cell (APC) is performed by the major histocompatibility complex (MHC) during an immune response. Similar to immunoglobulins and TCRs, MHC has a variable region that interacts with the antigen fragment and a constant region. This constant region can be exploited for specific staining using the capture systems provided herein resulting in the high resolution mapping of antigen presentation during an immune response. The mapping of antigen presentation is an invaluable tool in the early diagnosis of disease, bacterial or viral infection. If levels of a particular MHC increase, then a particular disease state may be present. Similarly, the effect of drug molecules or an alteration in the cellular conditions can be monitored by assessing the pattern of antigen presentation.

Specific staining also can be used to monitor changes in receptor landscapes. For example, a library of molecules, such as scFvs, which interact with cell surface receptors can be displayed on the arrays. The arrays are then exposed to a cellular sample. The interaction between the cell surface receptors and the scFvs displayed on the arrays can result in the transduction of a signal from the surface to the interior of the cell, resulting in a response. The response can be monitored in a specific or semi-specific manner. For example, a cytotoxic T cell activates a death-inducing caspase cascade in the target cell by interacting with transmembrane receptor proteins, Fas. Binding of the Fas ligand on the T cell to the Fas proteins on the target cell alters the Fas proteins so that their clustered cytosolic tails recruit procaspase-8 in the complex via an adaptor protein. The recruited procaspase-8 molecules cross-cleave and activate one another to begin the caspase cascade that leads to apoptosis. The death of the cell can be monitored by specific dyes that are released upon cell death, however, the cause of death is unknown due to the non-specific nature of the apoptosis visualization. Instead, scFv molecules can be displayed on arrays and exposed to cellular samples. The cells can then be fixed and permeablized such that a stain specific for caspase, such as the anti-Zap7O antibody, can enter the interior of the cell and be visualized. The presence of activated caspase, as indicated by the staining, highlights those cells where the caspase cascade has been activated by the interaction between the scFv library and the cell surface receptors of the proteins.

Similarly, but less specifically, the initiation of classes of enzymes, such as the kinases, can be monitored by specific staining. For example, arrayed capture agents displaying a tagged scFv library can be contacted to a cellular sample. The cells can then be fixed and permabilized. Upon permabilization, the arrays are stained with an anti-Phos Tyr antibody which is specific for peptides containing phosphorylated tyrosines. Cells which are visualized indicate a cellular system where the interaction of the scFv on the array resulting in a cellular signal that initiated kinase activity.

Another example demonstrates the use of specific stain, such as an anti-SH2/SH3 antibody, that is used to stain cells where a signaling pathway incorporating peptides with SH2 or SH3 domains has been initiated by interaction between the cell surface receptors and the scFv library.,

b. Molecules for Staining

There are many staining methods used to localize molecules that are known to those skilled in the art, and any can be used in the methods herein. Selection of the stain can be made by those of skill in the art and depends upon the particular application. For example, factors that affect the method chosen, include, for example, the type of sample, the degree of sensitivity needed and the processing time and cost requirements.

Staining of molecules can be performed directly or indirectly. Direct staining involves the staining and detection of a specific molecule or class of molecules of interest. Indirect staining involves the staining and detection of a molecule resulting from a secondary reaction of the molecule or class of molecules of interest, such as a signal transduction product or the product of an enzymatic reaction. Molecules used for staining can be any compound that is detectable or produces a detectable signal. Molecules that can be used for staining include, but are not limited to, an organic compound, inorganic compound, metal complex, receptor, enzyme, antibody, protein, nucleic acid, peptide nucleic acid, DNA, RNA, polynucleotide, oligonucleotide, oligosaccharide, lipid, lipoprotein, amino acid, peptide, polypeptide, peptidomimetic, carbohydrate, cofactor, drug, prodrug, lectin, sugar, glycoprotein, biomolecule, macromolecule, biopolymer, polymer, sub-cellular structure, sub-cellular compartment or any combination, portion, salt, or derivative thereof. These molecules can be detected directly or labelled with a detectable label, such as a luminescent molecule.

Molecules, such as antibodies, are commercially available conjugated to a detectable label or are synthetically producible for use in specific staining depending on the particular molecule or class of molecules of interest. Proteins which can be used as a detectable label include, but are not limited to, GFP, RFP and BFP. A wide variety of luminescent molecules are commercially available, and include, but are not limited to, FITC, fluorescein, rhodamine, Cascade Blue, Marina Blue, Alexa Fluor 350, red-fluorescent Alexa Fluor 594, Texas Red, Texas Red-X and the red- to infrared-fluorescent Alexa Fluor 633, Alexa Fluor 647, Alexa Fluor 660, Alexa Fluor 680, Alexa Fluor 700 and Alexa Fluor 750 dyes (Molecular Probes). Attachment of the luminescent molecule can be performed by any method known to those skilled in the art, such as with the Zenon One Mouse IgG₁ labeling kit from Molecular Probes. Conjugated antibodies also can be commercially purchased with the luminescent label already attached from companies such as Molecular Probes (www.probes.com), Invitrogen (www.invitrogen.com), Amersham Biosciences (www.amershambiosciences.com) and Pierce Biotechnologies (www.piercenet.com).

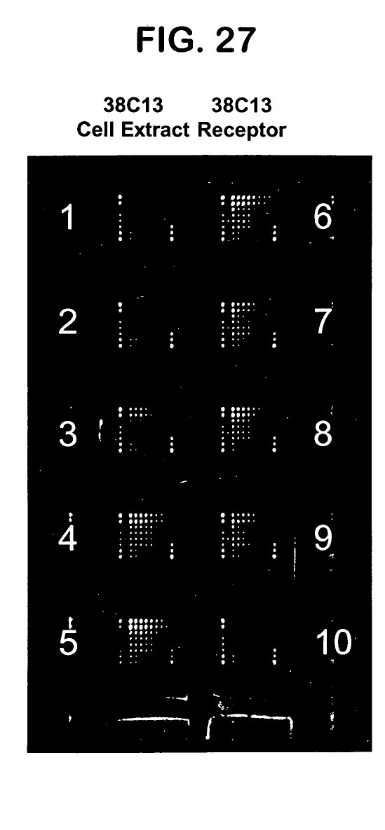

A particular embodiment of specific staining is exemplified in Example 9. Briefly, idiotype receptors can be used to identify lymphoma cells. These receptors are IgM molecules that reside on the surface of lymphoma cells. In order to identify a scFv that interacts with an idiotype receptor from a particular lymphoma cell, a sample lystate from a lymphoma culture is exposed to a capture system displaying a master library of tagged scFv molecules. Once lystate components are bound to the arrayed tagged scFv molecules, IgM molecules are specifically stained with a detection antibody, such as an anti-1 g-Fc antibody, that is specific for the constant domain of IgM molecules. The secondary antibody is then visualized by any method known to those skilled in the art, indicating which loci within the arrays contain IgM molecules from the lymphoma cells of the sample that are interacting with a scFv through the IgM receptor (FIG. 27).

c. Immunostaining

There are many immunostaining methods used to localize antigens are known to those skilled in the art. Many factors affect the method of choice including the type of sample, the degree of sensitivity needed and the processing time and cost requirements. Immunostaining of antigens can be performed directly or indirectly. Direct staining is a method in which an enzyme linked primary antibody reacts with the antigen in the sample. Subsequent use of substrate-chromagen concludes the reaction sequence and results in a detectable product. Indirect staining is a method in which an unconjugated primary antibody binds to an antigen. An enzyme-labelled secondary antibody directed against the primary antibody is then applied, followed by substrate-chromagen solution that results in a detectable product. The secondary antibody generally is prepared in a subject different from subject in which the primary antibody was prepared. For example, if the primary antibody is made in rabbit or mouse, the secondary antibody should be directed against rabbit or mouse immunoglobulins. Additional layers of secondary antibodies are also contemplated. The enzyme or enzymes can be attached to the antibody by any method known to those skilled in the art (Wild The Immunoassay Handbook, Nature Publishing Group (2001) and Van der Loos Immunoenzyme Multiple Staining Methods, Bios Scientific Pub Ltd (2000)) or can be purchased commercially as an enzyme-antibody conjugate. The reaction product can be detected by any method known to those skilled in the art including, but not limited to, colormetric, spectroscopic and electrochemical (Kulis et al. J. Electroanal. Chem. 382: 129 (1995); Bauer et al. Anal. Chem. 68: 2453 (1996); and Bagel et al. Anal. Chem. 69: 4688).

(1) Enzymes and Chromagens for Immunostaining

Most immunoenzymatic staining methods utilize enzyme-substrate reactions to convert colorless chromagens into colored end products. Any enzyme that can react with a chromagen directly or a substrate to yield a product that can then react with a chromagen to yield a detectable signal and can be attached to an antibody that interacts either directly or indirectly with an antigenic species can be used. Some exemplary enzymes include, but are not limited to, horseradish peroxidase (HRP) and calf intestine alkaline phosphatase (AP), galactosidase and glucose oxidase. Additionally, luminescent proteins such as green fluorescent protein (GFP), red fluorescent protein (RFP) and blue fluorescent protein (BFP) or other luminescent molecules, such as, FTIC, rhodamine, fluorscein and Alexa Fluor dyes (Molecular Probes), can be attached to the antibody being used and visualized directly.

i) Luminescent Labels

In immunostaining techniques, a luminescent label is a molecule that can be attached to either a primary or secondary antibody and visualized without the addition of a substrate or a chromagen. Proteins which can be used include, but are not limited to, GFP, RFP and BFP. A wide variety of luminescent molecules are commercially available, and include, but are not limited to, FITC, fluorescein, rhodamine, Cascade Blue, Marina Blue, Alexa Fluor 350, red-fluorescent Alexa Fluor 594, Texas Red, Texas Red-X and the red- to infrared-fluorescent Alexa Fluor 633, Alexa Fluor 647, Alexa Fluor 660, Alexa Fluor 680, Alexa Fluor 700 and Alexa Fluor 750 dyes (Molecular Probes). Attachment of the luminescent molecule can be performed by any method known to those skilled in the art, such as with the Zenon One Mouse IgG₁ labeling kit from Molecular Probes. Conjugated antibodies also can be commercially purchased with the luminescent label already attached from companies such as Molecular Probes (www.probes.com), Invitrogen (www.invitrogen.com), Amersham Biosciences (www.amershambiosciences.com) and Pierce Biotechnologies (www.piercenet.com).

ii) Horseradish Peroxidase (HRP)

HRP is a heme-containing enzyme isolated from the root of the horseradish plant. The heme substituent of HRP forms a complex with hydrogen peroxide, which then decomposes resulting in water and atomic oxygen. HRP oxidizes several substances, such as polyphenols and nitrates. HRP can be covalently or non-covalently attached to other proteins, such as antibodies, using any method known to those skilled in the art (see, e.g., Sternberger Immunocytochemistry (2nd Ed.) New York: Wiley, 1979) or can be purchased as part of a conjugated antibody-enzyme complex from commercial sources such as Invitrogen, Pierce Biotechnologies and Amersham Biosciences.

HRP activity in the presence of an electron donor, such as hydrogen peroxide, first results in the formation of an enzyme-substrate complex, and then in the oxidation of the electron donor. The electron donor provides the driving force in the continuing catalysis of hydrogen peroxide, while its absence effectively stops the reaction. Electron donors, called chromagens, become colored products when oxidized and include, but are not limited to, 3,3′-Diaminobenzidine (DAB), 3-Amino-9-ethylcarbazole (AEC), 4-Chloro-1-naphthol (CN), p-Phenylenediaminie dihydrochloride/pyrocatechol (Hanker-Yates reagent), chloro-1-naphthol, luminol, ECF substrate and 3,3′,5,5′-tetramethylbenzidine (TMB). These compounds can be synthetically prepared by any method known to those skilled in the art or can be purchased from commercial sources.

iii) Alkaline Phosphatase (AP)

Calf intestine alkaline phosphatase removes and transfers phosphate groups from organic esters by breaking the phosphate-oxygen bond. The chief metal activators are divalent magnesium, manganese and calcium. Alkaline phosphatase can be covalently or non-covalently attached to other proteins, such as antibodies, synthetically using any method known to those skilled in the art, or can be purchased as an antibody-enzyme complex.

In the immunoalkaline phosphatase staining method, the enzyme hydrolyzes naphthol phosphate esters (substrate) to phenolic compounds and phosphates. The phenols couple to colorless diazonium salts (chromagen) to produce insoluble, colored azo dyes. Substrates used in conjunction with alkaline phosphatase include, but are not limited to, Naphthol AS-MX phosphate, naphthol AS-BI phosphate, naphthol AS-TR phosphate and 5-bromo-4-chloro-3-indoxyl phosphate (BCIP). Chromagens used include, but are not limited to Fast Red TR, Fast Blue BB, new fuchsin, Fast Red LB, Fast Garnet GBC, Nitro Blue Tetrazolium (NBT) and iodonitrotetrazolium violet (INT). These compounds can be synthetically prepared by any method known to those skilled in the art or can be purchased from commercial sources.

(2) Avidin-Biotin Staining Methods

As described above, immunostaining can be accomplished either directly or indirectly using enzymatic reaction for visualization of the antigenic site. In an extension of these methods, the interaction between avidin and biotin has been exploited to develop an immunostaining method that has an inherent amplification of sensitivity when compared with other methods. Avidin (chicken egg) is a tetramer containing four identical subunits. Each subunit contains a high affinity binding site for biotin, an egg white protein, with a dissociation constant of approximately 10⁻¹⁵ M. The binding is undisturbed by extremes of pH, buffer salts or chaotropic agents such as guanidine hydrochloride. Streptavidin, from Streptomyces avidinii, can be exchanged for avidin in the interaction with biotin.

This strong interaction is the focus-of three immunostaining methods. The labelled avidin-biotin (LAB) method (Guesdon et al. J. Histochem. Cytochem. 27: 1131 (1983)) utilizes a biotinylated antibody which is reacted either with an antigen or a primary antibody, followed by a second layer of enzyme-labelled avidin. After the avidin-enzyme conjugate binds to the biotinylated antibody, chromagen is added to detect the antigen. The bridged avidin-biotin method (BRAB) (Guesdon et al. J. Histochem. Cytochem. 27: 1131 (1983)) is essentially the same as the LAB method, except that the avidin is not conjugated to an enzyme. The BRAB method utilizes avidin as a bridge between the biotinylated antibody and a biotinylated enzyme. Due to the multiple binding sites on avidin, more biotinylated enzymes can be complexed to increase the intensity of the chromagen color development. The avidin-biotin complex (ABC) method (Hsu et al. Am. J. Clin. Path. 75: 734-738 (1981); Hsu et al. Am. J. Clin. Path. 75: 816 (1981); and Hsu et al. J. Histochem. Cytochem. 29: 577-580 (1981)) utilizes the initial complex as in the LAB or BRAB system, but requires that the biotinylated enzyme be preincubated with the avidin, forming large complexes to be incubated with the biotinylated antibody. The avidin and biotinylated enzyme are mixed together in a specified ratio for about 15 minutes at room temperature to form these complexes. An aliquot of this solution is then added to the sample, and any remaining biotin-binding sites will bind to the biotinylated antibody. The result is a greater concentration of enzyme at the antigenic site in the sample and an increase in sensitivity.

(3) Chain Polymer-Conjugated Technology

To achieve high sensitivity, the most commonly used staining methods in immunohistochemistry to date have been based on a multi-layer technique. Conjugates used in multi-layer techniques normally consist of one or two enzyme molecules per antibody or avidin-strepavidin molecules. A biotinylated secondary antibody and an avidin-strepavidin conjugate are used to exploit the high affinity of avidin-strepavidin for biotin. Sensitivity is enhanced by increasing the number of enzyme molecules bound to the antigen through the detecting antibody. A technology recently developed by DAKO (www.dako.com) enables the coupling of a high number of molecules to a dextran backbone. This chemistry permits binding of a large number of enzyme molecules (e.g., horseradish peroxidase or alkaline phosphatase) to a secondary antibody via the dextran backbone. The resulting polymeric conjugate can consist of up to 100 enzyme molecules and up to 20 antibody molecules per backbone and is kept water-soluble by using hydrophilic, non-charged dextran as the backbone. The increase in the number of enzymes per antigen results in an increase in sensitivity, a minimization of non-specific background staining and a reduction in the total number of assay steps as compared to conventional technologies. Staining kits and reagents, such as the Enhanced Polymer One-Step Method (EPOS™) and EnVision systems, that utilize this technology can be purchased commercially from DAKO.

C. Use of the Collections of Capture systems, Collections of Binding Sites and Collections of Capture Agents for Profiling

The capture agent collections and capture agent collections with bound molecules containing polypeptide (epitope) or other tags (the capture system) can serve as devices for profiling samples, particularly biological samples for, for example, diagnostic, prognostic and drug discovery purposes. For example, a biological sample, such as a body fluid, a tissue or organ sample or a tumor sample, can be prepared and exposed to a collection of binding sites that display a library of molecules, such as a scFv or a T cell receptor library, and the binding profile assessed. Binding profiles can then be compared among samples and the presence or absence of the binding of components within the samples can be used to identify markers indicative of a particular disease state. Further, samples can be exposed to a perturbation, such as a candidate compound or a condition, and the binding profile reassessed. Alterations in the profile can be indicative of the effect of the perturbation on the sample and identify potential therapeutic compounds.

Any sample can be contacted with a capture agent collection or capture agent collection with bound molecules (collection of binding sites) containing tags, such as polypeptide tags. Bound moieties can be detected by any suitable method, such as by enzyme, fluorescent or immunological labeling. The result of the detection, or the output, is information, such as an image, a picture, a data spreadsheet, or a scatter plot, which can be used to compile a binding profile of the sample to the collection of binding sites. Each sample produces a characteristic profile, which can be used to identify a pattern in the information that can serve as an identifier of the source of a sample or components thereof. The patterns are arrangements of the information from the detection of the binding of the sample to the collection of binding sites, and the means of collection of the information is irrelevant to finding a pattern in the information. For example, if a particular sample from a diseased host is exposed to a collection of binding sites, wherein the tagged reagents include scFvs, a profile of components that bind to particular tagged reagents can be produces. This profile will show the same binding pattern, i.e., the same interactions among the tagged reagents and the sample components, regardless as to whether the collection of binding sites is positionally addressable on a solid support or addressable tagged with, for example, electronic labels. Further, the means of detection the binding profile, such as by luminescent detection or immunological detection, similarly does not effect the end result of the pattern of binding due to the interactions among the tagged reagents and the sample components. Alternatively, the loci in the collection that react with a particular sample can be identified, such as by virtue of the bound tag and used to produce sub-collections specific for a particular sample.

As in the embodiments for sorting (discussed below), the addressable collection of capture agents is a collection of such agents, such that each loci is identifiable. A loci can be an addressable position on an array or a detectable label, such as colored bead or nanobarcode or RF tag, linked or associated with a capture agent. For isolation and/or identification of molecules bound to the tagged-agents and other aspects of making and using, the addressable collection all of the methods described throughout the disclosure can be employed as needed in these embodiments.

For profiling, the collections are used either by themselves or with other reagents bound via their tags, such as epitope tags. In the latter embodiment, the reagents bound via the tags are not all the same, so that each loci represents a collection of such reactions, such as scFvs, bound via their tags. As described herein, the tags, such as polypeptide tags, are distributed such that the linked agents are different. The resulting collection provides a highly diverse collection of capture agent-tag-linked reagents for binding to any sample, such as a cell lysate, cells, blood, plasma, serum, cerebrospinal fluid, synovial fluid, urine, sweat and tissue and organ samples from animals and plants. Any method for sample preparation known to those of skill can be employed. Exemplary methods for sample preparation are provided in Example 10.

In some embodiments, a sample that has been subjected to a particular condition or treated with a particular agent is contacted with the collection, generally a collection of capture agents with tagged reagents, such as scFvs, bound thereto, and components of the sample, optionally labelled, are permitted to react with the collection. After reacting and washing away or otherwise removing unbound material, a profile is produced, which is characteristic of the sample and particular collection. The profile can be imaged and, if needed, compared to the profile that results from a control for such condition or in the absence of the agent. For example, the same reaction can be performed with a duplicate or replicate collection, except that the sample may not be treated with the same condition. The resulting profile serves as a control. The difference between the two profiles represents a profile for the particular condition or sample.

In addition, upon identifying particular capture agent/tag linked agent/sample component complexes specific for the test condition, the tagged reagents can be used to produce a sub-collection specific for the test condition. Such sub-collections can be repackaged as a collection, such as an array with a collection of binding agents, that when contacted with a sample provides a specific profile that is specific for a particular disorder or other test condition of interest. Also, since the tags are known and can be used to design primers to amplify, identify and recover the nucleic acids encoding the linked polypeptides, specific binding proteins can be identified and used in the repackaged product and/or new binding agents can be identified.

1. Exemplary Profiling Methods

In practicing the method, a random library of tagged binding agents, such as the scFvs, is layered on a collection of capture agents. Then each test material is labeled, before, during or after contacting, bound test material is detected, and labeled loci identified. The resulting labeled array provides a profile or fingerprint. Alternatively, the labeled loci are used to make sub-arrays characteristic of a particular test material to provide a diagnostic test for the condition or indicative of a condition or disorder.

The collections of capture agents permit massive display of a diversity of tagged reagents at addressable loci. As described for the sorting embodiments, collections of tagged reagents are contacted with the capture agents. At each locus, the capture agents are identical, but a plurality of different tagged reagents are present at each locus, resulting in a diverse collection of binding sites. By contacting each collection of capture agents with one or a plurality of collections of tagged reagents, each locus contains a plurality of different tagged reagents or binding agents, collections of tagged reagents for further binding are produced. Where a plurality of different tagged moieties are contacted with the capture agents, the result is a massive display such that many different binding sites are displayed on a single addressable locus. Hence, in this embodiment, advantage can be taken of the large variety of displayed agents (that contain binding sites), which can then serve for binding components of test samples.

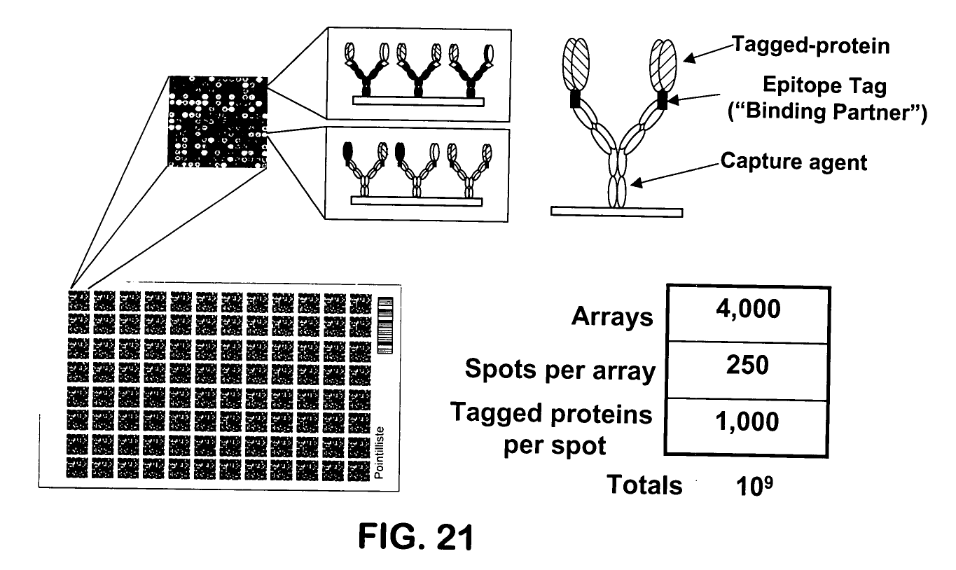

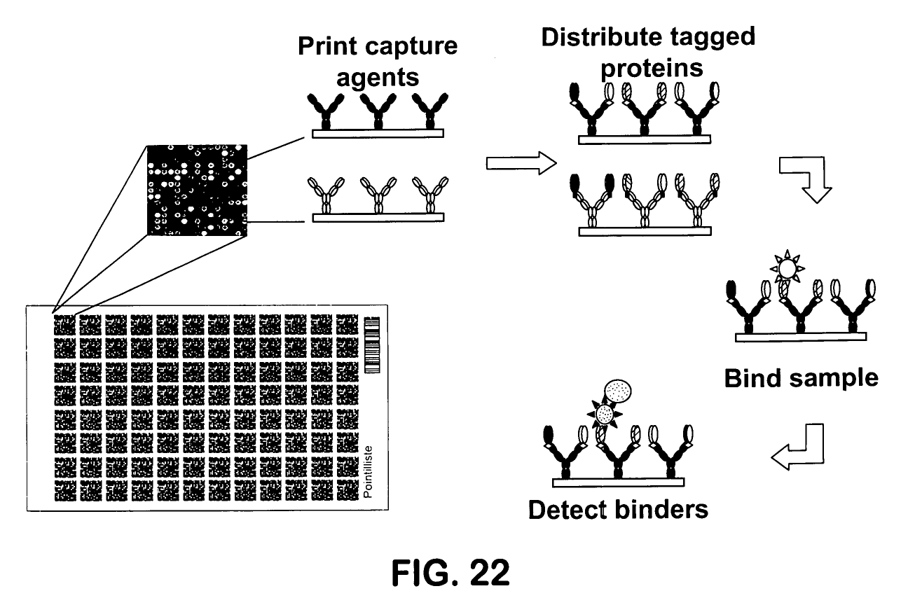

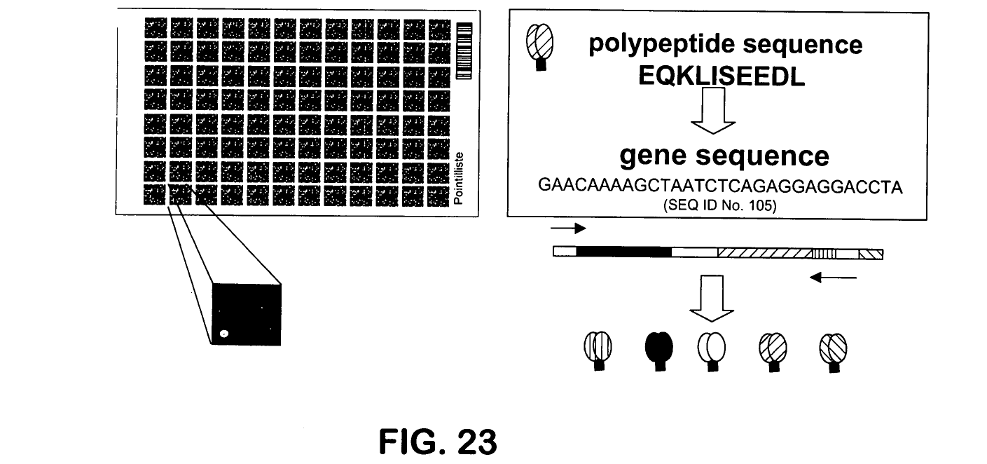

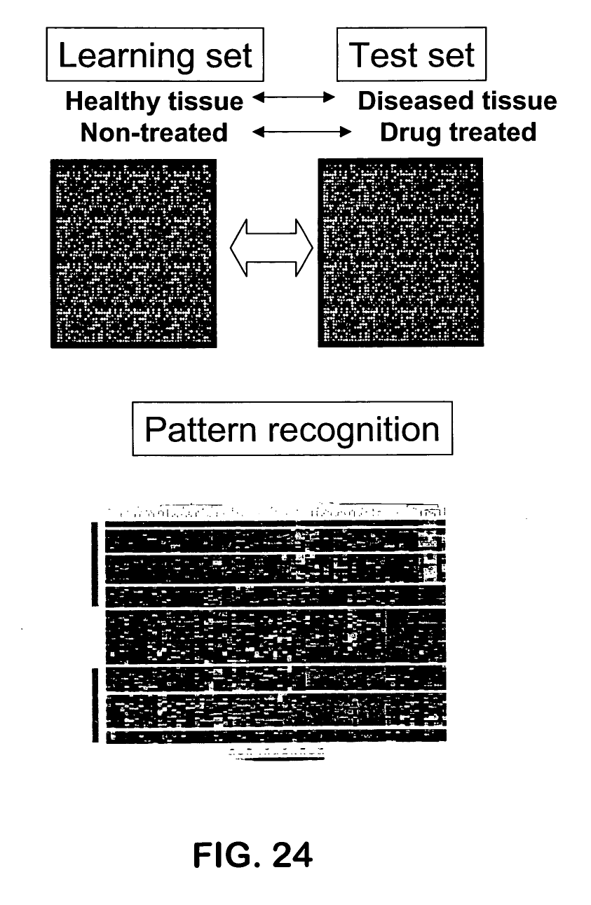

In an exemplary embodiment (see, also FIGS. 21-25), the method includes some or all of the following steps:

Step 1. Capture agents are provided as an addressable collection, such as a positionally addressable array or a collection of barcoded or color-coded capture agents, such as that the capture agents are addressable.

Step 2. A collection of tagged reagents that bind to the capture agents that include a tag, such as library of scFvs, is prepared as described herein. For example, a library is tagged with nucleic acid encoding the tags via subcloning or PCR amplification as described herein.

Step 3. Proteins are produced from, for example, the tag-encoding cDNA library, such that the library proteins are associated with the tags. These proteins constitute the collection of tagged reagents.

Step 4. The tagged library proteins (tagged reagents) are incubated with the addressable collection of capture agents such that the proteins are sorted out via the interaction of the tag with its cognate capture agent. In this way each “locus” or “address” corresponds to one of the tags. Many different library members are tagged with the same tag and therefore each “locus” has multiple different library members (potential sample binding proteins), thereby providing a diverse collection of binding sites for profiling.

Step 5. A labeled protein or labeled complex mixture is incubated with the arrayed capture agent-tagged library complexes. These labeled proteins will sort themselves out onto the library members. Many “loci” will have library members that bind to labeled items in the complex mixture. In the above, exemplification, the tagged reagents are bound to the capture agents followed by addition of a sample. Alternatively, instead of binding the tagged reagents to the capture agents, the tagged reagents can be mixed with the sample and the resulting mixture contacted with the collection of capture agents.

Step 6. The label is developed. For example, if the label is radioactive, the array is put onto X-ray film; if the label is a biotin molecule, the array is reacted with horseradish peroxidase-conjugated avidin and incubated with a chemiluminescent substrate, and observed with a CCD camera or X-ray film; if the label is a fluorescent molecule, the array is analyzed with a laser to excite the fluor and a reader to analyze the emitted light. Any suitable method for identification of a selected label can be employed.

Step 7. A plurality of the “loci” or “addresses” produce a signal such that the profile that is generated for a particular sample that is indicative of the overall sample. A sample profile or fingerprint is generated.

Step 8. If desired, a plurality of samples, such as labeled and unlabeled samples, can be mixed under a variety of conditions, such as at varying concentrations, pH, temperature, salt concentration and other conditions that alter binding, until a discernable profile, such as a pattern, emerges. Such conditions can be empirically determined.

Step 9. A profile that includes “loci” of interest is identified. When the sample is a complex mixture, such as a cell lysate and intact cells, and optimized conditions are ascertained as described in Step 8, then those “loci” that are different between or among the test conditions provide the profile.

Step 10. Using the addresses of the loci of interest, the identity of the capture agents and therefore the tags that bind to them are known. This identifies the oligonucleotide primer(s) that will be used to recover genes encoding the tagged reagents located at the loci of interest by PCR. This oligonucleotide primer can correspond directly to the amplification domain of the tag. Using this specific oligonucleotide, the polymerase chain reaction amplifies the cDNA that encodes the tagged proteins at the loci of interest.

Step 11. The amplified genes can be re-tagged with the whole panel (or subset thereof) of tags such that it is further subdivided and analyzed again. Alternately, the amplified genes can be analyzed individually by high throughput screening until the individual genes that encode the proteins responsible for the signal are identified.

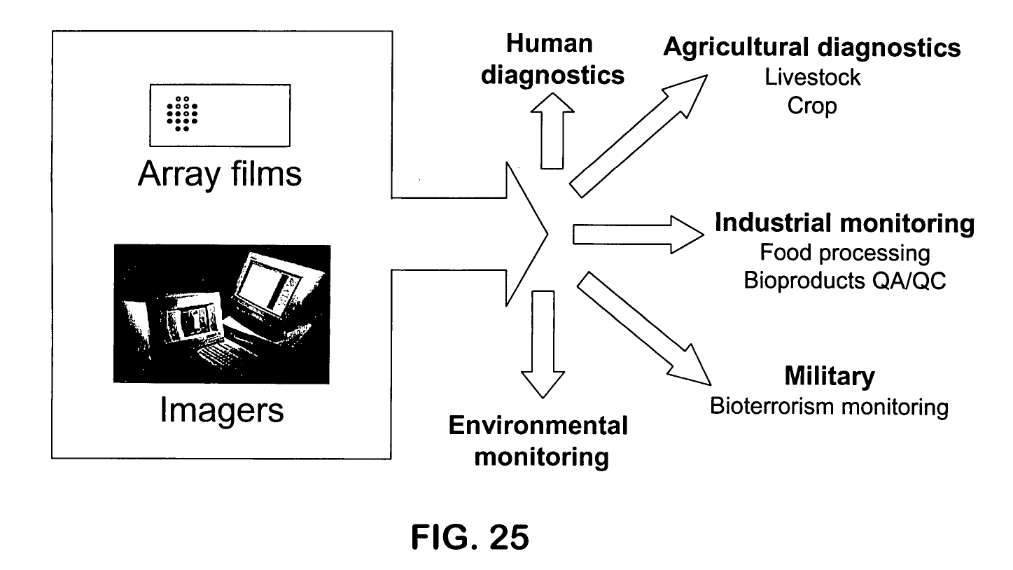

Step 12. Alternatively or in addition, those library members (in protein form) that were identified as of interest can be re-arrayed and packaged individually or in groups as a diagnostic test, used as reagents for research and development, tested as potential therapeutic agents or selected for other purposes (see, e.g., FIG. 25).

The description above references library members that are simple binding agents, such as single chain antibody libraries. The method and system, however, can be used for any collection, including any cDNA library that can be assayed for any function. The library members can be cDNA from a particular organism or semi-synthetic in nature. For example, screening for a new class of enzymes that catalyze the production of light from the luminol reagent, the substrate for horseradish peroxidase, can be screened. Proteases with a new substrate specificity using a substrate that becomes fluorescent upon cleavage can be screened using library members of cDNA from a particular organism or a collection of mutants, produced from processes such as DNA shuffling, of known proteases.

Unpurified or partially purified or fractionated samples can be contacted with the collection. For example, whole cells can be contacted with the collections. The cells can be treated with a condition, such as a small effector molecule, of interest, and the effects of the condition assessed by comparing the profile of treated and untreated cells.

Profiles can be identified using digital imaging systems and pattern recognition software, which are well known and readily available (see, e.g., U.S. Pat. No. 6,340,568 B2, U.S. Pat. No. 6,327,035 B1; PARTEK PRO2000® commercially available from Partek, Inc. St. Charles, Mo.; IMAGE-PRO® and other such software and products available from Media Cybernetics).

The resulting profiles can be provided as databases and used for assessing unknowns and for diagnostic purposes. Databases of profiles are provided. Unknown samples being tested for a particular condition can be compared to profiles of knowns to thereby identify components of the samples or effect a diagnosis or extract other information.

2. Prognosis and Diagnosis

The combinations, collections, kits and methods provided herein can be used to aid in diagnosing (or prognosing) or to provide a diagnostic (or prognostic) for a medical condition or for determining the risk for a disease. The collections of binding sites provided herein can be used as tools for the diagnosis or prognosis of a diseased state, which can be vital in combating illnesses, such as cancers, viral infections and bacterial infections. The diverse collections of binding sites and the methods for profiling provided herein can be used in diagnostics, particularly diagnosis of diseases and conditions, such as cancers, including, but not limited to cervical, colon, pancreatic, prostate, colon, ovary, cervix and breast cancers, viral infections, such as the common cold, influenza, infectious mononucleosis, Herpes simplex, Shingles, Rabies, Hemorrhagic fevers, Measles, Mumps and Pneumonia, and bacterial infections, such as Salmonella, Typhoid Fever, E. coli infections, Klebsiella infections, Yaws, Brucellosis, Campylobacter infections, Plague, Lyme disease, Staphylococcal infections, Streptococcal infections, Diptheria, Clostridium infections, such as Tetus, Botulism and Gas Gangrene, Tuberculosis, Leprosy, bacterial Meningitisis and Sepsis. Such collections of binding sites can be used in assays, such as immunoassays, to detect, prognose, diagnose, or monitor various conditions, diseases, and disorders affecting the binding profile resulting from interactions among the tagged reagents and the components in a sample. In particular, such a binding assay is carried out by a method including contacting a sample derived from a patient with disease or condition with a collection of binding sites under conditions such that specific binding can occur, and detecting or measuring the amount of any specific binding by components in the sample to the collection of binding sites, thereby producing a binding profile for a particular disease or condition.

Further, replicate arrays of collections of binding sites can be prepared for parallel or sequential experiments wherein the binding profile of the same or different samples under the same or different conditions can be compared. For example, the collections of binding sites provided herein can be used to identify antibodies or antigens with a particular characteristic, such as antigenic specificity or relation to a disease state, that are not present in a control sample, without requiring knowledge of the particular antibody or antigen to which the identified antibody or antigen binds. Identification of sub-sets of tagged reagents which bind to components of a sample in a particular diseased state allows for the identification of diagnostic antibodies or antigens present due to the diseased state and in the production of collections of binding sites that are disease specific and can be used for diagnosis of a particular disease or illness. Hence, the collections of binding sites provided herein can serve as alternatives to phage display and other similar panning technologies.

Kits for diagnostic use are also provided that contain diverse collections of binding sites for the identification of binding profiles or collections of binding sites that produce a known binding profile based on a particular disease or condition.

3. Drug Discovery

The combinations, collections, kits and methods provided herein can be used to identify or screen for potential or candidate therapeutic compounds, such as antibodies, antigens, drug compounds and proteins. The diverse collections of binding sites provided herein can be used to identify therapeutic compounds from among the binding sites, from within the sample, or from a perturbation, such as a candidate compound or condition. The collections of binding sites provided herein can also be used to identify targets for therapeutic compounds. For example, a collection of binding sites can be prepared from a collection of tagged scFv molecules and contacted with a sample from a host afflicted with a particular bacterial infection. The interaction among the tagged scFvs and the components of the sample can be detected to identify particular scFv molecules that interact with components of the sample that do not interact with a control sample. The identified scFv molecules are indicative of a particular disease or condition and can be used to initiate or enhance an immune response within the host. Similarly, a component from the sample, such as an antibody or a protein, that is diagnostic of the disease or condition, can be identified and isolated for use as a therapeutic compound. Perturbations and conditions can be identified as potential therapeutic compounds by causing an alteration in a binding profile, indicating an effect of the perturbation on the interactions among the collection of binding sites and the components of the sample.

In another example, a sample from a donor with a particular disease or condition is exposed to a collection of binding sites and the binding profile is produced. The host can then be exposed to a potential therapeutic compound. A second sample following exposure of the host to the compound can be exposed to a replicate collection of binding sites and the resulting binding profile compared to that of the pre-compound sample. Variations between the two profiles can be indicative of the effectiveness of the compound on the disease or condition.

The collections of binding sites provided herein can also be used to identify potential targets for drug discovery. For example, a collection of binding sites can be used to identify and isolate a component of a sample, such as a protein, that is only present when a disease state or condition is present. The identified component from the sample can isolated from the sample using the tagged reagent from the collection of binding sites or any other method known to those of skill in the art and can be used as a target for future therapeutic compounds.

D. Identification and Recovery of Tagged Molecules Using Nested Sorting

The methods described above for the use of collections of binding sites in the generation of binding profiles for samples can optionally include the step of recovery of the tagged molecule or molecules that are determined to be of interest based on the binding profile. For example, using the methods provided herein, two samples, a control sample and an experimental sample, are exposed to collections of binding sites, resulting in the generation of binding profiles for each sample. Comparison of the two profiles indicates differences in the interaction of the samples with the collection of binding sites. The identity of the capture agent, and therefore, the tag, such as a polypeptide tag, are determined based on the location of the variation between the profiles. With the tag identified, a sub-set epitope tagged molecules can be identified and recovered for further analysis.

Previous applications have described the sorting of tagged molecules based on interactions between a tag and a capture agent (see, e.g., published International PCT application No. WO 02/06834; published U.S. application Ser. No. US20020137053; U.S. provisional application Serial No. 60/422,923; and U.S. provisional application Serial No. 60/423,018). Here, methods for the sorting of tagged molecules are used to identify and recover a sub-set epitope tagged molecules determined to be of interest based on the binding profile generated from exposure of a sample, such as cell lysate, cells, blood, plasma, serum, cerebrospinal fluid, synovial fluid, urine, sweat and tissue and organ samples from animals and plants, to a collections of binding sites. These methods rely upon the use of collections of capture agents, such as a plurality of substantially identical, generally replicate, collections of agents, such as antibodies, that specifically bind to preselected sequences of amino acids (generally at least about 5 to 10, typically at least 7 or 8 amino acids, such as epitopes), that are linked to proteins in a target library or encoded by a target nucleic acid library. Combinations of the capture agents and polypeptide tags that contain the sequence of amino acids to which the capture agent or a binding portion thereof specifically binds are provided. The polypeptide tags can, in addition, contain sequences of amino acids or nucleotides for use in the amplification, identification and recovery of a particular sub-set of the collection of tagged molecules. The tags can be linked to members of a nucleic acid library or other library of molecules to be sorted and for identification and recovery purposes.

1. Overview

The addressable capture agent collections, such as an positionally addressable array, contain a collection of different capture agents, such as antibodies, that bind to pre-selected and/or pre-designed polypeptide tags, such as epitope tags, with high affinity and specificity. A typical collection contains at least about 30, 100, 500, and generally at least 1000 capture agents, such as antibodies, that are addressable, such as by occupying a unique locus on an array or by virtue of being bound to bar-coded support, color-coded, or RF-tag labeled support or other such addressable formats. Each locus or address contains a single type of capture agent, such as an antibody, that binds to a single specific tag. Tagged proteins are contacted with the collection of capture agents, such as antibodies in an array, under conditions suitable for complexation with the capture agent, such as an antibody, via the tag, such as an epitope tag. As a result, proteins are sorted according to the tag each possesses.

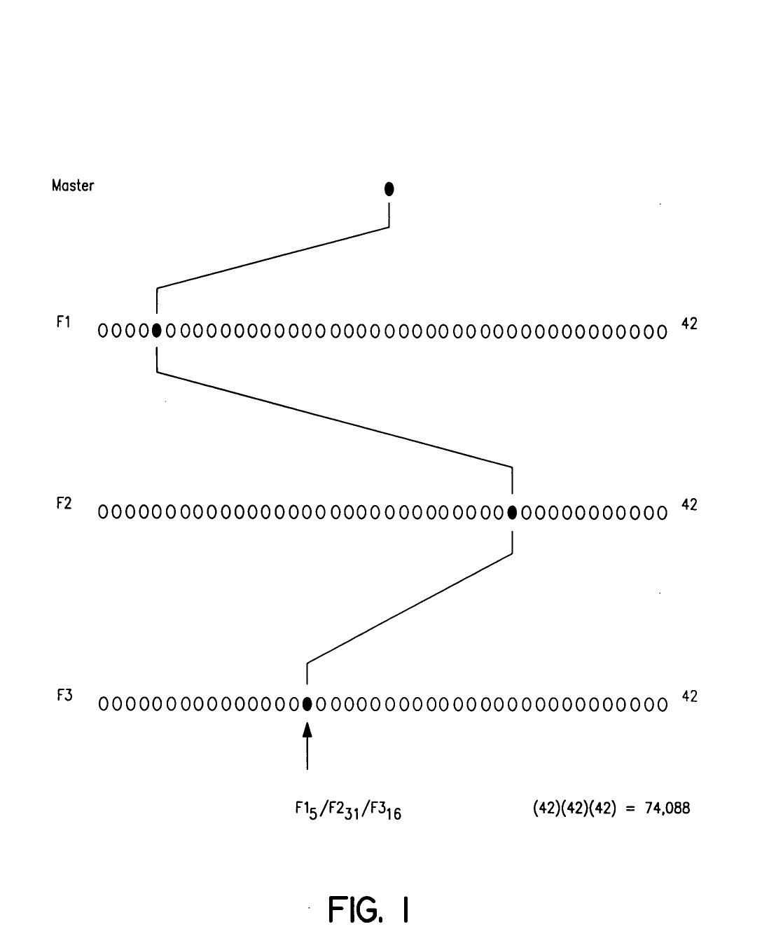

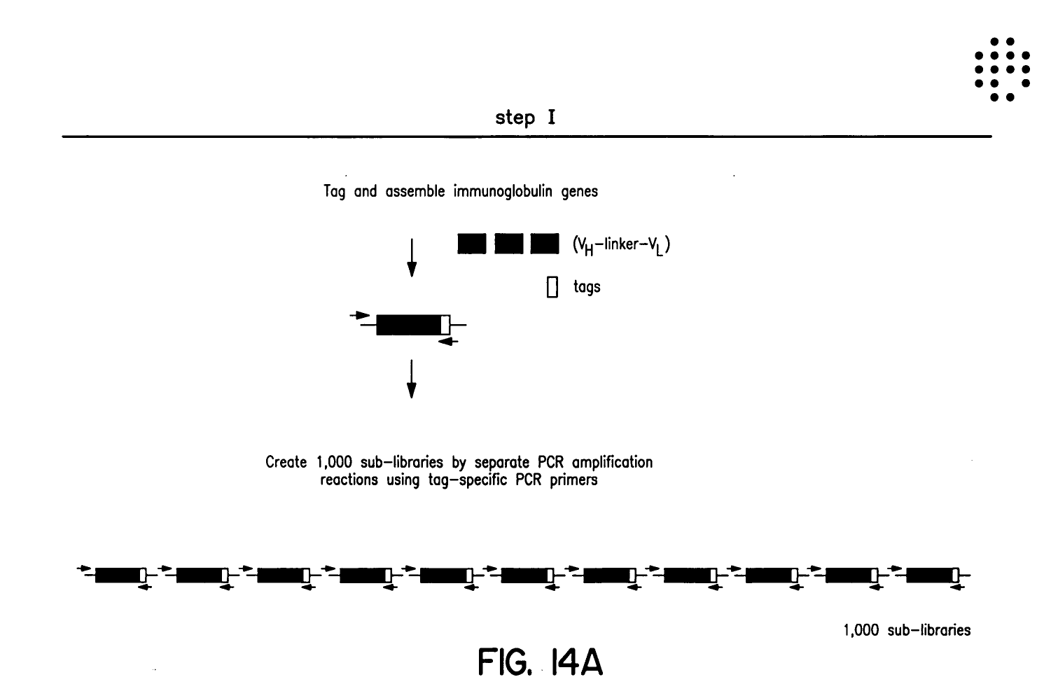

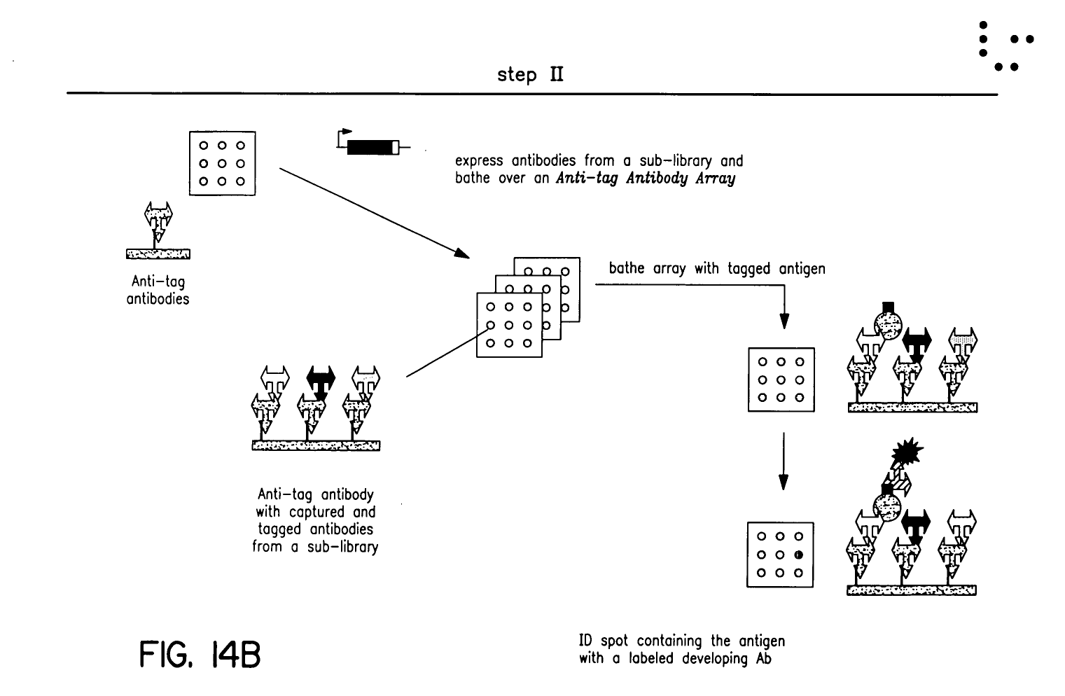

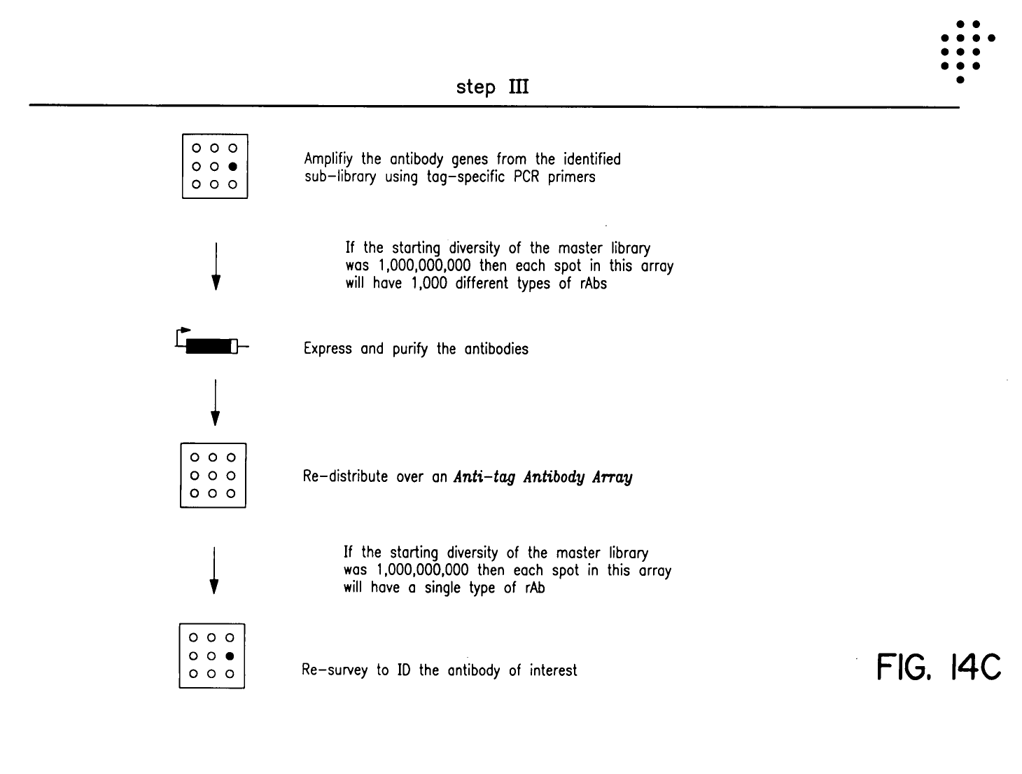

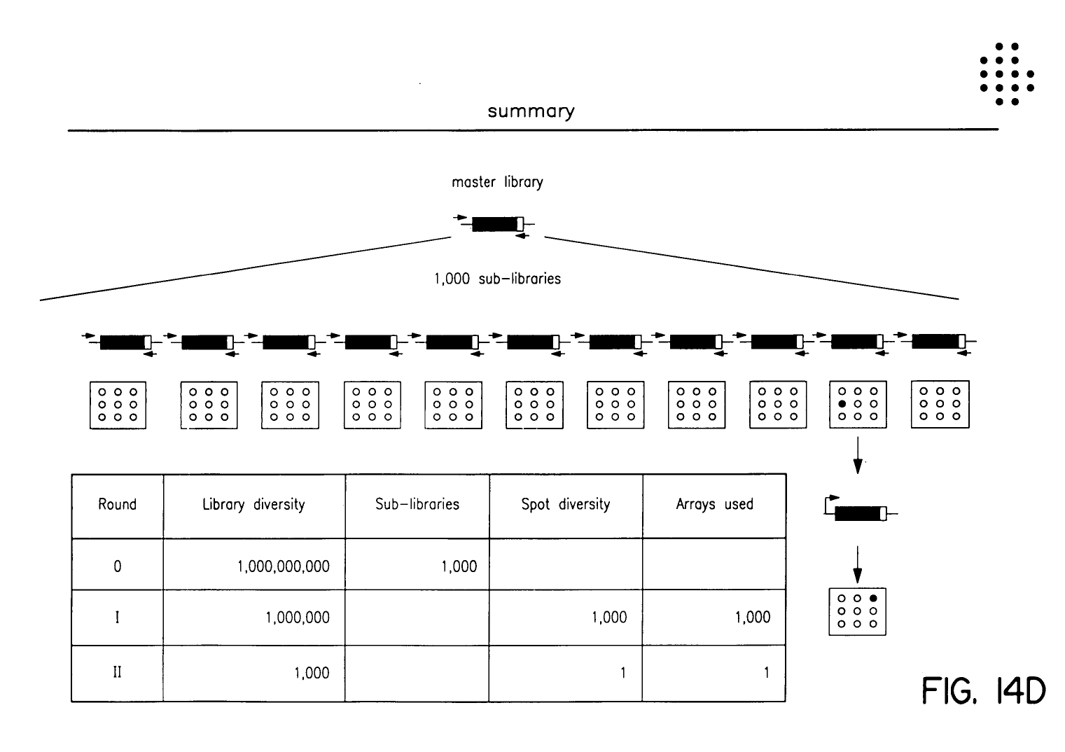

These addressable anti-tag antibody collections have a variety of applications in addition to sample profiling as discussed above, including, but not limited to, rapid identification of antibodies; for therapeutics, diagnostics, reagents, and proteomics affinity matrices; in enzyme engineering applications such as, but not limited to, gene shuffling methodologies; for identification of improved catalysts, for antibody affinity maturation; for identification of small molecule capture proteins, sequence-specific DNA binding proteins, for single chain T-cell receptor binding proteins, and for high affinity molecules that recognize MHC; and for protein interaction mapping. Exemplary protocols are depicted in FIGS. 1-4, 12, 14A-D and 15-18.

2. Recovery of Identified Tagged Molecules and/or Biological Particles

a. Design and Preparation of Oligonucleotides/Primers

Sorting large diversity libraries onto arrays and amplifying specific pools containing clones with the desired properties is dependent on the ability to uniquely tag a library with specific polypeptide tags. Oligonucleotide sets are chemically synthesized, randomly combined by overlapping sequences, and ligated together to produce a template for enzymatic synthesis of the collection of primers or linkers.

The oligonucleotides are either single-stranded or double-stranded depending upon the manner in which they are to be incorporated into the master library. For example, they can be incorporated by ligation of the double stranded version, such as through a convenient restriction site, followed by amplification with a common region, or they can be incorporated by PCR amplification, in which case the oligonucleotides are single-stranded.

(1) Primers

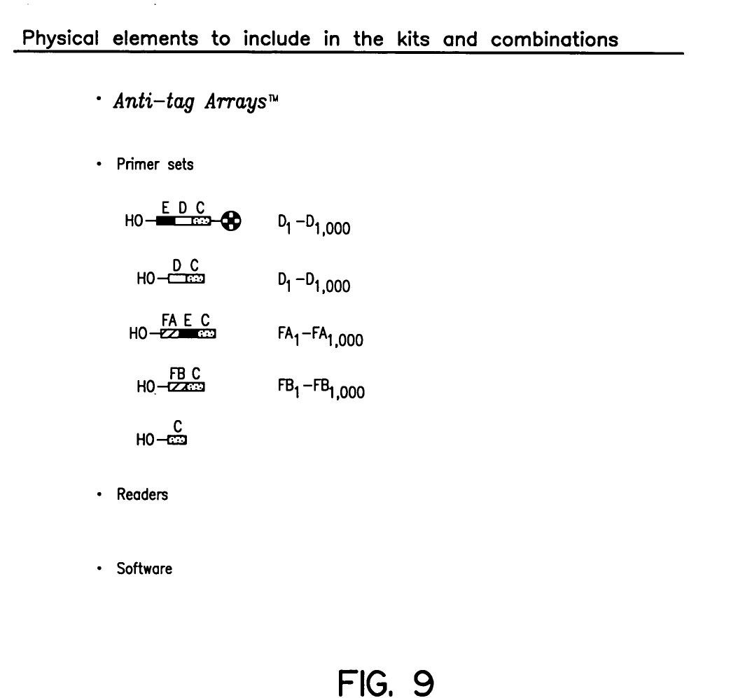

Provided herein are sets of nucleic acid molecules that are primers or double-stranded oligonucleotides, which are double-stranded versions of the primers, and combinations of sets of primers and/or double-stranded oligonucleotides. The selection of single-stranded or double-stranded primers for the use in the various steps of the methods provided herein depends upon the embodiment employed. The primers, which are employed in some of the embodiments of the methods for tagging molecules, are central to the practice of such methods. The primers contain oligonucleotides, which include the formulae as depicted in FIG. 9. The primers and double-stranded oligonucleotides can include restriction site(s) and for targeted amplifications, as exemplified below for example for antibody libraries, of sufficient portions of genes of interest. These primers can be forward or reverse primers, where the forward primer is that used for the first round in a PCR amplification. The primers, described below and depicted in the figure, are provided as sets. Also provided are combinations of one or more of each set. The primers are central to the methods provided herein.

(2) Preparation of the Oligonucleotides/Primers

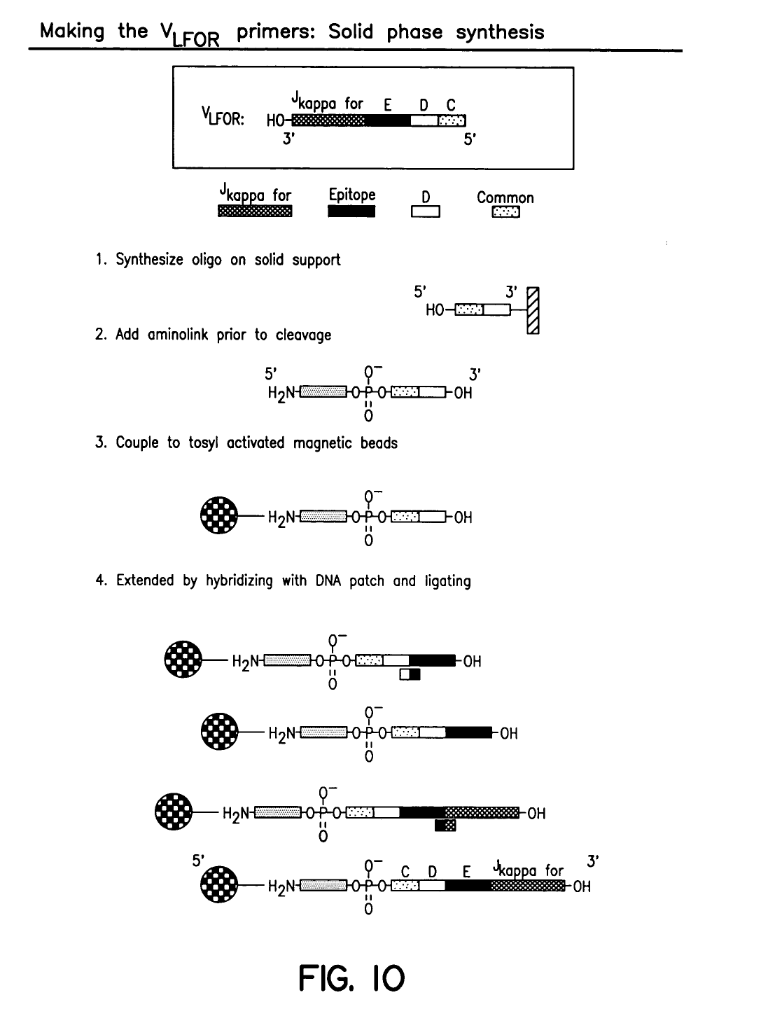

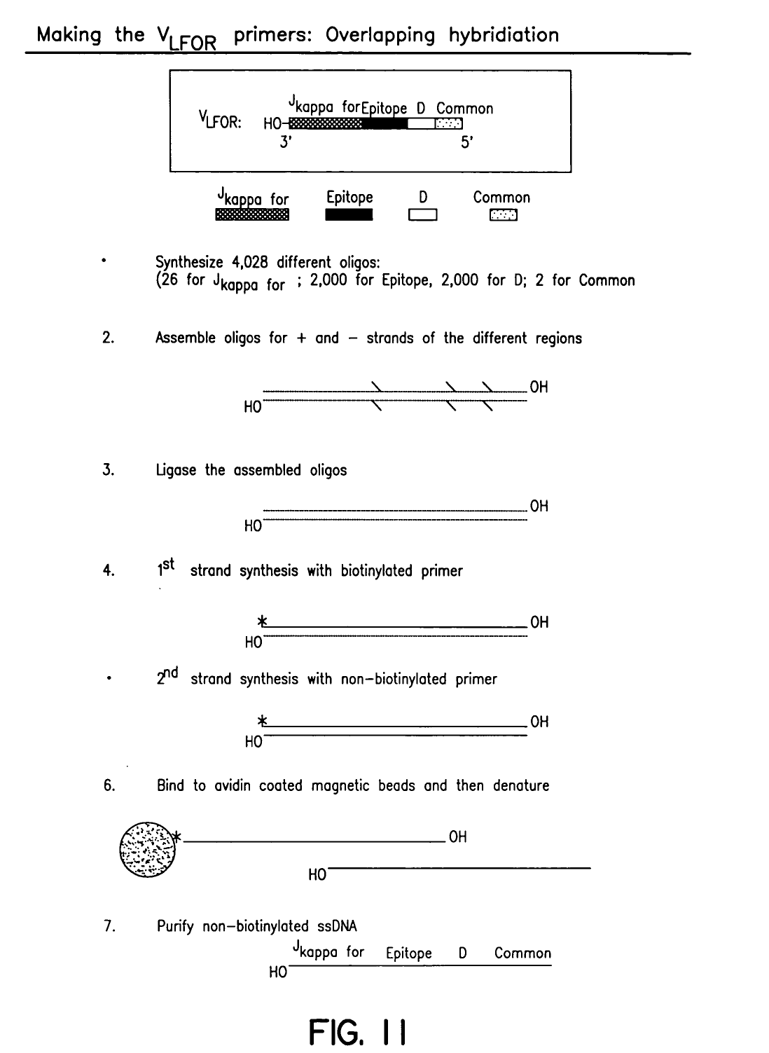

Any suitable method for constructing double-stranded or single-stranded oligonucleotides can be employed. Methods that can be adapted for preparing large numbers of such oligomers are particularly of interest. Two methods are depicted in FIGS. 10 and 11 and are discussed below.

FIG. 9 illustrates the physical elements for construction of a tagged library and use of the addressable anti-tag antibody collections for identification of genes (proteins) of interest. Four oligonucleotide/primer sets are provided in addition to the addressable collections, which, for exemplification purposes, are provided as arrays, an imaging system or reader to analyze the arrays and, optionally software to manage the information collected by the reader. In the embodiment depicted, the primer sets include EₘDₙC, where C is a portion in common amongst all of the oligonucleotides and can serve as a region for amplification of all tagged nucleic acids with differing E and/or D sequences (e.g., D₁ through Dₙ; E₁ through Eₘ); DC, with differing D sequences (D₁ through Dₙ), and an optional C, for common region, FAEC, with differing FA sequences (e.g., FA₁ through FAₙ); and FBC, with differing FB sequences (e.g., FB₁ through FBₙ). Each FA includes a portion of each epitope and can serve as a primer to amplify nucleic acids that encode a corresponding Eₘ, but the resulting amplified nucleic acids does not include the Eₘ epitope. FBₙ is similar to FAₙ, except that it can include Eₙ, if it is desired to retain the epitope.

FIG. 10 and FIG. 11 outline two different methods for constructing the ED, and EDC, FA and FB oligonucleotides/primers for antibody screening as an example. For example, synthesis of the V_{LFOR} primer, which combines n, such as a 1,000, different E sequences with m, such as 1,000 different D sequences and approximately 13 different Jₖₐₚₚₐ For sequences. This makes a total of (1,000)(1,000)(13)=13,000,000 different oligonucleotides. By randomly combining the different sequence regions in progressive synthesis steps, this large diverse collection of primers can be prepared.

The first method (FIG. 10) uses a solid-phase synthesis strategy. The second method (FIG. 11) uses the ability of DNA molecules to self-assemble based on overlapping complementary sequences. Solid-phase synthesis has the advantage that the immobilized product molecules can be easily purified from substrate molecules between reactions, allowing for greater control of the reaction conditions. The self assembly method has the advantage of requiring much less work.

FIG. 10 Oligonucleotides are chemically synthesized 3′ to 5′ from a solid support. In contrast, DNA is enzymatically synthesized 5′ to 3′. To create the V_{LFOR} primer, the C and D sequences are chemically synthesized using standard methods from a solid support. In order to couple the oligonucleotide to a solid-phase for further synthesis, a strong nucleophile is incorporated by addition of an aminolink prior to cleavage of the oligonucleotide from its substrate. The aminolink introduces a primary amine to the 5′ end of the oligonucleotide. The amine group on the aminolink then can be coupled to a solid support, such as paramagnetic beads, by reaction with amine reactive groups on the beads, such as tosyl, N-hydroxysuccinimide or hydrazine groups. The resulting oligonucleotides are covalently coupled to the beads with the C and D sequences in the proper 5′ to 3′ orientation.

A mixture of E sequences are added to the oligonucleotide by use of a DNA “patch” and the resulting nick is sealed with DNA ligase. Unincorporated substrate DNA is purified from the extended product and a mixture of J_{kappa for} sequences are added to the primer. Although the completed V_{LFOR} primer can be released from the bead, the beads do not interfere with the ability of oligonucleotides to prime cDNA synthesis.

The method illustrated in FIG. 11 relies on the oligonucleotides to self-assemble based on overlapping hybridization. A double stranded DNA molecule is first created from oligonucleotides encoding the + and − strands of the molecule. These oligonucleotides are combined and allowed to hybridize to produce a nicked double-stranded DNA molecule and the nicks on the molecule are sealed by the addition of DNA ligase. The sealed molecules are used as templates for enzymatic synthesis of a new DNA molecule. DNA synthesis is primed using an oligonucleotide with a group on its 5′ end to allow coupling to a solid support, such as biotin or the aminolink chemistry described above.

Incorporation of the reactive group during enzymatic synthesis enables purification of a single stranded molecule after the synthesis is complete. Although the completed V_{LFOR} primer can be released from the bead, the beads do not interfere with the ability of oligonucleotides to prime cDNA synthesis.

b. Use of Multiple Tags in a Single Fusion Protein

The system provided herein uses tags, such as polypeptide tags, to subdivide protein libraries, such as libraries of scFvs. For example, with 1000 tags and a library of 10⁹ scFvs, there are 10⁶ scFvs for each tag. To identify a single library member, such as an scFv of interest, either a large number of individual scFvs (10⁶) are screened or more than one subdivision is employed. Using a larger number of tags a library can be reduced to small number of proteins in fewer steps.

Using a combinatorial approach, a small set of capture agent-tag pairs can be used effectively as a much larger set. By incorporating multiple tags into a protein, such as a single scFv fusion protein, better use of fewer tags can be made. For comparison, if there are 300 capture-agent tag pairs, and a library of 10⁹ members, with a single tag appended to each member, the 300 tags divide the 10⁹ members such that each type of tag is attached to 3.3×10⁶ members. With three tags incorporated into each member in a combinatorial fashion such that ⅓ of the tags are used at each of three sites, there is a total of 100×100×100 (or 10⁶) combinations. Using these 10⁶ tag combinations the 10⁹ members are divided into 1000 members per tag. Therefore in a single step with a limited number of tags, the library is effectively subdivided.

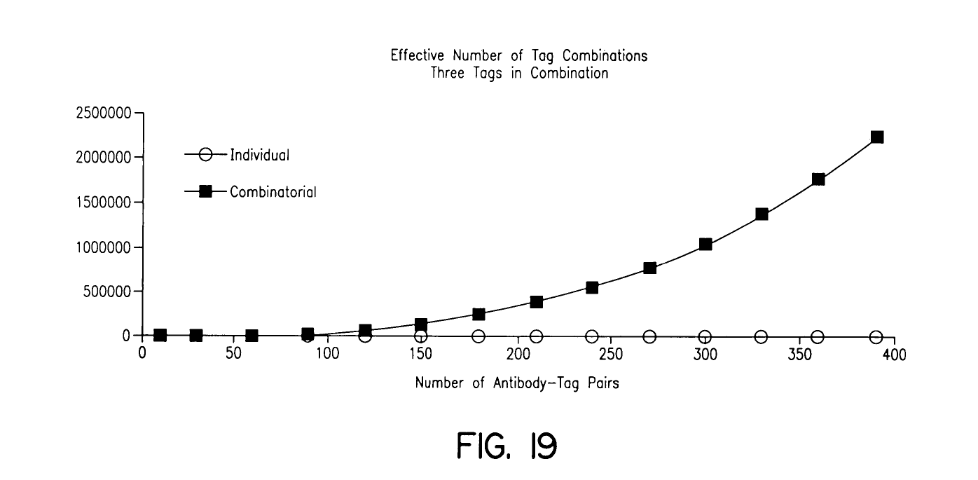

In its simplest embodiment, consider an example of x tags at site X, y tags at site Y, and z tags at site Z. If these tags are used individually, then there are x+y+z combinations. If these tags are used in combination then there are (x)(y)(z) combinations. Assuming that the number of tags at each site (x, y and z) is one third the total (n), then for the case of individual use, C=(n/3)×3=n or there are as many total combinations (C) as there are tags; whereas for combinatorial use, there are C=(n/3)³. As the number of individual tags at each site increases, the number of combinatorial tags increases at a much higher rate (See FIG. 19). With a greater number of effective tags, the number of members of the library per tag decreases. Fewer members per tag in the initial library results in either fewer sequential rounds of screening or lower numbers of clones that to be assessed with high throughput screening.

Whether using a single tag or multiple tags in combination, the procedure is substantially the same. The protein from the expressed library is subdivided by virtue of the tag binding to a capture agent, such as an antibody, against that tag. In the example presented above (using three tags in combination), each library member binds to three different anti-tag capture agents. Each combinatorial tag has its own set of addresses on an array instead of a single address. For example, if there are a total of 300 tags with 1-100 in site X, 101-200 in site Y and 201-300 in site Z a exemplary combinatorial tag has the address X27-Y132-Z289. Other combinatorial tags also use the X27 anti-tag capture agents or the Y132 or Z289 capture agents, but no other combination uses all three. If an antigen binds to a library member tethered to the three capture agents to which each tag binds, the combinatorial tag is now known and the library member can be recovered from the original library.

Recovery of a specific library pool with a combinatorial tag is done in substantially the way a library pool with a single tag is recovered. As described herein, one way to recover subpopulations from the library is to use the polymerase chain reaction. For exemplification, assuming that all three tags are at the C-terminus of an expressed protein such that the X tag is the most proximal to the library member, such as an scFv, followed by the Y tag and then the Z tag. The order of DNA segments on the coding strand of cDNA is:

5′Common>scFv>X>Y>Z 3′

A particular sub-population can be recovered by sequential rounds of PCR amplification starting with a common primer and a primer corresponding to the Z289 tag. The product from this reaction is used in the next reaction using the common primer and the Y132 tag primer. The product from this reaction is used in a subsequent reaction with the common primer and the X27 primer. After three sequential rounds of amplification, the products all correspond to library members, such as scFvs, that were originally tagged with the X27-Y132-Z289 combination.

Those skilled in the art understand that, as long as the library has multiple nested common sequences, multiple different common primers are used in the different rounds. Those skilled in the art also understand that the multiple tags can be at opposite ends of the encoding DNA and therefore the expressed protein. It is also understood that the expressed tags can be linear, constrained by disulfide bonds, constrained by a scaffold structure, expressed in loops of a fusion protein, contiguous or separated by flexible or inflexible linker sequences.

One embodiment uses, for example, a single scaffold fusion protein containing multiple sites with inserted tags. This spatially separates the epitopes and allows them all to be recognized without interference with one another. The following criteria are considered in selecting a protein scaffold: 1) known crystal structure to more easily identify surface exposed amino acids with high propensity for antigenicity, 2) free N and C-termini for fusion to the cDNA library of interest, 3) high levels of production and solubility in various protein expression systems (especially the E. coli periplasm), 4) capacity for in vitro transcription/translation, 5) absence of disulfide bonds, 6) wild-type protein is monomeric, 7) has capacity to increase solubility or function of scFvs. Using the crystal structure, positions are chosen for insertion of tag libraries. These sites should be spatially separated epitopes that are relatively linear in nature (e.g., one side of an alpha helix, a turn between beta strands or a loop between helices).

3. Sorting Methods

Methods of using the capture agent, such as antibody, collections for sorting molecules labeled with the tags, such as polypeptide tags, are provided. The methods include the steps of (1) creating a master tagged library by adding nucleic acids encoding the tags; (2) dividing a portion of the master library into N reactions; (3) amplifying each reaction with the nucleic acid encoding the divider sequences and translating to produce N translated reactions mixtures; (4) exposing each of the reactions mixtures, simultaneously or separately, with one collection of the capture agents, such as antibodies; and (5) identifying the proteins of interest by a suitable screen, such as exposure of the displayed tagged molecules to a sample and generation of a binding profile, thereby identifying the particular tag on the protein by virtue of the capture agent to which the tag on the protein of interest binds. The steps of created the tagged master library (1) and dividing the tagged master library into N reactions (2) can be performed in any order.

In some cases, it may be necessary or desirable to have the DNA sequences used for sub-division of a library or recovery of a sub-library be distinct from the protein encoding tags. Furthermore, particularly for certain applications, such as profiling, the tag is not required to be genetically fused to the library of interest such that a single protein is synthesized. It is possible to prepare tags, such as polypeptide tags, that are encoded as a separate protein that remains physically or otherwise associated with the library member.

The first sorting step substantially reduces diversity. If desired further sorts are performed or the resulting library is screened by any method known to those of skill in the art. The optional second sort, which is started from the nucleic acid reaction mixture that contains the nucleic acid from which the protein of interest was translated, is performed. In this step, a new set of the tags is added to the nucleic acid by amplification or ligation followed by amplification. Prior to, or simultaneously with this, the nucleic acid encoding the prior tag is removed either by cleavage, such as with a restriction enzyme or by amplification with a primer that destroys part or all of the epitope-encoding nucleic acid. The new tags are added, resulting nucleic acids are translated and are reacted with a single addressable collection of capture agents, such as antibodies. The proteins sort according to their polypeptide tag, and then exposed to a sample to generate a binding profile and identify the potential tagged molecules of interest.

At this point, the diversity of the molecules at the addressable locus of the capture agent, such as antibody, collection should be 1 (or on the order of 1 to 100, typically 1 to 10). The nucleic acids that contain the protein of interest are then amplified with a tag that amplifies nucleic acid molecules that contain nucleic acids encoding the identified tag, to thereby produce nucleic acid encoding a protein of interest. The primer for amplification includes all or only a sufficient portion of the tag to serve as a primer to thereby removing the epitope from the encoded protein. Hence the methods, provided herein permit sorting (i.e., reduction of diversity) of diverse collections and recovery of tagged molecules from the diverse collections. A sort that involves one step will substantially reduce diversity. The use of an optional sorting steps generally reduces diversity to less than 10, generally one.

### Dividing the Master Library

As noted above, the first step in the sorting processes herein includes dividing the master library into N sub-libraries. As described above, the “D” sequence and tags can be introduced into the master library, which is then subdivided using the different D's for amplification into “N” sublibraries.

As noted above, the inclusion of “D” is optional; division can be effected by physically dividing the master library into sublibraries, and then introducing the “E” tag-encoding or “EC” tag-encoding sequences into the sublibraries. This is generally done when the initial library is very large so that the resulting sublibraries are large to ensure a uniform distribution of tags.

4. Creating the Master Library for Sorting

In this step, tags that encode each of the epitopes linked to each of the divider sequences are incorporated into the master library, which is typically a cDNA library. Any way known to those of skill in the art to add and incorporate a double stranded DNA fragment into nucleic acid can be used. In particular, a variety of ways are contemplated herein. These include (1) using PCR amplification to incorporate them (exemplified herein); (2) ligating them directly or via linkers (see below), the ligated product, if needed, can be amplified; and (3) other methods described herein (see above) and that can be readily devised by those of skill in the art in light of the description herein.

In the initial tagging step, when adding the E, ED or EDC to a set of oligonucleotides on the constituent members of the nucleic acid library, the goal is to produce an even distribution of all Eₘ and all Dₙ and to have them on only one of each type of molecule. The tags can be randomly distributed among the different molecules. As long as the number of molecules is large compared to the number of tags (so that on the average only about one of each type of molecule in the collection gets each tag), the tags are evenly distributed. Hence it is desirable for most embodiments to have the total number of molecules in the collection in substantial excess compared to the number of tags. Such excess is at least 100-fold, generally 1000-fold. The exact ratios, if necessary, can be determined empirically. In practice there should be no more molecules in the reaction than the diversity. On the average each different molecule should have a different tag and only one of each different molecule should be tagged.

To practice the methods, a library of epitope-labeled molecules is prepared by randomly introducing the tags into an unlabeled library so that each tag is randomly distributed amongst the molecules. Experiments have demonstrated that the tags can be introduced randomly and equally into a cDNA library.

The master library is divided into pools, identified as D₁-Dₙ, reacted with n number of addressable collections of antibodies, each collection containing antibodies with m different epitope specificities. Each collection, such as an array, is associated with one of the pools, such as by an optical code, including a bar code, a notation or a symbol or a colored code, a nano-bar code, an electronic tag or other identifier, such as color or a identifiable chemical tag, on the collection or other such identifier. The reaction is performed under conditions whereby the epitopes bind to the antibodies specific therefor, and the resulting complexes of antibodies and epitope-tag-labeled molecules are screened using an assay that specifically identifies molecules that have a desired property. The particular collection(s) of antibodies and antibodies with a particular tag that includes molecules with the desired property are identified, thereby also identifying the particular Dₙ pool and tag on the molecule, thereby reducing the diversity of the collection by n×m.

5. The First Sorting step

For sorting in embodiments in which the proteins are encoded by a nucleic acid library, the proteins are produced from the nucleic acids that contain the pre-selected tags. At least one up to a series of sorting steps are performed. In the first step, a first tag is introduced into the nucleic acid by direct linkage or by primer incorporation of oligonucleotides that encode the epitope Eₘ and divider regions Dₙ to create a master library. Each nucleic acid molecule includes a region at one end that encodes one of the m epitopes and one of the n dividers.

In the next step, each of n samples is amplified with a primer that includes Dₙ to produce n sets of amplified nucleic acid samples, where each sample contains amplified sequences that contain primarily a single Dₙ and all of the E's (E₁-Eₘ). An aliquot or portion of all of each of the n samples is translated to produce n translated samples. Proteins from each of the “n” translated reactions are contacted with one of the capture agent, such as antibody, collections, where each of the capture agents in the collection specifically reacts with an Eₘ; and each of the capture agents, such as antibodies, can be identified and produces capture-agent-protein complexes via specific binding of the capture agents to the polypeptide tags.

The resulting complexes are screened, generally using a chromogenic, luminescent or fluorogenic reporter to identify those that have bound to a protein of interest, thereby identifying the Eₘ and Dₙ that is linked to a protein of interest.

6. The Second Sorting Step

If the diversity of the proteins to be sorted is such that multiple possible proteins are identified after the screening, additional sorting steps can be employed. Alternatively, routine or other screening methods can be used to identify proteins of interest from the identified proteins. If the diversity at this stage is relatively low (1 to about 5000 or so, for example), the sample that contains the identified Dₙ can be screened using routine or standard screening procedures, or subjected to a second sorting step to further reduce the diversity.

Thus, if the diversity after the first sort is fairly high (such as about 100 more, or 500 or more or 10³ or more, or, depending upon the application and desired result, whatever the skilled artisan deems too high to screen by other methods), additional sorting steps are performed.

For these additional steps, the nucleic acid in the sample that contains the identified Dₙ is amplified with a set of primers that each contains a portion (designated FA^{p}) of each epitope-encoding tag (each designated Eₚ) sufficient to amplify the linked nucleic acid, but insufficient to reintroduce Eₚ, where each primer includes or is of a sequence of nucleotides of formula HO-FA-Eₚ, where p is an integer of 1 to m. This amplification introduces a different one of the epitope-encoding sequences into the nucleic acid to produce a collection of cDNA clones (a sublibrary of the original) that again contains all of the epitopes distributed among the sublibrary members.

In this second sorting step, if amplification is used to introduce the new set of tags, concatamer formation can be minimized by using a low concentration of the FA primers followed by an excess of primers encoding the common region, which region is introduced by the FA primer. After the FA primer is used, the common primers out compete the FA primers for incorporation, since the C region will then be incorporated into the template nucleic acid molecule.

Alternatively, as noted above, the new set of epitope-encoding sequences can be ligated via linkers to the template. To do this the template can be cut with a unique restriction enzyme and the linkers ligated. This can get rid of the existing epitope encoding nucleic acid and replace it with a new set of epitopes. Ligation can be followed by amplification with the common region. Other methods can also be used.

In creating the sublibrary for the second sorting step, as with the master library, it is necessary to use conditions that ensure that on the average each different molecule has a different tag and one of each kind is tagged. In this round, one tag, on the average, should attach to each of the different molecules. In this round, however, the diversity is much lower, since the first sorting step achieves an m×n reduction in diversity. Any of the methods described above to attach and distribute polypeptide tag-encoding sequences among the sublibrary members can be used.

Selecting the appropriate stoichiometry assures that a different tag gets on each different member in the library. The number of epitope-encoding molecules should be small relative to the number of molecules in the sublibrary, thereby ensuring an even distribution thereof among the population of different molecules, such that the probability that any particular tag ends up on any particular library member is small. As with the first sorting step and preparation of the master library, particular ratios and concentrations can be empirically determined by varying them and testing.

The nucleic acids in the resulting sublibrary are translated and the translated proteins contacted, such as under western blotting conditions, with one collection of capture agents (or a plurality of replicas thereof), such as antibodies, to form capture agent-protein complexes. The proteins in the complexes are screened to identify the capture agent, such as antibody or receptor, locus (or loci) that binds to the epitope linked to the protein of interest, thereby identifying the “E”, the epitope sequence associated with the protein of interest. Nucleic acid molecules in the sublibrary that contain the identified “E”, epitope sequence, designated E_{q}, are specifically amplified, with primers that include the formula 5′FBS 3′ (or 5′CFB_{S}3′), where each FB is sufficient to amplify the linked nucleic acid using an Eₘ portion of the epitope sequence and includes all or a portion of the Eₘ. This specifically amplifies the nucleic acid molecule of interest.

In summary, the diversity (Div) equals the total number of different molecules in a library (i.e., 10⁸), N=number of divisions D₁-Dₙ, which is the number of different collections of capture agents, such as 10²; M=number of different tags (and capture agents) E₁-Eₘ, such as 10³. To start the method, a master tagged library is prepared, and divided N times. Portions of the N samples are translated and spotted onto N arrays each containing M capture agents (sort 1). At this stage M×N=10⁵. For the second sort, “M” new epitopes, such as 10³ are used, the nucleic acid is translated and sorted onto one array of 10³ capture agents, such as antibodies, thereby achieving a 10⁸ reduction in diversity. As a result, each locus (or member of a collection if provided linked to particulate identifiable supports) in the array has a single type of protein as well as a single capture agent. The number of sorting steps can be any desired number, but is typically one or two. If a higher number of sorts are performed, then the sensitivity of the detection assay at the first sort should be very high, since, as a result of the diversity, the concentration of the protein of interest will be low. As noted above, M and N can be different each sorting step.

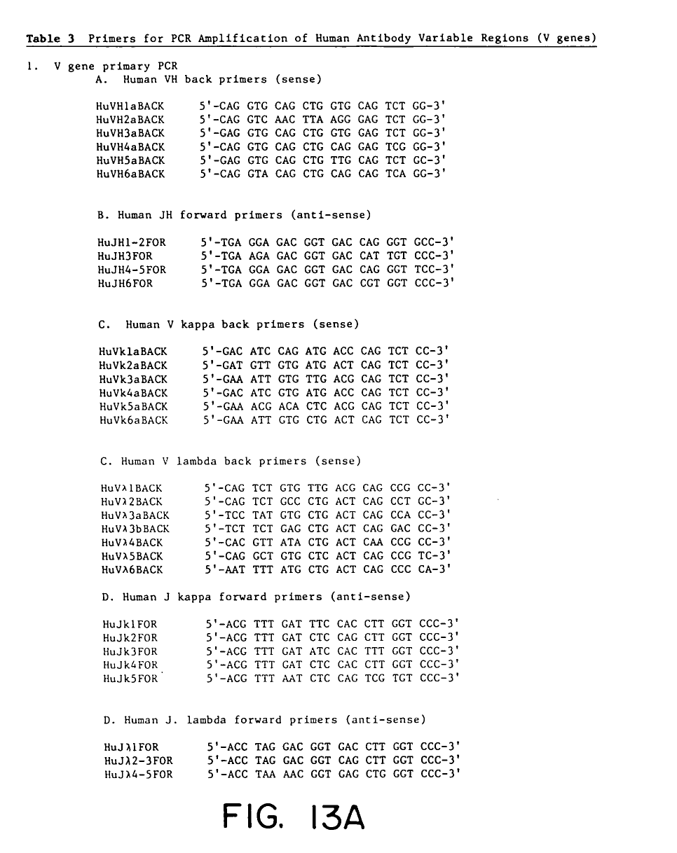

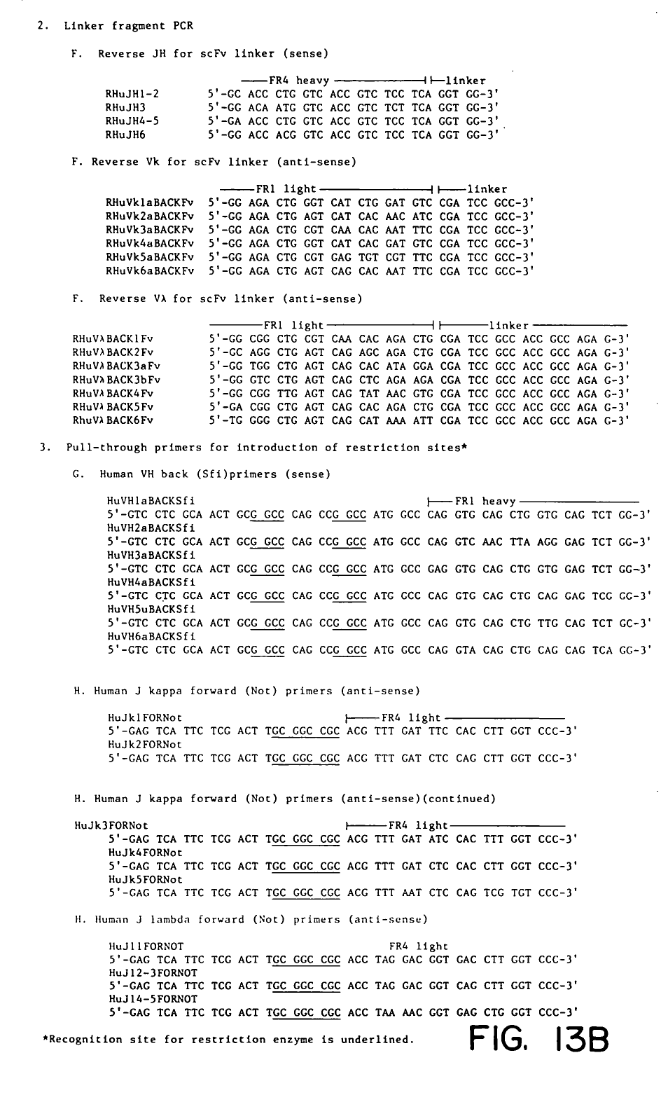

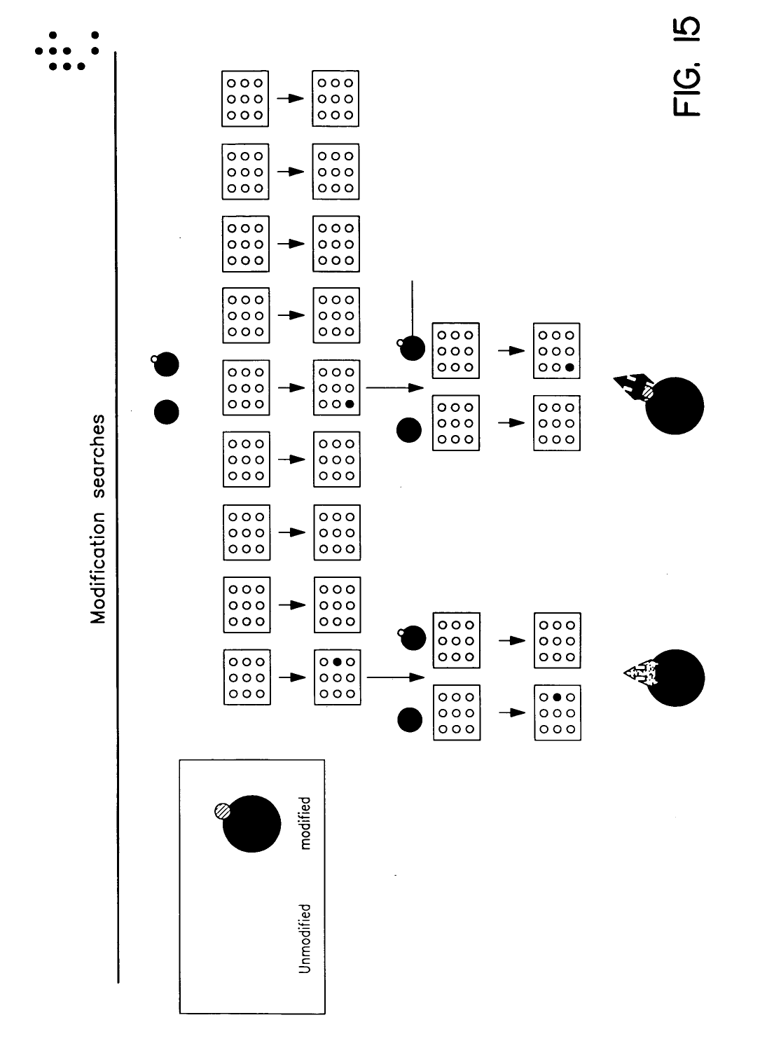

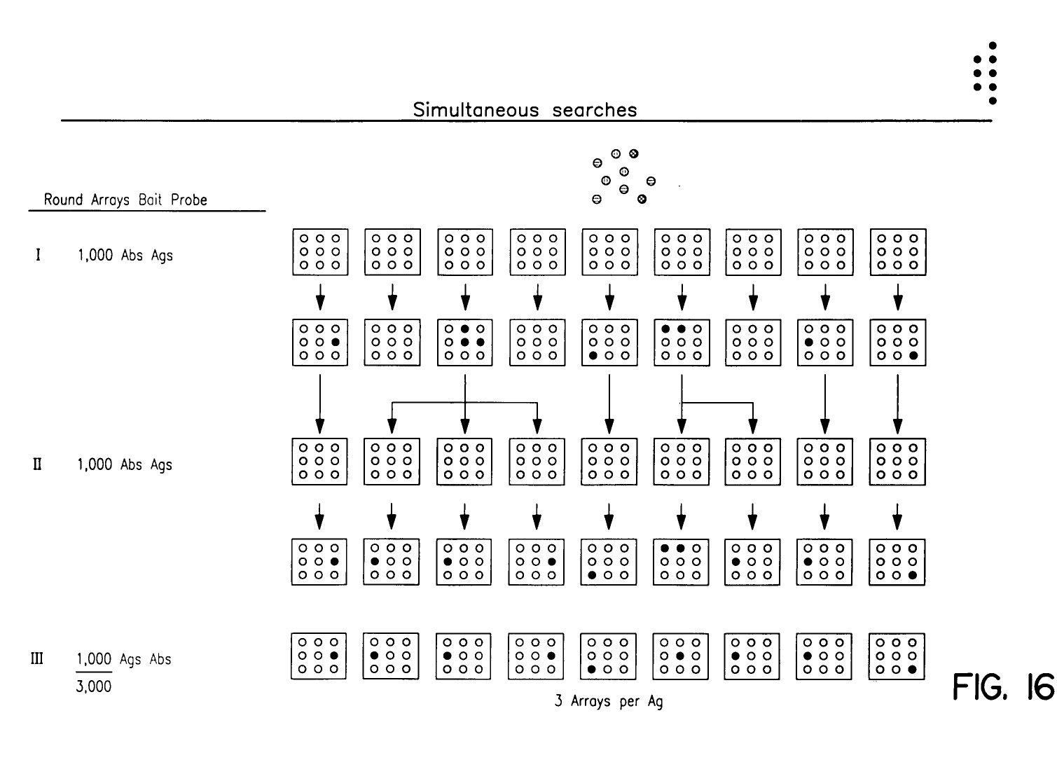

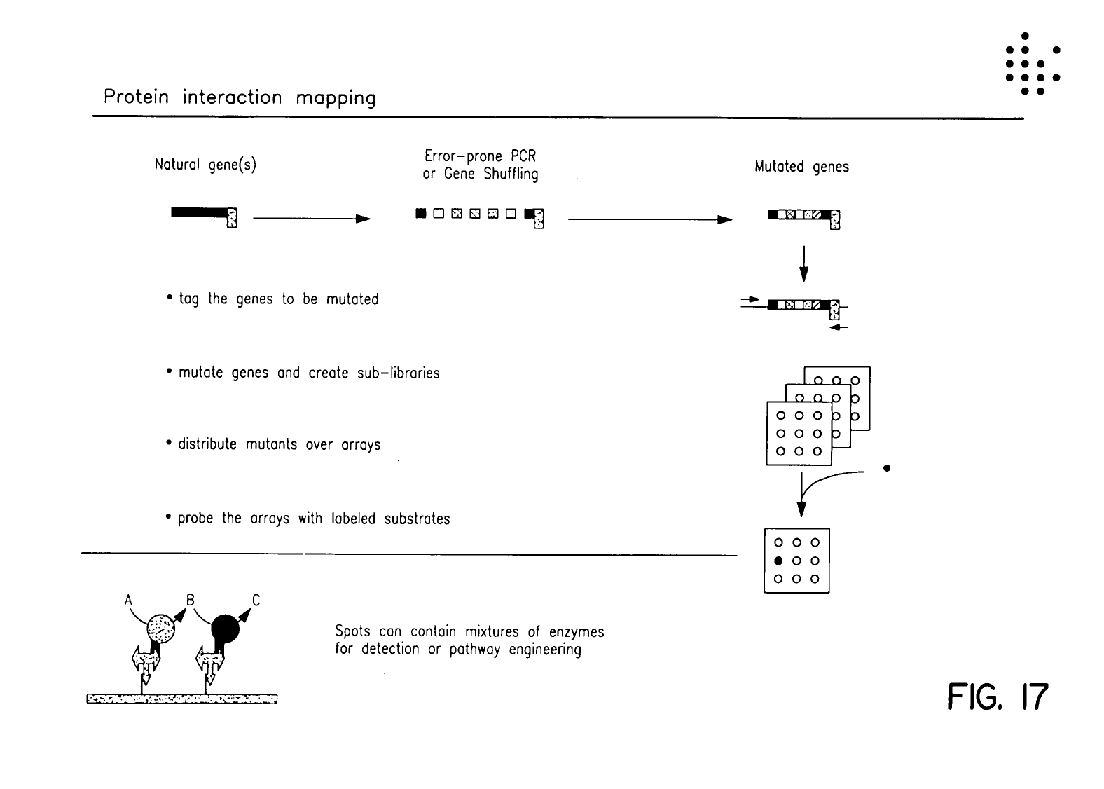

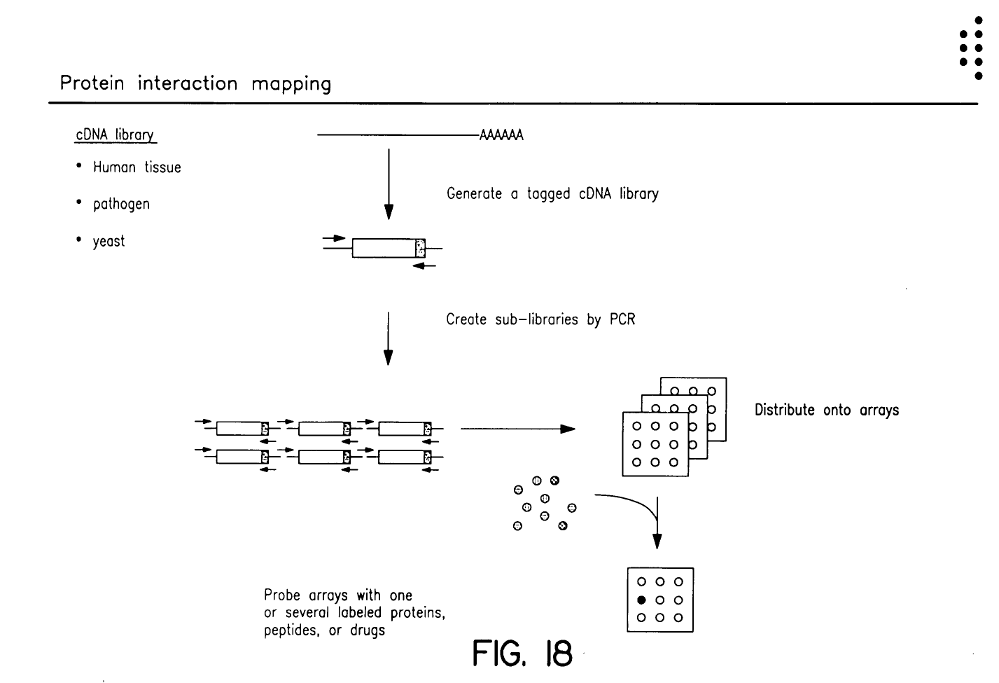

The process of nested sorting, which is applicable to sorting a variety of collections of molecules, particularly collections of proteins, DNA, small molecules and other collections is exemplified in FIGS. 1-18. The concept of nested sorting is illustrated in FIG. 1. In this example, a master collection containing 74,088 different items, such as cDNA, is searched by randomly dividing the collection into 42 sublibraries (F1 sublibraries). After identifying which of the 42 F1 sublibraries contains the item of interest, such as by binding or reaction with a probe or by a protein-protein specific interaction, that group is further divided randomly into 42 new sublibraries (F2 sublibraries) and again the sublibrary containing the item of interest is identified. A final division of the F2 sublibrary containing the item of interest produces 42 new groups, each containing only one item. The item of interest can be uniquely identified based on its sorting lineage.

In the example shown, the item of interest was identified in the fifth F1 sublibrary, the thirty first F2 sublibrary, and the sixteenth F3 sublibrary. Of the 74,088 items in the master collection, only one has the sort lineage F1₅/F2₃₁/F3₁₆.

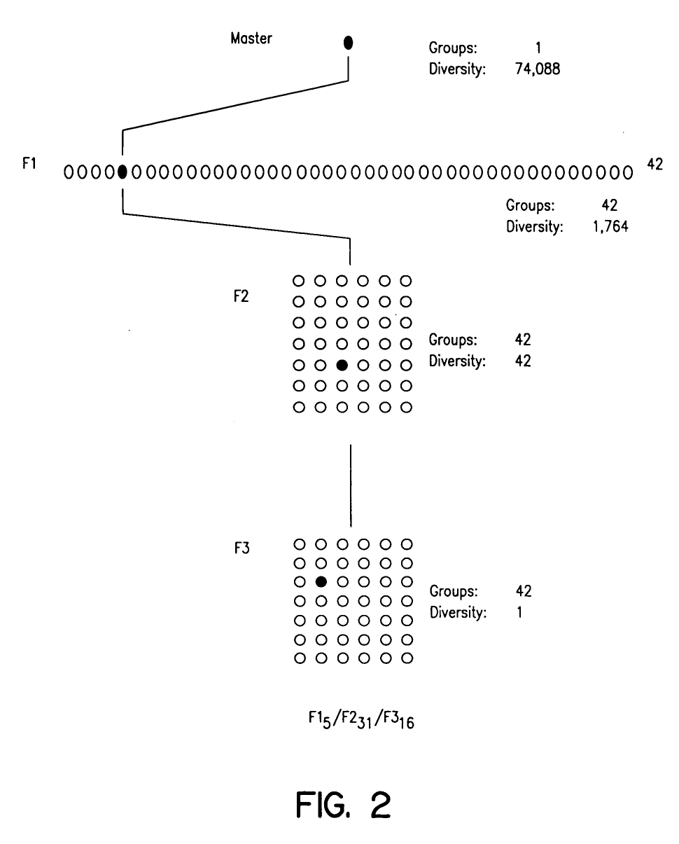

The sort illustrated in FIG. 2 is identical to the sort illustrated in FIG. 1 except that the F2 and F3 sublibraries have been arranged into arrays. This figure also illustrates that as the sort proceeds, the diversity of items within each sublibrary decreases; the exemplified master collection contains 74,088 items, the 42 F1 sublibraries contain 1,764 items each, the 42 F2 sublibraries contain 42 items, and the 42 F3 sublibraries contain only a single item. The first two figures illustrate a theoretical search based on nested sorting.

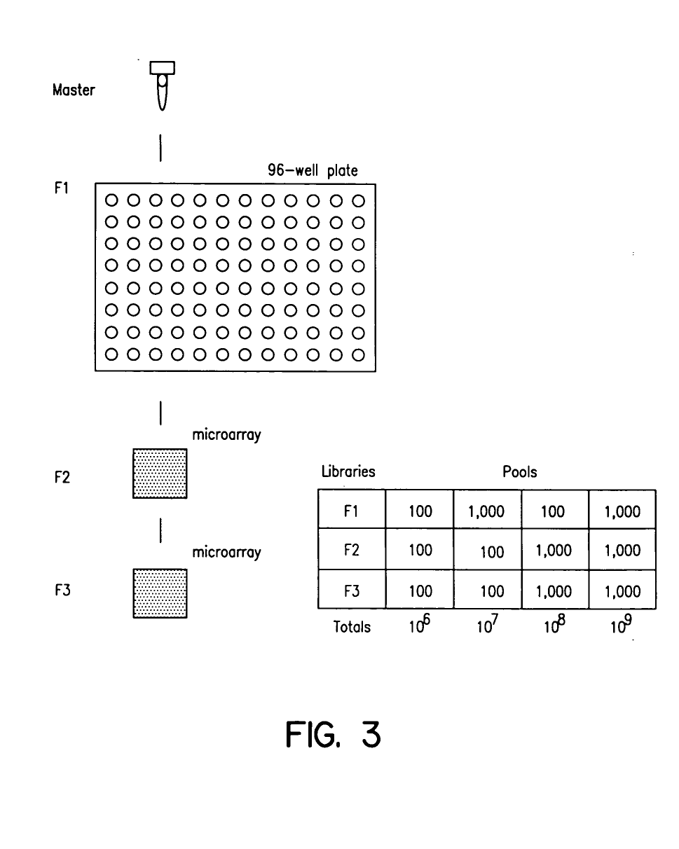

FIG. 3 illustrates the use of capture agent arrays, such as antibody arrays, as a tool for nested sorts of high diversity gene libraries. A master gene library is first randomly divided into a number of sublibraries by separate amplification, such as PCR, reactions. The amplification reactions use sets of unique sequences of nucleotides that encode preselected epitopes and incorporate these sequences into the genes by appropriate design of primers to specifically amplify different sublibraries of genes from the master template pool (F1 sublibraries). These amplification reactions are performed, for example, in 96-well (or 384-well or higher density) PCR plates with a compatible thermocycler.

The amplified genes in each well are translated into their protein products and samples from each are then applied to separate capture agent collections, such as arrays (i.e., proteins from each well in the 96-well plate are applied to one of 96 capture agent arrays). The proteins, such as antibodies, sort into defined locations on the array by binding to capture agents in the array that recognize the known unique amino acid sequences (the epitopes) that have been added to the proteins using the primers. After sorting, addresses on the array that contain the protein of interest are identified and nucleic acids from the sublibrary from which those proteins with the epitope encoding sequences that bind to the spot in the array are amplified, such as by PCR.

During this second amplification step, new sets of known epitopes are incorporated into the nucleic acid, so that they can be further sorted using additional capture agent arrays (F3).

The table in FIG. 3 illustrates how the number of initial divisions by PCR and the number of capture agents the array can be combined to search gene libraries containing, for example, from a million (10⁶) to over a billion (10⁹) different genes. For example, an initial gene library can be divided into 100 F1 sublibraries by amplification and then further divided using two sequential arrays with capture agents recognizing 100 different epitopes. If the initial gene library contained 10⁶ different genes, the F3 addresses in the sublibraries contain a single type of gene (10⁶/100/100/100=1). An initial gene library divided into 1,000 F1 sublibraries by PCR amplification and then further divided using arrays with capture agents recognizing 1,000 different epitopes to create the F2 and F3 sublibraries can be used to search 10⁹ different genes (10⁹/1,000/1,000/1,000=1).

Dividing the gene libraries into sublibraries is based on the ability of a PCR amplification reaction to specifically amplify DNA sequences using pairs of primers. Although both primers need to hybridize to sequences on either end of the template DNA, a subset of template sequences can be amplified using a primer pair in which one of the primers is common to all of the template sequences and the other primer is specific for the gene sequence of interest. For example, specific genes are often amplified from cDNA libraries using one primer that is specific for the gene of interest and another that hybridizes to the oligo(dA) tail common to all of the cDNA molecules.

E. Use of the Methods for Identification of Proteins of Desired Properties from a Library

1. Arraying Capture Agents

The capture agent molecules to which the tags, such as epitope tags, specifically bind are linked to supports, such as identifiable beads, such as microspheres, or solid surfaces. Linkage can be effected through any suitable bond, such as ionic, covalent, physical, van der Waals bonds. It can be effected directly or via a suitable linker. For exemplary purposes arraying on surfaces is described.

Purified antibodies (e.g., 1 μl at a concentration of 1-2 mg/ml in a buffer of 0.1 M PBS (phosphate buffered saline, pH 7.4) containing glycerol (1-20% vol/vol), are spotted onto a membrane (such as, for example, UltraBind membrane, Pall Gelman; FAST nitrocellulose coated slides, Schleicher & Schuell), chemically deactivated glass slides, superaldehyde slides (Telechem), polylysine coated glass, activated glass, or specific thin films and self-assembled monolayers International PCT application Nos WO 00/04389, WO 00/04382 and WO 00/04390). using an automated arraying tool (such as systems available from, for example, Microsys; PixSys NQ; Cartesian Technologies; BioChip Arrayer; Packard Instrument Company; Total Array System; BioRobotics; Affymetrix 417 Arrayer; Affymetrix, and others). The spots are allowed to air dry for a suitable period of time, 1-2 minutes or more, typically 30 min to 1 hr. Two membrane attachments are described. The UltraBind membrane (Pall Gelman) contains active aldehyde groups that react with primary amines to form a covalent linkage between the membrane and the capture agent, such as an antibody. Unreacted aldehydes are blocked by incubation with suitable blocking solution, such as a solution of 50 mM PBS, pH 7.4, 2% bovine serum albumin (BSA) or with BBSA-T (a protein-containing solution such as Blocker BSA (Pierce) diluted to 1×in phosphate-buffered saline (PBS) with Tween-20 (polyoxyethylenesorbitan monolaurate; Sigma) added to a final concentration of 0.05% (vol:vol)) for a suitable time, such as about 30 minutes. Buffers containing glycine, or other free amino groups are also suitable for blocking aldehyde containing surfaces. The filter can also be rinsed with PBS.

Capture agents, such as antibodies, also can be deposited onto membranes, such as, for example, nitrocellulose paper (Schliecher & Schuell) with, for example, an inkjet printer (i.e., Canon model BJC 8200, color inkjet printer), modified for this use and connected to a computer, such as a personal computer (PC). Such modifications, include, removal of the color ink cartridges from the print head and replacement with, for example, 1 milliliter pipette tips, which are hand-cut to fit in a sealed manner over the inkpad reservoir wells in the print head. Antibody solutions are pipetted into the pipette tips reservoirs that are seated on the inkpad reservoirs.

Printed images, using the modified printer, are generated, with, for example, Microsoft PowerPoint. The images are then printed onto nitrocellulose paper, which is cut to fit and then taped over the center of a sheet of printing paper. The set of papers is then fed into the printer immediately prior to printer.

Purified capture agents, such as antibodies, can also be spotted onto FAST nitrocellulose coated slides, (Schleicher & Schuell). Nitrocellulose binds proteins at approximately 100 μg per cm² by noncovalent adsorption. After binding of the capture agents, such as antibodies, the remaining binding sites are blocked by incubation with a solution of 50 mM PBS, pH 7.4, 2% bovine serum albumin (BSA) or BBSA-T for a suitable time, such as for 30 minutes.

Direct binding of antibodies to the nitrocellulose results in non-oriented binding. The percentage of active immobilized antibody molecules can be increased by binding to nitrocellulose that has been coated with an antibody capture protein (such as protein A, protein G or anti-IgG monoclonal antibody). The capture agents, such as antibodies, are bound to the nitrocellulose before application of the library proteins, such as tagged antibodies, with an arrayer. Biotinylated antibodies can also be printed onto surfaces coated with avidin or streptavidin. The size and spacing of the spots can be adjusted depending on the filter used and the sensitivity of the assay. Typical spots are about 300-500 μm in diameter with 500-800 μm pitch.

Antibodies can also be printed onto activated glass substrates. Prior to printing the glass is cleaned ultrasonically in succession with a 1:10 dilution of detergent in warm tap water for 5 minutes in Aquasonic Cleaning Solution (VWR), multiple rinses in distilled water and 100% methanol (HPLC grade) followed by drying in a class 100 oven at 45° C. Clean glass is chemically functionalized by immersion in a solution of 3-aminopropyltriethoxysilane (APTS) (5% vol/vol in absolute ethanol) for 10 minutes. The glass is then rinsed in 95% ethanol, allowed to air dry, and then heated to 80° C. in a vacuum oven for 2 hours to cure. The surface then can be further modified to bind primary amines or free sulfhydryl groups in the antibody or avidin or streptavidin linked to the antibody with biotin. To create an amine-reactive surface, the functionalized glass is treated with a solution of Bis[sulfosuccinimidyl]suberate (BS³)(5 mg/ml in PBS, pH 7.4) for 20 minutes at room temperature. The N-hydroxy-succinimide (NHS)-activated glass surface is rinsed with distilled water and placed in a 37° C. dust-free class 100 oven for 15 minutes to dry. Antibodies can be directly attached to this surface or the surface can be coated with a protein such as protein A that binds the antibodies, protein G or anti-IgG monoclonal antibody or avidin/streptavidin, to bind biotinylated proteins. To create a sulfhydryl-reactive surface, the functionalized glass is treated with a solution of sulfosuccinimidyl 4-[N-maleimidomethyl]-cyclohexane-1-carboxylate (Sulfo-SMCC) for 20 minutes at room temperature. The maleimide-activated glass surface is rinsed with distilled water and placed in a 37° C. dust-free class 100 oven for 15 minutes to dry. To create a biotinylated surface, the functionalized glass is treated with a solution of EZ-link Sulfo-NHS-LC-Biotin (Pierce) for 20 minutes at room temperature. The biotinylated glass surface is rinsed with distilled water and placed in a 37° C. dust-free class 100 oven for 15 minutes to dry. The same immobilization strategies described above also can be used in self-assembled monolayers formed on top of inorganic thin films.

2. Exemplary Use for Identification of Genes from a Library of Mutated Genes

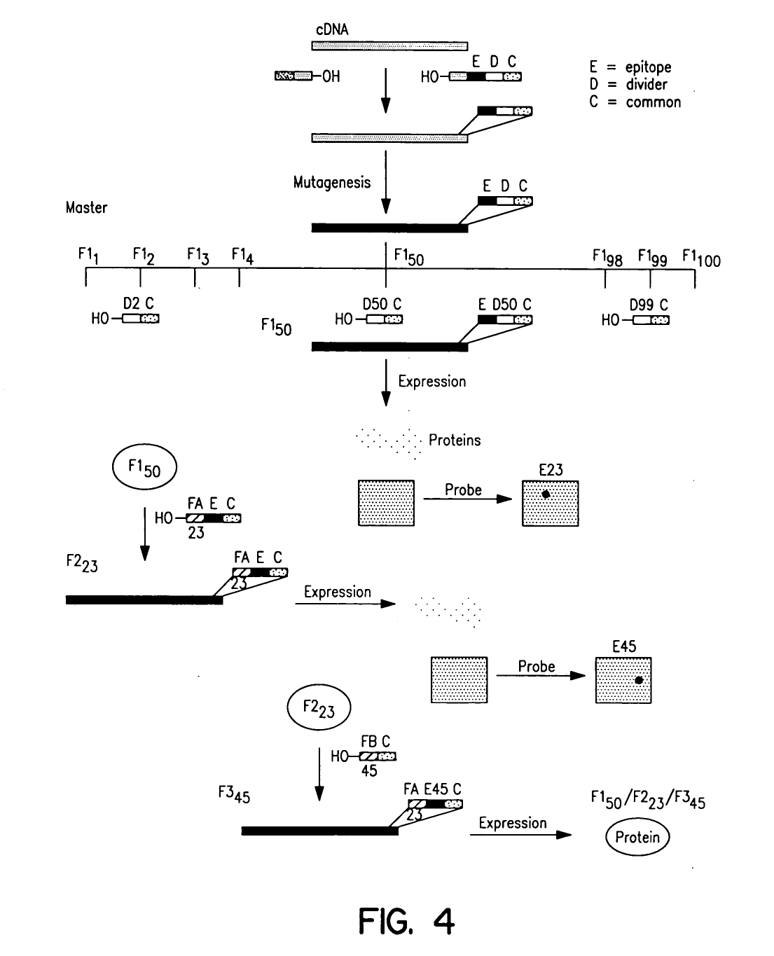

FIG. 4 illustrates the use of the methods herein to search a library of mutated genes. Mutation of specific gene regions by a variety of methods is often used to improve the properties of proteins encoded by the mutated genes, such as mutated genes produces by error-prone PCR or gene shuffling mutagenesis techniques to improve the binding affinity of a recombinant antibody. This technique coupled with selection by surface display has been used to improve the binding affinities of antibodies by several orders of magnitude. Mutation has also been used to improve the catalytic properties of enzymes. The methods herein provide methods to screen and identify mutated genes encoding proteins having desired properties.

Initially a set of oligonucleotides containing various functional domains are added to the 3′ ends of a gene to be mutated by incorporation of a primer that contains sequences of nucleotides that hybridize to the gene and also additional sets of sequences, designated E for “Epitopes” D for “Divider”, and C for “Common”. The E D C sequences constitute sets of sequences, each defined by the functions in the nucleic acid. As noted, the E sequences encode the epitopes specifically recognized by antibodies in the collection. They are incorporated in-frame with the coding sequences of the gene to be mutated and are expressed as a fusion with the parent protein. The D sequences are unique sequence sets downstream from the epitopes. They serve as specific priming sites to “Divide” the master group. They can be non-coding sequences and do not necessarily end up being part of the expressed mutated proteins. The C sequence is a sequence “Common” to all of the genes and provides a method for simultaneous PCR amplification of all the gene templates. As noted previously, in certain embodiments the D and/or C sequences are optional. Importantly, the E and D sequences are randomly distributed among the resulting DNA molecules. For example, 100 E sequences and 100 D sequences combine to create 10,000 (100×100=10,000) uniquely tagged cDNA molecules. Likewise, 1,000 E sequences and 1,000 D sequences combine to create 1,000,000 (1,000×1,000=1,000,000) uniquely tagged cDNA molecules.

Before, or after the E C and D sequences have been added to the ends of the molecule to be mutated, defined regions within the gene are mutated by a variety of standard methods. The mutation procedure should not produce mutations in the E D C sequences. After the mutagenesis has been completed, the mutated DNA is added as template to a first set of PCR reactions to create the F1 sublibrary. In addition to the template DNA, D C primer sets are separately added such that each PCR contains a primer complementary to a different D sequence. For example, in FIG. 4 the second PCR tube is identical to the rest of the tubes except it contains a D C primer containing only one of the 100 D sequences (D₂). In this illustration, tube 50 is identical to the rest of the F1 reaction tubes except it contains a different one of the 100 D sequences (D₅₀). The resulting PCR amplification products contain all of the 100 different E sequences randomly distributed among the genes but only containing one of the 100 D sequences. In the illustration, PCR tube 50 produces a sublibrary DNA molecules (F1₅₀) that all have the same D₅₀ sequences, the same C sequence but different E sequences randomly distributed among the molecules (ED₅₀ C).

The generated F1 DNA molecules are expressed in vitro using a transcription-translation extract. Appropriate regulatory DNA sequences, including promoters, ribosome binding sites and other such regulatory sequences known to those of skill in the art, for efficient in vitro transcription and translation are incorporated into the DNA fragments during the tagging process. As illustrated in FIG. 4, expression of the F1₅₀ DNA molecules produces a collection of proteins containing the various tags. Proteins produced in bacteria or in other in vivo systems also can be used.

The resulting expressed proteins are incubated with the antibody collection, such as in an array format under conditions that permit binding between the epitopes and the antibody(ies) specifically selected to bind to each of the epitopes. This results in specific binding of proteins to antibodies. If the antibodies are arranged in an array, this results in the distribution of the tagged proteins to locations on the array containing immobilized antibodies that bind the proteins cognate epitopes. After binding, the array is washed, probed, and analyzed by any method known to those of skill in the art, such as by enzymatic labeling, such as with luciferase. For example, analysis can be effected by photon collection using detectors, such as a photomultiplier tube, a photodiode array or generally charge coupled device (CCD)-based imaging detector to detect emitted light. Photons can be produced by local enzymatic chemiluminescent, particularly bioluminescent reactions. Photon collection is desirable, since it advantageously is relatively inexpensive, very sensitive and the sensitivity can be amplified by increased collection times.

As an example, if the search is used to identify mutations to the luciferase enzyme that confer increased activity, the array is washed, bathed in substrate and then analyzed for increased luciferase activity as measured by increased photon output. The “brightest spot” in the array has bound the enzyme with the most favorable mutations.

As another example, if the search is used to identify increased affinity of an antibody for its antigen, the array is washed then incubated with tagged antigen. The tag on the antigen is used to bind to a secondary detection reagent such as streptavidin conjugated HRP if the antigen is tagged with biotin, or an antibody-HRP complex, if the tag is a defined epitope. Again, the “brightest spot” contains the mutant antibody with the greatest affinity, having bound the greatest amount of antigen.

Knowing the location of the “brightest spot” and epitope binding specificity of the antibodies in that spot, identifies the E sequence associated with the mutant gene of interest. At this point in the sort, the template for the gene of interest (as illustrated in FIG. 4) is known to be in the F1₅₀ sublibrary and contain the E23 sequence (F1₅₀/F2₂₃).

Genes containing the E23 sequence can be amplified using template DNA from the F1₅₀ sublibrary and PCR primers with sequences corresponding to the E23 sequence (FA₂₃ E C). Like the D C set of primers used to initially divide the master library, the FA E C set of primers are used to amplify templates containing specific E sequences and at the same time re-distribute E sequences among the amplified genes. The FA E C primer is composed of 3 functional regions. The FA region contains sequences corresponding to an upstream fragment (Fragment A) of the E sequence present in the template. The FA region contains any amount of the E sequence that confers hybridization specificity, but that, upon translation, does not confer the epitope binding specificity. As before, the E region encodes epitope sequences and the C region encodes a common sequence for amplification. The FA and E sequences are in-frame with the coding region of the gene. The resulting amplified genes represent an F2 sublibrary (F2₂₃).

The amplified genes from the F2 sublibrary are expressed in vitro, incubated with the antibody array, re-probed and analyzed. As before, “bright spots” in this array identifies the E sequence associated with the mutant gene of interest. At this point in the sort, the gene of interest (as illustrated in FIG. 4) is known to be in the F1₅₀ and F2₂₃ sublibraries and contains the E45 sequence (F1₅₀/F2₂₃/F3₄₅). This information identifies a specific gene that can be amplified using a primer specific for the E45 sequence (FB₄₅ C). The FB C primer is composed of two functional regions. The FB region contains sequences corresponding to a downstream fragment (Fragment B) of the E sequence present in the template. FB can contain all or part of E; C is optional. FB contains any part, up to and including all of the E encoding sequence, to confer hybridization specificity. As before, the C region encodes a common sequence for amplification. The resulting amplified genes represent an F3 sublibrary (F3₄₅).

F. Identification of Recombinant Antibodies

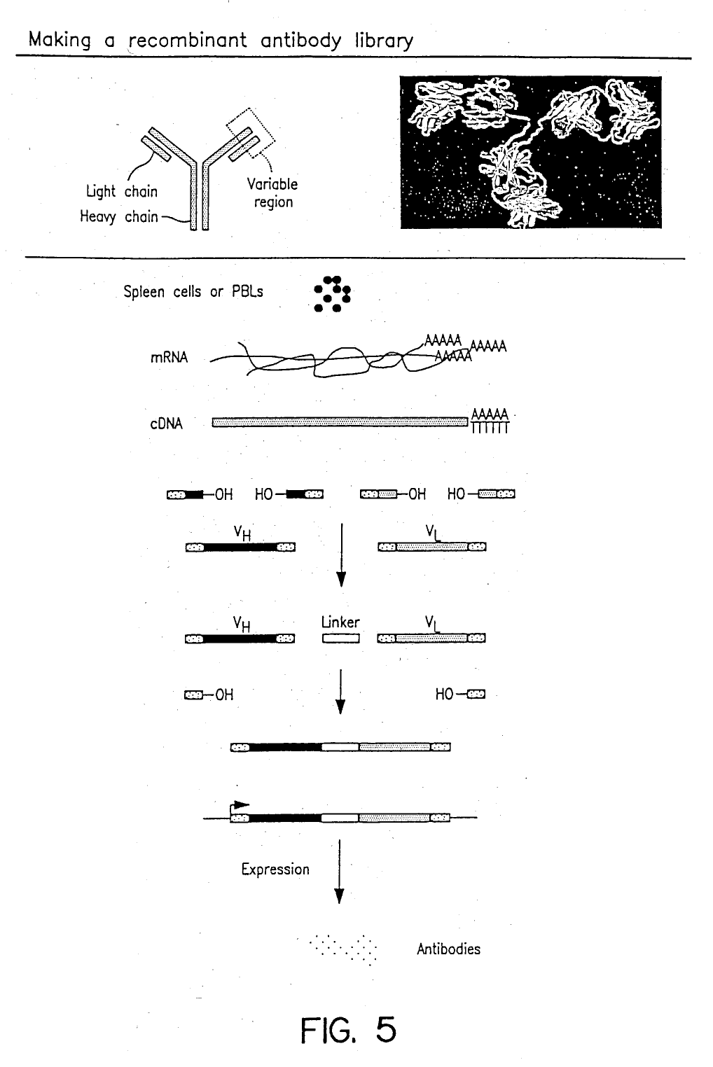

Another application of the technology is its use for the identification of recombinant antibodies. Antibodies with desired properties are sorted out of large pools of recombinant antibody genes. An overview of a standard method for constructing recombinant antibody libraries is illustrated in FIG. 5. The initial steps involve cloning recombinant antibody genes from mRNA isolated from spleenocytes or peripheral blood lymphocytes (PBLs). Functional antibody fragments can be created by genetic cloning and recombination of the variable heavy (V_{H}) chain and variable light (V_{L}) chain genes. The V_{H} and V_{L} chain genes are cloned by first reverse transcribing mRNA isolated from spleen cells or PBLs into cDNA. Specific amplification of the V_{H} and V_{L} chain genes is accomplished with sets of PCR primers that correspond to consensus sequences flanking these genes. The V_{H} and V_{L} chain genes are joined with a linker DNA sequence. A typical linker sequence for a single-chain antibody fragment (scFv) encodes the amino acid sequence (Gly₄Ser)₃. After the V_{H}-linker-V_{L} genes have been assembled and amplified by PCR, the products can be transcribed and translated directly or cloned into an expression plasmid and then expressed either in vivo or in vitro to produce functional recombinant antibody fragments.

The method of recombinant antibody library construction can be adapted for use with the sorting methods herein. This is accomplished by incorporating the E D C sequences into the V_{L} chain genes before assembly with the V_{H} chain and linker sequences. After the recombinant antibody library has been tagged with the E D C sequences, it is sorted by division into the F1 sublibraries followed by screening with the arrays as described above.

Two different methods are illustrated for incorporating the E D C sequences into the amplified V_{L} chain genes. In the first method, the E D C sequences are part of the first-strand cDNA synthesis primer and get incorporated during cDNA synthesis (FIG. 6) in the second method the E D C sequences are incorporated after cDNA synthesis (FIG. 7) by the addition of double-stranded DNA linker molecules.

FIG. 6 illustrates how E D C sequences are put onto the V_{L} chain genes by primer incorporation. The V_{H} chain genes are cloned using standard methods. The mRNA isolated from spleen cells or PBLs is converted to cDNA using a universal oligo dT primer or IG gene-specific primers. The V_{H} genes are then specifically amplified using a set of primers that are complementary to consensus sequences that flank these genes. The V_{HBACK} primer also contains promoter sequences that are required for in vitro transcription and translation of the assembled gene. and/or allows subcloning into plasmid vectors for in vivo expression in cells, such as, but are not limited to, bacterial, yeast, insect and mammalian cells.

The V_{L} gene is cloned using a set of reverse transcription primers (V_{L}FOR) that contain sets of sequences that are complementary to downstream consensus sequences flanking the V_{L} genes (J_{kappa for}) and the E D C sequences. The E D C sequences are located 5′ to the Jₖₐₚₚₐ for sequences in the V_{LFOR} primer. The second strand of the cDNA is primed using an oligonucleotide (V_{LBACK}) containing complementary sequences to the upstream consensus region of the V_{L} gene (V_{kappa back}). After the second strand cDNA synthesis the V_{L}genes are amplified with a combination of the V_{LBACK} and V_{LFOR-C} primers. The V_{LFOR-C} primer consists of sequences complementary to the C region of the E D C sequence.

After amplification of the V_{H} and V_{L} genes, the fragments are digested with a restriction enzyme to produce overlapping ends with the linker. The V_{H}-linker-V_{L} fragments are sealed with DNA ligase and then amplified using the V_{HBACK} and V_{LFOR-C} primers.

In the second method, illustrated in FIG. 7, the V_{H} genes are amplified as described above. This method differs from the first in that the V_{L} gene first-strand synthesis is primed with an oligonucleotide containing a unique restriction site 5′ to the J_{kappa for} sequences. This restriction site is incorporated into the 3′-end of the resulting cDNA such that a unique cohesive end can be produced by restriction enzyme digestion. The linkers are mixed with the cut cDNA, sealed with ligase and then amplified with a combination of the V_{HBACK} and V_{LFOR-C} primers.

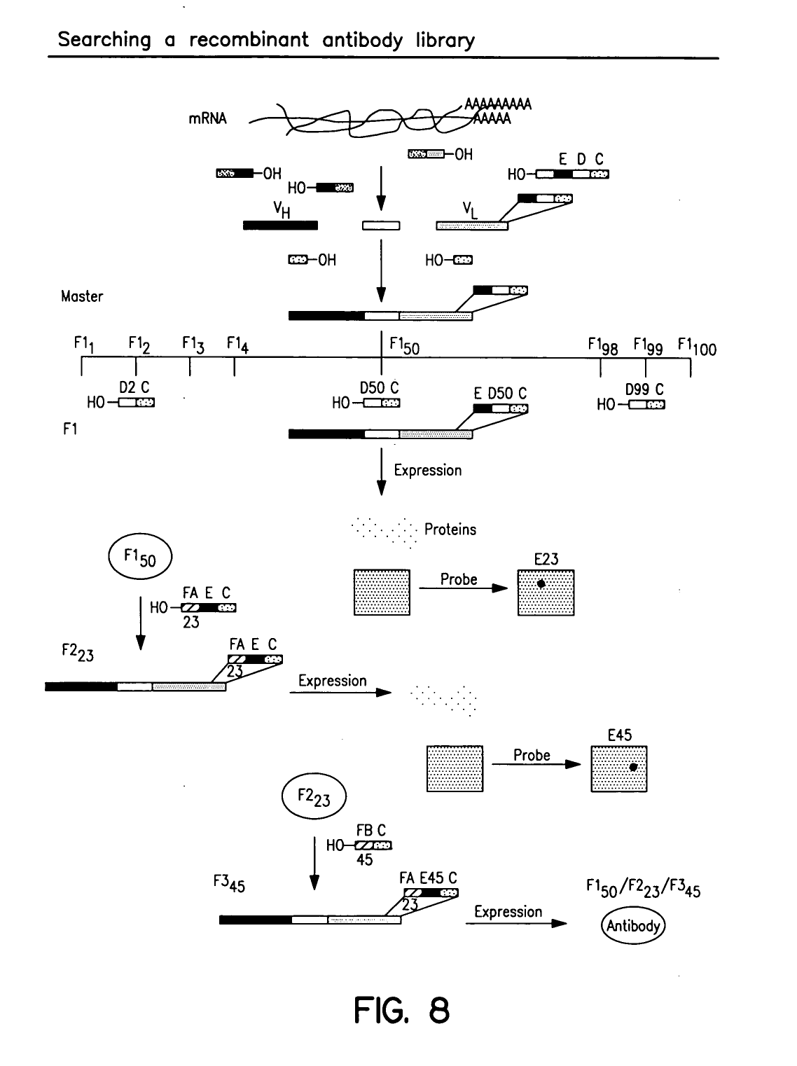

FIG. 8 outlines a method for searching a recombinant antibody library. The V_{H} and V_{L} genes are cloned as described above and the E D C sequences are added to the 3′-end of the antibody genes to create the master library. The F1 sublibraries are created using the D C set of PCR primers. The illustration depicts 100 F1 sublibraries, shows D C primers for F1₂, F1₅₀ and F1₉₉, and shows the amplified product from the F1₅₀ reaction.

Transcription and translation of the F1₅₀ sublibrary genes produces a variety of recombinant capture agents, such as antibodies, that can be randomly grouped according to the epitopes (E sequences) they contain. The expressed proteins are bathed over the array and allowed to sort onto spots in the array that contain antibodies that bind their specific tags, such as epitope tags. After the scFvs from sublibrary F1₅₀ are bound to the array, labeled antigen is bathed over the array. The label on the antigen can be a chemical tag, such as biotin, used to bind a secondary detection reagent such as streptavidin-conjugated HRP, or the antigen can be tagged and detection achieved with an anti-epitope antibody-HRP complex. After binding, the array is washed, probed, and analyzed. Analysis is typically by photon collection using a CCD-based imaging detector and photons are typically produced by local enzymatic chemiluminescent reactions. Again, the “brightest spot” can contain the recombinant antibody with the greatest affinity, having bound the greatest amount of antigen.

Knowing the location of the “brightest spot” and epitope binding specificity of the antibodies in that spot, identifies the E sequence associated with the recombinant antibody gene of interest. At this point in the sort, the template for the gene of interest (as illustrated in FIG. 8) is known to be in the F1₅₀ sublibrary and contain the E23 sequence.

Genes containing the E23 sequence can be amplified using template DNA from the F1₅₀ sublibrary and PCR primers with sequences corresponding to the E23 sequence (FA₂₃ E C). Like the D C set of primers used to initially divide the master library, the FA E C set of primers are used to amplify templates containing specific E sequences and at the same time re-distribute E sequences among the amplified genes. The FA₂₃ E C primer is used to amplify template DNA from the F1₅₀ sublibrary. The resulting amplified genes represent an F2 sublibrary, F2₂₃. The initial lineage for the antibody of interest is F1 ₅₀/F2₂₃.

The amplified genes from the F2 sublibrary are expressed in vitro or in in vivo systems, incubated with the antibody array, re-probed and analyzed. As previously, “bright spots” in this array identifies the E sequence associated with the recombinant antibody gene of interest. At this point in the sort, the gene of interest (as illustrated in FIG. 8) is known to be in the F1₅₀ and F2₂₃ sublibraries and contains the E45 sequence (F1₅₀/F2₂₃/F3₄₅). This information identifies a specific gene that can be amplified using a primer specific for the E45 sequence (FB₄₅ C). The resulting amplified genes represent an F3 sublibrary (F3₄₅77) that contains a single type of recombinant antibody.

### G. EXAMPLES

The following examples are included for illustrative purposes only and are not intended to limit the scope of the invention.

### Example 1

### Preparation of Capture Agent Collections

A. Generating a Collection of Capture Agent—Tag Pairs

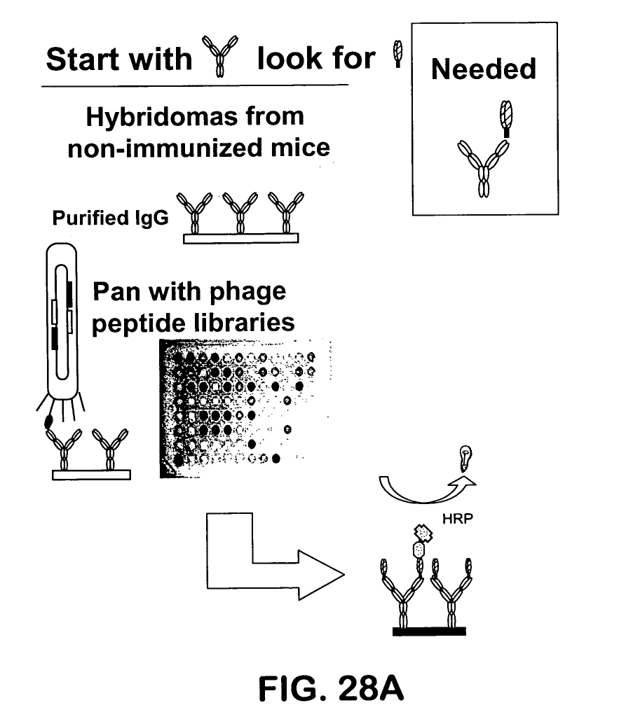

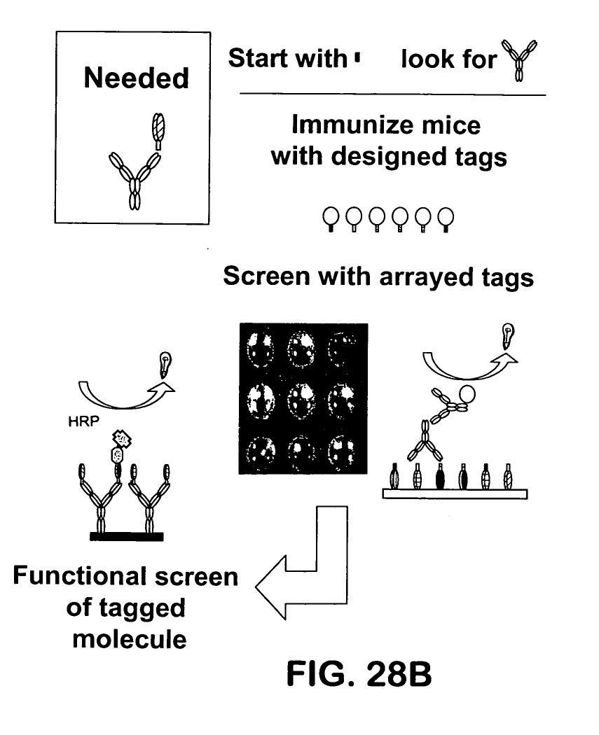

A collection of capture agents, such as antibodies, that bind tags, such as polypeptides, is used to sort molecules linked to the tags. The collection of antibodies that specifically bind to the polypeptide tags can be generated by a variety of methods. Two examples are described below and are exemplified in FIGS. 28A and 28B.

1. Hybridoma Screening

In the first example, high affinity and high specificity antibodies for the array are identified by screening a randomly selected collection of individual hybridoma cells against a phage display library expressing a random collection of peptide epitopes. The hybridoma cells are created by fusion of spleenocytes isolated from a naive (non-immunized) mouse with myeloma cells. After a stable culture is generated, approximately 10-30,000 individual cell clones (monoclonals) are isolated and grown separately in 96-well plates. The culture supernatants from this collection are screened by ELISA with an anti-IgG antibody to identify cultures secreting significant amounts of antibody. Cultures with low antibody production are discontinued. Antibodies from this monoclonal collection are separately affinity purified from culture supernatants using high throughput 96-well purification methods and the amounts purified and quantified.

The purified antibodies are arrayed by robotic spotting onto a filter and are also separately mixed then bound to paramagnetic beads to create a substrate for panning high affinity epitopes from a filamentous M13 bacteriophage library displaying random cysteine-constrained heptameric amino acid sequences. The phage library is enriched for phage displaying high affinity epitopes by mixing the phage library with the antibody-coated beads and washing away loosely-bound phage from the beads (“panning”). Several rounds of panning leads to a highly enriched library containing phage that tightly bind to the monoclonal antibodies present in the collection. To separate and identify high affinity phage-antibody pairs, the enriched phage library is incubated with the filter containing the arrayed antibodies under high stringency binding conditions. Phage bound to antibodies on the filter are identified by staining with HRP-conjugated anti-phage antibodies and a chemiluminescent substrate to produce a luminescent signal. The signal is quantified using a high resolution CCD camera imaging device. High affinity binding phage are recovered from the filter and propagated. Several independent phage clones recovered from each spot are sequenced to identify consensus high-affinity epitopes for the corresponding antibodies.

a. Making Hybridomas

Hybridoma cells are prepared by well known methods known to those of skill in the art (see, e.g., Harlow et al. (1988) Antibodies: A Laboratory Manual, Cold Spring Harbor Laboratory, Cold Spring Harbor). Hybridoma cells are created by the fusion of mouse spleenocytes and mouse myeloma cells. For the fusion, antibody-producing cells isolated from the spleen of a non-immunized mouse are mixed with the myeloma cells and fused. Alternatively, the hybridoma cells are created from spleenocytes isolated from a mouse previously immunized with a recombinant protein (e.g., dihydrofolate reductase, DHFR) containing a mixture of different tags or synthetic peptides conjugated to a carrier (i.e., Keyhole limpet hemocyanin, KLH). The tags are random cysteine-constrained peptides expressed as part of a genetic fusion to the DHFR gene. The random peptides are encoded by a DNA insert assembled from synthetic degenerate oligonucleotides and cloned into the gene III protein (gill) of the filamentous bacteriophage M13. DNA encoding the peptide library is available commercially (Ph.D.-C7C™ Disulfide Constrained Peptide Library Kit, New England Biolabs). The Ph.D.-C7C™ library contains approximately 3.7×10⁹ different peptides

After fusion, cells are diluted into selective media and plated into multiwell tissue culture dishes. A healthy, rapidly dividing culture of mouse myeloma cells are diluted into 20 ml of medium containing 20% fetal bovine serum (FBS) and 2×OPI. Medium is typically Dulbecco's modified Eagle's (DME) or RPMI 1640 medium. Ingredients of mediums are well known (see, e.g., Harlow et al (1988) Antibodies: A Laboratory Manual, Cold Spring Harbor Laboratory, Cold Spring Harbor). Antibody producing cells are prepared by aseptic removal of a spleen from a mouse and disruption of the spleen into cells and removal of the larger tissue by washing with 2×OPI medium. A typical mouse spleen contains approximately 5×10⁷ to 2×10⁸ lymphocytes. If the hybridomas being prepared are not enriched by immunization to any antigen, spleens from more than one mouse can be used and the cells mixed. Equal numbers of spleen cells and myeloma cells are pelleted by centrifugation (400×g for 5 min) and the pellets separately resuspended 5 ml of medium without serum and then combined. Polyethylene glycol (PEG) is added to 0.84% from a 43% solution. The cells are gently resuspended in the PEG-containing medium and then repelleted by centrifugation at 400×g for 5 minutes, washed by resuspension in 5 ml of medium containing 20% FBS, repelleted and washed a second time in medium supplemented with 20% FBS, 1×OPI, and 1×AH (AH is a selection medium; 1×AH contains 5.8 μM azaserine and 0.1 mM hypoxanthine). Cells are incubated at 37° C. in a CO₂ incubator. Clones should be visible by microscopy after 4 days.

b. Isolating Hybridoma Cells

Stable hybridomas are selected by growth for several days in poor medium. The medium is then replaced with fresh medium and single hybridomas are isolated by limited dilution cloning. Because hybridoma cells have a very low plating efficiency, single cell cloning is done in the presence of feeder cells or conditioned medium. Freshly isolated spleen cells can be used as feeder cells as they do not grow in normal tissue culture conditions and are lost during expansion of the hybridoma cells. In this procedure a spleen is aspectically removed from a mouse and disrupted. Released cells are washed repeatedly in medium containing 10% FBS. A spleen typically produces 100 ml of 10⁶ cells per ml. The feeder cells are plated in 96-well plates, 50 μl per well, and grown for 24 hrs. Healthy hybridoma cells are diluted in medium containing 20% FBS, 2×OPI to a concentration of 20 cells per ml. Cells should be as free of clumps as possible. Add 50 μl of the diluted hybridoma cells to the feeder cells, final volume is 100 μl. Clones begin to appear in 4 days. Alternatively single cells can be isolated by single-cell picking by individually pipetting single cells and then depositing in wells containing feeder cells. Single cells can also be obtained by growth in soft agar. Once healthy, stable cultures are achieved the cells are maintained by growth in DME (or RPMI 1640) medium supplemented with 10% FBS. Stable cells can be stored in liquid nitrogen by slow freezing in medium containing a cryoprotectant such as dimethylsulfoxide (DMSO). The amount of antibody being produced by the cells is determined by measuring the amount of antibody in the culture supernatants by the ELISA method.

2. Purification of Antibodies from Hybridoma Culture Supernatants

Purification of antibodies from the individual culture supernatants is achieved by affinity binding. A number of affinity binding substrates are available. The procedure described below is based on commercially available substrates containing immobilized protein L (Pierce) and follows the manufacturers suggested procedure. Briefly, dilute the culture supernatant 1:1 with Binding buffer (0.1 M phosphate, 0.15 M sodium chloride (NaCl), pH 7.2) and apply up to 0.2 ml of the diluted sample to a Reacti-Bind™ Protein L Coated plate (Pierce) pre-equilibrated with Binding buffer. Wash the wells with 3×0.2 ml of binding buffer. Elute the bound antibodies with 2×0.1 ml of Elution buffer (0.1 M glycine, pH 2.8) and combine with 20 μl of 1 M Tris, pH 7.5. Desalt the purified antibodies using Sephadex G-25 gel filtration in combination with 96-well filter plates (Nalgene Nunc).

To create the phage panning substrates, antibodies separately purified as described above can be combined. Alternatively, purified antibody mixtures can be obtained by batch purification from pooled culture supernatants. Purification of antibodies from the pooled culture supernatants is also achieved by affinity binding. A number of affinity binding substrates are available. The procedure described below is based on commercially available substrates containing immobilized protein L (Pierce) and follows the manufacturers suggested procedure. Briefly, dilute the culture supernatant 1:1 with Binding buffer and apply up to 4 ml of the diluted sample to an Affinity Pack™ Immobilized Protein L Column (Pierce) pre-equilibrated with Binding buffer. Wash the column with 20 ml of Binding buffer, or until the absorbance at 250 nm has returned to background. Elute the bound antibodies with 6-10 ml of Elution buffer and collect into 1 ml fractions containing 100 μl of 1 M Tris, pH 7.5. Monitor release of bound proteins by absorbance at 280 nm and pool appropriate fractions. Desalt the purified antibodies using an Excellulose™ Desalting Column (Pierce).

3. Arraying Antibodies onto Filters

The antibodies purified from individual hybridoma cultures are spotted onto a membrane (such as; UltraBind membrane, Pall Gelman; FAST nitrocellulose coated slides, Schleicher & Schuell) 1 μl at a concentration of 1 μg-1 mg/ml in a buffer of 0.1 M PBS (phosphate buffered saline), pH 7.4, using an automated arraying tool (such as; PixSys NQ nanoliter dispensing workstation, Cartesian Technologies; BioChip Arrayer; Packard Instrument Company; Total Array System; BioRobotics; Affymetrix 417 Arrayer; Affymetrix). The spots are allowed to air dry 1-2 minutes. The UltraBind membrane contains active aldehyde groups that react with primary amines to form a covalent linkage between the membrane and the antibody. Unreacted aldehydes are blocked by incubation with a solution of 50 mM PBS, pH 7.4, 2% bovine serum albumin (BSA) for 30 minutes. The filter can be rinsed with 50 mM PBS and then air dried completely.

4. Panning a Phage Display Library on Paramagnetic Beads

A phage library containing random cysteine-constrained peptides expressed as part of an N-terminal genetic fusion to the gene III protein (gill) of the filamentous bacteriophage M13 is constructed essentially as described (Kay et al. (1996) Phage Display of Peptides and Proteins: A Laboratory Manual, Academic Press, San Diego). The random peptides are encoded by a DNA insert assembled from synthetic degenerate oligonucleotides and cloned into gill. These libraries are available commercially (Ph.D.-C7C™ Disulfide Constrained Peptide Library Kit, New England Biolabs). The Ph.D.-C7C™ library contains approximately 3.7×10⁹ independent clones.

Combine 2×10¹¹ phage virions from the Ph.D.-C7C™ library with 300 μg of the purified antibodies and 300 ng of the human IgG4 monoclonal antibody specific for the Fc domain of mouse IgG (Dynal; this monoclonal does not bind to human antibodies) to a final volume of 0.2 ml with TBST (50 mM Tris-HCl (pH 7.4), 150 mM NaCl, 0.1% Tween-20). The final concentration of antibody is approximately 10 nM. Incubate at room temperature for 20 minutes.

Combine the phage-antibody solution with Dynabeads Pan Mouse IgG (Dynal). The beads are supplied as a suspension in PBS, pH 7.4, 0.1% BSA, 0.02% sodium azide. The beads are washed with TBS (50 mM Tris-HCl (pH 7.4), 150 mM NaCl) several times prior to mixing with phage. The beads are separated from the solution by application of a magnet (Magnetic Particle Concentrator, Dynal). Add the phage-antibody solution to a concentration of 0.1 μg/10⁷ beads and incubate at 4° C. for 30 minutes with gentle tilting and rotation. Inclusion of the human antibody prevents selection of phage that bind to the human antibody immobilized on the Dynabeads. Additionally, inclusion of human proteins from a lysed human cell as a blocker prevents the selection of phage epitopes also present in human cells. The selected antibody-phage pairs should not be competed with proteins naturally present in the samples to be tested.

In the next step of the method, remove the fluid using the magnet and resuspend the beads in a Wash buffer of 1 ml of TBST. Repeat wash step 10 times. After the last wash step, elute the captured phage by suspending the beads in 1 ml of 0.2 M glycine-HCl, pH 2.2, 1 mg/ml BSA and incubating for 10 minutes at room temperature before recovering the fluid. The pH of the recovered fluid is immediately neutralized with the addition of 0.15 ml of 1 M Tris, pH 9.1. A small aliquot of the eluate is titered by infecting ER2738 Escherichia coli (E. coli) cells on LB-Tet plates.

Amplify the eluate by the addition of 20 ml of a mid-log culture of ER2738 E. coli and continue to grow in LB-Tet for 4.5 hours. Separate phage virions from E. coli cells by centrifugation at 10,000 rpm, 10 minutes, and transfer to fresh tube. Repeat, transferring the upper 80% of the supernatant to a fresh tube. Concentrate the phage by the addition of ⅙ volume of PEG/NaCl (20% w/v polyethylene glycol-8000, 2.5 M NaCl) followed by precipitation overnight at 4° C. The phage are recovered by centrifugation at 10,000 rpm for 15 minutes and the pellet is resuspended in 1 ml of TBS. Re-precipitate the phage in a microcentrifuge tube with PEG/NaCl and resuspend the pellet in 0.2 ml TBS, 0.02% sodium azide. Microcentrifuge for 1 minute to remove any residual material. The supernatant is the amplified eluate. Titer the amplified eluate and repeat the panning as described above 3 times. With each round of panning and amplification, the pool of phage becomes enriched for phage that bind the antibodies. If the concentration of phage used as input is kept constant, an increase in the number of phage recovered should occur. Phage can be stored at 4° C. or diluted 1:1 with sterile glycerol and stored at −20° C.

5. Staining the Antibody Array with Phage

The filter containing arrayed antibodies prepared from individual culture supernatants is probed with the enriched phage library. This method is similar to standard Western blotting or Dot blotting procedures. Briefly, the blocked filter is re-hydrated in TBST, pH 7.4, 0.1% v/v Tween-20, 1 mg/ml BSA, and incubated for 1 hour at 4° C. Phage are added to a concentration of 2×10¹¹ phage/ml and incubated with the filter for 30 minutes at room temperature. The hybridization solution is recovered and the filter is washed extensively with Blocking solution (TBST, pH 7.4, 0.1% v/v Tween-20, 1 mg/ml BSA and soluble proteins from human cells). To the Blocking solution add HRP-conjugated anti-M13 antibody (available commercially from, for, example, Amersham) diluted 1:100,000 to 1:500,000 in blocking buffer from a 1 mg/ml stock concentration and incubate for 1 hour with gentle shaking. Wash the membrane at least 4 to 6 times with TBST. Completely wet the blot in SuperSignal West Femto Substrate Working Solution (Pierce) for 5 minutes. The filter can be imaged by exposure to autoradiographic film (Kodak) or imaged using an imaging device such as a phosphoimager (BioRad) or charged coupled device (CCD) camera (Alphainnotech; Kodak).

6. Recovery of Phage from Filter and Sequencing the Epitopes

Phage can be recovered from the filter by cutting out the spots containing phage identified from the imaging. Phage are eluted from the filter by suspending the filter piece in 0.5 ml of 0.2 M glycine-HCl, pH 2.2, 1 mg/ml BSA and incubating for 10 minutes at room temperature before recovering the fluid. The pH of the recovered fluid is immediately neutralized with the addition of 0.075 ml of 1 M Tris, pH 9.1. A small aliquot of the eluate is titered by infecting ER2738 E. coli cells on LB-Tet plates. Isolated plaques (typically 10 plaques) are picked for DNA isolation and sequenced to define a consensus epitope. Plaques are amplified by inoculating 1 ml cultures of ER2738 E. coli cells freshly diluted 1:100 from a healthy mid-log culture, using a sterile pipet tip or toothpick and incubated at 37° C. for 4 to 5 hours with shaking. Phage are recovered by microcentrifugation for 30 seconds, and 0.5 ml of the supernatant transferred to a fresh tube and 0.2 ml of PEG/NaCl is added and allowed to stand at room temperature after gentle mixing for 10 minutes. Pellet the phage by centrifugation for 10 minutes at top speed in a microcentrifuge. Discard any remaining supernatant and thoroughly suspend the pellet in 0.1 ml iodine buffer and 0.25 ml ethanol to precipitate single-stranded DNA. The DNA pellets are washed in 70% ethanol and air-dried. DNA is sequenced by standard methods.

B. Selective Infection

Selective infection technologies, such as phage display, are used to. identify interacting protein-peptide pairs. These systems take advantage of the requirement for protein-protein interactions to mediate the infection process between a bacteria and an infecting virus (phage). The filamentous Ml 3 phage normally infects E. coli by first binding to the F pilus of the bacteria. The virus binds to the pilus at a distinct region of the F pilin protein encoded by the traA gene. This binding is mediated by the minor coat protein (protein 3) on the tip of the phage. The phage binding site on the F pilin protein (a 13 amino acid sequence on the traA gene) can be engineered to create a large population of bacteria expressing a random mixture of phage binding sites.

The phage coat protein (protein 3) can also be engineered to display a library of diverse single chain antibody structures. Infection of the bacteria and internalization of the virus is therefore mediated by an appropriate antibody-peptide epitope interaction. By placing appropriate antibiotic resistance markers on the bacteria and virus DNA, individual colonies can be selected that contain both genes for the antibody and its corresponding peptide epitope. The recombinant antibody phage display library prepared from non-immunized mice and the bacterial strains containing a random peptide sequence in the phage binding site in the traA gene are commercially available (Biolnvent, Lund, Sweden). Creation of a recombinant antibody library is described below.

C. Expression and Purification of Antibodies

Purification of antibodies from hybridoma supernatants is achieved by affinity binding. A number of affinity binding substrates are available. The procedure described below is based on commercially available substrates containing immobilized protein L (Pierce) and follows the manufacturers suggested procedure. Briefly, dilute the culture supernatant 1:1 with Binding buffer (0.1 M phosphate, 0.15 M sodium chloride (NaCl), pH 7.2) and apply up to 4 ml of the diluted sample to an Affinity Pack™ Immobilized Protein L Column (Pierce) pre-equilibrated with Binding buffer. Wash the column with 20 ml of Binding buffer, or until the absorbance at 250 nm has returned to background. Elute the bound antibodies with 6-10 ml of Elution buffer (0.1 M glycine, pH 2.8) and collect into 1 ml fractions containing 100 μl of 1 M Tris, pH 7.5. Monitor release of bound proteins by absorbance at 280 nm and pool appropriate fractions. Desalt the purified antibodies using an Excellulose™ Desalting Column (Pierce). The purification can be scaled as appropriate. Alternatively, antibodies can be purified by affinity chromatography using protein A (or protein G) HiTrap columns (Amersham Pharmacia) and an FPLC chromatographic system (Amersham Pharmacia). Following the manufacturers suggested protocols.

Recombinant antibodies are expressed and purified as described (McCafferty et al. (1996) Antibody engineering: A practical Approach, Oxford University Press, Oxford). Briefly, the gene encoding the recombinant antibody is cloned into an expression plasmid containing an inducible promoter. The production of an active recombinant antibody is dependant on the formation of a number of intramolecular disulfide bonds. The environment of the bacterial cytoplasm is reducing, thus preventing disulfide bond formation. One solution to this problem is to genetically fuse a secretion signal peptide onto the antibody which directs its transport to the non-reducing environment of the periplasm (Hanes et al. (1997) Proc. Natl. Acad. Sci. U.S.A. 94:4937-4942).

Alternatively, the antibodies can be expressed as insoluble inclusion bodies and then refolded in vitro under conditions that promote the formation of the disulfide bonds. Inoculate 0.5 liters of LB medium containing an appropriate antibiotic and shake for 10 hours at 32° C. Use the starter culture to inoculate 9.5 liters of production medium (3 g ammonium sulfate, 2.5 g potassium phosphate, 30 g casein, 0.25 g magnesium sulfate, 0.1 mg calcium chloride, 10 ml M-63 salts concentrate, 0.2 ml MAZU 204 Antifoam (Mazer Chemicals), 30 g glucose, 0.1 mg biotin, 1 mg nicotinamide, appropriate antibiotic, per liter, pH 7.4). Ferment using a Chemap (or like) fermenter at pH 7.2, aeration at 1:1 v/v Air to medium per minute, 800 rpm agitation, 32° C. When the absorbance at 600 nm reaches 18-20, raise temperature to 42° C. for 1 hour then cool to 10° C. for 10 minutes before harvesting cell paste by centrifugation at 7,000×g for 10 minutes. Recovery is typically 200-300 g wet cell paste from a 10 liter fermentation and should be kept frozen.

The recombinant antibody is solubilized from the thawed cell paste by resuspension in 2.5 liters cell lysis buffer (50 mM Tris-HCl, pH 8.0, 1.0 mM EDTA, 100 mM KCl, 0.1 mM phenylmethylsulfonyl fluoride; PMSF) and kept at 4° C. The resuspended cells are passed through a Manton-Gaulin cell homogenizer 3 times and the insoluble antibodies recovered by centrifugation at 24,300×g for 30 minutes at 6° C. The pellet is resuspended in 1.2 liters of cell lysis buffer and the homogenization and recovery is repeated as described above 5 times. The washed pellet can be stored frozen. The recombinant antibody is renatured by resolubilization in 6 ml denaturing buffer (6 M guanidine hydrochloride, 50 mM Tris-HCl, pH 8.0, 10 mM calcium chloride, 50 mM potassium chloride) per gram of cell pellet. The supernatant from a centrifugation at 24,300×g for 45 minutes at 6° C. is diluted to optical density of 25 at 280 nm with denaturing buffer and slowly diluted into cold (4-10° C.) refolding buffer (50 mM Tris-HCl, pH 8.0, 10 mM calcium chloride, 50 mM potassium chloride, 0.1 mM PMSF) until a 1:10 dilution is achieved over a 2 hour period. The solution is left to stand for at least 20 hours at 4° C. before filtering through a 0.45 μm microporous membrane. The filtrate is then concentrated to about 500 ml before final purification using an HPLC.

The filtrate is dialyzed against HPLC buffer A (60 mM MOPS, 0.5 mM calcium acetate, pH 6.5) until the conductivity matches that of HPLC buffer A. The dialyzed sample (up to 60 mg) is loaded onto a 21.5 mm×150 mm polyaspartic acid PolyCAT column, equilibrated with HPLC buffer A and eluted from the column with a 50 minute linear gradient between HPLC buffers A and B (HPLC buffer B is 60 mM MOPS, 0.5 mM calcium acetate, pH 7.5). Remaining protein is eluted with HPLC buffer C (60 mM MOPS, 100 mM calcium acetate, pH 7.5). The collected fractions are analyzed by SDS-PAGE.

D. Exemplary Array and Use Thereof for Capture of Proteins with Tags and Detection Thereof.

As also described in EXAMPLE 8, to demonstrate the functioning of the methods herein, capture antibodies, specific, for example, for various peptide epitopes, such as human influenza virus hemagglutinin (HA) protein epitope, which has the amino acid sequence YPYDVPDYA (SEQ ID No. 92), are used to tag, for example, scFvs. For example, an scFv with antigen specificity for human fibronectin (HFN) is tagged with an HA epitope, thus generating a molecule (HA-HFN), which is recognized by an antibody specific for the HA peptide and which has antigen specificity of HFN.

After depositing the capture antibodies, including anti-HA tag capture antibodies onto a membrane, such as a nitrocellulose membrane, they are dried at ambient temperature and relative humidity for a suitable time period (e.g., 10 minutes to 3 hr, which can be determined empirically). After drying, membranes with deposited and dried anti-HA capture antibodies are blocked, if necessary, with a protein-containing solution such as Blocker BSA™” (Pierce) diluted to 1×in phosphate-buffered saline (PBS) with Tween-20 (polyoxyethylenesorbitan monolaurate; Sigma) added to a final concentration of 0.05% (vol:vol) to eliminate background signal generated by non-specific protein binding to the membrane. For subsequent description contained herein, blocking agent is referred to as BBSA-T, and PBS with 0.05% (vol:vol) Tween-20 is referred to as PBS-T. Blocking times can be varied from 30 mm to 3 hr, for example. For all subsequent incubations (except for washes) described below for this procedure, incubation times are varied from about 20 min to 2 hr. Likewise, incubation temperatures can be varied from ambient temperature to about 37° C. In all instances, the precise conditions can be determined empirically.

After blocking the membranes containing the deposited anti-HA capture antibodies, an incubation with peptide tagged scFvs can be performed. Purified scFvs (or bacterial culture supernatants, or various crude subcellular fractions obtained during purification of such scFvs from E. coli cultures harboring plasmid constructs that direct the expression of such scFvs upon induction, for example HA-HFN scFv, containing the HA peptide tag, can be diluted to various concentrations (for example, between 0.1 and 100 μg/ml) in BBSA-T. Membranes with deposited anti-peptide tag capture antibodies are then incubated with this HA-HFN scFv antigen solution. Membranes with deposited anti-HA capture antibodies and bound HA-HFN scFv antigen are then washed one or more times (e.g., 3 times) with PBST, for suitable periods of time (e.g., 3-5 min per wash), at various temperatures.

Membranes with deposited anti-HA capture antibodies and bound HA-HFN scFcv antigen is then washed a plurality (typically 3 times) with PBS-T, for suitable times (typically 3 to 5 min per wash, for example), at various temperature. Membranes with deposited anti-HA capture antibodies and bound HA-HFN scFv are then incubated with, for purposes of demonstration, biotinylated human fibronectin (Bio-HFN), which is an antigen that is recognized by the capture HA-HFN scFv. Bio-HFN is serially diluted (e.g., from 1 to 10 μg/ml) in BBSA-T. The resulting membranes are washed a suitable number of time (typically 3) with PBS-T for a suitable period of time (typically 3 to 5 min per wash) at various temperatures, and are then incubated with Neutravidin•HRPO (Pierce) serially diluted (e.g., 1:1000 to 1:100,000 in BBSA-T). The resulting membranes are washed as before, rinsed with PBS and developed with Supersignal™ ELISA Femto Stable Peroxide Solution and Supersignal™ ELISA Femto Lumino Enhancer Solution (Pierce), and then imaged using an imaging system, such as, for example, a Kodak Image Station 440CF or other such imaging system. A 1:1 mixture of peroxide solution:luminol is prepared and a small volume is plated on the platen of the image station.

Membranes are then placed array-side down into the center of the platen, thus placing the surface area of the antibody-containing portion of the membrane into the center of the imaging field of the camera lens. In this way the small volume of developer, present on the platen, can then contact the entire surface area of the antibody-containing portion of the slide. The Image Station cover is then closed for antibody array image capture. Camera focus (zoom) varies depending on the size of the membrane being imaged. Exposure times can vary depending on the signal strength (brightness) emanating from the developed membrane. Camera f-stop settings are infinitely adjustable between 1.2 and 16.

Archiving and analysis of array images can be performed, for example, using the Kodak ID 3.5.2 software package. Regions of interest (ROIs) are drawn using the software to frame groups of capture antibodies (printed at known locations on the arrays). Numerical ROI values, representing net, sum, minimum, maximum, and mean intensities, as well standard deviations and ROI pixel areas, for example, are automatically calculated by the software. These data then are transformed, for example into Microsoft Excel, for statistical analyses.

### Example 2

Preparation of a Tagged cDNA Library and Preparation of Primers

The array of antibodies to tags is used as a sorting device. Proteins from a cDNA library are bathed over the surface of the array and bind to spots containing antibodies that specifically recognize and bind peptide epitopes that have been genetically fused to the library proteins. Key to this system is the ability to randomly attach and evenly distribute a relatively small number of tags (approximately 1,000) onto a relatively large number of genes (approximately 10⁶ to 10⁹). To ensure that the tags are evenly distributed among the genes in the library, the tags should be incorporated into the genes before amplification by PCR. A variety of methods are described herein to accomplish this task.

To create a cDNA library, message RNA (mRNA) is first isolated from cells and then converted into DNA in two steps. In the first step, the enzyme RNA-dependant DNA polymerase (reverse transcriptase; RTase) is used to produce a RNA:DNA duplex molecule. The RNA strand is then replaced by a newly synthesized DNA strand using DNA-dependant DNA polymerase (DNA polymerase or a fragment of the polymerase such as the Klenow fragment). The DNA:DNA duplex molecule is then be amplified by PCR.

One method relies on the use of a collection of primers for the first strand cDNA synthesis that contain DNA sequences for the tags. In this case, the primers are single stranded oligonucleotides and the tags are incorporated before the second strand cDNA synthesis. After the second strand cDNA synthesis the resulting molecules are amplified by PCR. In another method, the DNA:DNA duplex molecule is created using primers that incorporate a unique restriction enzyme cut site at the 3′-end of the new molecule which is cut to leave a defined nucleotide overhang. A collection of linker DNA molecules containing a complementary overhang and DNA sequences for the tags is ligated onto the DNA molecules of the cDNA library and then amplified by PCR. In the second method, the linkers are double stranded molecules and the tags are incorporated after the second strand cDNA synthesis. Both methods depend on the generation of a large diverse collection of molecules as either primers or linkers. The preparation of these molecules is described below.

A. Method I: Primer Extension

Library construction starts with the isolation of mRNA. Direct isolation of mRNA is done by affinity purification using oligo dT cellulose. Kits containing the reagents for this method are commercially available from a number of suppliers (Invitrogen, Stratagene, Clonetech, Ambion, Promega, Pharmacia) and is isolated according to manufacturers suggested methods. Additionally, mRNA purified from a number of tissues can also be obtained directly from these suppliers.

The cDNA library construction is done essentially as described (Sambrook et al. (1989) Molecular Cloning: A Laboratory Manual, 2nd Edition, Cold Spring Harbor Laboratory Press). First strand synthesis is done by mixing the following at 4° C. to 50 μl final volume; 10 μg mRNA (poly(A)⁺ RNA), 10 μg of V_{LFOR}-common primer mix (V_{LFOR}-common is described below), 50 mM Tris-HCl, pH 7.6, 70 mM potassium chloride, 10 mM magnesium chloride, dNTP mix (1 mM each), 4 mM dithiothreitol, 25 units RNase inhibitor, 60 units murine reverse transcriptase (Pharmacia). Incubate for 1 hour at 37° C. For the second strand synthesis a mixture of the following is directly added to the first strand synthesis solution to a final volume of 142 μl; 5 mM magnesium chloride, 70 mM Tris-HCl, pH 7.4, 10 mM ammonium sulfate, 1 unit RNAse H, 45 units E. coli DNA polymerase 1, and allowed to incubate at room temperature for 15 minutes. To this mix is added 5 μl of 0.5 M EDTA, pH 8.0, to stop the reaction. The final volume should be 150 μl. The newly synthesized cDNA is purified by extraction with an equal volume of phenol:chloroform and the unincorporated dNTPs are separated by chromatography through Sephadex G-50 equilibrated in TE buffer (10 mM Tris-HCl, 1 mM EDTA), pH 7.6, containing 10 mM sodium chloride. The eluted DNA is precipitated by the addition of 0.1× volume 3 M sodium acetate (pH 5.2) and 2 volumes of ethanol incubated at 25° C. for at least 15 minutes and recovered by centrifugation at 12,000 g for 15 minutes at 4° C., washed with 70% ethanol, air dried, then redissolved in 80 μl of TE (pH 7.6).

An alternative method involves the generation of a cDNA library using solid-phase synthesis (McPherson et al. (1995) PCR 2: A Practical Approach. Oxford University Press, Oxford). In this method the primer used for first strand cDNA synthesis is coupled to a solid support (such as paramagnetic beads, agarose, or polyacrylamide). The mRNA is captured by hybridization to the immobilized oligonucleotide primer and reverse transcribed. Immobilization of the cDNA has the advantage of facilitating buffer and primer changes. Further, cDNA immobilized to a solid phase increases the stability of the cDNA enabling the same library to be amplified multiple times using different sets of primers. Generation of primers using solid-phase PCR is described herein; any method for generating such primers is contemplated.

B. Method II: Linker Fusion

As with Method I, library construction starts with the isolation of mRNA. Direct isolation of mRNA is done by affinity purification using oligo dT cellulose. Kits containing the reagents for this method are commercially available from a number of suppliers (Invitrogen, Stratagene, Clonetech, Ambion, Promega, Pharmacia) and is isolated according to manufacturers suggested methods. Additionally, mRNA purified from a number of tissues can also be obtained directly from these suppliers.

The cDNA library construction is done essentially as described (Sambrook et al (1989) Molecular Cloning: A Laboratory Manual, 2nd Edition, Cold Spring Harbor Laboratory Press). First strand synthesis is done by mixing the following at 4° C. to 50 μl final volume; 10 μg mRNA (poly(A)⁺ RNA), 10 μg of 5′-restriction sequence-oligo(dT)₁₂₋₁₈ primers, 50 mM Tris-HCl, pH 7.6, 70 mM potassium chloride, 10 mM magnesium chloride, dNTP mix (1 mM each), 4 mM dithiothreitol, 25 units RNase inhibitor, 60 units murine reverse transcriptase (Pharmacia). Incubate for 1 hour at 37° C. For the second strand synthesis, a mixture of the following is directly added to the first strand synthesis solution to a final volume of 142 μl; 5 mM magnesium chloride, 70 mM Tris-HCl, pH 7.4, 10 mM ammonium sulfate, 1 unit RNAse H, 45 units E. coli DNA polymerase I, 1 U of the restriction enzyme recognizing the site on the 5′-end of the oligo (dT) primer and allowed to incubate at room temperature for 15 minutes. To this mix is added 5 μl of 0.5 M EDTA, pH 8.0, to stop the reaction. The final volume should be 150 μl. The newly synthesized cDNA is purified by extraction with an equal volume of phenol:chloroform and the unincorporated dNTPs are separated by chromatography through Sephadex G-50 equilibrated in TE buffer (10 mM Tris-HCl, 1 mM EDTA), pH 7.6, containing 10 mM sodium chloride. The eluted DNA is precipitated by the addition of 0.1× volume 3 M sodium acetate (pH 5.2) and 2 volumes of ethanol incubated at 25 C for at least 15 minutes and recovered by centrifugation at 12,000 g for 15 minutes at 4° C., washed with 70% ethanol, air dried, then redissolved in 80 μl of TE (pH 7.6) and the DNA concentration measured by absorption at 260 nm. The cDNA library is then tagged by the addition of unique linkers to the restriction digested 3′-end of the cDNA molecules. Linkers are prepared as described below and ligated to the purified cDNA in a reaction containing an equal number of cDNA and linker molecules, 10 U T4 DNA ligase (100 U/μl), 1 μl 10 mM ATP, 1 μl Ligation buffer (0.5 M Tris-HCl, pH 7.6, 100 mM MgCl₂, 100 mM DTT, 500 μg BSA), and water to 10 μl final volume, and incubated for 4 hours at 16° C. After ligation the cDNA is amplified using a linker specific primer. The PCR conditions are; 35 μl of water, 5 μl of Taq buffer (100 mM Tris-HCl, pH 8.3, 500 mM KCl, 15 mM MgCl₂, and 0.01% (w/v) gelatin), 1.5 μl 5 mM dNTP mix (equimolar mixture of dATP, dCTP, dGTP, dTTP with a concentration of 1.25 mM each dNTP), 2.5 μl of linker specific primers (10 pmol/μl), 2.5 μl of V_{HBACK} primers (10 pmol/μl), 2.5 μl of cDNA and overlay 2 drops of mineral oil. Heat to 94° C. and add 1 U of Taq DNA polymerase. Amplify using 30 cycles of 94° C. for 1 minute, 57° C. for 1 minute, 72° C. for 2 minutes. To the PCR reaction add 7.5M ammonium acetate to a final concentration of 2 M and precipitate the DNA by the addition of 1 volume of isopropanol and incubate at 25° C. for 10 minutes. Pellet the DNA by centrifugation (13,000 rpm, 10 minutes) and dissolve the pellet in 100 μl of 0.3 M sodium acetate and reprecipitate by the addition of 2.5 volumes of ethanol. Incubate at −20° C. for 30 minutes. Pellet the DNA by centrifugation (13,000 rpm, 10 minutes) and rinse the pellet with 70% ethanol. Dry the pellet in vacuo for 10 minutes then redissolve the dried pellets in 10⁻¹⁰⁰ μl of TE buffer to 0.2-1.0 mg/ml. Determine the DNA concentration by absorbance at 260 nm.

### Example 3

Recombinant Antibodies

Antibodies are highly valuable reagents with applications in therapeutics, diagnostics and basic research. There is a need for new technologies that enable the rapid identification of highly specific, high affinity antibodies. The most valuable antibodies are those that can be directly used in the treatment of disease. Therapeutic antibodies have become an accepted part of the pharmaceutical landscape. Recombinant antibodies can be made from human antibody genes to create antibodies that are less immunogenic than non-human monoclonal antibodies. For example, Herceptin, a recombinant humanized antibody that binds to the ectodomain of the p₁₈₅^{HER2/neu} oncoprotein, is now an accepted and important therapy for the treatment of breast cancer.

Other examples of therapeutic antibodies include; OKT3 for the treatment of kidney transplant rejection; Digibind for the treatment of digoxin poisoning; ReoPro for the treatment of angioplasty complications; Panorex for the treatment of colon cancer; Rituxan for the treatment of non-Hodgkin's lymphoma; Zenapax for the treatment of acute kidney transplant rejection; Synagis for the treatment of infectious diseases in children; Simulect for the treatment of kidney transplant rejection; Remicade for the treatment of Crohn's disease. Current methods to discover therapeutic antibodies are laborious and time intensive.

Antibodies have transformed the medical diagnostics industry. The specificity of antibodies for their substrates has enabled their use in clinical tests for a wide variety of protein disease markers such as prostate specific antigen, small molecule metabolites and drugs. New antibody-based diagnostic tools aid physicians in making better diagnostic assessments of disease stages and prognostic predictions.

Antibodies are also powerful research reagents used to purify proteins, to measure the amounts of specific proteins and other biomolecules in a sample, to identify and measure protein modifications, and to identify the location of proteins in a cell. The current knowledge of the complex regulatory and signaling systems in cells is largely due to the availability of research antibodies.

As part of our bodies immune defense system, antibodies are designed to specifically recognize and tightly bind other proteins (antigens). The body has evolved an elegant system of combinatorial gene shuffling to produce an enormous diversity of antibody structures. Our bodies use a combination of negative selection (apoptosis) and positive selection (clonal expansion) to identify useful antibodies and eliminate billions of non-useful structures. The binding of the antibody for its antigen is further refined in a second phase of selection known as “affinity maturation”. In this process further diversity is created by fortuitous somatic mutations that are selected by clonal expansion (i.e., cells expressing antibodies of higher affinity proliferate at faster rates than cells producing weaker antibodies). These processes can now be mimicked in a test tube.

Antibodies are composed of four separate protein chains held strongly together by chemical bridges; two longer “heavy” chains and two shorter “light” chains. The extreme range of antigen recognition by antibodies is accomplished by the structural variation in the antigen recognition sites at the ends of the antibody molecules where the “heavy” and “light” chains come together (called the “variable region”). The antibody producing cells of the immune system randomly rearrange their DNA to produce a single combination of variable heavy (V_{H}) and variable light (V_{L}) chain genes.

The process of antibody assembly can now be accomplished using recombinant DNA technology. Consensus DNA sequences flanking the V_{H} and V_{L} chain genes can serve as priming regions that allow amplification of these genes by PCR from mRNA purified from populations of human cells and the amplified genes can be randomly assembled in a test tube mimicking the natural process of recombination. The assembled recombinant antibody genes form a collection, or “library”, that typically contains over a billion different combinations.

To identify the desired antibody clones in the library a variety of selection schemes have been developed. Protein display technologies link genotypes (the genetic material or DNA) with phenotypes (the structural expression of the genetic material or proteins). The ability to express proteins on the surfaces of viruses or cells can be coupled with affinity selection techniques. This powerful combination enables proteins with the highest affinities to be selected out of large diverse populations, often containing over a billion different structural variations.

In filamentous bacteriophage display systems, antibody gene libraries are expressed on the tips of bacteria viruses (phage) and those displaying high affinity antibodies are selected by binding to immobilized antigens. Repeated rounds of selection enriches for antibodies containing the desired properties. However, phage display is limited by the DNA uptake ability of bacterial cells and artificial selection biases.

In ribosome display, cloned antibody genes are transcribed into mRNA and then translated in vitro such that the translated proteins remain attached to their cognate mRNAs through association with the ribosomes. The antibody-ribosome-mRNA complexes are selected by affinity purification and amplified by PCR. Repeated rounds of selection enriches for antibodies containing the desired properties. Another approach uses mRNA-protein fusions created by covalent puromycin linkage of the mRNA to its transcribed protein and the resulting hybrid molecules are selected by affinity enrichment.

A. Tagging a Recombinant Antibody cDNA Library

The following describes the method for tagging a recombinant antibody cDNA library. The tagging primer, V_{LFOR}, includes five different functional units (J_{kappa for}, Epitope, D, and Common) (FIGS. 10 and 11). The J_{kappa for} region functions to specifically recognize and amplify consensus sequences located on mRNA encoding the immunoglobulin genes. Natural immunoglobulin molecules are made up of two identical heavy chains (H chains) and two identical light chains (L chains). B-cells express H and L chain genes as separate mRNA molecules. The H and L chain mRNAs are composed of functional regions: variable regions and constant regions. The variable heavy chain region (V_{H}) is created by recombination of variable, diversity, and joining genes (referred to as VDJ recombination). The variable light chain region (V_{L}) is created by recombination of variable and joining genes (referred to as VJ recombination). The joining genes precede the constant region genes of the light chain.

The J_{kappa for} sequences constitute a set of 25 different DNA sequences that have been identified and used to amplify a large number of V_{L} genes. These sequences are commonly used in the creation of recombinant antibody libraries and serve as primers to initiate amplification of the V_{L} genes by PCR.

The functional region “D” refer to sequences which are used to “divide” the library by providing sequences for specific PCR amplification. They are composed of a known sequences. The D sequences should be designed for optimal primer binding to result in specific amplification of genes containing the D sequences. Design and selection of the D sequences can be accomplished using well known standard procedures. An example is the sequence 5′-GATC(A)(T)GATC(G)TC(C)GA(A)G-3′ SEQ ID No. 1 in which the positions in parenthesis vary. Oligonucleotides encoding the D sequences are designed to provide a minimum of sequence identity among each other and among known sequences in the database, to maximize specific amplification during the PCR. Incorporating these sequences in the tags enables the library to be divided by PCR amplification using primers that are specific for the various sequences. For example, if the library has been tagged with the above sequence, a primer containing the sequence 5′-GATC(A)(T)GATC(G)TC(C)GA(A)G-3′ SEQ ID No. 2 specifically amplifies one group of tagged molecules; whereas a primer containing the sequence 5′-GATC(G)(G)GATC(A)TC(A)GA(A)G-3′ SEQ ID No. 3 amplifies a different group of tagged molecules.

The functional region “Epitope” contains sequences encoding the peptide “epitopes” specifically recognized by the capture agents, such as antibodies, in the array. These sequences are joined to the J_{kappa for} sequences in-frame so that a functional peptide tag results. A termination sequence follows the epitope.

The functional region “common” (C) contains a non-variable sequence that includes termination sequences for transcription and translation. As this sequence is common to all the tags, it can be used to amplify the entire collection of molecules in the tagged cDNA library. The possible number of different sequences that can be used for creating the primer/linker collection is extremely large and can be readily deduced.

B. Solid Phase PCR for Generation of Primers and Other Methods

Solid phase PCR for generation of primers is exemplified for use in this method. In this method, the upstream oligonucleotide is coupled to a solid phase (such as paramagnetic beads, agarose, or polyacrylamide). Coupling is achieved by first coupling an aminolink to the 5′-end of the oligonucleotide prior to cleavage of the oligonucleotide from the synthesizer support. The amino link then can be reacted with an activated solid phase containing NHS-, tosyl-, or hydrazine reactive groups.

An alternative method involves using (+) strand and (−) strand oligonucleotides separately synthesized by micro-scale chemical DNA synthesis for the 4 functional regions. The oligonucleotides are designed to contain overlapping regions such that when mixed in equal amounts, they combine by hybridization to form a collection of “nicked” double-stranded DNA molecules. The nicks are enzymatically sealed with DNA ligase. The sealed double stranded molecules are used as a template for DNA synthesis using a biotinylated oligonucleotide as the primer. To generate single-stranded molecules for primers, the biotinylated strand is purified by binding to streptavidin-coated paramagnetic beads. The non-biotinylated strand is separated after denaturation.

### Example 4

### Construction of Recombinant Antibody Libraries

A. Preparation of Recombinant Antibodies

Recombinant antibody libraries are prepared by methods known to those of skill in the art (see, e.g., et al. (1996) Phage Display of Peptides and Proteins: A Laboratory Manual, Academic Press, San Diego); McCafferty et al. (1996) Antibody engineering: A practical Approach, Oxford University Press, Oxford). Functional antibody fragments can be created by genetic cloning and recombination of the variable heavy (V_{H}) chain and variable light (V_{L}) chain genes from a mouse or human. The V_{H} and V_{L} chain genes are cloned by reverse transcribing poly(A)RNA isolated from spleen tissue and then using specific primers to amplify the V_{H} and V_{L} chain genes by PCR. The V_{H} and V_{L} chain genes are joined by a linker region (a typical linker to produce a single-chain antibody fragment, scFv, includes DNA sequences encoding the amino acid sequence (Gly₄Ser)₃). After the V_{H}-linker-V_{L} genes have been assembled and amplified by PCR, the products are transcribed and translated directly or cloned into an expression plasmid and then expressed either in vivo or in vitro.

Library construction starts with the isolation of mRNA. Direct isolation of mRNA is done by affinity purification using oligo dT cellulose. Kits containing the reagents for this method are commercially available from a number of suppliers (Invitrogen, Stratagene, Clonetech, Ambion, Promega, Pharmacia) and is isolated according to manufacturers suggested methods. The mRNA purified from a number of tissues can also be obtained directly from these suppliers. The first strand cDNA synthesis is essentially as described above.

Amplification of the V_{H} and V_{L} chain genes is accomplished with sets of PCR primers that correspond to consensus sequences flanking these genes (McCafferty et al. (1996) Antibody engineering: A practical Approach, Oxford University Press, Oxford). In a 0.5 ml microcentrifuge tube mix the following; 35 μl of water, 5 μl of Taq buffer (100 mM Tris-HCl, pH 8.3, 500 mM KCl, 15 mM MgCl₂, and 0.01% (w/v) gelatin), 1.5 μl 5 mM dNTP mix (equimolar mixture of dATP, dCTP, dGTP, dTTP with a concentration of 1.25 mM each dNTP), 2.5 μl of FOR primers (10 pmol/μl), 2.5 μl of BACK primers (10 pmol/μl). The mixture is irradiated with UV light at 254 nm for 5 minutes. In a new 0.5 ml tube add 47.5 μl of the irradiated mix to 2.5 μl of cDNA and optionally overlay 2 drops of mineral oil. Heat to 94° C. and add 1 U of Taq DNA polymerase. Amplify using 30 cycles of 94° C. for 1 minute, 57° C. for 1 minute, 72° C. for 2 minutes. Isolate and purify the amplified DNA from the primers by electrophoresis in a low melting temperature agarose gel. Estimate the quantities of purified V_{H} and V_{L} chain DNA. For a mouse antibody library set up the following reaction; approximately 50 ng each of V_{H} and V_{L} chain DNA and linker DNA, 2.5 μl of Taq buffer, 2 μl of 5 mM dNTP mix, water up to 25 μl, and 1 U of Taq DNA polymerase (1U/μl). Amplify using 20 cycles of 94° C. for 1.5 minute, 65° C. for 3 minutes.

To the reaction add 25 μl of the following mixture; 2.5 μl of Taq buffer, 2 μl of 5 mM dNTP, 5 μl of V_{HBACK} primers (10 pmol/μl), 5 μl of V_{LFOR} primers (10 pmol/μl), water and 1 U of Taq DNA polymerase. Amplify using 30 cycles of 94° C. for 1 minute, 50° C. for 1 minute, 72° C. for 2 minutes and a final extension step at 72° C. for 10 minutes. Isolate and purify the amplified DNA from the primers by electrophoresis in a low melting temperature agarose gel. A further amplification is done using primers that incorporate DNA sequences required for efficient transcription and translation of the gene or appropriate restriction sites for cloning into an expression plasmid. The amplification is essentially as described above. After amplification the DNA is purified and transcribed/translated or digested with a restriction enzyme and cloned.

B. Expression and Purification of Recombinant Antibodies

For in vitro transcription/translation with E. coli S30 systems (McPherson et al. (1995) PCR 2: A Practical Approach, Oxford University Press, Oxford; Mattheakis et al. (1994) Proc. Natl. Acad. Sci. U.S.A. 91; 9022-9026) amplify with an upstream primer containing T7 RNA polymerase initiation sites and an optimally positioned Shine-Dalgarno sequence (AGGA) such as: 5′-gaattctaatacgactcactataGGGTTAACTTTAAGAAGGAGATATACAT ATGATGGTCCAGCT(G/T)CTCGAGTC-3′ (SEQ ID NO. 4, non-transcribed sequences in lowercase). PCR products used for in vitro transcription/translation are purified as follows. To the PCR reaction add 7.5M ammonium acetate to a final concentration of 2 M and precipitate the DNA by the addition of 1 volume of isopropanol and incubate at 25° C. for 10 minutes. Pellet the DNA by centrifugation (13,000 rpm, 10 minutes) and dissolve the pellet in 100 μl of 0.3 M sodium acetate and reprecipitate by the addition of 2.5 volumes of ethanol. Incubate at −20° C. for 30 minutes. Pellet the DNA by centrifugation (13,000 rpm, 10 minutes) and rinse the pellet with 70% ethanol. Dry the pellet in vacuo for 10 minutes then redissolve the dried pellets in 10⁻¹⁰⁰ μp of TE buffer to 0.2-1.0 mg/ml. Determine the DNA concentration by absorbance at 260 nm. Coupled transcription/translation is carried out with the following reaction. To a 0.5 ml tube on ice add 20 μl of Premix (87.5 mM Tris-acetate, pH 8.0, 476 mM potassium glutamate, 75 mM ammonium acetate, 5 mM DTT, 20 mM magnesium acetate, 1.25 mM each of 20 amino acids, 5 mM ATP, 1.25 mM each of CTP, TTP, GTP, 50 mM phosphoenolpyruvate(trisodium salt), 2.5 mg/ml E. coli tRNA, 87.5 mg/ml polyethylene glycol (8000 MW), 50 μg/ml folinic acid, 2.5 mM cAMP), purified PCR product (approximately 1 μg in TE), 40 U phage RNA polymerase (40 U/ul), water to give final volume of 35 μl. Add 15 μl of S30, mix gently and incubate at 37° C. for 60 minutes. Terminate reaction by cooling back down to 0° C.

For in vitro transcription/translation with rabbit reticulocyte lysates (Makeyev et al. (1999) FEBS Letters 444:177-180) the assembled V_{H}-linker-V_{L} gene fragments are amplified in a fresh PCR mixture containing 250 nM of each T7V_{H} and V_{LFOR} primers and amplified for 25 cycles of 94° C. for 1 minute, 64° C. for 1 minute, 72° C. for 1.5 minutes. The upstream primer, T7V_{H} has the sequence: 5′-taatacgactcactataGGGAAGCTTGGCCACCATGGTCCAGCT(G/T)CTCGA GTC-3′ (SEQ ID No. 5), which includes a T7 RNA polymerase promoter (lower case) and an optimally positioned ATG start codon.

Alternatively, the recombinant antibodies can be expressed in vivo in a variety of expression systems, such as, but are not limited to: bacterial, yeast, insect and mammalian systems and cells. Expression in E. coli is described above.

### Example 5

### Creation and Production of scFvs

The HFN7.1 hybridoma (HFN7.1 deposited under ATCC accession no. CRL-1606) and 10F7MN hybridomas (10F7MN deposited under ATCC accession no. HB-8162) are obtained from American Tissue type collection. The IgG produced by HFN7.1 recognizes human fibronectin, while the IgG produced by 10F7MN recognizes human glycophorin-MN. Cells are expanded by growth in culture (Covance, Richmond Calif.) and provided as a frozen pellet. Messenger RNA is prepared using the mRNA direct kit (Qiagen) according to the manufacturer's instructions. Five hundred nanograms of purified mRNA is diluted to 25 ng/μl in sterile RNAse free H₂O and denatured at 65° C. for 10 minutes, then cooled on ice for 5 minutes. First strand cDNA is created using the reagents and methods described in the “Mouse scFv Module” (Amersham Pharmacia).

This kit is also used essentially as described for creation of single chain fragment-variable antigen binding molecules (see, e.g., U.S. Pat. No. 4,946,778, which describes construction of scFvs described). Briefly, the variable regions of the immunoglobulin heavy and light chain genes are amplified during 30 cycles with Pfu Turbo polymerase (Stratagene, 94° C., 1:00; 55° C., 1:00; 72° C., 1:00), the products are separated on a 2% agarose gel and DNA is purified from agarose slices by phenol/chloroform extraction and precipitation. Following quantification of heavy and light chain fragments, they are assembled with a linker (provided by Amersham-Pharmacia in the Mouse scFv Module) by 7 cycles of amplification (94° C., 1:00; 63° C., 4:00). Primers are added and 30 additional cycles (94° C., 1:00; 55° C., 1:00; 72° C., 1:00) are performed to append the SfiI and NotI restriction enzyme sites to the scFv.

The pBAD/gIII vector (Invitrogen) is modified for expression of scFvs by alteration of the multiple cloning sites to make it compatible with the SfiI and NotI sites used for most scFv construction protocols. The oligonucleotides SfiINotIFor and SfiINotIRev are hybridized and inserted into NcoI and HindIII digested pBAD/gIII DNA by ligation with T4 DNA ligase. The resultant vector (pBADmyc) permits insertion of scFvs in the same reading frame as the gene III leader sequence and the tag. Other features of the pBAD/gIII vector include an arabinose inducible promoter (araBAD) for tightly controlled expression, a ribosome binding sequence, an ATG initiation codon, the signal sequence from the M13 filamentous phage gene III protein for expression of the scFv in the periplasm of E. coli, a myc tag for recognition by the 9E10 monoclonal antibody, a polyhistidine region for purification on metal chelating columns, the rrnB transcriptional terminator, as well as the araC and beta-lactamase open reading frames, and the ColE1 origin of replication.

Additional vectors are created to contain the HA epitope (pBADHA, for recognition of fusion proteins with the HA11, 12CA5 or HA7 monoclonal antibodies) or FLAG epitope (pBADM2, for recognition of fusion proteins with the FLAG-M2 antibody) in place of the myc epitope.

The scFvs derived from the hybridomas and the pBADmyc expression vector are digested sequentially with SfiI and NotI and separated on agarose gels. DNA fragments are purified from gel slices and ligated using T4 DNA ligase. Following transformation into E. coli, and overnight growth on ampicillin containing LB-agar plates, individual colonies are inoculated into 2×YT medium (YT medium is 0.5% yeast extract, 0.5% NaCl, 0.8% bacto-tryptone) with 100 μg/ml ampicillin and shaken at 250 rpm overnight at 37° C. Cultures are diluted 2 fold into 2×YT containing 0.2% arabinose and shaken at 250 rpm for an additional 4 hours at 30° C. Cultures are then screened for reactivity to antigen in a standard ELISA.

Briefly, 96-well polystyrene plates are coated overnight with 1 O/g/ml antigen (Sigma) in 0.1M NaHCO₃, pH 8.6 at 4° C. Plates are rinsed twice with 50 mM Tris, 150 mM NaCl, 0.05% Tween-20, pH 7.4 (TBST), and then blocked with 3% non-fat dry milk in TBST (3% NFM-TBST) for 1 hour at 37° C. Plates are rinsed 4× with TBST and 40 μl of unclarified culture is added to wells containing 10 μl 10% NFM in 5×PBS. Following incubation at 37° C. for 1 hour, plates are washed 4× with TBST. The 9E10 monoclonal (Covance) recognizing the myc tag is diluted to 0.5 μg/ml in 3% NFM-TBST and incubated in wells for 1 hour at 37° C. Plates are washed 4× with TBST and incubated with horseradish peroxidase conjugated goat-anti-mouse IgG (Jackson Immunoresearch, 1:2500 in 3% NFM-TBST) for 1 hour at 37° C. After 4 additional washes with TBST, the wells are developed with o-phenylene diamine substrate (Sigma, 0.4 mg/ml in 0.05 Citrate phosphate buffer pH 5.0) and stopped with 3N HCl. Plates are read in a microplate reader at 492 nm. Cultures eliciting a reading above 0.5 OD units are scored positive and retested for lack of reactivity to a panel of additional antigens. Those clones that lack reactivity to other antigens, and repeat reactivity to the specific antigen are grown, DNA is prepared and the scFv is subcloned by standard methods into the pBADHA and pBADM2 vectors.

For large scale preparation of purified scFv, osmotic shock fluid from an induced culture is reacted with a metal chelate to capture the polyhistidine tagged scFv. Briefly, a single colony representing the desired clone is inoculated into 400 mls of 2×YT containing 100 μg/ml ampicillin and shaken at 250 rpm overnight at 37° C. The culture is diluted to 800 mls of 2×YT containing 0.1% arabinose and 100 μg/ml ampicillin. This culture is now shaken at 250 rpm for 4 hours at 30° C. to allow expression of the scFv. Bacteria are pelleted at 3000×g at 4° C. for 15 minutes, and resuspended in 20% sucrose, 20 mM Tris-HCl, 2.5 mM EDTA, pH 8.0 at 5.0 OD Units (absorbance at 600 nm). Cells are incubated on ice for 20 minutes and then pelleted at 3000×g for 10 minutes at 4° C. The supernatant is removed and saved. Following resuspension in 20 mM Tris-HCl, 2.5 mM EDTA, pH 8.0 at 5.0 OD units, cells are incubated on ice for 10 minutes and then pelleted at 3000×g for 10 minutes at 4° C. The supernatant from this step is combined with the previous supernatant and NaCl, imidazole, and MgCl₂ are added to final concentrations of 1M, 10 mM, and 10 mM respectively. Nickel-nitriloacetic acid agarose beads (Ni-NTA, Qiagen) are stirred with the combined supernatants overnight at 4° C. The beads are collected with centrifugation at 3000×g for 10 minutes at 4° C., and resuspended in 50 mM NaH₂PO₄, 20 mM imidazole, 300 mM NaCl, pH 8.0 and loaded into a column. After allowing the resin to pack and this wash buffer to flow through, the scFv is eluted with successive 0.5 ml fractions of 50 mM NaH₂PO₄, 250 mM Imidazole, 300 mM NaCl, 50 mM EDTA, pH 8.0. Fractions are analyzed by SDS-PAGE and staining with GelCode Blue (Pierce-Endogen) and those containing sufficient quantities of scFv are pooled and dialyzed vs PBS overnight at 4° C. Purified scFv is quantified using a modified Lowry assay (Pierce-Endogen) according to the manufacturer's instructions and stored in PBS+20% glycerol at −80° C. until use.

### Example 6

### Construction of a scFv Master Library

A. mRNA Isolation

Immunized mouse spleens with an ELISA titer within the range of 100,000. Spleens were either quick frozen immediately upon removal by immersion in liquid nitrogen and stored at −80° C. after fast freeze. The mouse spleens were then weighed without thawing. Total RNA was isolated using Stratagene's RNA Isolation kit according to manufacture's protocol. For a naïve library, the mRNA was isolated from total RNA using Stratagene's Poly(A) quick mRNA isolation kit according to manufacture's protocol. The concentration of mRNA was determined by making an appropriate dilution in RNAse-Free H₂O and measuring the optical density at 260 nm in a spectrophotometer. The quality of the RNA was tested by setting up one reaction of first strand cDNA synthesis and amplifying with a pair of primers for Fab or scFv light chain (see below).

B. First Strand cDNA Synthesis

Library generation by PCR was performed in laminar flow hood which was irradiated with UV light for more than 30 min prior to use. A RNA/primer mixture was prepared in sterile 0.2 ml PCR tubes on ice as follows:

| Component | Sample |
|---|---|
| 2 μg total RNA | x μl |
| Random hexamers (50 ng/μl) | 2 μl |
| 10 mM dNTP mix | 1 μl |
| DEPC-treated dH₂O | x μl |
| total volume | 10 μl |

The sample was incubated 65° C. in a thermal cycler for 5 min and then chilled on ice for at least 1 minute. The following mixture was prepared on ice by adding each component in the order indicated below:

| Component | each reaction | 4 reactions |
|---|---|---|
| 10X RT buffer | 2 μl | 8 μl |
| 25 mM MgCl₂ | 4 μl | 16 μl |
| 0.1 M DTT | 2 μl | 8 μl |
| RNase OUT recombinant | 1 μl | 4 μl |
| RNase inhibitor | | |

Nine μl of reaction mix was added to each RNA/primer mixture, mixed gently and then spun briefly. The reaction was incubated at 25° C. in a thermal cycler for 2 minute. One μl (50 units) of Superscript II RT was added to each tube, mixed gently and then spun quickly. The mixture was incubated for 10 minutes at 25° C., for 50 min at 42° C. and for 15 min at 70° C. The reaction was then chilled on ice. The reaction was spun briefly, 1 μl of RNase H was added to each tube and then incubated at 37° C. for 20 minutes. Samples were then used in the amplification section below or stores at −80° C.

C. Amplification of First Strand cDNA

1. PCR Reactions

Working dilutions of the mouse primers were prepared. Each primer was diluted to 100 pmol/μl (to be stored at −80° C. stock) and 10 pmol/μl (to be stored at −20° C. stock) with 10 mM Tris pH 8.0 (RNase free). Ten pmol/μl of primer mix were prepared of each variant at equal molar concentration as shown in Table 5 below:

TABLE 5

| | | | Volume of variant | Total volume in |
|---|---|---|---|---|
| Primer Mix | SEQ ID NO. | Common Name | at 10 pmol/μl | mix |
| MK 1-5 | 103 | MK1 | 10 μl | 100 μl |
| | 104 | MK2 | 20 μl | |
| | 105 | MK3 | 10 μl | |
| | 106 | MK4 | 20 μl | |
| | 107 | MK5 | 40 μl | |
| MK 6-10 | 108 | MK6 | 20 μl | 120 μl |
| | 109 | MK7 | 40 μl | |
| | 110 | MK8 | 20 μl | |
| | 111 | MK9 | 30 μl | |
| | 112 | MK10 | 10 μl | |
| MK 11-15 | 113 | MK11 | 10 μl | 120 μl |
| | 114 | MK12 | 20 μl | |
| | 115 | MK13 | 10 μl | |
| | 116 | MK14 | 40 μl | |
| | 117 | MK15 | 40 μl | |
| MK 16-20 | 118 | MK16 | 40 μl | 110 μl |
| | 119 | MK17 | 10 μl | |
| | 120 | MK18 | 30 μl | |
| | 121 | MK19 | 20 μl | |
| | 122 | MK20 | 10 μl | |
| MK 21-25 | 123 | MK21 | 20 μl | 100 μl |
| | 124 | MK22 | 20 μl | |
| | 125 | MK23 | 20 μl | |
| | 126 | MK24 | 20 μl | |
| | 127 | MK25 | 20 μl | |
| MKR 1-4 | 128 | MKR1 | 40 μl | 160 μl |
| | 129 | MKR2 | 40 μl | |
| | 130 | MKR3 | 40 μl | |
| | 131 | MKR4 | 40 μl | |
| MH 1-5 | 132 | MH1 | 40 μl | 180 μl |
| | 133 | MH2 | 40 μl | |
| | 134 | MH3 | 40 μl | |
| | 135 | MH4 | 20 μl | |
| | 136 | MH5 | 40 μl | |
| MH 6-10 | 137 | MH6 | 20 μl | 180 μl |
| | 138 | MH7 | 60 μl | |
| | 139 | MH8 | 40 μl | |
| | 140 | MH9 | 40 μl | |
| | 141 | MH10 | 20 μl | |
| MH 11-15 | 142 | MH11 | 10 μl | 190 μl |
| | 143 | MH12 | 40 μl | |
| | 144 | MH13 | 60 μl | |
| | 145 | MH14 | 40 μl | |
| | 146 | MH15 | 40 μl | |
| MH 16-20 | 147 | MH16 | 20 μl | 130 μl |
| | 148 | MH17 | 20 μl | |
| | 149 | MH18 | 40 μl | |
| | 150 | MH19 | 40 μl | |
| | 151 | MH20 | 10 μl | |
| MH 21-25 | 152 | MH21 | 80 μl | 200 μl |
| | 153 | MH22 | 60 μl | |
| | 154 | MH23 | 40 μl | |
| | 155 | MH24 | 10 μl | |
| | 156 | MH25 | 10 μl | |
| MHR 1-4 | 157 | MHR1 | 40 μl | 160 μl |
| | 158 | MHR2 | 40 μl | |
| | 159 | MHR3 | 40 μl | |
| | 160 | MHR4 | 40 μl | |

The mixtures were stored at −20° C. PCR reaction mixtures were prepared on ice in 0.2 ml PCT tubes using Clontech's Advantage HF2 polymerase as follows in Tables 6 and 7:

TABLE 6
scFv-HC

| | | | | template | | |
|---|---|---|---|---|---|---|
| 10X HF2 | 10X HF2 | F-primer | R-primer | (1st strand | Polymerase | |
| buffer | dNTP mix | (10 pmol/μl) | (10 pmol/μl) | cDNA) | Mix | dH₂O |
| 5 μl | 5 μl | 1 μl MH1-5 | 1 μl MHR1-4 | 2 μl | 1 μl | 35 μl |
| 5 μl | 5 μl | 1 μl MH6-10 | 1 μl MHR1-4 | 2 μl | 1 μl | 35 μl |
| 5 μl | 5 μl | 1 μl MH11-15 | 1 μl MHR1-4 | 2 μl | 1 μl | 35 μl |
| 5 μl | 5 μl | 1 μl MH16-20 | 1 μl MHR1-4 | 2 μl | 1 μl | 35 μl |
| 5 μl | 5 μl | 1 μl MH21-25 | 1 μl MHR1-4 | 2 μl | 1 μl | 35 μl |

TABLE 7
scFv-LC

| | | | | template | | |
|---|---|---|---|---|---|---|
| 10X HF2 | 10X HF2 | F-primer | R-primer | (1st strand | Polymerase | |
| buffer | dNTP mix | (10 pmol/μl) | (10 pmol/μl) | cDNA) | Mix | dH₂O |
| 5 μl | 5 μl | 1 μl MK1-5 | 1 μl MKR1-4 | 2 μl | 1 μl | 35 μl |
| 5 μl | 5 μl | 1 μl MK6-10 | 1 μl MKR1-4 | 2 μl | 1 μl | 35 μl |
| 5 μl | 5 μl | 1 μl MK11-15 | 1 μl MKR1-4 | 2 μl | 1 μl | 35 μl |
| 5 μl | 5 μl | 1 μl MK16-20 | 1 μl MKR1-4 | 2 μl | 1 μl | 35 μl |
| 5 μl | 5 μl | 1 μl MK21-25 | 1 μl MKR1-4 | 2 μl | 1 μl | 35 μl |

The reactions were mixed gently then spun briefly. The tubes were then set in the thermal cycler preheated to 94° C. and the following cycle was started: 94° C. for 2 min, 94° C. for 1 min, 55° C. for 1 min, 72° C. for 1 min, 72° C. for 10 min for 30 cycles and then held at 4° C. The reactions were then spun briefly and proceed to gel purification steps

2. Gel Purification of PCR Products

A 1% low melting point agarose gel was prepared. Ten 10 μl of 6× loading buffer was added to each 50 μl PCR reaction. The entire sample was loaded onto 1% agarose gel. The gels were run at 100 volts until the dark blue dye runs ⅔ length of the gel. The gels were then photographed. Working quickly, the gels were visualized with UV light and the bands excised at the appropriate size

scFv-HC: ˜350 bp

scFv-LC: ˜325 bp

3. Frozen Phenol Purification of DNA from Low Melt Agarose

The appropriate bands were cut out and placed into eppendorf tubes (450 μl each tube) or in 15 ml conical tubes (4.5 ml each tube). The volume of agarose slice was estimated. 1/10^{th} volume 3 M NaOAc, pH 5.2 and 1/10^{th} volume 1 M Tris, pH 8.0, was added to the tube containing the excised slice. The slice was then melted at 65° C. in a heat block. Once the slice was completely melted, an equal volume of room temperature phenol was added. The solution was well-vortexed (30 seconds) until all chunks of agarose were dissolved. The solution was then frozen on dry ice until solid.

To separate the phases, the solution was spun for 15 min at maximum speed at RT. The aqueous phase was transferred to a fresh tube without disturbing the interface. The separation and transfer steps were repeated once, followed by extraction by chloroform. The aqueous phase was transferred to fresh tube and 1 μl of glycogen (20 mg/ml) was added. Two volumes of 100% EtOH were added. The solution was then incubated at −20° C. for 2 hours to overnight. Solution can optionally be incubated for 30 min at −80° C.). The DNA was pelleted at 4° C. for 15 min at maximum speed, then washed with 70% EtOH once. The pellet was resuspended in dH₂O or 10 mM Tris pH 8.0. The purified PCR product was quantified. The purified DNA was then stored at −20° C.

D. Antibody Fragment Assembly

1. The scFv Linker

The scFv linker was generated using Clontech's Advantage HF2 polymerase kit as outlined by the manufacturer's instructions. Briefly, PCR mix was prepared in a 0.2 ml PCR tube on ice with the following:

5 μl 10×HF2 buffer

4 μl 10×HF2 dNTP mix

2 μl 10 pmol/μl of LinkF (SEQ ID No. 164)

2 μl 10 pmol/μl of LinkR (SEQ ID No. 165)

25 ng of pBADHA-HFN clone 10

1 μl polymerase mix

add dH₂O to total volume of 50 μl

The tubes were set in the thermal cycle block and the following cycle was started: 94° C. for 2 min; 94° C. for 1 min/55° C. for 1 min/72° C. for 1 min for 30 cycles then 72° C. for 10 min and holding at 4° C.

The prepared assembled scFv linker was then purified by gel electrophoresis. A 2% agarose gel was prepared. Ten μl of 6×loading buffer was added to each 50 μl PCR mix and load onto the gel. The gel was run at 100 volts until the dark blue dye ran ⅔ down the length of the gel. The scFv linker band (at ˜50 bp) was excised from the gel.

The PCR product was purified from the excised gel slice using the MERmaid kit (Qbiogene, Carlsbad Calif.) according to the manufacture's instruction. Optionally, the PCR product can be purified using “Frozen phenol” purification. The purified scFv linker was quantified using Picogreen quantitation kit (Molecular Probes) according to the manufacturer's protocol.

2. scFv Assembly

Two PCR mixtures were prepared in 0.2 ml PCR tubes on ice as follows:

4 μl 10×HF2 buffer

4 μl 10×HF2 dNTP mix

5 ng purified scFv-HC fragment

5 ng purified scFv-LC fragment

2 ng purified scFv-linker (from step above)

0.8 μl Advantage polymerase mix

bring to 40 μl with dH₂O

The tubes were placed in a thermal cycler block and the following cycle was started: 94° C. for 3 min; 94° C. for 30 seconds/55° C. for 30 seconds/72° C. for 1 min for 7 cycles; and hold at 4° C. The tubes were then spun briefly and placed on ice. A mixture of following components was prepared:

1 μl 10×HF2 buffer

1 μl 10×HF2 dNTP mix

2 μl primer SfiFor (SEQ ID No. 166)

2 μl primer NotRev (SEQ ID No. 167)

0.2 μl Advantage polymerase mix

bring to total of 10 μl with dH₂O

Ten μl of the mixture was added to each of the 40 μl PCR reactions. The solutions were mixed and then spun. The tubes were then placed in a thermal cycler block preheated to 94° C. and the following cycle was started: 94° C. for 2 min; 94° C. for 1 min/55° C. for 1 min/72° C. for 2 min for 30 cycles; 72° C. for 10 min; and held at 4° C.

The assembled scFv fragment was purified by gel electrophoresis. A 1% low melting agarose gel was prepared. Ten μl of 6×loading buffer was added to each 50 μl PCR mix and loaded onto the gel. The gel was run at 100 volts until the dark blue dye ran 2/3 down the length of the gel. Working quickly, the gel was visualized with UV light and the scFv band at ˜700 bp was excised. The DNA was extracted from the gel slice using Frozen Phenol purification of DNA from low melt agarose. The amount of purified scFv fragment was quantitated using the Picogreen kit (Molecular Probes).

E. Generate Fab and scFv Library in pBADHA or Equivalent

1. Generation of SfiI/NotI Digested pBADHA (or Equivalent)

Digestion reaction mix was prepared in a 1.5 ml eppendorf tubes as follows:

X μl pBADHA (˜20 μg)

20 μl 10× buffer #2 (NEB)

20 μl 10×BSA (100× stock)

10 μl SfiI (20 units/μl)

X μl dH₂O for a total of 200 μl

The solution was incubated at 50° C. for 4 hours. Following the incubation, the solution was spun briefly and he following components were added to each tube:

5 μl 10× buffer #3 (NEB)

5 μl 10×BSA (NEB, 100× stock)

8 μl 1 M Tris pH 8.0

2 μl 5 M NaCl

10 μl NotI

20 μl dH₂O

The solution was then incubated at 37° C. for 4 hours.

For dephosphorylation, the following components were added to above digestion reaction:

5 μl 10× buffer #3

20 μl CIP alkaline phosphatase (1 unit/μl)

25 μl dH₂O

The solution was then incubated for 30 min at 37° C. The digested and dephosphorylated DNA was run on 1% agarose gel for purification. The SfiI/NotI fragment band was excised from the gel and the DNA was purified from the slice by extraction using Frozen Phenol purification of DNA from low melt agarose. The Picogreen kit from Molecular Probes was used for quantitation of the purified pBADHA (SfiI/NotI/CIP) DNA.

The background of purified pBADHA (SfiI/NotI/CIP) DNA was determined. Briefly, the following ligation was prepared:

X μl 5 ng of pBADHA (SfiI/NotI/CIP) DNA

0.5 μl T4 DNA ligase buffer

0.5 μl T4 DNA ligase (NEB; 400 units/μl)

add dH₂O to bring to total of 5 μl

The ligation reaction was incubated at 16° C. for ˜16 hours. The reaction was then chilled on ice for 5 min and spun briefly.

Electroporation cuvettes (VWR; 1 mm gap) and 0.5 ml eppendorf tubes were prechilled on ice. The frozen electrocompetent XL1-blue cells (with transformation efficiency at about 1×10⁸) were thawed on ice. Forty μl of cells were transferred to the 0.5 ml tube on ice and 1 μl of ligation (1 ng DNA) mix was added to the tube. In addition, 1 ng of pBADHA uncut was placed in a separate tube as a control. The mixtures were placed on ice for ˜1 minute. The transformation mix were transferred to the prechilled electroporation cuvettes on ice and shaken to the bottom of the cuvette. The mixtures were electroporated once at 1.7 KV. Following the electroporation, 300 μl of 2×YT/glucose medium was added to the cuvettes. The solution was transferred to a 5 ml Falcon tube with a transfer pipette. The culture was incubated for 1 hour at 37° C. with shaking at 250 rmp. One μl, 10 μl and 30 μl of the transformed cells were plated onto 3 separate 2×YT/glucose/amp plates (100 mm) using sterile glass beads. Once dry, the plates were invert and incubated at 37° C. overnight. The colony number on each plate was observed visually (pBADHA (SfiI/NotI/CIP) to ensure less than 10 colonies per plate. DNA should give the same or fewer colonies than uncut pBADHA.

2. Generation of SfiI/NotI Digested Fab or ScFv Fragment

A digestion reaction mix was prepared in a 1.5 ml eppendorf tube as follows:

X μl Purified Fab or scFv DNA (˜1 μg)

5 μl 10× buffer #2 (NEB)

5 μl 10×BSA

2 μl SfiI (NEB; 20 units/μl)

add dH₂O to bring total volume of 50 μl

The digestion reaction was incubated at 50° C. for 2 hours. The reaction was then spun briefly and the following components were added to each tube:

5 μl 10× buffer #3 (NEB)

5 μl 10×BSA

2 μl 1 M Tris pH 8.0

0.5 μl 5 M NaCl

4 μl NotI (NEB; 10 units/μl)

add 33.5 μl of dH₂O

The solution was then incubated at 37° C. for 2 hours. The digested DNA was then run on 1% agarose gel and the Fab (˜1.4 Kb) and scFv (˜700 bp) bands were excised. The DNA from the gel slices was purified by extraction using Frozen Phenol purification of DNA from low melt agarose. The purified Fab and scFv DNA was quantitated using the Picogreen kit from Molecular Probes.

3. Ligation of scFv Fragment into Vector

The scFv DNA was ligated to pBADHA using the following ligation mix (keep the molar ratio of insert versus vector at 1-2:1)

X μl pBADHA (SfiI/NotI cut; 820 ng for scFv)

X μl Fab or ScFv (SfiI/NotI cut; 180 ng for ScFv)

5 μl T4 DNA ligase buffer

5 μl T4 DNA ligase (NEB; 400 units/μl)

add dH₂O to bring to total of 50 μl

The ligation reaction was incubated at 16° C. for 16 hours, then chilled on ice for 5 min and spun briefly. The ligation mixture was buffer exchanged using Princeton Separations's Centri-Spin 20 columns (Princeton Separations, Adelphia N.J.) according to manufacture's instruction. Briefly, the centri-spin 20 columns were hydrated with 650 μl ddH₂O at room temperature for at least 30 minutes. The ligation mix was heated to 66-68° C. for 10 min to inactivate the ligase and linearize any non-ligated molecules. The centri-spin 20 columns were placed in the 2 ml wash tube and spun at 750×g for 2 minutes. The ligation mix (20-50 μl) was added on the top of the gel bed (be careful not to disturb the gel bed). The column was placed in the collection tube (1.5 ml tube) and spun at 750×g for 2 min to collect the sample.

4. Transformation

The electroporation cuvettes (VWR; 1 mm gap) and 0.5 ml eppendorf tubes were prechilled on ice. The frozen electrocompetent cells were thawed on ice. Forty μl 1-Blue or TG1 cells were added to a 0.5 ml tube on ice, followed by addition of 1 μl of ligation mix to the tube. The tubes were placed on ice for ˜1 minute.

The transformation mix was then transferred to the prechilled electroporation cuvettes on ice and shaken to the bottom of the cuvettes. The mixture was electroporated once at 1.7 KV (1.66 KV for DH12S from GIBCO). Immediately following electorporation, 300 μl of 2×YT/2% glucose medium was added to the cuvette. The transformation steps above were repeated 49 more times for total of 50 individual samples for each ligation.

The contents of the 50 cuvettes (˜15 ml) was transferred to a 50 ml tube with transfer pipette (need two tubes). The culture was incubated for 1 hour at 37° C. with shaking at 250 rmp. Fifty μl for was set aside for titering (see below). Three hundred μl of the transformed cells were plated onto 50 separate 2×YT/2% glucose/Amp (0.1 mg/ml) plates (150 mm) using sterile glass beads. Once dry, the plates were inverted and incubated at 37° C. overnight. The cells were removed from the plates by flooding each plate with 5 ml 2×YT and scraping the cells into medium with a sterile spreader. Five ml of cells were reserved for phage rescue (see below). Frozen cell stock was prepared by adding glycerol to a final concentration of 15% and storing at −80° C. in 1 ml aliquots (10 aliquots is sufficient).

For cell titering, 1 μl, 10 μl and 30 μl of transformants from the above transformation were plated on 2×YT/2% glucose/Amp (0.1 mg/ml) plates (100 mm). The plated were incubated overnight at 37° C. Following the incubation, the colonies were visually counted and the colony forming units determined.

5. Rescue of the Library

One ml of the scraped cells were transferred to a 500 ml shake flask. The cells were diluted to OD600=0.2 with 2×YT/2% glucose. The culture was incubated for 1 hour at 37° C. with shaking at 250 rpm and measured the OD₆₀₀. M13K07 (Stratagene, San Diego Calif.; Veira et al. (1987) Meth. Enz. 153:3) helper phage was added to the culture at a multiplicity of infection (moi) of 5:1 (moi) of 5:1 (10D600=8×10⁸ cells). The culture was incubated for 1 hour at 37° C. with shaking at 250 rpm, then spun at 1000×g for 20 minutes. Following the centrifugation, the supernatant was carefully remove and discarded. The pellet was gently resuspended in 500 ml of 2×YT/Amp/Kan medium in a 2 L shake flask. The culture was incubated overnight at 30° C.

Following the incubation, the cells were centrifuged at 8000 rmp for 30 min at 4° C. The resulting supernatant, which contained the recombinant phage, was transferred to 500 ml centrifuge bottles (2 bottles total). 4-(2-aminoethyl)benzenesulfonyl fluoride (AEBSF) was added to a final concentration of 0.2 μM.

### Example 7

### Creation and Production of scFv Libraries with Even Distribution of Polypeptide Tags

A. Preparation of pBAD: Tag Expression Vectors

1. The pBAD: Tag Vector

The A form of the pBAD/gIII vector (Invitrogen, Carlsbad, Calif.) was modified for expression of scFvs by alteration of the multiple cloning sites to make it compatible with the SfiI and NotI sites used for most scFv construction protocols. The oligonucleotides SfiINotIFor and SfiINotIRev (SEQ ID Nos. 6 and 7) were hybridized and inserted into NcoI and HindIII digested pBAD/gIII DNA by ligation with T4 DNA ligase. The resultant vector (pBADmyc) permits insertion of scFvs in the same reading frame as the gene III leader sequence and the polypeptide tag, which has a sequence of EQKLISEEDL (SEQ ID No. 91).

For insertion of the scFv, the vector was incubated for 2 hours at 50° C. in a volume of 100 μl with 100 Units of SfiI (New England Biolabs) in 50 mM NaCl, 10 mM Tris-HCl, 10 mM MgCl₂, 1 mM dithiothreitol (DTT) pH 7.9 supplemented with 100 μg/ml bovine serum albumin (BSA). Following digestion with SfiI, the reaction was supplemented with additional H₂O, MgCl₂, Tris-HCl, NaCl, DTT, BSA, and NotI (New England Biolabs) such that the reaction volume is 150 μl containing 100 Units of NotI in 100 mM NaCl, 50 mM Tris-HCl, 10 mM MgCl₂, 1 mM DTT pH 7.9 and 100 μg/ml BSA. This reaction was incubated at 37° C. for 2 hours. Calf intestinal phosphatase (25 Units CIP, New England Biolabs) was added to the reaction and incubated at 37° C. for an additional 1 hour. Simultaneously, the scFv sublibrary was digested with Other features of the pBAD/gIII vector include an arabinose inducible promoter (araBAD) for tightly controlled expression, a ribosome binding sequence, an ATG initiation codon, the signal sequence from the M13 filamentous phage gene III protein for expression of the scFv in the periplasm of E. coli, a myc polypeptide tag for recognition by the 9E10 monoclonal antibody, a polyhistidine region for purification on metal chelating columns, the rrnB transcriptional terminator, as well as the araC and beta-lactamase open reading frames, and the ColE1 origin of replication.

Additional vectors were created to contain the following polypeptide tags in place of the myc epitope (Table 8):

TABLE 8
Epitopes Peptides

| Epitope | Sequence | SEQ ID No. |
|---|---|---|
| myc | EQKLISEEDL | 91 |
| HA | YPYDVPDYA | 92 |
| FLAG | DYKDDDDK | 93 |
| GluGlu | EEEEYMPME | 94 |
| V5 | GKPIPNPLLGLDST | 95 |
| T7 | MASMTGGQQMG | 96 |
| HSV | QPELAPEDPED | 97 |
| S-tag | KETAAAKFERQHMDS | 98 |
| KT3 | KPPTPPPEPET | 99 |
| E-tag | GAPVPYPDPLEPR | 100 |
| VSV-g | YTDIEMNRLGK | 101 |
| B34 | DLHDERTLQFKL | 106 |
| VSV-1 | HPNLPETRRYAL | 107 |
| VSV-2 | SYTGIEFDRLSN | 108 |
| 4C10 | MVDPEAQDVPKW | 109 |
| AB2 | LTPPMGPVIDQR | 110 |
| AB4 | QPQSKGFEPPPP | 111 |
| AB3 | YEYAKGSEPPAL | 112 |
| AB6 | AGTQWCLTRPPC | 113 |
| KT3-A | KLMPNEFFGLLP | 114 |
| KT3-B | KLIPTQLYLLHP | 115 |
| KT3-C | SFMPIEFYARKL | 116 |
| 7.23 | TNMEWMTSHRSA | 117 |
| S1 | NANNPDWDF | 118 |
| E2 | SSTSSDFRDR | 119 |
| His tag | HHHHHHGS | 120 |
| AU1 | DTYRYI | 121 |
| AU5 | TDFYLK | 122 |
| IRS | RYIRS | 123 |
| NusA | NusA Protein | 124 |
| MBP | Maltose Binding Protein | 125 |
| TBP | TATA-box Binding Protein | 126 |
| TRX | Thioredoxin | 127 |
| HOPC1 | MPQQGDPDWVVP | 128 |

2. Screening for Antigen Reactivity

Cultures were screened for reactivity to antigen in a standard ELISA. Briefly, 96-well polystyrene plates were coated overnight with 10 μg/ml antigen (Sigma) in 0.1 M NaHCO₃, pH 8.6 at 4° C. Plates were rinsed twice with 50 mM Tris, 150 mM NaCl, 0.05% Tween-20, pH 7.4 (TBST), and then blocked with 3% non-fat dry milk in TBST (3% NFM-TBST) for 1 hour at 37° C. Plates were rinsed 4 times with TBST and 40 μl of unclarified culture was added to wells containing 10 μl 10% NFM in 5×PBS. Following incubation at 37° C. for 1 hour, plates were washed 4 times with TBST. The 9E10 monoclonal antibody (Covance) recognizing the myc polypeptide tag was diluted to 0.5 μg/ml in 3% NFM-TBST and incubated in wells for 1 hour at 37° C. Plates ware washed 4 times with TBST and incubated with horseradish peroxidase conjugated goat-anti-mouse IgG (Jackson Immunoresearch, 1:2500 in 3% NFM-TBST) for 1 hour at 37° C. After 4 additional washes with TBST, the wells were developed with o-phenylene diamine substrate (Sigma, 0.4 mg/ml in 0.05 Citrate phosphate buffer pH 5.0) and stopped with 3N HCl. Plates were read in a microplate reader at 492 nm. Cultures eliciting a reading above 0.5 OD units were scored positive and retested for lack of reactivity to a panel of additional antigens. Those clones that lacked reactivity to other antigens, and repeat reactivity to the specific antigen were grown up in culture. The DNA was prepared and the scFv was subcloned by standard methods into the pBADHA and pBADM2 vectors.

B. Cloning of scFv Fragments into pBAD: Tag Vectors

1. Generation of SfiI/NotI Digested scFv Fragments and Digested pBAD: Tag Vector

Purified scFv DNA (1 μg×n where n is the number of tags) was digested with 4 μl SfI (20 units/μl) in a total volume of 100 PI in 10 mM Tris-HCl, 10 mM MgCl₂, 50 mM NaCl, 1 mM DTT buffer (pH 7.9) for 2 hours at 50° C. The tube was spun briefly and the pH adjusted to 8.0. The DNA was then digested with 8 μl NotI (10 units/μl) in a total volume of 200 μl in a 50 mM Tris-HCl, 10 mM MgCl₂, 100 mM NaCl, 1 mM DTT buffer at 37° C. for 2 hours. The digested DNA was electrophoresed on a 1% agarose gel and the scFv band (˜700 bp) excised. The DNA was purified and quantified according to standard procedures well known to those with skill in the art.

Each of the pBAD: Tag Vectors (where each vector has a unique tag representing a single epitope) was separately digested with SfiI and NotI as described above. The digested DNA was electrophoresed on a 1% agarose gel and the linear vector band was excised. The DNA was purified and quantified according to standard procedures well known to those with skill in the art.

2. Ligation of scFv Fragment into pBAD: Tag Vectors

Ligation mixtures were prepared such that the molar ratio of insert to vector was kept at 1-2:1. The digested scFv fragments were divided into a number of aliquots (equal to the number of pBAD: tag vectors) to which an aliquot of the SfiI/NotI digested pBAD: tag vector was added. The scFv was ligated into the vector by addition of T4 DNA ligase (400 units/μl) in 50 mM Tris-HCl (pH 7.5), 10 mM MgCl₂, 10 mM DTT, 1 mM ATP, 25 μg/ml bovine serum albumin buffer in a total volume of 50 μl. The ligation reaction was incubated at 16° C. for ˜16 hours, followed by chilling the reaction on ice for 5 min and a brief spin.

3. Transformation into E. coli and Growth of Recombinant Expression Vector

Freshly thawed frozen electro-competent Top 10 E. coli cells (40/μl; Invitrogen) were added to pre-chilled electroporation cuvettes (1 mm gap) along with 1 μl of each ligation reaction (the number of transformations will equal the number of ligations and hence the number of tags) and the cuvettes were placed on ice for ˜1 minute. The cells were transformed by electroporation at 1.7 KV (1.66 KV for DH12S from GIBCO) and recovered by the immediate addition of 500 μl of SOC medium to the cuvette. The content of each cuvette was transferred to snap-cap culture tubes and the cells incubated for 45 minutes at 37° C. with shaking at 260 RPM. Frozen stocks of each of the transformed cells were prepared by adding glycerol to a final concentration of 15% followed by storage at −80° C. in 0.1 ml aliquots.

4. Titering

An aliquot of each of the transformed cells was thawed and 5 μl aliquots were plated on LB/Amp (0.1 mg/ml) plates (100 mm). The plates were incubated overnight at 37° C. and the titer determined. The titer for each single tag library (single tag library is an aliquot of the scFv library cloned into each pBAD: tag vector) was the number of colony forming units (cfu) per ml of transformed cells.

C. Distribution of Tagged scFv Libraries into Pools

1. Normalization of Titers

After the titers were determined as described above, a frozen aliquot of each single tag library was thawed and 2×YT/2% glucose was added such that the titers are all normalized to be similar to the single tag library with the lowest titer.

2. Pooling the Tagged Libraries

The tagged libraries were pooled by either determining the diversity of scFvs to be displayed (e.g., 10⁹) or by determining the number of tags to be used for displaying the scFvs (e.g., 10²). The amount of aliquot of each normalized tagged library to be pooled was calculated using the formula: diversity to be displayed/number of tags (e.g., 10⁹/10²=10⁷). The calculated amount of each aliquot for each tag was added to a 15 ml tube and kept on ice.

3. Splitting the Mixed Library

The mixed library was split into aliquots such that 1000 scFvs were represented per tag within each aliquot (e.g., for 10² tags, each aliquot will have 1000 scFvs per tag which corresponds to a total of 10⁵ scFvs per aliquot). Each of these aliquots was called an array library.

D. Expression of scFv Array Libraries

1. Starter Culture for scFv Protein Expression

Each array library was inoculated into 1 ml 2×YT supplemented with 50 μg/mL of carbenicillin. The culture was grown at 37° C. for 4 hours with shaking at 260 RPM. The culture was then added to 100 ml of 2×YT containing carbenicillin and grown at 37° C. for an additional 16 hours.

2. Preparation of Glycerol Stocks

Sterile glycerol was added to a final concentration of 15% to a 5 ml aliquot of the culture and stored at −80° C. in 0.5 ml aliquots.

3. Induction and Harvesting of E. coli Cells

Each of the starter cultures was diluted 4-fold by adding 300 mL 2×YT supplemented with 50 μg/mL of carbenicillin. To induce expression, arabinose was added to a final concentration of 0.1% and the cultures were grown at 30° C. with shaking at 260 RPM for 12 hours. Cells were harvested by centrifugation at 5000 g for 20 min at 4° C.

E. Periplasmic Extraction of scFvs

Each pellet was resuspended in 12 mL of Periplasting Buffer (200 mM Tris-HCl, pH 7.5, 20% sucrose, 1 mM EDTA) followed by addition of 6 μl of lysozyme (to a final concentration of 30 units/μL) and incubation at room temperature for 5 minutes. The tubes were then placed on ice, with 36 mL of chilled, pure H₂O added to each tube followed by incubation on ice for 10 minutes. Periplasmic lysates were clarified by centrifugation at 10,000 g for 20 minutes. The supernatants were then transferred into clean tubes.

F. Parallel Purification of scFv Array Libraries

1. Preparation and Equilibration of Affinity Columns

The following components were added to the periplasmic lysate described above such that the final concentration of each component was as indicated below:

500 mM NaCl

10 mM MgCl₂

20 mM Tris, pH 8.0

5 mM Imidazole

For each 50 ml of periplasmic lysate, 1 ml of Ni-NTA slurry was added. Pre-equilibration of the Ni-NTA was performed by adding the required amount of resin in a centrifuge tube, followed by centrifugation at 4000 g for 5 minutes. The supernatant was aspirated off and an equal volume of Lysis Buffer (50 mM NaH₂PO₄ (pH 8), 300 mM NaCl, and 10 mM imidazole) was added to resuspend the resin. The resin was centrifuged again at 4000 g for 5 min followed by aspiration of the supernatant. An equal volume of Lysis Buffer was used to resuspend the resin and the appropriate volume of slurry (corresponding to 1 mL Ni-NTA) was added to each lysate. Binding of scFv to the Ni-NTA was allowed to occur by incubation overnight at 4° C. on a rocker.

2. Manifold Chromatography

The columns were placed on the manifold (up to 20 columns can be accommodated per batch) with the stopcocks in the closed position before beginning. Syringes were placed on each column and the slurry poured into the syringes. Vacuum (˜0.1 bar) was applied and the stopcock opened to allow flow through the columns. Once the entire load volume has passed through the column, the stopcock was closed. (Once the load has passed through the column, it is important to shut the stopcock immediately to avoid drying the resin). Wash Buffer (50 mM NaH₂PO₄ (pH 8), 300 mM NaCl, 20 mM imidazole; 3 ml) was poured into the syringe and the vacuum applied as before. Once the entire Wash Buffer passed through the columns, the stopcocks were closed and the vacuum turned off. The manifold was opened and collection tubes were placed under each column. Elution Buffer (50 mM NaH₂PO₄ (pH 8), 300 mM NaCl, 250 mM imidazole, 50 mM EDTA; 1 ml) was applied to each column and a vacuum was applied. Once the entire aliquot of Elution Buffer passed through the column, the stopcocks were closed and the vacuum turned off. The tubes containing the elution material were capped and stored on ice until buffer exchange.

3. Buffer Exchange and Storage of scFv Array Libraries

Ten μL of 10% Tween-20 solution was added to each elution tube. The eluate was then added to a dialysis cassette, which was placed in 1 L of phosphate buffered saline, pH 7.4 (PBS). The buffer exchange was allowed to take place overnight with stirring at 4° C. Glycerol was added to each dialyzed sample to a final concentration of 20% and each sample was aliquoted and stored at −80° C.

### Example 8

Preparation of Arrays and Use Thereof for Capturing Antibodies Sandwich Assay ELISA Kits

Enzyme-linked immunosorbent assay (ELISA) CytoSets™ kits, available for the detection of human cytokines, were used to generate “sandwich assays” for certain experiments. The “sandwich” is composed of a bound capture antibody, a purified cytokine antigen, a detector antibody, and streptavidin•HRPO. These kits, obtained from BioSource, allowed for the detection of the following human cytokines: human tumor necrosis factor alpha (Hu TNF-α; catalog # CHC1754, lot # 001901) and human interleukin 6 (Hu IL-6; catalog # CHCl₂₆₄, lot # 002901).

Anti-Tag Capture Antibodies

For microarray analyses of scFv function and specificity, capture antibodies specific for hemalgglutinin (HA. 11, specific for the influenza virus hemagglutinin epitope YPYDVPDYA (SEQ ID No. 92); Covance catalog # MMS-101 P, lot # 139027002) and Myc (9E10, specific for the EQKLISEEDL (SEQ ID No. 91) amino acid region of the Myc oncoprotein; Covance catalog # MMS-150P, lot # 139048002) were used. A negative control mouse IgG antibody (FLOPC-21; Sigma catalog # M3645) was also included in these assays.

Preparation of CytoSets™ Capture Antibodies for Printing with Either a Modified Inkjet Printer or a Pin-Style Microarray Printer

Prior to printing CytoSets™ antibodies using a modified inkjet printer or a pin-style microarray printer (see below), capture antibodies from these kits were diluted in glycerol (Sigma catalog # G-6297, lot # 20K0214) to 1-2 mg/ml, in a final glycerol concentration of 1% or 10%. Typically these mixtures were made in bulk and stored in microcentrifuge tubes at 4° C.

Preparation of Anti-Peptide Tag Capture Antibodies for Printing with a Pin-Style Microarray Printer

Capture antibodies specific for peptide tags present on certain scFvs were prepared by serial two-fold dilution. Capture antibody stocks (1 mg/ml) were diluted into a final concentration of 20% glycerol to yield typical final capture antibody concentrations of from 800 to 6 μg/ml. Capture antibody dilutions were prepared in bulk and stored in microcentrifuge tubes at 4° C. and loaded into 96-well microtiter plates (VWR catalog # 62406-241) immediately prior to printing. Alternatively, capture antibody dilutions were made directly in a 96-well microtiter plate immediately prior to printing.

Capture Antibody Printing Using a Modified Inkjet Printer

CytoSets™ capture antibodies were printed with an inkjet printer (Canon model BJC 8200 color inkjet) modified for this application. The six color ink cartridges were first removed from the print head. One-milliliter pipette tips were then cut to fit, in a sealed fashion, over the inkpad reservoir wells in the print head. Various concentrations of capture antibodies, in glycerol, were then pipetted into the pipette tips which were seated on the inkpad reservoirs (typically the pad for the black ink reservoir was used).

For generation of printed images using the modified printer, Microsoft PowerPoint was used to create various on-screen images in black-and-white. The images were then printed onto nitrocellulose paper (Schleicher and Schuell (S&S) Protran BA85, pore size 0.45 μm, VWR catalog # 10402588, lot # CF0628-1) which was cut to fit and taped over the center of an 8.5×11 in piece of printer paper. This two-paper set was hand fed into the printer immediately prior to printing. After printing of the image, the antibodies were dried at ambient temperature for 30 minutes. The nitrocellulose was then removed from the printer paper, and processed as described below (see Basic protocol for antibody and antigen incubations: FAST slides and nitrocellulose filters printed with CytoSets™ capture antibodies).

Capture Antibody Printing Using a Pin-Style Microarray Printer

Capture antibody dilutions were printed onto nitrocellulose slides (Schleicher and Schuell FAST™ slides; VWR catalog # 10484182, lot # EMDZ018) using a pin-printer-style microarrayer (MicroSys 5100; Cartesian Technologies; TeleChem Arraylt™ Chipmaker 2 microspotting pins, catalog # CMP2). Printing was performed using the manufacturer's printing software program (Cartesian Technologies' AxSys version 1, 7, 0, 79) and a single pin (for some experiments), or four pins (for some experiments). Typical print program parameters were as follows: source well dwell time 3 sec; touch-off 16 times; microspots printed at 0.5 mm pitch; pins down speed to slide (start at 10 mm/sec, top at 20 mm/sec, acceleration at 1000 mm/sec²); slide dwell time 5 millisec; wash cycle (2 moves+5 mm in rinse tank; vacuum dry 5 sec); vacuum dry 5 sec at end. Microarray patterns were pre-programmed (in-house) to suit a particular microarray configuration. In many cases, replicate arrays were printed onto a single slide, allowing subsequent analyses of multiple analyte parameters (as one example) to be performed on a single printed slide. This in turn maximized the amount of experimental data generated from such slides. Microtiter plates (96-well for most experiments, 384-well for some experiments) containing capture antibody dilutions were loaded into the microarray printer for printing onto the slides. Based on the reported print volume (post-touch-off, see above) of 1 nl/microspot for the Chipmaker 2 pins, the capture antibody concentrations contained in the printed microspots typically ranged from 800 to 6 μg/microspot.

In some experiments, arrays of capture antibodies were printed onto the bottoms of plastic microtiter plates. For these experiments, 96-well plates (Nunc Maxisorb) were coated overnight with a solution of goat antibody recognizing the Fc region of mouse IgG (Jackson Immuno-Research, 20 μg/ml in 0.1M NaHCO₃ pH 8.6). Plates are incubated overnight at 4° C., washed three times with distilled H₂O, and allowed to air dry. Capture antibody diluted into PBS containing 20% glycerol and 0.00625% Tween-20 (capture antibody at 10 μg/ml to 1 ng/ml) was aliquoted into individual wells of a source plate for printing onto the coated, dried plates. Based on the reported print volume (post-touch-off, see above) of 1 nl/microspot for the Chipmaker 2 pins, the capture antibody concentrations contained in the printed microspots typically ranged from 10 μg/microspot to 10 fg/microspot.

Printing was performed at 50-55% relative humidity (RH) as recommended by the microarray printer manufacturer. RH was maintained at 50-55% via a portable humidifier built into the microarray printer. Average printing times ranged from 5-15 min; print times were dependent on the particular microarray that was printed. When printing was completed, slides were removed from the printer and dried at ambient temperature and RH for 30 minutes.

Blocking Agent, PBS, and PBS-T

Following capture antibody printing, blocking of slides was done with Blocker BSA™ (10% or 10× stock; Pierce catalog # 37525) diluted to in phosphate-buffered saline (PBS) (BupH™ modified Dulbecco's PBS packs; Pierce catalog # 28374). Tween-20 (polyoxyethylene-sorbitan monolaurate; Sigma catalog # P-7949) was then added to a final concentration of 0.05% (vol:vol). The resulting blocker is hereafter referred to as BBSA-T, while the resulting PBS with 0.05% (vol:vol) Tween-20 is referred to as PBS-T.

Incubation Chamber Assemblies for FAST Slides

For isolation of individual microarrays of capture antibodies on a single FAST slide, slotted aluminum blocks were machined to match the dimensions of the FAST™ slides. Silicone isolator gaskets (Grace BioLabs; VWR catalog #s 10485011 and 10485012) were hand-cut to fit the dimensions of the slotted aluminum blocks. A “sandwich” consisting of a printed slide, gasket, and aluminum block was then assembled and held together with 0.75 inch binder clips. The minimum and maximum volumes for one such isolation chamber, isolating one antibody microarray, were 50-200 μl.

Basic Protocol for Antibody and Antigen Incubations: FAST Slides and Nitrocellulose Filters Printed with CytoSets™ Capture Antibodies

After printing CytoSets™ capture antibodies onto FAST slides or nitrocellulose filters, these support media were allowed to dry as described. Slides and filters were then blocked with BBSA-T, for 30 min to 1 hr, at ambient temperature (filters) or 37° C. (slides). All incubations were done on an orbital table (ambient temperature incubations) or in a shaking incubator (37° C. incubations).

Purified, recombinant cytokine antigen (contained in each kit) was then diluted to various concentrations (typically between 1-10 ng/ml) in BBSA-T. Slides or filters, containing CytoSets™ capture antibodies, were then incubated with this antigen solution at ambient temperature (filters) or 37° C. (slides). Slides and filters were then washed three times with PBS-T, 3-5 min per wash, at ambient temperature. These slides and filters, containing capture antibody with bound antigen, were then incubated with detector antibody (contained in each kit) diluted 1:2500 in BBSA-T for 1 hr, at ambient temperature (filters) or 37° C. (slides). Slides and filters were then washed with PBS-T as described above.

These slides and filters, containing capture antibody, bound antigen, and bound detector antibody, were then incubated with streptavidin•HRPO (contained in each kit) diluted 1:2500 in BBSA-T for 1 hr, at ambient temperature (filters) or 37° C. (slides). Slides and filters were then washed with PBS-T as described above. The slides and filters were then developed and imaged as described below.

Basic Protocol for Antibody and Antigen Incubations: FAST Slides Printed with Anti-Peptide Tag Capture Antibodies

After printing anti-peptide tag capture antibodies onto FAST slides, the slides were allowed to dry as described. Slides were then blocked with BBSA-T, for 30 min to 1 hr, at 37° C. in a shaking incubator (37° C. incubations).

Purified scFvs, containing peptide tags, were then diluted to various concentrations (typically between 0.1 and 100 μg/ml) in BBSA-T. Slides containing anti-peptide tag capture antibodies were then incubated with this antigen solution for 1 hr at 37° C. Slides were then washed three times with PBS-T, 3-5 min per wash, at ambient temperature.

Slides containing anti-peptide tag capture antibodies and bound scFvs were then incubated with biotinylated human fibronectin or biotinylated human glycophorin (as antigens) diluted to various concentrations (typically 1-10 μg/ml) in BBSA-T, for 1 hr at 37° C. Slides were then washed with PBS-T as described above.

Slides containing anti-peptide tag capture antibodies, bound scFvs, and bound biotinylated antigens were then incubated with Neutravidin•HRPO diluted 1:1000 or 1:100,000 in BBSA-T, for 1 hr at 37° C. Slides were then washed with PBS-T as described above. These slides were then developed and imaged as described below.

Developing and Imaging of FAST™ Slides and Nitrocellulose Filters Containing Antibody Microarrays

After washing in PBS-T, slides containing anti-peptide tag antibodies, bound scFvs, antigens, and Neutravidine•HRPO, or nitrocellulose filters containing CytoSets™ antibodies, bound cytokine antigens, detector antibody, and streptavidin•HRPO, were rinsed with PBS, then developed with Supersignal™ ELISA Femto Stable Peroxide Solution and Supersignal™ ELISA Femto Luminol Enhancer Solution (Pierce catalog # 37075) following the manufacturer's recommendations.

FAST™ slides and filters were imaged using the Kodak Image Station 440CF. A 1:1 mixture of peroxide solution:luminol was prepared, and a small volume of this mixture was placed onto the platen of the image station. Slides were then placed individually (microarray-side down) into the center of the platen, thus placing the surface area of the nitrocellulose-containing portion of the slide (containing the microarrays) into the center of the imaging field of the camera lens. In this way the small volume of developer, present on the platen, then contacted the entire surface area of the nitrocellulose-containing portion of the slide. Nitrocellulose filters were treated in the same manner, using somewhat larger developer volumes on the platen. The Image Station cover was then closed and microarray images were captured. Camera focus (zoom) was set to 75 mm (maximum; for FAST™ slides) or 25 mm for filters. Exposure times ranged from 30 sec to 5 minutes. Camera f-stop settings ranged from 1.2 to 8 (Image Station f-stop settings are infinitely adjustable between 1.2 and 16).

Archiving and Analysis of Microarray Images

Archiving and analysis of microarray images is done using the Kodak 1 D 3.5.2 software package. Regions of interest (ROIs) were drawn to frame groups of capture antibodies (printed at known locations on the microarrays), typically in groups of four (two-by-two) or 64 (eight-by-eight) microspots. Numerical ROI values, representing net, sum, minimum, maximum, and mean intensities, as well standard deviations and ROI pixel areas, were automatically calculated by the software. These data were then transformed into Microsoft Excel for statistical analyses.

Results

Two microarray-type patterns of human tumor necrosis factor a (TNF-α) capture antibody (from CytoSets™ kit) were printed onto nitrocellulose with a modified inkjet printer using Microsoft PowerPoint. TNF-α capture antibody was diluted to 1.25 ng/ml in 1% glycerol for printing. After drying, the filter was blocked with BBSA-T. The microarrays were then probed with purified recombinant human TNF-α (5.65 ng/ml) as antigen. The filter was then washed with PBS-T. Detector antibody and streptavidine•HRPO were then used for detection of bound antigen. After washing in PBS-T, the microarrays were developed using chemiluminescence and imaged on a Kodak Image Station 440CF. High resolution images were gerature with feature sizes below 50 μm.

A single microarray of human interleukin-6 (IL-6) capture antibody (from CytoSets™ kit) was printed onto a FAST™ slide with a pin-style microarray printer (4-pin print pattern) programmed to print the pattern depicted in the figure. IL-6 capture antibody was diluted to 0.5 mg/ml in 10% glycerol. One nanoliter microspots of capture antibody were printed which contained 500 μg/microspot. After drying, the slide was blocked with BBSA-T. The microarray was then probed with purified recombinant human IL-6 (5 ng/ml) as antigen. The slide was then washed with PBS-T. Detector antibody and streptavidin•HRPO were then used for detection of bound antigen. After washing in PBS-T, the microarrays were developed using chemiluminescence and imaged on a Kodak Image Station 440CF. The method produced bright images with array feature sizes corresponding to 300 μm spots. In additional experiments, dilution of capture antibody or antigen gave increased or reduced signals corresponding to a direct relationship between the amount of antigen bound and the signal produced.

Microarrays (8-by-8 microspots) of anti-peptide tag capture antibodies (HA.11, specific for the influenza virus hemagglutinin epitope YPYDVPDYA (SEQ ID No. 92); 9E10, specific for the EQKLISEEDL (SEQ ID No. 91) amino acid region of the Myc oncoprotein; and FLOPC-21, a negative control antibody of unknown specificity) were printed onto a FAST™ slide with a pin-style microarray printer (4-pin print pattern) programmed to print the pattern depicted in the figure. Capture antibodies were diluted to 0.5 mg/ml in 20% glycerol. One nanoliter microspots were printed which contained serial two-fold dilutions of 500, 250, 125, and 62.5 μg/microspot. After drying, the filter was blocked with BBSA-T. The microarrays were then successively probed with aliquots of culture supernatant and periplasmic lysate harvested from an E. coli strain harboring the plasmid construct which directs the expression of the HA-HFN scFv upon arabinose induction. The slide was then washed with PBS-T. The microarrays were then probed with biotinylated human fibronectin (3.3 μg/ml). After washing with PBS-T, the microarrays were probed with excess Neutravidin•HRPO (1:1000). After washing in PBS-T, the microarrays were developed using chemiluminescence and imaged on a Kodak Image Station 440CF.

Microarrays of human interleukin-6 (IL-6) capture antibody (from CytoSets™ kit) were printed onto a FAST™ slide, and 4 different surfaces, with a pin-style microarray printer (4-pin print pattern) programmed to print the pattern depicted in the figure. Human IL-6 capture antibody was diluted in 20% glycerol and printed to yield serial three-fold dilutions ranging from 300, 100, 33, 11, 3.6, 1, 0.3, and 0.1 μg/microspot. A negative control capture antibody, specific for human interferon-α (IFN-α) was also printed at 50 μg/microspot. After drying, the slide was blocked with BBSA-T. The microarrays were then probed with purified recombinant human IL-6 (5 ng/ml) as antigen. The slide was then washed with PBS-T. Detector antibody and streptavidin•HRPO were then used for detection of bound antigen. After washing in PBS-T, the microarrays were developed using chemiluminescence and imaged on a Kodak Image Station 440CF. Signal was seen from spots containing 1 μg/spot and higher concentrations.

### Example 9

### Determination of Anti-Idiotype

A. MicroArray Printing

Stock solutions of the anti-IgM antibody (S1 C5; anti-idiotype monoclonal antibody), the goat anti-mouse Fc antibody (this antibody recognizes the constant (Fc) regions of mouse antibodies) and anti-flag antibody were prepared at a concentration of 1 mg/ml or greater in PBS. For printing, the antibodies were brought to 800 μg/ml in 1×Print Buffer (1×PBS, 20% glycerol, 0.001% Tween-20) by adding ¼ volume of 4×Print Buffer (4×PBS, 80% glycerol, 0.004% Tween-20) to 3/4 volume of a 1 mg/ml antibody solution in PBS. Two-fold serial dilutions were made of each antibody such that all antibodies were at 9 different concentrations in 1×Print Buffer (Table 9). Forty μl of antibody solution was transferred to a 96-well PCR plate.

Each of the antibodies were printed on FAST™ nitrocellulose—coated glass slides (Schleicher and Schuell) using a Telechem pin (CM-2) in a Cartesian printer (MicroSys 5100). Printing was performed at 55 to 60% relative humidity. The slides were subsequently incubated overnight at 4° C. for maximum adsorption to the nitrocellulose.

B. Preparation of 38C13 Cell Extract

B cells (38C13) were grown in culture (Growth medium: RPMI 1640, 10% fetal calf serum, 55 μl 2-mercaptoethanol, penicillin and streptomycin) in 5% CO₂, 90% relative humidity and 37° C. to a density of 0.7×10⁶ cells/ml. A 2.5 ml aliquot (1.75×10⁶ cells total) was spun down at 1200 rpm for 5 minutes at 4° C. The pellet was then washed one time with 4 ml of RPMI 1640 (Gibco), and spun down again at 1200 rpm for 5 minutes at 4° C. The pellet was then resuspended at 4° C. in 175 μl of RPMI 1640 (Gibco), giving a concentration of 10⁶ cells per 100 μl. Resuspension was carried out by gently pipeting up and down 3-4 times.

Small (less than 1 ml) aliquots of tissue culture cells (38C13 and C6V_{L} cells) prepared as described above were stored frozen in liquid nitrogen or at −80° C. in Freezing Medium (frequently 90% fetal calf serum/10% DMSO). The frozen cells were thawed quickly by rolling tube containing the aliquot between the palms. The cells were diluted immediately 10-fold with 4° C. PBS and centrifuged at 1200 rpm for 5 minutes at 4° C. Cells were then washed three times with 4° C. PBS at a density of 10⁶ cells/ml, based on the number of cells that were frozen for storage. The resuspended cells were used immediately for capture.

TABLE 9
Array Map (μg/ml)

| | 1 | 2 | 3 | 4 | 5 | 6 | 7 | 8 | 9 | 10 | 11 |
|---|---|---|---|---|---|---|---|---|---|---|---|
| A | NV-HRP 400 | — | S1C5 400 | S1C5 200 | S1C5 100 | S1C5 50 | S1C5 25 | S1C5 12.5 | S1C5 6.25 | S1C5 3.12 | — |
| B | NV-HRF 200 | — | S1C5 400 | S1C5 200 | S1C5 100 | S1C5 50 | S1C5 25 | S1C5 12.5 | S1C5 6.25 | S1C5 3.12 | — |
| C | NV-HRP 100 | — | g a-m Fc | g a-m Fc | g a-m Fc | g a-m Fc | g a-m Fc | g a-m Fc | g a-m Fc | g a-m Fc | — |
| | | | 121.9 | 60.95 | 30.475 | 15.238 | 7.619 | 3.809 | 1.905 | 0.952 | |
| D | — | — | g a-m Fc | g a-m Fc | g a-m Fc | g a-m Fc | g a-m Fc | g a-m Fc | g a-m Fc | g a-m Fc | — |
| | | | 121.9 | 60.95 | 30.475 | 15.238 | 7.619 | 3.809 | 1.905 | 0.952 | |
| E | — | — | g a-m Fc | g a-m Fc | g a-m Fc | g a-m Fc | g a-m Fc | g a-m Fc | g a-m Fc | g a-m Fc | — |
| | | | 121.9 | 60.95 | 30.475 | 15.238 | 7.619 | 3.809 | 1.905 | 0.952 | |
| F | NV-HRP 50 | — | g a-m Fc | g a-m Fc | g a-m Fc | g a-m Fc | g a-m Fc | g a-m Fc | g a-m Fc | g a-m Fc | NV-HRP 100 |
| | | | 121.9 | 60.95 | 30.475 | 15.238 | 7.619 | 3.809 | 1.905 | 0.952 | |
| G | NV-HRP 100 | — | anti-Flag | anti-Flag | anti-Flag | anti-flag | anti-Flag | anti-Flag | anti-Flag | anti-Flag | NV-HRP 200 |
| | | | 121.9 | 60.95 | 30.475 | 15.238 | 7.619 | 3.809 | 1.905 | 0.952 | |
| H | NV-HRP 200 | — | anti-Flag | anti-Flag | anti-Flag | anti-flag | anti-Flag | anti-Flag | anti-Flag | anti-Flag | NV-HRP 400 |
| | | | 121.9 | 60.95 | 30.475 | 15.238 | 7.619 | 3.809 | 1.905 | 0.952 | |

C. Array Incubations

The printed slides were brought to room temperature and washed three rimes each for one minute with PBS. Following the wash step, the slides were blocked with 1 ml of Block Buffer (3% NMF/PBS/1% Triton X-100) on an orbital shaker in a humidified chamber for 1 hour at room temperature. The slides were then incubated with 38C13 cell extract and control 38C13 purified antibody as shown in Table 10 below. The extract was diluted 1:1 with Block Buffer for the highest concentration, then serially by factors of 10. Fifty μl of each sample was added to the wells and incubated with the array for 1 hour at room temperature on an orbital shaker.

TABLE 10

| Array Number | Sample | Array Number | Sample |
|---|---|---|---|
| 1 | Block Buffer control | 6 | 38C13 Ab 10 μg/ml |
| 2 | Extract (1:2000) | 7 | 38C13 Ab 1 μg/ml |
| 3 | Extract (1:200) | 8 | 38C13 Ab 0.1 μg/ml |
| 4 | Extract (1:20) | 9 | 38C13 Ab 0.01 μg/ml |
| 5 | Extract (1:1) | 10 | Block Buffer Control |

Following the incubation, the wells were then washed three times with 200 μl of PBS/1% Triton X-100 for one minute on an orbital shaker. Fifty microliters of detection antibody (goat anti-mouse IgM HRP 1:5,000 in Block Buffer) were then added to each well and incubated for one hour at room temperature on an orbital shaker. The wells were then washed again three times with 200 μl of PBS/1% Triton X-100 for one minute on an orbital shaker. The slides were then removed from the chamber and rinsed with 500 μl PBS/1% Triton X-100. The arrays were then imaged on Kodak IS1000 in a petri dish, raised from the surface of the dish with two layers of plastic cover slips, with about 1 ml of luminol as shown in FIG. 27.

D. Results

The purified IgM antibody (38C13) gave a strong signal on the S1C5 monclonal antibody loci, down to a concentration of 25 μg/ml spotted protein and at an IgM concentration of 0.1 μg/ml, the lowest IgM concentration used. The 38C13 IgM in the 38C13 cell extracts were detected at a 1:2000 dilution of the extract, the lowest used, down to a concentration of 50 μg/ml printed S1C5. The 38C13 IgM did not bind to the anti-Flag monoclonal negative control, though non-specific binding of the Goat anti-Mouse IgM—HRP antibody can be seen (FIG. 27).

### Example 10

### Preparation and Use of Biological Samples

Preparation of Sample

Sample acquisition—Biological samples, can be obtained by any suitable method, including, but are not limited to, biopsy, laser capture micro-dissection, cells grown in culture, whole blood draw, other bodily fluids, soil samples and other samples that contain biological materials or molecules derived from living sources.

Crude fractionation of sample A preliminary fractionation of the sample for enrichment of the cells or biomolecules of interest from the remainder of the material can be performed Subcellular fractionation A population of cells is often divided into membrane, nuclear, cytoplasmic, microsomal, mitochondrial, or other fractions to examine the location of particular proteins of interest or examine the proteins contained in a location of interest. This subcellular fractionation to enrich that particular compartment therefore increasing the relative concentration of constituents in that compartment compared to the initial sample.

Exemplary Embodiment A: Analysis of Nuclear Proteins in T-Lymphocytes.

An anticoagulant treated blood sample is mixed with an equal volume of phosphate buffered saline (PBS) without Ca²⁺ or Mg²⁺ and the mixture is carefully layered onto an equal volume of ficoll-paque (Amersham Biosciences). The sample is centrifuged at 400×g for 30 minutes such that erythrocytes and granulocytes are pelleted while peripheral blood mono-nuclear cells (PBMC) and platelets remain at the interface supported on the cushion of ficoll-paque. The PBMCs are collected with a Pasteur pipette and transferred to a clean centrifuge tube. Add three volumes of PBS+0.1% BSA, mix gently then centrifuge at 100×g for 10 minutes. Remove the supernatant and resuspend in 6-8 mls of PBS+0.1% BSA. The sample is again centrifuged at 100×g for 10 minutes and the supernatant removed. At this point about 95% of the cells are mononucleocytes.

T cells are negatively isolated from a mononuclear cell (MNC) sample by depletion of B cells, NK cells, monocytes, activated T cells and granulocytes (if present). This is an indirect method to remove the unwanted cells. A mixture of monoclonal antibodies for CD14, CD16 (specific for CD16a and CD16b), CD56 and HLA Class II DR/DP (T cell Kit, Dynal Biotech) is added to the PBMCs and then paramagnetic beads coated with an Fc specific human IgG4 antibody against mouse IgG. (Depletion Dynabeads, Dynal Biotech) are added to capture the antibody bound cells. These coated cells are then separated with a magnet (Dynal MPC®) and discarded.

Resuspend prepared MNC at 1×10⁷ PBMCs in 100-200 μl PBS+0.1% BSA. Add 20 μl heat inactivated FCS. Add 20 μl Antibody Mix (T Cell Kit, Dynal Biotech) per 1×10⁷ PBMCs. Incubate for 10 minutes at 2-8° C. Wash cells by adding 1 ml of PBS/0.1% BSA per 1-5×10⁷ PBMC and centrifuge for 8 minutes at 500×g. Remove supernatant with a pipette. Resuspend cells in 0.9 ml of PBS+0.1% BSA per 1×10⁷ PBMC. Add washed beads to the cells. Use 100 μl Depletion Dynabeads per 1×10⁷ PBMC. Total volume for cell and bead incubation should be 1 ml per 1×10⁷ PBMC. Incubate for 15 minutes at 20° C. with gentle tilting and rotation (incubation at 2-8° C. will reduce the efficiency of monocyte depletion). Resuspend rosettes by careful pipetting 5-6 times, before increasing the volume by adding 1-2 ml of PBS+0.1% BSA per 1×10⁷ PBMC. Place in the Dynal MPC for 2 minute and pipette supernatant (negatively isolated T cells) to a fresh tube.

To prepare nuclear and cytoplasmic extracts, 1-2×10⁸ cells are harvested by centrifugation, washed 3 times with calcium-deficient phosphate-buffered saline, and resuspended to 2.5×10⁷ cells/ml in a buffer containing 10 mM Tris, pH 7.4, 10 mM NaCl, 3 mM MgCl₂, 0.5 mM dithiothreitol, 2.5 mM EGTA, protease inhibitors (5 μg/ml aprotinin, 5 μg/ml antipain, 100 μM benzamidine, 5 μg/ml leupeptin, 5 μg/ml pepstatin, 5 μg/ml soybean trypsin-chymotrypsin inhibitor, and 1 mM phenylmethylsulfonyl fluoride), and phosphatase inhibitors (50 mM NaF and 20 mM sodium pyrophosphate). Resuspended cells are lysed by adding 5% Nonidet P-40 to bring the final concentration of Nonidet P-40 to 0.05% and incubated on ice for 10 minutes. The cell lysates are centrifuged at 300×g for 10 min to separate nuclei from cytoplasmic fraction (see, Park et al. (1995) J. Biol. Chem. 270:20653-20659).

Nuclear pellets are washed once with 1 ml of the same buffer, and resuspended in 300-400 μl of a nuclear extraction buffer containing 20 mM Hepes, pH 7.9, 0.42 M NaCl, 1.5 mM MgCl₂, 25% (v/v) glycerol, 0.2 mM EDTA, 0.5 mM dithiothreitol, and the above protease inhibitors. Resuspended nuclei are incubated on ice for 30 min with occasional shaking to extract the nuclear proteins and finally spun down in a microcentrifuge for 5 minutes. The supernatant with nuclear proteins are dialyzed against PBS, pH 7.4, containing 0.2 mM EDTA, 20% (v/v) glycerol, 1 mM phenylmethylsulfonyl fluoride, and 0.5 mM dithiothreitol.

For preparation of cytoplasmic fractions, the 300×g supernatant are further centrifuged at 100,000×g for 1 h. Cytoplasmic proteins in the supernatant can be labeled directly or precipitated at 1.5 M ammonium sulfate for 30 min on ice, and the precipitated proteins are collected by centrifugation at 100,000×g for 30 minutes. The protein pellets are resuspended in PBS supplemented with the above protease inhibitors, and dialyzed extensively against PBS. If necessary, the protein concentration is determined using a Bio-Rad protein assay kit with bovine serum albumin as a standard.

Exemplary Embodiment B: Examination of Proteins in Eggs of Soybean Cyst Nematodes (SCN, Heterodera glycines)

The procedure has two stages: extraction of the cysts from the soil, and crushing of the cysts to release the eggs (see, e.g., www.extension.iastate.edu/Pages/plantpath/tylka/Frames.html, a website by Gregory L. Tylka, Department of Plant Pathology, Iowa State University). The technique used to recover the cysts of soybean cyst nematode from soil is a combination of wet-sieving and decanting. It is a modification of a mycological technique used to recover large spores of soil-inhabiting fungi (see, e.g., Gerdemann et al. (1955) Mycologia 47:619-632) and is based on the fact that the size range for soybean cyst nematode cysts is 470-790 μm by 210-580 μm. The procedure is as follows: Obtain a well-mixed 100 cc soil sample (approx. 1/2 cup). Fill a bucket with 2 quarts of water. Pour the soil into the water, break any clumps with your fingers, and mix the soil suspension well for 15 seconds. Let the suspension settle for 15 seconds. Pour the soil suspension through an 8-inch-diameter #20 (850 μm pore) sieve nested over a #60 (250 μm pore) sieve. Any sediment that settles out in the bottom of the bucket should be discarded. Rinse, with water, the debris caught on the top sieve, then discard its contents. Carefully wash the cysts and accompanying sediments trapped on the #60 sieve into a clean, properly labeled beaker or directly into a 100 ml polypropylene grinding tube, using as little water as possible.

The result of the above technique is a suspension of SCN cysts, along with organic debris and sediments similar in size to the cysts. Eggs of soybean cyst nematode average 47 μm by 100 μm in size. The cysts are crushed to release and recover the eggs as follows (see, Niblack et al. (1993) Supplement to the Journal of Nematology 25:880-886):

Wash the cyst suspension from the beaker into a 100 ml polypropylene grinding tube. Do not fill the tube more than half full. Grind the cysts carefully between the inside surface of the tube and the 1-mm-deep grooves on a stainless steel pestle attached to a Talboys Model 101 motorized laboratory stirrer. Grind the cysts for exactly 60 seconds at 3,500 RPM. Rinse the pestle thoroughly with a wash bottle when finished grinding. Alternatively, cysts can be crushed in a blender for 60 seconds at medium speed, provided a small canister is used atop the blender. The blender canister should hold no more than 500 ml or so for blending to be effective in rupturing the cysts. After grinding or rupturing the cysts, pour the suspension in the tube or blender canister through a stainless steel, 3-inch-diameter #200 (75 μm pore) sieve over a #500 (25 μm pore) sieve. Rinse the tube or canister several times with tap water, each time pouring the contents through the sieves. Carefully rinse with water the sediments caught on the #200 sieve, then discard. Finally, carefully wash sediments and eggs caught on the #500 sieve into a clean beaker with as little water as possible. Collected eggs are then homogenized at 4° C. in 1 ml buffer L (10 mM HEPES, pH 7.8, 1.5 mM MgCl₂, 0.1 mM EGTA, 0.5 mM DTT, 5% glycerol) and 100 μg/ml leupeptin. This homogenate can be directly labeled or sub-fractionated further.

Sample Labeling

Many different methods of labeling a biological sample are known. These include, but are not limited to, use of fluorescent (Molecular Probes) and radioactive probes (ICN, New England Nuclear), resonance light scattering particles (Genicon Sciences), nano-barcodes (SurroMed), and attachment of haptens, such as biotin. The avidin-biotin interaction is one of the strongest known non-covalent biological interactions between a protein and a ligand (K_{d}=10⁻¹⁵ M). This interaction has been extensively utilized for the isolation and identification of labeled proteins. Biotin molecules with a variety of different linkage chemistries are available from several different companies (Pierce Chemical, Molecular Probes, etc.). In this example an N-hydroxysuccinimide (NHS) ester-modified biotin will be used to conjugate to the primary amines of a protein sample. The concentration of a protein sample is determined by any number of common methods such as a modified Lowry assay (Pierce Chemical). In complex mixtures, the molar concentration can be estimated by using a molecular weight of 50,000 Daltons as an average. A solution of NHS-Biotin is added to an aliquot of protein (2-10 mg/ml in PBS), such that the reactive biotin is at a 10-20 fold molar excess (or other as determined empirically). The sample is incubated on ice for 2 hours to allow the formation of an amide bond between the biotin and the protein prior to removal of the unreacted biotin via dialysis or desalting column.

Additional chemistries include maleimide or iodoacetyl modified biotin for formation of thioether bonds through the sulfhydryl groups of proteins and hydrazide modified biotins to allow creation of a hydrozone bond to an oxidized carbohydrate. Biotins with photoactivatable groups are also available for the conjugation to DNA, RNA, carbohydrates and proteins. Additional crosslinkers such as EDC allow the activation of a carboxyl group to allow coupling to an amino group. Such methods are well-known, (see, e.g., Pierce Chemical catalog or web site, sections on “non-radioactive labeling” and “cross-linking reagents”). Most of these chemistries are also available using fluors with different excitation and emission characteristics (Molecular Probes) as well as radioactive probes (Pierce Chemical).

Pattern Recognition

Pattern recognition software is well known and readily available (see, e.g., U.S. Pat. No. 6,340,568 B2, U.S. Pat. No. 6,327,035 B1; PARTEK PRO2000® commercially available from Partek, Inc. St. Charles, Mo.; IMAGE-PRO® and other such software and products available from Media Cybernetics).

The resulting profiles can be provided as databases and used for assessing unknowns and for diagnostic purposes. Databases of profiles are provided. Unknown samples being tested for a particular condition can be compared to profiles of knowns to thereby identify components of the samples or effect a diagnosis or extract other information. Databases can be stored on computer-readable media, such floppy disks, compact disks, digital video disks, computer hard drives and other such media.

Since modifications will be apparent to those of skill in this art, it is intended that this invention be limited only by the scope of the appended claims.

What is claimed is:

1. A combination, comprising:
a) an addressable collection of binding sites, comprising:
i) a plurality of capture agents, wherein each capture agent is preselected to specifically bind to a pre-selected tag; and
ii) a plurality of tagged reagents, each comprising one of the pre-selected tags, wherein:
each locus in the collection comprises the same capture agent;
the tagged reagent comprises a molecule and a tag;
each tag is pre-selected to specifically bind to a capture agent;
each tag is bound to a capture agent thereby forming a complex of the tagged reagent with the capture agent;
each locus comprises a plurality of tagged reagents; and
each of the different molecules at each locus comprises the same pre-selected tag; and
b) one or more of software comprising instructions for pattern recognition and an imager for detecting patterns.

2. The combination of claim 1 that is packaged as a kit that optionally includes instructions for profiling.

3. The combination of claim 1, wherein the capture agents and/or tags are polypeptides.

4. The combination of claim 3, wherein the polypeptides are antibodies or fragments thereof.

5. The combination of claim 4, wherein the tagged reagents comprises scFvs.

6. The combination of claim 1, wherein the tagged reagents comprise scFvs.

7. The combination of claim 1, wherein the capture agent is selected from the group consisting of capture agents that comprise a polypeptide, a nucleic acid, a carbohydrate, a lipid, a polysaccharide, a metal, an antibody, a cell membrane receptor, antiserum reactive with specific antigenic determinants, a lectin, a sugar, a polysaccharides, a cell, a cellular membranes and an organelle.

8. The combination of claim 1, wherein the tagged reagent is selected from the group consisting of tagged reagents that comprise a polypeptide, a nucleic acid, a carbohydrate, a lipid, a polysaccharide, a metal, an antibody, a cell membrane receptor, antiserum reactive with specific antigenic determinants, a lectin, a sugar, a polysaccharides, a cell, a cellular membrane and an organelle.

9. The combination of claim 1, wherein the capture agents are antibodies, and the pre-selected tags comprise polypeptides to which the capture agents bind.

10. The combination of claim 1, wherein the capture agents are arranged in an array.

11. The combination of claim 1, wherein the capture agents are linked directly or indirectly to a solid support.

12. The combination of claim 1, wherein a tagged reagent and capture agent in the collection are covalently linked.

13. The combination of claim 11, wherein the support is particulate.

14. The combination of claim 13, wherein the particles are optically encoded.

15. The combination of claim 1, wherein the capture agents are addressably tagged by linking them to electronic, chemical, optical or color-coded labels.

16. The combination of claim 10, wherein the array is addressable.

17. The combination of claim 1, wherein the tag is encoded by a nucleic acid molecule that comprises two domains:
the first domain encodes a sequence of amino acids that specifically binds to a capture agent; and
the second domain comprises a sequence of nucleic acids for amplification of genes containing the sequence of amino acids encoded by the first domain.

18. The combination of claim 1, wherein
each of the tags is encoded by oligonucleotides that comprises at least two regions, wherein the regions are a divider region that contains a sequence of nucleotides that comprise a sequence unique to a target library, and an polypeptide-encoding region (E) that encodes a sequence of amino acids to which a capture agent binds.

19. The combination of claim 18, wherein the divider region is 3′ of the polypeptide-encoding region.

20. The combination of claim 18, wherein the divider and E regions comprise at least about 10 nucleotides.

21. The combination of claim 20, wherein the divider and E regions comprise at least about 15 nucleotides.

22. The combination of claim 18, wherein each of the oligonucleotides further comprises a common region, wherein the common region is shared by each of the oligonucleotides in the set, and is of a sufficient length to serve as a unique priming site for amplifying nucleic acid molecules that comprise the sequence of nucleotides that comprises the common region.

23. The combination of claim 22, wherein the common region is 3′ of the polypeptide-encoding region (E) and/or of the divider region.

24. The combination of claim 1, wherein the capture agents are immobilized at discrete loci on a solid support, wherein the capture agents at each loci specifically bind to one of the preselected tagged reagents.

25. The combination of claim 24, wherein the capture agents are antibodies; and the preselected tagged reagents comprise an polypeptide or plurality thereof to which the antibodies bind.

26. The combination of claim 1 that comprises from 3 up to 10⁶ capture agents that specifically bind to different tags.

27. The combination of claim 22, wherein the length of each of the divider, E region and common regions is at least about 14 nucleotides.

28. The combination of claim 18, wherein the length of each of the divider and E regions is independently at least about 14 nucleotides.

29. The combination of claim 28, wherein the length of each of the divider and E regions is independently at least about 16 nucleotides.

30. The combination of claim 1, wherein the tagged reagents comprise a tagged library, produced by a method comprising:
incorporating each one of a set of oligonucleotides into a nucleic acid molecule in a library of nucleic acid molecules to create a tagged library, wherein the set of oligonucleotides has the formula:
5′-Dₙ-Eₘ-3′
wherein:
each D is a unique sequence among the set of oligonucleotides and contains at least about 10 nucleotides;
each E encodes an a sequence of amino acids that comprises a polypeptide that specifically binds to a capture agent in the collection;
each polypeptide that specifically binds is unique in the set;
each polypeptide comprises a sequence of amino acids to which a capture agent binds;
n is 0 or is an integer of 2 or higher;
m is an integer of 2 or higher; and
the oligonucleotides are single-stranded, double-stranded, and/or partially double-stranded.

31. The combination of claim 30, wherein m×n is between about 10 to about 10¹², inclusive.

32. The combination of claim 30, wherein m×n is between about 10 to about 10⁹, inclusive.

33. The combination of claim 30, wherein m×n is from about 10 up to about 10⁶, inclusive.

34. The combination of claim 30, wherein the library of nucleic acid molecules encodes a library comprising scFvs or T cell receptors.

35. The combination of 30, wherein each oligonucleotide further comprises a common region C, and comprises formula:
5′C-Dₙ-Eₘ3′,
wherein the common region is shared by each of the oligonucleotides in the set, and is of a sufficient length to serve as a unique priming site for amplifying nucleic acid molecules that comprise the sequence of nucleotides that comprises the common region.

36. The combination of claim 35, wherein the library of nucleic acid molecules encodes a library comprising scFvs or T cell receptors.

37. A system for profiling samples, comprising:
a) a combination of claim 1; and
b) a computer system programmed with the software for pattern recognition.

38. The system of claim 37 that comprises an imager for detecting and/or digitizing the patterns.

39. A method for profiling a sample, comprising:
a) providing an addressable collection comprising a plurality binding sites, wherein the collection comprises:
i) a plurality of capture agents, wherein each capture agent is preselected to specifically bind to a pre-selected tag; and
ii) a plurality of tagged reagents, each comprising one of the pre-selected tags, wherein:
each locus in the collection comprises the same capture agent;
the tagged reagent comprises a molecule and a tag;
each tag is a moiety pre-selected to specifically bind to a capture agent;
each tag is bound to a capture agent thereby forming a complex of the molecule with a capture agent;
each locus comprises a plurality of different molecules;
each of the different molecules at each locus comprises the same pre-selected tag;
b) contacting the collection with a sample under conditions whereby components of the sample specifically bind to binding sites of the collection; and
c) detecting binding of the components, wherein loci to which the components bind provides a profile of the sample.

40. The method of claim 39, wherein the collection of addressable binding sites is produced by mixing capture agents and tagged reagents, where the each tagged reagent is specific for only one capture agent.

41. The method of claim 39, wherein the collection of addressable binding sites is produced by mixing capture agents and tagged reagents, and steps a) and b) are performed simultaneously so that sample is added with the tagged reagents to a collection of capture agents, whereby the collection of addressable binding sites with bound sample components is produced.

42. The method of claim 39, further comprising detecting or identifying the pattern of loci to which components of the sample bind.

43. The method of claim 42, wherein the pattern is produced by comparing the results from the test sample to a control.

44. The method of claim 39, wherein the profile is stored in a database.

45. A computer system or computer readable medium, comprising the database produced by the method of claim 44.

46. The method of claim 39, wherein the tag is encoded by a nucleic acid molecule that comprises two domains:
the first domain encodes a sequence of amino acids that specifically binds to a capture agent; and
the second domain comprises a sequence of nucleic acids for specific amplification of genes containing the sequence of amino acids encoded by the first domain.

47. The method of claim 39, wherein
each of the tags is encoded by oligonucleotides that comprises at least two regions, wherein the regions are a divider region that contains a sequence of nucleotides that comprise a sequence unique to a target library, and an polypeptide-encoding region (E) that encodes a sequence of amino acids to which a capture agent binds.

48. The method of claim 47, wherein the divider region is 3′ of the polypeptide-encoding region (E).

49. The method of claim 47, wherein the divider and polypeptide (E) regions comprise at least about 10 nucleotides.

50. The method of claim 49, wherein the divider and polypeptide (E) regions comprise at least about 15 nucleotides.

51. The method of claim 47, wherein each of the oligonucleotides further comprises a common region, wherein the common region is shared by each of the oligonucleotides in the set, and is of a sufficient length to serve as a unique priming site for amplifying nucleic acid molecules that comprise the sequence of nucleotides that comprises the common region.

52. The method of claim 51, wherein the common region is 3′ of the polypeptide (E)-encoding region and/or of the divider region.

53. The method of claim 39, wherein the capture agents are immobilized at discrete loci on a solid support, wherein the capture agents at each loci specifically bind to one of the preselected tagged reagents.

54. The method of claim 53, wherein the capture agents are antibodies; and the pre-selected tags comprise a polypeptide or plurality thereof to which the antibodies bind.

55. The method of claim 54, wherein the tagged reagents further comprise scFvs or T cell receptors.

56. The method of claim 39, wherein the collection in the combination comprises from 3 up to 10⁶ capture agents that specifically bind to different tags.

57. The method of claim 47, wherein the length of each of the divider, polypeptide (E) and common regions is at least about 14 nucleotides.

58. The method of claim 48, wherein the length of each of the divider, polypeptide (E) and common regions is at least about 14 nucleotides.

59. The method of claim 39, wherein the capture agents are antibodies; and the pre-selected tags comprise polypeptide (E)s to which the capture agents bind.

60. The method of claim 54, wherein the collection comprises up to about 10³ antibodies.

61. The method of claim 59, wherein the collection comprises up to about 10³ antibodies.

62. The method of claim 47, wherein the length of each of the divider and polypeptide (E) regions is independently at least about 14 nucleotides.

63. The method of claim 48, wherein the length of each of the divider and polypeptide (E) regions is independently at least about 14 nucleotides.

64. The method of claim 47, wherein the length of each of the divider and polypeptide (E) regions is independently at least about 16 nucleotides.

65. The method of claim 39, wherein the tagged reagents comprise a tagged library, produced by a method comprising:
incorporating each one of a set of oligonucleotides into a nucleic acid molecule in a library of nucleic acid molecules to create a tagged library, wherein the set of oligonucleotides has the formula:
5′-Dₙ-Eₘ-3′
wherein:
each D is a unique sequence among the set of oligonucleotides and contains at least about 10 nucleotides;
each E encodes an a sequence of amino acids that comprises a polypeptide that specifically binds to a capture agent in the collection;
each polyeptide that specifically binds to a capture agent is unique in the set;
each polyeptide that specifically binds to a capture agents comprises a sequence of amino acids to which a capture agent binds;
n is 0 or is an integer of 2 or higher;
m is an integer of 2 or higher; and
the oligonucleotides are single-stranded, double-stranded, and/or partially double-stranded.

66. The method of claim 65, wherein the library of nucleic acid molecules encodes a library comprising scFvs or T cell receptors.

67. The method of claim 65, wherein m×n is between about 10 to about 10¹², inclusive.

68. The method of claim 65, wherein m×n is between about 10 to about 10⁹, inclusive.

69. The method of claim 65, wherein m×n is from about 10 up to about 10⁶, inclusive.

70. The method of claim 65, wherein each oligonucleotide further comprises a common region C, and comprises formula:
5′C-Dₙ-Eₘ3′,
wherein the common region is shared by each of the oligonucleotides in the set, and is of a sufficient length to serve as a unique priming site for amplifying nucleic acid molecules that comprise the sequence of nucleotides that comprises the common region.

71. The method of claim 70, wherein the library of nucleic acid molecules encodes a library comprising scFvs or T cell receptors.

72. The method of claim 39, wherein the capture agents and/or tags are polypeptides.

73. The method of claim 72, wherein the polypeptides comprise antibodies or fragments thereof.

74. The method of claim 73, wherein the tagged reagents comprise scFvs or T cell receptors.

75. The method of claim 39, wherein the tagged reagents comprise scFvs.

76. The method of claim 39, wherein the capture agent is selected from the group consisting of a agents that comprise a polypeptide, a nucleic acid, a carbohydrate, a lipid, a polysaccharide, a metal, an antibody, a cell membrane receptor, antiserum reactive with specific antigenic determinants, a lectin, a sugar, a polysaccharides, a cell, a cellular membranes and an organelle.

77. The method of claim 39, wherein the tag is selected from the group consisting of a polyeptide tags that comprise a polypeptide to which a capture agent binds, a nucleic acid, a carbohydrate, a lipid, a polysaccharide, a metal, an antibody, a cell membrane receptor, antiserum reactive with specific antigenic determinants, a lectin, a sugar, a polysaccharides, a cell, a cellular membranes and an organelle.

78. The method of claim 39, wherein the capture agents are arranged in an array.

79. The method of claim 39, wherein the capture agents are linked directly or indirectly to a solid support.

80. The method of claim 39, wherein a tagged reagent and capture agent in the collection are covalently linked.

81. The method of claim 79, wherein the support is particulate.

82. The method of claim 81, wherein the particles are optically encoded.

83. The method of claim 78, wherein the array is addressable.

84. A method for preparing a capture system that displays a collection of binding sites, comprising:
a) providing an addressable collection of a plurality of capture agents, wherein each capture agent is pre-selected to specifically bind to a pre-selected tag, wherein:
each locus in the collection comprises the same capture agent;
b) providing a plurality of tagged reagents, each comprising one of the pre-selected tags, wherein:
each tagged reagent comprises a molecule and a tag; and
each tag is a moiety pre-selected to specifically bind to a capture agent;
c) contacting the plurality of tagged reagents to the addressable collection of the plurality of capture agents to form a capture system that displays a diverse collection of binding sites, wherein:
each tag is bound to a capture agent thereby forming a complex of the molecule with the capture agent;
each locus comprises a plurality of different molecules; and
each of the different molecules at each locus comprises the same pre-selected tag, thereby preparing a capture system that displays a diverse collection of binding sites.

85. The method of claim 84, wherein the diversity of the binding sites is selected from the group consisting of 10², 10³, 10⁴, 10⁵, 10⁶, 10⁷, 10⁸, 10⁹, 10¹⁰, 10¹¹ and 10¹².

86. The method of claim 84, wherein the capture agents are antibodies, and the pre-selected tags comprise polyepeptides to which the capture agents bind.

87. The method of claim 86, wherein the tagged reagent comprises a polypeptide.

88. The method of claim 87, wherein the polypeptide comprises a scFv.

89. The method of claim 87, wherein the polypeptide comprises a T cell receptor (TCR) or fragment thereof.

90. The method of claim 84, wherein the addressable collection is positionally addressable; and
each locus comprises a spot on a solid support.

91. The method of claim 90, wherein the solid support comprises a well or pit or plurality thereof on the surface.

92. The method of claim 90, wherein the solid support is selected from the group consisting of plates, beads, microbeads, whiskers, combs, hybridization chips, membranes, single crystals, ceramics and self-assembling monolayers.

93. The method of claim 90, wherein the solid support is selected from the group consisting of silicon, celluloses, metal, polymeric surfaces and radiation grafted supports.

94. The method of claim 93, wherein the solid support is selected from the group consisting of gold, nitrocellulose, polyvinyidiene fluoride (PVDF), radiation grafted polytetrafluoroethylene, polystyrene, glass and activated glass.

95. The method of claim 84, wherein the addressable collection of capture agents are addressably tagged by linking them to electronic, chemical, optical or color-coded labels.

96. The method of claim 84, wherein the tag is encoded by a nucleic acid molecule that comprises two domains:
the first domain encodes a sequence of amino acids that specifically binds to a capture agent; and
the second domain comprises a sequence of nucleic acids for specific amplification of genes containing the sequence of amino acids encoded by the first domain.

97. The method of claim 84, wherein
each of the tags is encoded by oligonucleotides that comprises at least two regions, wherein the regions are a divider region that contains a sequence of nucleotides that comprise a sequence unique to a target library, and a polypeptide-encoding region that encodes a sequence of amino acids to which a capture agent binds.

98. The method of claim 84, wherein each of the oligonucleotides further comprises a common region, wherein the common region is shared by each of the oligonucleotides in the set, and is of a sufficient length to serve as a unique priming site for amplifying nucleic acid molecules that comprise the sequence of nucleotides that comprises the common region.

99. The method of claim 84, wherein the tagged reagents comprise a tagged library, produced by a method comprising:
incorporating each one of a set of oligonucleotides into a nucleic acid molecule in a library of nucleic acid molecules to create a tagged library, wherein the set of oligonucleotides has the formula:
5′-Dₙ-Eₘ-3′
wherein:
each D is a unique sequence among the set of oligonucleotides and contains at least about 10 nucleotides;
each E encodes an a sequence of amino acids that comprises an polypeptide that specifically binds to a capture agent in the collection;
each epitope is unique in the set;
each epitope is a sequence to which a capture agent binds;
n is 0 or is an integer of 2 or higher;
m is an integer of 2 or higher; and
the oligonucleotides are single-stranded, double-stranded, and/or partially double-stranded.

100. The method of claim 99, wherein the library of nucleic acid molecules encodes a library comprising scFvs or T cell receptors.

101. The method of claim 99, wherein m×n is between about 10 to about 10¹², inclusive.

102. The method of claim 99, wherein m×n is between about 10 to about 10⁹, inclusive.

103. The method of claim 99, wherein m×n is from about 10 up to about 10⁶, inclusive.

104. The method of claim 99, wherein each oligonucleotide further comprises a common region C, and comprises formula:
5′C-Dₙ-Eₘ3′,
wherein the common region is shared by each of the oligonucleotides in the set, and is of a sufficient length to serve as a unique priming site for amplifying nucleic acid molecules that comprise the sequence of nucleotides that comprises the common region.

105. The method of claim 104, wherein the library of nucleic acid molecules encodes a library comprising scFvs or T cell receptors.

106. A positionally addressable collection of binding sites, comprising:
a) a plurality of capture agents bound to a solid support, wherein:
each capture agent is preselected to specifically bind to a pre-selected tag; and
each locus that comprises the capture agents is within 1 mm or less from a neighboring locus; and
b) a plurality of tagged reagents, each comprising one of the pre-selected tags, wherein:
each locus in the collection comprises the same capture agent;
the capture agents at each locus are different;
the tagged reagent comprises a molecule and a tag;
each tag is re-selected to specifically bind to a capture agent;
each tag is bound to a capture agent thereby forming a complex of the tagged reagent with the capture agent;
each locus comprises a plurality of tagged reagents; and
each of the different molecules at each locus comprises the same pre-selected tag.

107. The method of claim 106, wherein the molecules in the tagged reagents are selected from the group consisting of a polypeptide, a nucleic acid, a carbohydrate, a lipid, a polysaccharide, a metal, an antibody, a cell membrane receptor, antiserum reactive with specific antigenic determinants, a lectin, a sugar, a polysaccharides, a cell, a cellular membranes and an organelle.

108. The method of claim 106, wherein the molecules are antibodies or binding fragments thereof.

109. The method of claim 106, wherein the molecules are scFvs.

110. The method of claim 106, wherein the diversity of the molecules is 10¹² or higher.

111. The method of claim 106, wherein the diversity of the molecules is 10¹³ or higher.

112. The method of claim 106, wherein the diversity of the molecules is 10¹⁴ or higher.

113. The method of claim 106, wherein the diversity of the molecules is 10¹⁵ or higher.

114. The method of claim 106, wherein the capture agents are antibodies or fragments thereof; and the tags comprise sequences of amino acids to which the antibodies bind.

115. The method of claim 109, wherein the capture agents are antibodies or fragments thereof; and the tags comprise sequences of amino acids to which the antibodies bind.

116. A method for screening samples, comprising:
a) providing the collection of binding sites of claim 106;
b) contacting the collection of binding sites with a sample under conditions whereby components of the sample specifically bind to binding sites of the collection;
c) removing components of the sample which are not bound to the collection of binding sites; and
d) identifying components that are bound to the collection of binding sites.

117. The method of claim 116, wherein steps a) through d) are repeated one or a plurality of times with a sub-set of tagged molecules identified from step d) until diversity of tagged reagents is reduced to a predetermined number.

118. The method of claim 116, wherein the sample is selected from the group consisting of cell lystates, cells, blood, plasma, serum, cerebrospinal fluid, synovial fluid, urine, sweat, tissues, organs, soil, water, viruses, bacteria, fungi algae, protozoa and components thereof.

119. The method of claim 116, wherein capture agents are antibodies; and the pre-selected tagged reagents comprise a polypeptide or plurality thereof to which the antibodies bind.

120. The method of claim 116 that comprises from 3 up to 10⁶ capture agents that specifically bind to different tags.

121. The method of claim 106, wherein the tagged reagents comprise scFvs.

122. The method of claim 106, wherein the tagged reagents comprise T cell receptors.

123. A combination, comprising:
a) an addressable collection of binding sites, comprising:
i) a plurality of capture agents, wherein each capture agent is preselected to specifically bind to a pre-selected tag; and
ii) a plurality of tagged reagents, each comprising one of the pre-selected tags, wherein:
each locus in the collection comprises the same capture agent;
the tagged reagent comprises a biological particle and a tag;
each tag is pre-selected to specifically bind to a capture agent;
each tag is bound to a capture agent thereby forming a complex of the tagged reagent with the capture agent;
each locus comprises a plurality of tagged reagents; and
each of the different molecules at each locus comprises the same pre-selected tag; and
b) one or more of software comprising instructions for pattern recognition and an imager for detecting patterns.

124. The method of claim 117, wherein the predetermined number is about 1 or about 5, or about 10 or about 100, or about 500 or about 1000.

125. The method of claim 115, wherein the identified components are candidate therapeutic compounds, are diagnostic or prognostic of a disease or condition or a target for a therapeutic.


SEQUENCE LISTING (140 sequences)

SEQ ID NO: 1 - DNA, 18 bp, Artificial Sequence
  Primer
  variation 5,6,11,14,17: N is any
gatcnngatcntcngang

SEQ ID NO: 2 - DNA, 18 bp, Artificial Sequence
  Primer
  variation 5,6,11,14,17: N is any
gatcnngatcntcngang

SEQ ID NO: 3 - DNA, 18 bp, Artificial Sequence
  Primer
  variation 5,6,11,14,17: N is any
gatcnngatcntcngang

SEQ ID NO: 4 - DNA, 74 bp, Artificial Sequence
  Primer
  variation 66: N is G or T
  misc_feature 39-42: Shine-Dalgarno sequence (AGGA)
gaattctaatacgactcactatagggttaactttaagaaggagatatacatatgatggtccagctnctcgagtc

SEQ ID NO: 5 - DNA, 53 bp, Artificial Sequence
  Primer
  variation 45: N is G or T
  misc_feature (1)...(17): T7 RNA polymerase promotor
  misc_feature 34-36: Start codon
taatacgactcactatagggaagcttggccaccatggtccagctnctcgagtc

SEQ ID NO: 6 - DNA, 34 bp, Artificial Sequence
  Oligonucleotide: SfilNotIFor
catggcggcccagccggcctaatgagcggccgca

SEQ ID NO: 7 - DNA, 34 bp, Artificial Sequence
  Oligonucleotide: SfilNotIRev
agcttgcggccgctcattaggccggctgggccgc

SEQ ID NO: 8 - DNA, 43 bp, Artificial Sequence
  Oligonucleotide: HAFor
ctagaatatccgtatgatgtgccggattatgcgaatagcgccg

SEQ ID NO: 9 - DNA, 43 bp, Artificial Sequence
  Oligonucleotide: HARev
tcgacggcgctattcgcataatccggcacatcatacggataaa

SEQ ID NO: 10 - DNA, 40 bp, Artificial Sequence
  Oligonucleotide: M2For
ctagaagattataaagatgacgacgataaaaatagcgccg

SEQ ID NO: 11 - DNA, 40 bp, Artificial Sequence
  Oligonucleotide: M2Rev
tcgacggcgctatttttatcgtcgtcatctttataatcaa

SEQ ID NO: 12 - DNA, 23 bp, Artificial Sequence
  Primer: HuVH1aBACK
caggtgcagctggtgcagtctgg

SEQ ID NO: 13 - DNA, 23 bp, Artificial Sequence
  Primer:HuVH2aBACK
cagctcaacttaagggagtctgg

SEQ ID NO: 14 - DNA, 23 bp, Artificial Sequence
  Primer:HuVH3aBACK
gaggtgcagctggtggagtctgg

SEQ ID NO: 15 - DNA, 23 bp, Artificial Sequence
  Primer:HuVH4aBACK
caggtgcagctgcaggagtcggg

SEQ ID NO: 16 - DNA, 23 bp, Artificial Sequence
  Primer:HuVH5aBACK
gaggtgcagctgttgcagtctgc

SEQ ID NO: 17 - DNA, 23 bp, Artificial Sequence
  Primer:HuVH6aBACK
caggtacagctgcagcagtcagg

SEQ ID NO: 18 - DNA, 24 bp, Artificial Sequence
  Primer:HuJH1-2FOR
tgaggagacggtgaccagggtgcc

SEQ ID NO: 19 - DNA, 24 bp, Artificial Sequence
  Primer: HuJH3FOR
tgaagagacggtgaccattgtccc

SEQ ID NO: 20 - DNA, 24 bp, Artificial Sequence
  Primer: HuJH4-5FOR
tgaggagacggtgaccagggttcc

SEQ ID NO: 21 - DNA, 24 bp, Artificial Sequence
  Primer: HuJH6FOR
tgaggagacggtgaccgtggtccc

SEQ ID NO: 22 - DNA, 23 bp, Artificial Sequence
  Primer: HuVkappa1aBACK
gacatccagatgacccagtctcc

SEQ ID NO: 23 - DNA, 23 bp, Artificial Sequence
  Primer: HuVkappa2aBACK
gatgttgtgatgactcagtctcc

SEQ ID NO: 24 - DNA, 23 bp, Artificial Sequence
  Primer: HuVkappa3aBACK
gaaattgtgttgacgcagtctcc

SEQ ID NO: 25 - DNA, 23 bp, Artificial Sequence
  Primer: HuVkappa4aBACK
gacatcgtgatgacccagtctcc

SEQ ID NO: 26 - DNA, 23 bp, Artificial Sequence
  Primer: HuVkappa5aBACK
gaaacgacactcacgcagtctcc

SEQ ID NO: 27 - DNA, 23 bp, Artificial Sequence
  Primer: HuVkappa6aBACK
gaaattgtgctgactcagtctcc

SEQ ID NO: 28 - DNA, 23 bp, Artificial Sequence
  Primer: HuVlambda1BACK
cagtctgtgttgacgcagccgcc

SEQ ID NO: 29 - DNA, 23 bp, Artificial Sequence
  Primer: HuVlambda2BACK
cagtctgccctgactcagcctgc

SEQ ID NO: 30 - DNA, 23 bp, Artificial Sequence
  Primer: HuVlambda3aBACK
tcctatgtgctgactcagccacc

SEQ ID NO: 31 - DNA, 23 bp, Artificial Sequence
  Primer: HuVlambda3bBACK
tcttctgagctgactcaggaccc

SEQ ID NO: 32 - DNA, 23 bp, Artificial Sequence
  Primer: HuVlambda4BACK
cacgttatactgactcaaccgcc

SEQ ID NO: 33 - DNA, 23 bp, Artificial Sequence
  Primer: HuVlambda5BACK
caggctgtgctcactcagccgtc

SEQ ID NO: 34 - DNA, 23 bp, Artificial Sequence
  Primer: HuVlambda6BACK
aattttatgctgactcagcccca

SEQ ID NO: 35 - DNA, 24 bp, Artificial Sequence
  Primer: HuJKappa1FOR
acgtttgatttccaccttggtccc

SEQ ID NO: 36 - DNA, 24 bp, Artificial Sequence
  Primer: HuJKappa2FOR
acgtttgatctccagcttggtccc

SEQ ID NO: 37 - DNA, 24 bp, Artificial Sequence
  Primer: HuJKappa3FOR
acgtttgatatccactttggtccc

SEQ ID NO: 38 - DNA, 24 bp, Artificial Sequence
  Primer: HuJKappa4FOR
acgtttgatctccaccttggtccc

SEQ ID NO: 39 - DNA, 24 bp, Artificial Sequence
  Primer: HuJKappa5FOR
acgtttaatctccagtcgtgtccc

SEQ ID NO: 40 - DNA, 24 bp, Artificial Sequence
  Primer: HuJlambda1FOR
acctaggacggtgaccttggtccc

SEQ ID NO: 41 - DNA, 24 bp, Artificial Sequence
  Primer: HuJlambda2-3FOR
acctaggacggtcagcttggtccc

SEQ ID NO: 42 - DNA, 24 bp, Artificial Sequence
  Primer: HuJlambda4-5FOR
acctaaaacggtgagctgggtccc

SEQ ID NO: 43 - DNA, 28 bp, Artificial Sequence
  Primer: RHuJH1-2
gcaccctggtcaccgtctcctcaggtgg

SEQ ID NO: 44 - DNA, 28 bp, Artificial Sequence
  Primer: RHuJH3
ggacaatggtcaccgtctcttcaggtgg

SEQ ID NO: 45 - DNA, 28 bp, Artificial Sequence
  Primer: RHuJH3
gaaccctggtcaccgtctcctcaggtgg

SEQ ID NO: 46 - DNA, 28 bp, Artificial Sequence
  Primer: RHuJH6
ggaccacggtcaccgtctcctcaggtgg

SEQ ID NO: 47 - DNA, 32 bp, Artificial Sequence
  Primer: RHuVkappa1aBACKFv
ggagactgggtcatctggatgtccgattcgcc

SEQ ID NO: 48 - DNA, 32 bp, Artificial Sequence
  Primer: RHuVkappa2aBACKFv
ggagactgagtcatcacaacatccgatccgcc

SEQ ID NO: 49 - DNA, 32 bp, Artificial Sequence
  Primer: RHuVkappa3aBACKFv
ggagactgcgtcaacacaatttccgatccgcc

SEQ ID NO: 50 - DNA, 32 bp, Artificial Sequence
  Primer: RHuVkappa4aBACKFv
ggagactgggtcatcacgatgtccgatccgcc

SEQ ID NO: 51 - DNA, 32 bp, Artificial Sequence
  Primer: RHuVkappa5aBACKFv
ggagactgcgtgagtgtcgtttccgatccgcc

SEQ ID NO: 52 - DNA, 32 bp, Artificial Sequence
  Primer: RHuVkappa6aBACKFv
ggagactgagtcagcacaatttccgatccgcc

SEQ ID NO: 53 - DNA, 42 bp, Artificial Sequence
  Primer: RHuVlambdaBACK1Fv
ggcggctgcgtcaacacagactgcgatccgccaccgccagag

SEQ ID NO: 54 - DNA, 42 bp, Artificial Sequence
  Primer: RHuVlambdaBACK2Fv
gcaggctgagtcagagcagactgcgatccgccaccgccagag

SEQ ID NO: 55 - DNA, 42 bp, Artificial Sequence
  Primer: RHuVlambdaBACK3aFv
ggtggctgagtcagcacataggacgatccgccaccgccagag

SEQ ID NO: 56 - DNA, 42 bp, Artificial Sequence
  Primer: RHuVlambdaBACK3bFv
gggtcctgagtcagctcagaagacgatccgccaccgccagag

SEQ ID NO: 57 - DNA, 42 bp, Artificial Sequence
  Primer: RHuVlambdaBACK4Fv
ggcggttgagtcagtataacgtgcgatccgccaccgccagag

SEQ ID NO: 58 - DNA, 42 bp, Artificial Sequence
  Primer: RHuVlambdaBACK5Fv
gacggctgagtcagcacagactgcgatccgccaccgccagag

SEQ ID NO: 59 - DNA, 42 bp, Artificial Sequence
  Primer: RHuVlambdaBACK6Fv
tggggctgagtcagcataaaattcgatccgccaccgccagag

SEQ ID NO: 60 - DNA, 56 bp, Artificial Sequence
  Primer: HuVH1aBACKSfi
gtcctcgcaactgcggcccagccggccatggcccaggtgcagctggtgcagtctgg

SEQ ID NO: 61 - DNA, 56 bp, Artificial Sequence
  Primer: HuVH2aBACKSfi
gtcctcgcaactgcggcccagccggccatggcccaggtcaacttaagggagtctgg

SEQ ID NO: 62 - DNA, 56 bp, Artificial Sequence
  Primer:HuVH3aBACKSfi
gtcctcgcaactgcggcccagccggccatggccgaggtgcagctggtggagtctgg

SEQ ID NO: 63 - DNA, 56 bp, Artificial Sequence
  Primer: HuVH4aBACKSfi
gtcctcgcaactgcggcccagccggccatggcccaggtgcagctgcaggagtcggg

SEQ ID NO: 64 - DNA, 56 bp, Artificial Sequence
  Primer: HuVH5aBACKSfi
gtcctcgcaactgcggcccagccggccatggcccaggtgcagctgttgcagtctgc

SEQ ID NO: 65 - DNA, 56 bp, Artificial Sequence
  Primer: HuVH6aBACKSfi
gtcctcgcaactgcggcccagccggccatggcccaggtacagctgcagcagtcagg

SEQ ID NO: 66 - DNA, 48 bp, Artificial Sequence
  Primer: HuJKappa1FORNot
gagtcattctcgacttgcggccgcacgtttgatttccaccttggtccc

SEQ ID NO: 67 - DNA, 48 bp, Artificial Sequence
  Primer: HuJKappa2FORNot
gagtcattctcgacttgcggccgcacgtttgatctccagcttggtccc

SEQ ID NO: 68 - DNA, 48 bp, Artificial Sequence
  Primer: HuJKappa3FORNot
gagtcattctcgacttgcggccgcacgtttgatatccactttggtccc

SEQ ID NO: 69 - DNA, 48 bp, Artificial Sequence
  Primer: HuJKappa4FORNot
gagtcattctcgacttgcggccgcacgtttgatctccaccttggtccc

SEQ ID NO: 70 - DNA, 48 bp, Artificial Sequence
  Primer: HuJKappa5FORNot
gagtcattctcgacttgcggccgcacgtttaatctccagtcgtgtccc

SEQ ID NO: 71 - DNA, 48 bp, Artificial Sequence
  Primer: HuJlambda1FORNot
gagtcattctcgacttgcggccgcacctaggacggtgaccttggtccc

SEQ ID NO: 72 - DNA, 48 bp, Artificial Sequence
  Primer: HuJlambda2-3FORNot
gagtcattctcgacttgcggccgcacctaggacggtcagcttggtccc

SEQ ID NO: 73 - DNA, 48 bp, Artificial Sequence
  Primer: HuJlambda4-5FORNot
gagtcattctcgacttgcggccgcacctaaaacggtgagctgggtccc

SEQ ID NO: 74 - DNA, 43 bp, Artificial Sequence
  Oligonucleotide: HARev2
tcgacggcgctattcgcataatccggcacatcatacggatatt

SEQ ID NO: 75 - DNA, 58 bp, Artificial Sequence
  Oligonucleotide: V5for
ctagaaggtaagcctatccctaaccctctcctcggtctcgattctacgaatagcgccg

SEQ ID NO: 76 - DNA, 58 bp, Artificial Sequence
  Oligonucleotide: V5rev
tcgacggcgctattcgtagaatcgagaccgaggagagggttagggataggcttacctt

SEQ ID NO: 77 - DNA, 58 bp, Artificial Sequence
  Oligonucleotide: StagFor
ctagaaaaagaaaccgctgctgctaaattcgaacgccagcacatggacagcagcgccg

SEQ ID NO: 78 - DNA, 58 bp, Artificial Sequence
  Oligonucleotide: StagRev
tcgacggcgctgctgtccatgtgctggcgttcgaatttagcagcagcggtttcttttt

SEQ ID NO: 79 - DNA, 49 bp, Artificial Sequence
  Oligonucleotide: HSVtagFor
ctagaacagccggaactggcgccggaagatccggaagataatagcgccg

SEQ ID NO: 80 - DNA, 49 bp, Artificial Sequence
  Oligonucleotide: HSVtagRev
tcgacggcgctattatcttccggatcttccggcgccagttccggctgtt

SEQ ID NO: 81 - DNA, 49 bp, Artificial Sequence
  Oligonucleotide: T7tagFor
ctagaaatggctagcatgactggtggacagcaaatgggtaatagcgccg

SEQ ID NO: 82 - DNA, 49 bp, Artificial Sequence
  Oligonucleotide: T7tagRev
tcgacggcgctattacccatttgctgtccaccagtcatgctagccattt

SEQ ID NO: 83 - DNA, 43 bp, Artificial Sequence
  Oligonucleotide: GluGluFor
ctagaagaagaggaggaatatatgccgatggaaaatagcgccg

SEQ ID NO: 84 - DNA, 43 bp, Artificial Sequence
  Oligonucleotide: GluGluRev
tcgacggcgctattttccatcggcatatattcctcctcttctt

SEQ ID NO: 85 - DNA, 49 bp, Artificial Sequence
  Oligonucleotide: KT3For
ctagaaaaaccgccgaccccgccgccggaaccggaaaccaatagcgccg

SEQ ID NO: 86 - DNA, 49 bp, Artificial Sequence
  Oligonucleotide: KT3Rev
tcgacggcgctattggtttccggttccggcggcggggtcggcggttttt

SEQ ID NO: 87 - DNA, 55 bp, Artificial Sequence
  Oligonucleotide: EtagFor
ctagaaggtgcgccggtgccgtatccggatccgctggaaccgcgtaatagcgccg

SEQ ID NO: 88 - DNA, 55 bp, Artificial Sequence
  Oligonucleotide: EtagRev
tcgacggcgctattacgcggttccagcggatccggatacggcaccggcgcacctt

SEQ ID NO: 89 - DNA, 49 bp, Artificial Sequence
  Oligonucleotide: VSVGfor
ctagaatacaccgacatcgaaatgaaccgtctgggtaaaaatagcgccg

SEQ ID NO: 90 - DNA, 49 bp, Artificial Sequence
  Oligonucleotide: VSVGrev
tcgacggcgctatttttacccagacggttcatttcgatgtcggtgtatt

SEQ ID NO: 91 - protein, 10 aa, Artificial Sequence
  Epitope:myc
EQKLISEEDL

SEQ ID NO: 92 - protein, 9 aa, Artificial Sequence
  Epitope:HA
YPYDVPDYA

SEQ ID NO: 93 - protein, 8 aa, Artificial Sequence
  Epitope:FLAG
DYKDDDDK

SEQ ID NO: 94 - protein, 9 aa, Artificial Sequence
  Epitope:GluGlu
EEEEYMPME

SEQ ID NO: 95 - protein, 14 aa, Artificial Sequence
  Epitope:V5
GKPIPNPLLGLDST

SEQ ID NO: 96 - protein, 11 aa, Artificial Sequence
  Epitope:T7
MASMTGGQQMG

SEQ ID NO: 97 - protein, 11 aa, Artificial Sequence
  Epitope:HSV
QPELAPEDPED

SEQ ID NO: 98 - protein, 15 aa, Artificial Sequence
  Epitope:S-tag
KETAAAKFERQHMDS

SEQ ID NO: 99 - protein, 11 aa, Artificial Sequence
  Epitope:KT3
KPPTPPPEPET

SEQ ID NO: 100 - protein, 13 aa, Artificial Sequence
  Epitope:E-tag
GAPVPYPDPLEPR

SEQ ID NO: 101 - protein, 11 aa, Artificial Sequence
  Epitope:VSV-g
YTDIEMNRLGK

SEQ ID NO: 102 - protein, 10 aa, Artificial Sequence
  Consensus sequence for SH3 binding domains
  VARIANT 1, 3, 4, 9: Xaa is any amino acid
XPXXPPPFXP

SEQ ID NO: 103 - protein, 12 aa, Artificial Sequence
  Epitope:AB2
LTPPMGPVIDQR

SEQ ID NO: 104 - protein, 12 aa, Artificial Sequence
  Epitope:AB4
QPQSKGFEPPPP

SEQ ID NO: 105 - DNA, 30 bp, Artificial Sequence
  myc Peptides Gene Sequence
gaacaaaagctaatctcagaggaggaccta

SEQ ID NO: 106 - protein, 12 aa, Artificial Sequence
  B34 Peptide
DLHDERTLQFKL

SEQ ID NO: 107 - protein, 12 aa, Artificial Sequence
  P5D4-A Peptide
HPNLPETRRYAL

SEQ ID NO: 108 - protein, 12 aa, Artificial Sequence
  P5D4-B Peptide
SYTGIEFDRLSN

SEQ ID NO: 109 - protein, 12 aa, Artificial Sequence
  4C10 Peptide
MVDPEAQDVPKW

SEQ ID NO: 110 - protein, 12 aa, Artificial Sequence
  AB2 Peptide
LTPPMGPVIDQR

SEQ ID NO: 111 - protein, 12 aa, Artificial Sequence
  AB4 Peptide
QPQSKGFEPPPP

SEQ ID NO: 112 - protein, 12 aa, Artificial Sequence
  AB3 Peptide
YEYAKGSEPPAL

SEQ ID NO: 113 - protein, 12 aa, Artificial Sequence
  AB6 Peptide
AGTQWCLTRPPC

SEQ ID NO: 114 - protein, 12 aa, Artificial Sequence
  KT3-A Peptide
KLMPNEFFGLLP

SEQ ID NO: 115 - protein, 12 aa, Artificial Sequence
  KT3-B Peptide
KLIPTQLYLLHP

SEQ ID NO: 116 - protein, 12 aa, Artificial Sequence
  KT3-C Peptide
SFMPIEFYARKL

SEQ ID NO: 117 - protein, 12 aa, Artificial Sequence
  7.23 Peptide
TNMEWMTSHRSA

SEQ ID NO: 118 - protein, 9 aa, Artificial Sequence
  S1 Peptide
NANNPDWDF

SEQ ID NO: 119 - protein, 10 aa, Artificial Sequence
  E2 Peptide
SSTSSDFRDR

SEQ ID NO: 120 - protein, 8 aa, Artificial Sequence
  His-tag Peptide
HHHHHHGS

SEQ ID NO: 121 - protein, 6 aa, Artificial Sequence
  AU1 Peptide
DTYRYI

SEQ ID NO: 122 - protein, 6 aa, Artificial Sequence
  AU5 Peptide
TDFYLK

SEQ ID NO: 123 - protein, 5 aa, Artificial Sequence
  IRS Peptide
RYIRS

SEQ ID NO: 124 - protein, 495 aa, Escherichia coli CFT073 [sequence omitted]

SEQ ID NO: 125 - protein, 396 aa, Escherichia coli
  ref <308>: DATABASE ACCESSION NUMBER: Genbank AAC43128
  ref <309>: DATABASE ENTRY DATE: 1993-09-03
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITK

SEQ ID NO: 126 - protein, 318 aa, Mus musculus
  ref <308>: DATABASE ACCESSION NUMBER: Genbank NP_037812
  ref <309>: DATABASE ENTRY DATE: 2002-01-07
MDQNNSLPPYAQGLASPQGAMTPGIPIFSPMMPYGTGLTPQPIQNTNSLSILEEQQRQQQQQQQQQQQQQQQAVATAAASVQQSTSQQPTQGASGQTPQLFHSQTLTTAPLPGTTPLYPSPMTPMTPITPATPASESSGIVPQLQNIVSTVNLGCKLDLKTIALRARNAEYNPKRFAAVIMRIREPRTTALIFSSGKMVCTGAKSEEQSRLAARKYARVVQKLGFPAKFLDFKIQNMVGSCDVKFPIRLEGLVLTHQQFSSYEPELFPGLIYRMIKPRIVLLIFVSGKVVLTGAKVRAEIYEAFENIYPILKGFRKTT

SEQ ID NO: 127 - protein, 105 aa, Mus musculus
  ref <308>: DATABASE ACCESSION NUMBER: Genbank BAA04881
  ref <309>: DATABASE ENTRY DATE: 2002-12-25
MVKLIESKEAFQEALAAAGDKLVVVDFSATWCGPCKMIKPFFHSLCDKYSNVVFLEVDVDDCQDVAADCEVKCMPTFQFYKKGQKVGEFSGANKEKLEASITEYA

SEQ ID NO: 128 - protein, 12 aa, Artificial Sequence
  HOPC1 Peptide
MPQQGDPDWVVP

SEQ ID NO: 129 - DNA, 43 bp, Artificial Sequence
  Ab2For Primer
ctagaattgactcctcctatgggtcctgttattgatcagcggc

SEQ ID NO: 130 - DNA, 43 bp, Artificial Sequence
  Ab2Rev Primer
tcgagccgctgatcaataacaggacccataggaggagtcaatt

SEQ ID NO: 131 - DNA, 43 bp, Artificial Sequence
  Ab4For Primer
ctagaatataatatggaatcgtatctgtggtatttggcgccgc

SEQ ID NO: 132 - DNA, 43 bp, Artificial Sequence
  Ab4Rev Primer
tcgagcggcgccaaataccacagatacgattccatattatatt

SEQ ID NO: 133 - DNA, 43 bp, Artificial Sequence
  B34For Primer
ctagaagatcttcatgatgagcgtactcttcagtttaagcttc

SEQ ID NO: 134 - DNA, 43 bp, Artificial Sequence
  B34Rev Primer
tcgagaagcttaaactgaagagtacgctcatcatgaagatctt

SEQ ID NO: 135 - DNA, 43 bp, Artificial Sequence
  P5D4aFor Primer
ctagaacatccgaatttgcctgagactcgtcgttatgcgctgc

SEQ ID NO: 136 - DNA, 43 bp, Artificial Sequence
  P5D4aRev Primer
tcgagcagcgcataacgacgagtctcaggcaaattcggatgtt

SEQ ID NO: 137 - DNA, 43 bp, Artificial Sequence
  P5D4bFor Primer
ctagaatcttatactgggattgagtttgatcgtttgtcgaatc

SEQ ID NO: 138 - DNA, 43 bp, Artificial Sequence
  P5D4bRev Primer
tcgagattcgacaaacgatcaaactcaatcccagtataagatt

SEQ ID NO: 139 - DNA, 43 bp, Artificial Sequence
  4C10For Primer
ctagaaatggtggatcctgaggcgcaggatgtgccgaagtggc

SEQ ID NO: 140 - DNA, 43 bp, Artificial Sequence
  4C10Rev Primer
tcgagccacttcggcacatcctgcgcctcaggatccaccattt